(19) United States
(12) Patent Application Publication
(10) Pub. No.: US 2013/0310379 A1
(43) Pub. Date: Nov. 21, 2013

(54) MODULATORS OF METHYL MODIFYING ENZYMES, COMPOSITIONS AND USES THEREOF

(71) Applicants: Brian K. Albrecht, Cambridge, MA (US); James Edmund Audia, Zionsville, IN (US); Alexandre Gagnon, Beaconsfield (CA); Jean-Christophe Harmange, Andover, MA (US); Christopher G. Naveschuk, Stoneham, MA (US)

(72) Inventors: Brian K. Albrecht, Cambridge, MA (US); James Edmund Audia, Zionsville, IN (US); Alexandre Gagnon, Beaconsfield (CA); Jean-Christophe Harmange, Andover, MA (US); Christopher G. Naveschuk, Stoneham, MA (US)

(73) Assignee: CONSTELLATION PHARMACEUTICALS, Cambridge, MA (US)

(21) Appl. No.: 13/988,180

(22) PCT Filed: Nov. 21, 2011
(86) PCT No.: PCT/US11/61740

§ 371 Date: Aug. 5, 2013

Related U.S. Application Data
(60) Provisional application No. 61/415,713, filed on Nov. 19, 2010.

(51) Int. Cl. C07D 211/58; C07D 451/04; C07D 471/04; C07D 207/14; C07D 401/12; C07D 405/12

(52) U.S. Cl. CPC C07D 211/58; C07D 401/12; C07D 405/12; C07D 471/04; C07D 207/14; C07D 451/04
USPC 514/235.5; 544/238; 514/252.03; 546/224; 514/329; 544/335; 514/256; 514/252.01; 546/194; 514/318; 544/312; 514/274; 544/408; 514/255.05; 546/223; 546/201; 514/323; 544/130; 546/113; 514/300; 548/557; 514/426; 546/124; 514/304

(57) ABSTRACT

Agents for modulating methyl modifying enzymes, compositions and uses thereof are provided herein.

### PRIORITY

The present application claims priority to U.S. Provisional Application No. 61/415,713, filed Nov. 19, 2011, the entire contents of which are hereby incorporated herein by reference.

### BACKGROUND OF THE INVENTION

Eukaryotic chromatin is composed of macromolecular complexes called nucleosomes. A nucleosome has 147 base pairs of DNA wrapped around a protein octamer having two subunits of each of histone protein H2A, H2B, H3, and H4. Histone proteins are subject to post-translational modifications which in turn affect chromatin structure and gene expression. One type of post-translational modification found on histones is methylation of lysine and arginine residues. Histone methylation plays a critical role in the regulation of gene expression in eukaryotes. Methylation affects chromatin structure and has been linked to both activation and repression of transcription (Zhang and Reinberg, Genes Dev. 15:2343-2360, 2001). Enzymes that catalyze attachment and removal of methyl groups from histones are implicated in gene silencing, embryonic development, cell proliferation, and other processes.

### SUMMARY OF THE INVENTION

The present disclosure encompasses the recognition that methyl modifying enzymes are an attractive target for modulation, given their role in the regulation of diverse biological processes. It has now been found that compounds of this invention, and pharmaceutically acceptable compositions thereof, are effective as agents that stimulate activity of histone methyl modifying enzymes, including histone methylases and histone demethylases. Such compounds have the general formula I:

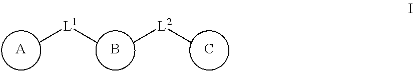

or a pharmaceutically acceptable salt thereof, wherein Ring A, Ring B, Ring C, L¹ and L² are as defined herein.

Compounds of the present invention, and pharmaceutically acceptable compositions thereof, are useful for treating a variety of diseases, disorders or conditions, associated with a methyl modifying enzyme. Such diseases, disorders, or conditions include those described herein.

Compounds provided by this invention are also useful for the study of methyl modifying enzymes in biological and pathological phenomena; the study of intracellular signal transduction pathways mediated by methyl modifying enzymes and the comparative evaluation of new methyl modifying enzyme modulators.

### DETAILED DESCRIPTION OF CERTAIN EMBODIMENTS

### 1. General Description of Compounds of the Invention

In certain embodiments, the present invention provides a compound of formula I:

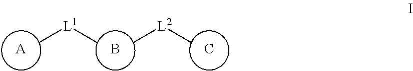

or a pharmaceutically acceptable salt thereof, wherein:
Ring A is an optionally substituted group selected from a 5-6 membered monocyclic heteroaryl ring having 1-3 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, an 8-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
Ring B is an optionally substituted bivalent ring selected from phenylene, a 4-7 membered saturated or partially unsaturated heterocyclic ring having 1-2 heteroatoms independently selected from nitrogen, oxygen, or sulfur, 5-6 membered monocyclic heteroarylene ring having 1-3 heteroatoms independently selected from nitrogen, oxygen, or sulfur; an 8-10 membered bicyclic aryl carbocyclic ring, an 8-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
Ring C is an optionally substituted group selected from phenyl, a 3-7 membered saturated or partially unsaturated carbocyclic ring, an 8-10 membered bicyclic aryl carbocyclic ring, a 4-7 membered saturated or partially unsaturated heterocyclic ring having 1-2 heteroatoms independently selected from nitrogen, oxygen, or sulfur, a 5-6 membered monocyclic heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
each of L¹ and L² is independently a covalent bond or an optionally substituted bivalent C₁₋₆ hydrocarbon chain, wherein one or more methylene units of L¹ or L² are optionally and independently replaced by -Cy-, —O—, —S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —OC(O)—, or —C(O)O—;
each R′ is independently —R, —C(O)R, —CO₂R, or —SO₂R, or:
   two R′ on the same nitrogen are taken together with their intervening atoms to form a 4-7 membered heterocyclic ring having 1-2 heteroatoms independently selected from nitrogen, oxygen, and sulfur;
each R is hydrogen, or an optionally substituted group selected from C₁₋₆ aliphatic, phenyl, a 3-7 membered saturated or partially unsaturated carbocyclic ring, an 8-10 membered bicyclic saturated, partially unsaturated or aryl carbocyclic ring, a 5-6 membered monocyclic heteroaryl ring having 1-3 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, an 8-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur; and
-Cy- is an optionally substituted bivalent ring selected from phenylene, a 3-7 membered saturated or partially unsaturated carbocyclylene, a 4-7 membered saturated or partially unsaturated heterocyclylene having 1-2 heteroatoms independently selected from nitrogen, oxygen, or sulfur, or a 5-6 membered heteroarylene having 1-3 heteroatoms independently selected from nitrogen, oxygen, or sulfur.

### 2. Compounds and Definitions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw-Hill, N.Y., 1962); Wilen, S. H. Tables of Resolving Agents and Optical Resolutions p. 268 (E. L. Eliel, Ed., Univ. of Notre Dame Press, Notre Dame, Ind. 1972).

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

As used herein a “direct bond” or “covalent bond” refers to a single, double or triple bond. In certain embodiments, a “direct bond” or “covalent bond” refers to a single bond.

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-6 carbon atoms. In some embodiments, aliphatic groups contain 1-4 carbon atoms, and in yet other embodiments aliphatic groups contain 1-3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “alkynylene” refers to a bivalent alkynyl group.

The term “alkyl,” as used herein, refers to a monovalent saturated, straight- or branched-chain hydrocarbon radical derived from an aliphatic moiety containing between one and six carbon atoms by removal of a single hydrogen atom. In some embodiments, alkyl contains 1-5 carbon atoms. In another embodiment, alkyl contains 1-4 carbon atoms. In still other embodiments, alkyl contains 1-3 carbon atoms. In yet another embodiment, alkyl contains 1-2 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, and the like.

The term “alkenyl,” as used herein, denotes a monovalent group derived from a straight- or branched-chain aliphatic moiety having at least one carbon-carbon double bond by the removal of a single hydrogen atom. In certain embodiments, alkenyl contains 2-6 carbon atoms. In certain embodiments, alkenyl contains 2-5 carbon atoms. In some embodiments, alkenyl contains 2-4 carbon atoms. In another embodiment, alkenyl contains 2-3 carbon atoms. Alkenyl groups include, for example, ethenyl (“vinyl”), propenyl (“allyl”), butenyl, 1-methyl-2-buten-1-yl, and the like.

The term “alkynyl,” as used herein, refers to a monovalent group derived from a straight- or branched-chain aliphatic moiety having at least one carbon-carbon triple bond by the removal of a single hydrogen atom. In certain embodiments, alkynyl contains 2-6 carbon atoms. In certain embodiments, alkynyl contains 2-5 carbon atoms. In some embodiments, alkynyl contains 2-4 carbon atoms. In another embodiment, alkynyl contains 2-3 carbon atoms. Representative alkynyl groups include, but are not limited to, ethynyl, 2-propynyl (“propargyl”), 1-propynyl, and the like.

The term “aryl” used alone or as part of a larger moiety as in “aralkyl”, “aralkoxy”, or “aryloxyalkyl”, refers to monocyclic and bicyclic ring systems having a total of five to 10 ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenantriidinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted. The term “heteroarylene” refers to a bivalent mono- or bicyclic heteroaryl ring.

As used herein, the terms “heterocycle”, “heterocyclyl”, “heterocyclic radical”, and “heterocyclic ring” are used interchangeably and refer to a stable 4- to 7-membered monocyclic or 7-10-membered bicyclic heterocyclic moiety that is either saturated or partially unsaturated, and having, in addition to carbon atoms, one or more, preferably one to four, heteroatoms, as defined above. In certain embodiments, a “heterocycle”, group is a 1,1′-heterocyclylene group (i.e., a spiro-fused ring). When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, 2-azabicyclo[2.2.1]heptanyl, octahydroindolyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond between ring atoms but is not aromatic. The term “partially unsaturated” is intended to encompass rings having multiple sites of unsaturation, but is not intended to include aryl or heteroaryl moieties, as herein defined.

As used herein, the terms “carbocyclylene” or “cycloalkylene” are used interchangeably and refer to a bivalent carbocyclyl or cycloalkyl group. In certain embodiments, a carbocyclylene or cycloalkylene group is a 1,1-cycloalkylene group (i.e., a spiro-fused ring). Exemplary 1,1-cycloalkylene groups include

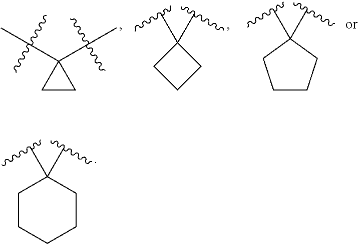 [SMILES: *C1(*)CC1 ; *C1(*)CCC1 ; *C1(*)CCCC1 ; *C1(*)CCCCC1]

In other embodiments, a cycloalkylene group is a 1,2-cycloalkylene group or a 1,3-cycloalkylene group. Exemplary 1,2-cycloalkylene groups include

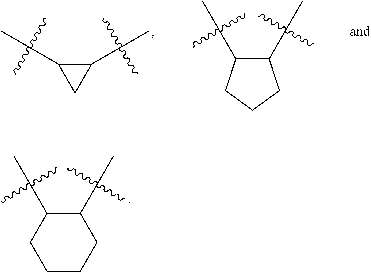 [SMILES: *C1CC1* ; *C1CCCC1* ; *C1CCCCC1*]

Exemplary 1,3-cycloalkylene groups include

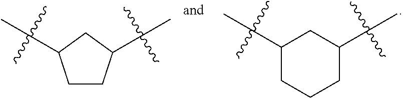 [SMILES: *C1CCC(*)C1 ; *C1CCCC(*)C1]

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR^{◯}, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SRO, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; —SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “inhibitor” is defined as a compound that binds to and/or inhibits a target S-adenosylmethionine (SAM) utilizing enzyme with measurable affinity. In certain embodiments, an inhibitor has an IC₅₀ and/or binding constant of less about 50 μM, less than about 1 μM, less than about 500 nM, less than about 100 nM, or less than about 10 nM.

The terms “measurable affinity” and “measurably inhibit,” as used herein, means a measurable change in activity of at least one SAM utilizing enzyme between a sample comprising a provided compound, or composition thereof, and at least one SAM dependent enzyme, and an equivalent sample comprising at least one SAM dependent enzyme, in the absence of said compound, or composition thereof.

### 3. Description of Exemplary Compounds

In certain embodiments, the present invention provides a compound of formula I:

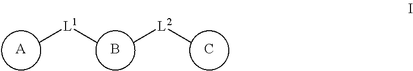

or a pharmaceutically acceptable salt thereof, wherein each of Ring A, Ring B, Ring C, L¹ and L² is as defined above and described herein.

As defined generally above, Ring A is an optionally substituted group selected from a 5-6 membered monocyclic heteroaryl ring having 1-3 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, an 8-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur.

In some embodiments, Ring A is an optionally substituted 5-membered monocyclic heteroaryl ring having 1-3 heteroatoms independently selected from nitrogen, oxygen, or sulfur. In some embodiments, Ring A is an optionally substituted 5-membered monocyclic heteroaryl ring having 1 heteroatom selected from nitrogen, oxygen, or sulfur. In some embodiments, Ring A is selected from pyrrolyl, furanyl, or thiophenyl.

In some embodiments, Ring A is an optionally substituted 5-membered heteroaryl ring having 2 heteroatoms selected from nitrogen, oxygen, or sulfur. In certain embodiments, Ring A is an optionally substituted 5-membered heteroaryl ring having 1 nitrogen atom, and an additional heteroatom selected from sulfur or oxygen. Exemplary Ring A groups include optionally substituted pyrazolyl, imidazolyl, thiazolyl, isothiazolyl, oxazolyl or isoxazolyl.

In some embodiments, Ring A is an optionally substituted 5-membered heteroaryl ring having 3 heteroatoms selected from nitrogen, oxygen or sulfur. In certain embodiments, Ring A is an optionally substituted 5-membered heteroaryl ring having 1 nitrogen atom, and two additional heteroatoms selected from sulfur or oxygen. In other embodiments, Ring A is an optionally substituted 5-membered heteroaryl ring having 2 nitrogen atoms, and an additional heteroatom selected from sulfur or oxygen. Exemplary Ring A groups include optionally substituted triazolyl, thiadiazolyl, oxadiazolyl.

In some embodiments, Ring A is a 6-membered heteroaryl ring having 1-3 nitrogens. In other embodiments, Ring A is an optionally substituted 6-membered heteroaryl ring having 1-2 nitrogens. In some embodiments, Ring A is an optionally substituted 6-membered heteroaryl ring having 2 nitrogens. In certain embodiments, Ring A is an optionally substituted 6-membered heteroaryl ring having 1 nitrogen. Exemplary Ring A groups include optionally substituted pyridinyl, pyrimidinyl, pyrazinyl, pyridazinyl, triazinyl, or tetrazinyl.

In certain embodiments, Ring A is a 4-7 membered saturated or partially unsaturated heterocyclic ring having 1-2 heteroatoms independently selected from nitrogen, oxygen, or sulfur. In certain embodiments, Ring A is oxiranylene, oxetanylene, tetrahydrofuranylene, tetrahydropyranylene, oxepaneylene, aziridineylene, azetidineylene, pyrrolidinylene, piperidinylene, azepanylene, thiiranylene, thietanylene, tetrahydrothiophenylene, tetrahydrothiopyranylene, thiepanylene, dioxolanylene, oxathiolanylene, oxazolidinylene, imidazolidinylene, thiazolidinylene, dithiolanylene, dioxanylene, morpholinylene, oxathianylene, piperazinylene, thiomorpholinylene, dithianylene, dioxepanylene, oxazepanylene, oxathiepanylene, dithiepanylene, diazepanylene, dihydrofuranonylene, tetrahydropyranonylene, oxepanonylene, pyrrolidinonylene, piperidinonylene, azepanonylene, dihydrothiophenonylene, tetrahydrothiopyranonylene, thiepanonylene, oxazolidinonylene, oxazinanonylene, oxazepanonylene, dioxolanonylene, dioxanonylene, dioxepanonylene, oxathiolinonylene, oxathianonylene, oxathiepanonylene, thiazolidinonylene, thiazinanonylene, thiazepanonylene, imidazolidinonylene, tetrahydropyrimidinonylene, diazepanonylene, imidazolidinedionylene, oxazolidinedionylene, thiazolidinedionylene, dioxolanedionylene, oxathiolanedionylene, piperazinedionylene, morpholinedionylene, thiomorpholinedionylene, tetrahydropyranylene, tetrahydrofuranylene, morpholinylene, thiomorpholinylene, piperidinylene, piperazinylene, pyrrolidinylene, tetrahydrothiophenylene, or tetrahydrothiopyranylene.

In certain embodiments, Ring A is an optionally substituted 5-6 membered partially unsaturated monocyclic ring having 1-2 heteroatoms independently selected from nitrogen, oxygen, or sulfur. In certain embodiments, Ring A is an optionally substituted tetrahydropyridinyl, dihydrothiazolyl, dihydrooxazolyl, or oxazolinyl group. In some embodiments, Ring A is an optionally substituted 8-10 membered partially unsaturated bicyclic ring having 1-3 heteroatoms independently selected from nitrogen, oxygen, or sulfur. In some embodiments, Ring A is an optionally substituted indoline. In some embodiments, Ring A is an optionally substituted isoindoline.

In certain embodiments, Ring A is an optionally substituted 7-10 membered bicyclic heteroaryl ring having 1-4 heteroatoms independently selected from nitrogen, oxygen, or sulfur. In some embodiments, Ring A is an optionally substituted 5,6-fused heteroaryl ring having 1-4 heteroatoms independently selected from nitrogen, oxygen, or sulfur. In other embodiments, Ring A is an optionally substituted 5,6-fused heteroaryl ring having 1-2 heteroatoms independently selected from nitrogen, oxygen, or sulfur. In certain embodiments, Ring A is an optionally substituted 5,6-fused heteroaryl ring having 1 heteroatom independently selected from nitrogen, oxygen, or sulfur. In some embodiments, Ring A is an optionally substituted indolyl. In some embodiments, Ring A is an optionally substituted azabicyclo[3.2.1]octanyl. In certain embodiments, Ring A is an optionally substituted 5,6-fused heteroaryl ring having 2 heteroatoms independently selected from nitrogen, oxygen, or sulfur. In some embodiments, Ring A is an optionally substituted azaindolyl. In some embodiments, Ring A is an optionally substituted benzimidazolyl. In some embodiments, Ring A is an optionally substituted benzothiazolyl. In some embodiments, Ring A is an optionally substituted benzoxazolyl. In some embodiments, Ring A is an optionally substituted indazolyl. In certain embodiments, Ring A is an optionally substituted 5,6-fused heteroaryl ring having 3 heteroatoms independently selected from nitrogen, oxygen, or sulfur.

In certain embodiments, Ring A is an optionally substituted 6,6-fused heteroaryl ring having 1-4 heteroatoms independently selected from nitrogen, oxygen, or sulfur. In some embodiments, Ring A is an optionally substituted 6,6-fused heteroaryl ring having 1-2 heteroatoms independently selected from nitrogen, oxygen, or sulfur. In other embodiments, Ring A is an optionally substituted 6,6-fused heteroaryl ring having 1 heteroatom independently selected from nitrogen, oxygen, or sulfur. In some embodiments, Ring A is an optionally substituted quinolinyl. In some embodiments, Ring A is an optionally substituted isoquinolinyl. According to one aspect, Ring A is an optionally substituted 6,6-fused heteroaryl ring having 2 heteroatoms independently selected from nitrogen, oxygen, or sulfur. In some embodiments, Ring A is a quinazoline or a quinoxaline.

As defined generally above, Ring B is an optionally substituted bivalent ring selected from phenylene, a 4-7 membered saturated or partially unsaturated heterocyclic ring having 1-2 heteroatoms independently selected from nitrogen, oxygen, or sulfur, 5-6 membered monocyclic heteroarylene ring having 1-3 heteroatoms independently selected from nitrogen, oxygen, or sulfur; an 8-10 membered bicyclic aryl carbocyclic ring, an 8-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur.

In some embodiments, Ring B is optionally substituted phenylene. In some embodiments, Ring B is optionally substituted

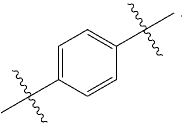 [SMILES: *c1ccc(*)cc1]

In some embodiments, Ring B is optionally substituted

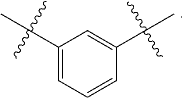 [SMILES: *c1cccc(*)c1]

In some embodiments, Ring B is optionally substituted

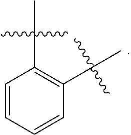 [SMILES: *c1ccccc1*]

In some embodiments, Ring B is a 4-7 membered saturated or partially unsaturated heterocyclic ring having 1-2 heteroatoms independently selected from nitrogen, oxygen, or sulfur. In some embodiments, Ring B is a 4-7 membered saturated heterocyclic ring having 1-2 heteroatoms independently selected from nitrogen, oxygen, or sulfur. In some embodiments, Ring B is a 4-membered saturated heterocyclic ring having 1 heteroatom selected from nitrogen, oxygen, or sulfur. In some embodiments, Ring B is a 5-membered saturated heterocyclic ring having 1-2 heteroatoms independently selected from nitrogen, oxygen, or sulfur. In some embodiments, Ring B is a 6-membered saturated heterocyclic ring having 1-2 heteroatoms independently selected from nitrogen, oxygen, or sulfur. Exemplary Ring B groups include azetidinylene, pyrrolidinylene, tetrahydrofuranylene, piperidinylene, piperazinylene and morpholinylene.

In some embodiments, Ring B is a 5-6 membered heteroarylene ring having 1-3 heteroatoms independently selected from oxygen, nitrogen and sulfur. In some embodiments, Ring B is a 5-membered heteroarylene ring having 1-3 heteroatoms independently selected from oxygen, nitrogen and sulfur. In some embodiments, Ring B is a 5-membered heteroarylene ring having 1-3 heteroatoms independently selected from oxygen, nitrogen and sulfur. In some embodiments, Ring B is a 5-membered heteroarylene ring having 1-2 heteroatoms independently selected from oxygen, nitrogen and sulfur. In some embodiments, Ring B is a 5-membered heteroarylene ring having 2 heteroatoms independently selected from oxygen, nitrogen and sulfur. In some embodiments, Ring B is a 5-membered heteroarylene ring having 1 heteroatom selected from oxygen, nitrogen and sulfur. Exemplary Ring B groups include pyrrolylene, furanylene, thiophenylene, oxazolylene, imidazolylene, pyrazolylene, oxadiazolylene, triazolylene, tetrazolylene, thiazolylyene and thiadiazolylene.

In some embodiments, Ring B is a 6-membered heteroarylene ring having 1-3 nitrogens. In other embodiments, Ring B is an optionally substituted 6-membered heteroarylene ring having 1-2 nitrogens. In some embodiments, Ring B is an optionally substituted 6-membered heteroarylene ring having 2 nitrogens. In certain embodiments, Ring B is an optionally substituted 6-membered heteroarylene ring having 1 nitrogen. Exemplary Ring B groups include optionally substituted pyridinyl, pyrimidinyl, pyrazinyl, pyridazinyl, triazinyl, or tetrazinyl.

In some embodiments, Ring B is an 8-10 membered bicyclic aryl carbocyclic ring. In some embodiments, Ring B is naphthalene.

In some embodiments, Ring B is an 8-10 membered bicyclic heteroaryl ring having 1-4 heteroatoms independently selected from nitrogen, oxygen or sulfur. In some embodiments, Ring B is a 9-membered bicyclic heteroaryl ring having 1-4 heteroatoms independently selected from nitrogen, oxygen or sulfur. Ring B is a 10-membered bicyclic heteroaryl ring having 1-4 heteroatoms independently selected from nitrogen, oxygen or sulfur. Exemplary Ring B groups include indole, azaindole, quinoline, isoquinoline, and pyrrolopyrimidine.

As defined generally above, Ring C is an optionally substituted group selected from phenyl, a 3-7 membered saturated or partially unsaturated carbocyclic ring, a 4-7 membered saturated or partially unsaturated heterocyclic ring having 1-2 heteroatoms independently selected from nitrogen, oxygen, or sulfur, a 8-10 membered bicyclic saturated, partially unsaturated or aryl carbocyclic ring, a 5-6 membered monocyclic heteroaryl ring having 1-3 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 8-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur.

In some embodiments, Ring C is optionally substituted phenyl.

In some embodiments, Ring C is optionally substituted 3-7 membered saturated or partially unsaturated carbocyclic ring. In some embodiments, Ring C is optionally substituted 3-7 membered saturated carbocyclic ring. In some embodiments, Ring C is optionally substituted 3-7 membered partially unsaturated carbocyclic ring. Exemplary Ring C groups include cyclopropyl, cyclobutyl, cyclopentyl, cyclohexyl, cycloheptyl, cyclobutenyl, cyclopentenyl, cyclohexenyl and cycloheptenyl.

In some embodiments, Ring C is an optionally substituted 4-7 membered saturated heterocyclic ring having 1-2 heteroatoms independently selected from nitrogen, oxygen, or sulfur. In some embodiments, Ring C is an optionally substituted 4-7 membered saturated heterocyclic ring having 2 heteroatoms independently selected from nitrogen, oxygen, or sulfur. In some embodiments, Ring C is an optionally substituted 4-7 membered saturated heterocyclic ring having 1 heteroatom selected from nitrogen, oxygen, or sulfur.

In some embodiments, Ring C is an optionally substituted 4-7 membered partially unsaturated heterocyclic ring having 1-2 heteroatoms independently selected from nitrogen, oxygen or sulfur. In some embodiments, Ring C is an optionally substituted 4-7 membered partially unsaturated heterocyclic ring having 2 heteroatoms independently selected from nitrogen, oxygen or sulfur. In some embodiments, Ring C is an optionally substituted 4-7 membered partially unsaturated heterocyclic ring having 1 heteroatom selected from nitrogen, oxygen or sulfur. Exemplary Ring C groups include aziridinyl, oxiranyl, thiiranyl, azetidinyl, oxetanyl, thietanyl, pyrrolidinyl, furanyl, imidazolidinyl, pyrazolidinyl, oxazolidinyl, isoxazolidinyl, thiazolidinyl, isothiazolidinyl, dioxolanyl, oxathiolanyl, dithiolanyl, piperidinyl, tetrahydropyranyl, thianyl, pyranyl, thiopyranyl, piperazinyl, morpholinyl, dithianyl, and dioxanyl.

In some embodiments, Ring C is an optionally substituted 5-6 membered monocyclic heteroaryl ring having 1-3 heteroatoms independently selected from nitrogen, oxygen, or sulfur. In some embodiments, Ring C is an optionally substituted 5-membered monocyclic heteroaryl ring having 1-3 heteroatoms independently selected from nitrogen, oxygen, or sulfur. In some embodiments, Ring C is an optionally substituted 5-membered monocyclic heteroaryl ring having 1 heteroatom selected from nitrogen, oxygen, or sulfur. In some embodiments, Ring C is selected from pyrrolyl, furanyl, thiophenyl or pyridinyl.

In some embodiments, Ring C is an optionally substituted 5-membered heteroaryl ring having 2 heteroatoms selected from nitrogen, oxygen, or sulfur. In certain embodiments, Ring C is an optionally substituted 5-membered heteroaryl ring having 1 nitrogen atom, and an additional heteroatom selected from sulfur or oxygen. Exemplary Ring C groups include optionally substituted pyrazolyl, imidazolyl, tetrazolyl, thiazolyl, isothiazolyl, oxazolyl or isoxazolyl.

In some embodiments, Ring C is an optionally substituted 5-membered heteroaryl ring having 3 heteroatoms selected from nitrogen, oxygen or sulfur. In certain embodiments, Ring C is an optionally substituted 5-membered heteroaryl ring having 1 nitrogen atom, and two additional heteroatoms selected from sulfur or oxygen. In other embodiments, Ring C is an optionally substituted 5-membered heteroaryl ring having 2 nitrogen atoms, and an additional heteroatom selected from sulfur or oxygen. Exemplary Ring C groups include optionally substituted triazolyl, thiadiazolyl, oxadiazolyl.

In some embodiments, Ring C is a 6-membered heteroaryl ring having 1-3 nitrogens. In other embodiments, Ring C is an optionally substituted 6-membered heteroaryl ring having 1-2 nitrogens. In some embodiments, Ring C is an optionally substituted 6-membered heteroaryl ring having 2 nitrogens. In certain embodiments, Ring C is an optionally substituted 6-membered heteroaryl ring having 1 nitrogen. Exemplary Ring C groups include optionally substituted pyridinyl, pyrimidinyl, pyrazinyl, pyridazinyl, triazinyl, or tetrazinyl.

In certain embodiments, Ring C is an optionally substituted 7-10 membered bicyclic heteroaryl ring having 1-4 heteroatoms independently selected from nitrogen, oxygen, or sulfur. In some embodiments, Ring C is an optionally substituted 5,6-fused heteroaryl ring having 1-4 heteroatoms independently selected from nitrogen, oxygen, or sulfur. In other embodiments, Ring C is an optionally substituted 5,6-fused heteroaryl ring having 1-2 heteroatoms independently selected from nitrogen, oxygen, or sulfur. In certain embodiments, Ring C is an optionally substituted 5,6-fused heteroaryl ring having 1 heteroatom independently selected from nitrogen, oxygen, or sulfur. In some embodiments, Ring C is an optionally substituted indole. In certain embodiments, Ring C is an optionally substituted 5,6-fused heteroaryl ring having 2 heteroatoms independently selected from nitrogen, oxygen, or sulfur. In certain embodiments, Ring C is an optionally substituted azaindole. In some embodiments, Ring C is an optionally substituted benzimidazole. In some embodiments, Ring C is an optionally substituted benzothiazole. In some embodiments, Ring C is an optionally substituted benzoxazole. In some embodiments, Ring C is an optionally substituted indazole. In certain embodiments, Ring C is an optionally substituted 5,6-fused heteroaryl ring having 3 heteroatoms independently selected from nitrogen, oxygen, or sulfur.

In certain embodiments, Ring C is an optionally substituted 6,6-fused heteroaryl ring having 1-4 heteroatoms independently selected from nitrogen, oxygen, or sulfur. In some embodiments, Ring C is an optionally substituted 6,6-fused heteroaryl ring having 1-2 heteroatoms independently selected from nitrogen, oxygen, or sulfur. In other embodiments, Ring C is an optionally substituted 6,6-fused heteroaryl ring having 1 heteroatom independently selected from nitrogen, oxygen, or sulfur. In some embodiments, Ring C is an optionally substituted quinoline. In some embodiments, Ring C is an optionally substituted isoquinoline. According to one aspect, Ring C is an optionally substituted 6,6-fused heteroaryl ring having 2 heteroatoms independently selected from nitrogen, oxygen, or sulfur. In some embodiments, Ring C is a quinazoline or a quinoxaline.

As defined generally above, L¹ is independently a covalent bond or an optionally substituted bivalent C₁₋₆ hydrocarbon chain, wherein one or more methylene units of L¹ are optionally and independently replaced by -Cy-, —O—, —S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —OC(O)—, or —C(O)O—; wherein -Cy-, R and R′ are as defined above and described herein.

In some embodiments, L¹ is a covalent bond. In some embodiments, L¹ is an optionally substituted bivalent C₁₋₆ hydrocarbon. In some such embodiments, L¹ is an optionally substituted bivalent C₁₋₄ hydrocarbon. In some embodiments, L¹ is an optionally substituted bivalent C₁₋₂ hydrocarbon. In some embodiments, L¹ is —CH₂—. In some embodiments, L¹ is —CH₂CH₂—. In certain embodiments, L¹ is —CH(CH₃)—. In some embodiments, L¹ is —CH(CH₂CH₃)—. In some embodiments, L¹ is —CH₂C(O)—. In some embodiments, L¹ is —C(O)CH₂—. In some embodiments, L¹ is —OC(O)—. In some embodiments, L¹ is —C(O)O—. In some embodiments, L¹ is —N(R′)C(O)—. In some embodiments, L¹ is —C(O)N(R′)—. In some embodiments, L¹ is —C(O)N(H)—. In some embodiments, L¹ is —N(H)C(O)—. In some embodiments, L¹ is —C(O)N(CH₃)—. In some embodiments, L¹ is —N(CH₃)C(O)—. In some embodiments, L¹ is —S(O)₂N(R′)—. In some embodiments, L¹ is —N(R′)S(O)₂—. In some embodiments, L¹ is —N(R′)CH₂—. In some embodiments, L¹ is —O—. In some embodiments, L¹ is —N(R′)—. In some embodiments, L¹ is —N(CH₃)—. In some embodiments, L¹ is —N(H)—. In some embodiments, L¹ is —S—. In some embodiments, L¹ is —CH₂O—. In some embodiments, L¹ is —CH₂N(R′)—. In some embodiments, L¹ is —CH₂N(CH₃)—. In some embodiments, L¹ is —CH₂N(H)—. In some embodiments, L¹ is —CH₂S—. In some embodiments, L¹ is —OCH₂—. In some embodiments, L¹ is —N(CH₃)CH₂—. In some embodiments, L¹ is —N(H)CH₂—. In some embodiments, L¹ is —SCH₂—. In some embodiments, L¹ is —CH₂CH₂O—. In some embodiments, L¹ is —CH₂OCH₂—. In some embodiments, L¹ is —OCH₂CH₂—. In some embodiments, L¹ is —CH(CH₃)O—.

In some embodiments, L¹ is optionally substituted C₂₋₆ hydrocarbon, wherein at least one carbon-carbon bond is unsaturated. In some embodiments, L¹ is optionally substituted C₂ hydrocarbon, wherein the carbon-carbon bond is unsaturated. In some such embodiments, L¹ is optionally substituted ethenylene or ethynylene. In some embodiments, L¹ is optionally substituted C₃ hydrocarbon, wherein at least one carbon-carbon bond is unsaturated. In some such embodiments, L¹ is optionally substituted propenylene, also known as allylene, or propynylene. In some embodiments, L¹ is optionally substituted C₄ hydrocarbon, wherein at least one carbon-carbon bond is unsaturated. In some such embodiments, L¹ is optionally substituted butenylene, 2-methyl-propenylene, 1,3-butadienylene or butynylene. In some embodiments, L¹ is optionally substituted C₅ hydrocarbon, wherein at least one carbon-carbon bond is unsaturated. In some such embodiments, L¹ is optionally substituted pentenylene, isoamylenyl or pentynylene. In some embodiments, L¹ is optionally substituted C₆ hydrocarbon, wherein at least one carbon-carbon bond is unsaturated. In some such embodiments, L¹ is optionally substituted hexenylene or hexynylene.

As defined generally above, L² is a covalent bond or an optionally substituted bivalent C₁₋₆ hydrocarbon chain, wherein one or more methylene units of L² are optionally and independently replaced by -Cy-, —O—, —S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —OC(O)—, or —C(O)O—; wherein -Cy-, R and R′ are as defined above and described herein.

In some embodiments, L² is a covalent bond. In some embodiments, L² is optionally substituted bivalent C₁₋₆ hydrocarbon. In some such embodiments, L² is an optionally substituted bivalent C₁₋₄ hydrocarbon. In some embodiments, L² is an optionally substituted bivalent C₁₋₂ hydrocarbon. In some embodiments, L² is —CH₂—. In some embodiments, L² is —O—. In some embodiments, L² is —N(R′)—. In some embodiments, L² is —N(CH₃)—. In some embodiments, L² is —N(H)—. In some embodiments, L² is —S—. In some embodiments, L² is —CH₂O—. In some embodiments, L² is —CH₂N(R′)—. In some embodiments, L² is —CH₂N(CH₃)—. In some embodiments, L² is —CH₂N(H)—. In some embodiments, L² is —CH₂S—. In some embodiments, L² is —OCH₂—. In some embodiments, L² is —N(R′)CH₂—. In some embodiments, L² is —N(CH₃)CH₂—. In some embodiments, L² is —N(H)CH₂—. In some embodiments, L² is —SCH₂—. In some embodiments, L² is —CH₂CH₂O—. In some embodiments, L² is —CH₂OCH₂—. In some embodiments, L² is —OCH₂CH₂—. In some embodiments, L² is —CH(CH₃)O—. In some embodiments, L² is —C(O)O—. In some embodiments, L² is —OC(O)—. In some embodiments, L² is —C(O)N(R′)—. In some embodiments, L² is —N(R′)C(O)—.

In some embodiments, L² is optionally substituted C₂₋₆ hydrocarbon, wherein at least one carbon-carbon bond is unsaturated. In some embodiments, L² is optionally substituted C₂ hydrocarbon, wherein the carbon-carbon bond is unsaturated. In some such embodiments, L² is optionally substituted ethenylene or ethynylene. In some embodiments, L² is optionally substituted C₃ hydrocarbon, wherein at least one carbon-carbon bond is unsaturated. In some such embodiments, L² is optionally substituted propenylene, also known as allylene, or propynylene. In some embodiments, L² is optionally substituted C₄ hydrocarbon, wherein at least one carbon-carbon bond is unsaturated. In some such embodiments, L² is optionally substituted butenylene, 2-methyl-propenylene, 1,3-butadienylene or butynylene. In some embodiments, L² is optionally substituted C₅ hydrocarbon, wherein at least one carbon-carbon bond is unsaturated. In some such embodiments, L² is optionally substituted pentenylene, isoamylenyl or pentynylene. In some embodiments, L² is optionally substituted C₆ hydrocarbon, wherein at least one carbon-carbon bond is unsaturated. In some such embodiments, L² is optionally substituted hexenylene or hexynylene.

In some embodiments of formula I, Ring A is a 6-membered saturated heterocyclic ring having 1 heteroatom selected from nitrogen, oxygen or sulfur. In some embodiments, Ring A is optionally substituted piperidinyl. Accordingly, in certain embodiments, the present invention provides a compound of formula II:

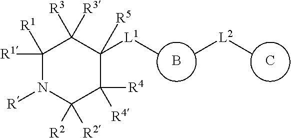

or a pharmaceutically acceptable salt thereof, wherein:
each R¹, R^{1′}, R², R^{2′}, R³, R^{3′}, R⁴, R^{4′} and R⁵ is independently —R′, halogen, —CN, —NO₂, —OR, —N(R′), —SR; or
   each of R¹ and R^{1′}, R² and R^{2′}, R³ and R^{3′}, or R⁴ and R^{4′} is optionally and independently taken together to form ═X, wherein X is ═O, ═S, ═NR′, ═N—N—OR or ═N—NR′; or
   each of R¹ or R^{1′} and R² or R^{2′}, R³ or R^{3′} and R⁴ or R^{4′}, R¹ or R^{1′} and R³ or R^{3′}, R² or R^{2′} and R⁴ or R^{4′}, R² or R^{2′} and R³ or R^{3′}, R¹ or R^{1′} and R⁴ or R^{4′}, R¹ or R^{1′} and R′, R² or R^{2′} and R′, and R′ and R⁵ is optionally and independently taken together with their intervening atoms to form a 3-7 membered saturated or partially unsaturated carbocyclic ring, or a 4-7 membered saturated or partially unsaturated heterocyclic ring having 1-2 heteroatoms independently selected from nitrogen, oxygen, and sulfur; and
each of Ring B, Ring C, R, R′, L¹ and L² is as defined above and described herein.

In some embodiments of formula II, Ring B is a 5-6 membered heteroarylene ring having 1-3 heteroatoms independently selected from nitrogen, oxygen or sulfur. In some embodiments of formula II, Ring B is a 6-membered heteroarylene ring having 1-3 heteroatoms independently selected from nitrogen, oxygen or sulfur. In some embodiments of formula II, Ring B is pyridine. In some embodiments of formula II, Ring B is pyrimidine. In some embodiments of formula II, Ring B is pyridazine.

In some embodiments of formula I, Ring A is a 5-membered saturated heterocyclic ring having 1 heteroatom selected from nitrogen, oxygen or sulfur. In some embodiments, Ring A is optionally substituted pyrrolidinyl. Accordingly, in certain embodiments, the present invention provides a compound of formula III:

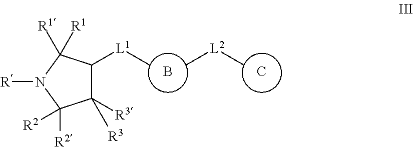

or a pharmaceutically acceptable salt thereof, wherein each of Ring B, Ring C, R¹, R^{1′}, R², R^{2′}, R³, R^{3′}, R′, L¹ and L² is as defined above and described herein.

In some embodiments of formula II, Ring B is optionally substituted phenyl. Accordingly, in some embodiments, the present invention provides a compound of formula IV:

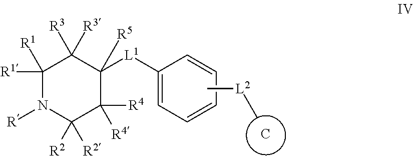

or a pharmaceutically acceptable salt thereof, wherein Ring C, R¹, R^{1′}, R², R^{2′}, R³, R^{3′}, R⁴, R^{4′}, R⁵, R′, L¹ and L² is as defined above and described herein.

In some embodiments of formula IV, Ring C is optionally substituted phenyl.

In some embodiments of formula IV, Ring C is phenyl substituted with one or more halogens. In some embodiments of formula IV, Ring C is phenyl substituted with —F. In some embodiments of formula IV, Ring C is phenyl substituted with —Cl. In some embodiments of formula IV, Ring C is phenyl substituted with —Br. In some embodiments of formula IV, Ring C is phenyl substituted with —I.

In some embodiments of formula IV, Ring C is phenyl substituted with —OR. In some embodiments of formula IV, Ring C is phenyl substituted with —OH. In some embodiments of formula IV, Ring C is phenyl substituted with —OCH₃. In some embodiments of formula IV, Ring C is phenyl substituted with —OCH₂CH₃.

In some embodiments of formula IV, Ring C is phenyl substituted with —N(R′)₂. In some embodiments of formula IV, Ring C is phenyl substituted with —NH₂. In some embodiments of formula IV, Ring C is phenyl substituted with —N(CH₃)₂. In some embodiments of formula IV, Ring C is phenyl substituted with —NHCH₃.

In some embodiments of formula IV, Ring C is phenyl substituted with —CN. In some embodiments of formula IV, Ring C is phenyl substituted with —NO₂.

In some embodiments of formula IV, Ring C is phenyl substituted with one or more aliphatic groups. In some embodiments of formula IV, Ring C is phenyl substituted with C₁₋₆ aliphatic. In some embodiments of formula IV, Ring C is phenyl substituted with C₁₋₅ aliphatic. In some embodiments of formula IV, Ring C is phenyl substituted with C₁₋₄ aliphatic. In some embodiments of formula IV, Ring C is phenyl substituted with C₁₋₃ aliphatic. In some embodiments of formula IV, Ring C is phenyl substituted with C₁₋₂ aliphatic. In some embodiments of formula IV, Ring C is phenyl substituted with C₁ aliphatic. In some embodiments of formula IV, Ring C is phenyl substituted with one or more —CH₃ groups. In some embodiments of formula IV, Ring C is phenyl substituted with —CH₃. In some embodiments of formula IV, Ring C is phenyl substituted with two —CH₃. In some embodiments of formula IV, Ring C is phenyl substituted with three —CH₃. In some embodiments of formula IV, Ring C is phenyl substituted with —CH₂CH₃. In some embodiments of formula IV, Ring C is phenyl substituted with at least one —CH₃.

In some embodiments of formula IV, Ring C is phenyl substituted with —CO₂R. In some embodiments of formula IV, Ring C is phenyl substituted with —CO₂H. In some embodiments of formula IV, Ring C is phenyl substituted with —CO₂CH₃. In some embodiments of formula IV, Ring C is phenyl substituted with —CO₂CH₂CH₃.

In some embodiments of formula IV, Ring C is phenyl substituted with —C(O)N(R′)₂. In some embodiments of formula IV, Ring C is phenyl substituted with —C(O)NH₂. In some embodiments of formula IV, Ring C is phenyl substituted with —C(O)N(CH₃)₂. In some embodiments of formula IV, Ring C is phenyl substituted with —C(O)N(H)CH₃.

In some embodiments of formula IV, Ring C is phenyl substituted with —N(R′)C(O)R. In some embodiments of formula IV, Ring C is phenyl substituted with —N(H)C(O)R. In some embodiments of formula IV, Ring C is phenyl substituted with —N(H)C(O)CH₃. In some embodiments of formula IV, Ring C is phenyl substituted with —N(CH₃)C(O)R.

In some embodiments of formula IV, Ring C is phenyl substituted with an optionally substituted phenyl.

In some embodiments of formula IV, Ring C is phenyl substituted with an optionally substituted 5-6 membered monocyclic heteroaryl ring having 1-3 heteroatoms independently selected from nitrogen, oxygen, or sulfur. In some embodiments of formula IV, Ring C is phenyl substituted with an optionally substituted 5-membered monocyclic heteroaryl ring having 1-3 heteroatoms independently selected from nitrogen, oxygen, or sulfur. In some embodiments of formula IV, Ring C is phenyl substituted with an optionally substituted 5-membered monocyclic heteroaryl ring having 1-2 heteroatoms independently selected from nitrogen, oxygen, or sulfur. In some embodiments of formula IV, Ring C is phenyl substituted with an optionally substituted 5-membered monocyclic heteroaryl ring having 2 heteroatoms independently selected from nitrogen, oxygen, or sulfur. In some embodiments of formula IV, Ring C is phenyl substituted with an optionally substituted 5-membered monocyclic heteroaryl ring having 1 heteroatom selected from nitrogen, oxygen, or sulfur. In some embodiments of formula IV, Ring C is phenyl substituted with an optionally substituted pyrrazole. In some embodiments of formula IV, Ring C is phenyl substituted with an optionally substituted thiophene.

In some embodiments of formula IV, Ring C is phenyl substituted with an optionally substituted 6-membered monocyclic heteroaryl ring having 1-3 heteroatoms independently selected from nitrogen, oxygen, or sulfur. In some embodiments of formula IV, Ring C is phenyl substituted with an optionally substituted 6-membered monocyclic heteroaryl ring having 1-2 heteroatoms independently selected from nitrogen, oxygen, or sulfur. In some embodiments of formula IV, Ring C is phenyl substituted with an optionally substituted 6-membered monocyclic heteroaryl ring having 2 heteroatoms independently selected from nitrogen, oxygen, or sulfur. In some embodiments of formula IV, Ring C is phenyl substituted with an optionally substituted 6-membered monocyclic heteroaryl ring having 1 heteroatom selected from nitrogen, oxygen, or sulfur. In some embodiments of formula IV, Ring C is phenyl substituted with an optionally substituted pyridine. In some embodiments of formula IV, Ring C is phenyl substituted with an optionally substituted pyrimidine.

In some embodiments of formula IV, Ring C is phenyl substituted with an optionally substituted 8-10 membered bicyclic heteroaryl ring having 1-3 heteroatoms independently selected from nitrogen, oxygen or sulfur. In some embodiments of formula IV, Ring C is phenyl substituted with an optionally substituted 9-membered bicyclic heteroaryl ring having 1-3 heteroatoms independently selected from nitrogen, oxygen or sulfur. In some embodiments of formula IV, Ring C is phenyl substituted with an optionally substituted 9-membered bicyclic heteroaryl ring having 1-2 heteroatoms independently selected from nitrogen, oxygen or sulfur. In some embodiments of formula IV, Ring C is phenyl substituted with an optionally substituted 9-membered bicyclic heteroaryl ring having 2 heteroatoms independently selected from nitrogen, oxygen or sulfur. In some embodiments of formula IV, Ring C is phenyl substituted with an optionally substituted 9-membered bicyclic heteroaryl ring having 1 heteroatom selected from nitrogen, oxygen or sulfur. In some embodiments of formula IV, Ring C is phenyl substituted with an optionally substituted azaindole. In some embodiments of formula IV, Ring C is phenyl substituted with an optionally substituted indole.

In some embodiments of formula IV, Ring C is phenyl substituted with an optionally substituted 10-membered bicyclic heteroaryl ring having 1-3 heteroatoms independently selected from nitrogen, oxygen or sulfur. In some embodiments of formula IV, Ring C is phenyl substituted with an optionally substituted 10-membered bicyclic heteroaryl ring having 1-2 heteroatoms independently selected from nitrogen, oxygen or sulfur. In some embodiments of formula IV, Ring C is phenyl substituted with an optionally substituted 10-membered bicyclic heteroaryl ring having 2 heteroatoms independently selected from nitrogen, oxygen or sulfur. In some embodiments of formula IV, Ring C is phenyl substituted with an optionally substituted 10-membered bicyclic heteroaryl ring having 1 heteroatom selected from nitrogen, oxygen or sulfur. In some embodiments of formula IV, Ring C is phenyl substituted with an optionally substituted quinoline. In some embodiments of formula IV, Ring C is phenyl substituted with an optionally substituted quinazoline.

In some embodiments of formula IV, Ring C is phenyl substituted with an optionally substituted 4-7 membered saturated or partially unsaturated heterocyclic ring having 1-3 heteroatoms independently selected from nitrogen, oxygen or sulfur. In some embodiments of formula IV, Ring C is phenyl substituted with an optionally substituted 6-membered saturated heterocyclic ring having 1-3 heteroatoms independently selected from nitrogen, oxygen or sulfur. In some embodiments of formula IV, Ring C is phenyl substituted with an optionally substituted 6-membered saturated heterocyclic ring having 1-2 heteroatoms independently selected from nitrogen, oxygen or sulfur. In some embodiments of formula IV, Ring C is phenyl substituted with an optionally substituted 6-membered saturated heterocyclic ring having 2 heteroatoms independently selected from nitrogen, oxygen or sulfur. In some embodiments of formula IV, Ring C is phenyl substituted with an optionally substituted 6-membered saturated heterocyclic ring having 1 heteroatom selected from nitrogen, oxygen or sulfur. In some embodiments of formula IV, Ring C is phenyl substituted with an optionally substituted morpholinyl. In some embodiments of formula IV, Ring C is phenyl substituted with an optionally substituted piperidinyl.

In some embodiments of formula IV, Ring C is a 5-6 membered monocyclic heteroaryl ring having 1-3 heteroatoms independently selected from nitrogen, oxygen, or sulfur. In some embodiments of formula IV, Ring C is a 5-membered monocyclic heteroaryl ring having 1-3 heteroatoms independently selected from nitrogen, oxygen or sulfur. In some embodiments of formula IV, Ring C is a 5-membered monocyclic heteroaryl ring having 1 heteroatom selected from nitrogen, oxygen or sulfur. In some embodiments of formula IV, Ring C is a 5-membered monocyclic heteroaryl ring having 2 heteroatoms independently selected from nitrogen, oxygen or sulfur. In some embodiments of formula IV, Ring C is a 5-membered monocyclic heteroaryl ring having 3 heteroatoms independently selected from nitrogen, oxygen or sulfur.

In some embodiments of formula IV, Ring C is a 6-membered monocyclic heteroaryl ring having 1-3 heteroatoms independently selected from nitrogen, oxygen, or sulfur. In some embodiments of formula IV, Ring C is a 6 membered monocyclic heteroaryl ring having 1 nitrogen. In some such embodiments of formula IV, Ring C is optionally substituted pyridinyl.

In some embodiments of formula IV, Ring C is pyridinyl substituted with —CN.

In some embodiments of formula IV, Ring C is pyridinyl substituted with one or more halogens. In some embodiments of formula IV, Ring C is pyridinyl substituted with —F. In some embodiments of formula IV, Ring C is pyridinyl substituted with —Cl. In some embodiments of formula IV, Ring C is pyridinyl substituted with —Br. In some embodiments of formula IV, Ring C is pyridinyl substituted with —I.

In some embodiments of formula IV, Ring C is pyridinyl substituted with aliphatic. In some embodiments of formula IV, Ring C is pyridinyl substituted with C₁₋₆ aliphatic. In some embodiments of formula IV, Ring C is pyridinyl substituted with C₁₋₅ aliphatic. In some embodiments of formula IV, Ring C is pyridinyl substituted with C₁₋₄ aliphatic. In some embodiments of formula IV, Ring C is pyridinyl substituted with C₁₋₃ aliphatic. In some embodiments of formula IV, Ring C is pyridinyl substituted with C₁₋₂ aliphatic. In some embodiments of formula IV, Ring C is pyridinyl substituted with C₁ aliphatic. In some embodiments of formula IV, Ring C is pyridinyl substituted with —CH₃. In some embodiments of formula IV, Ring C is pyridinyl substituted with at least one —CH₃. In some embodiments of formula IV, Ring C is pyridinyl substituted with two —CH₃.

In some embodiments of formula IV, Ring C is pyridinyl substituted with —N(R′)₂. In some embodiments of formula IV, Ring C is pyridinyl substituted with —NH₂. In some embodiments of formula IV, Ring C is pyridinyl substituted with —N(CH₃)₂. In some embodiments of formula IV, Ring C is pyridinyl substituted with —NHCH₃.

In some embodiments of formula IV, Ring C is pyridinyl substituted with —C(O)N(R′)₂. In some embodiments of formula IV, Ring C is pyridinyl substituted with —C(O)NH₂. In some embodiments of formula IV, Ring C is pyridinyl substituted with —C(O)NHCH₃. In some embodiments of formula IV, Ring C is pyridinyl substituted with —C(O)N(CH₃)₂.

In some embodiments of formula IV, Ring C is a 6 membered monocyclic heteroaryl ring having 2 nitrogen atoms. In some embodiments of formula IV, Ring C is optionally substituted pyridazinyl. In some embodiments of formula IV, Ring C is optionally substituted pyrimidinyl.

In some embodiments of formula IV, Ring C is an 8-10 membered bicyclic heteroaryl ring having 1-4 heteroatoms independently selected from nitrogen, oxygen, or sulfur. In some embodiments of formula IV, Ring C is indolyl. In some embodiments of formula IV, Ring C is quinolinyl.

In some embodiments of formula IV, Ring C is an 8-10 membered bicyclic aryl carbocyclic ring. In some embodiments of formula IV, Ring C is a 10-membered bicyclic aryl carbocyclic ring. In some embodiments of formula IV, Ring C is naphthyl.

In some embodiments, Ring C is selected from the group consisting of:

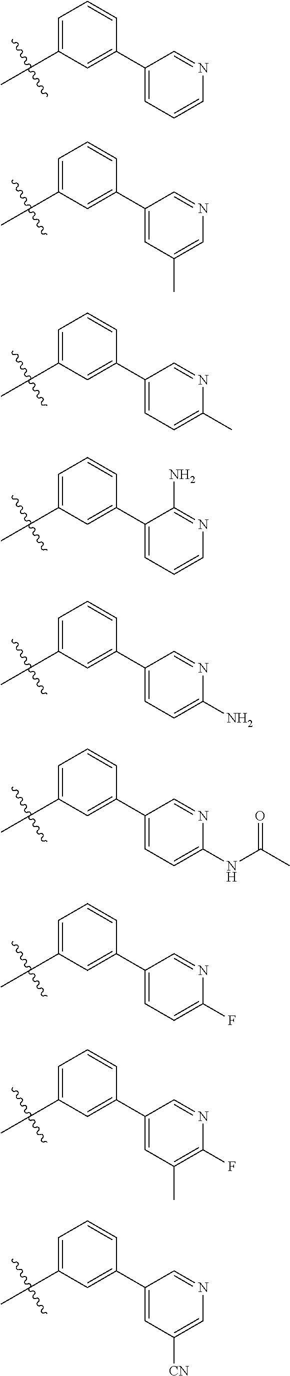

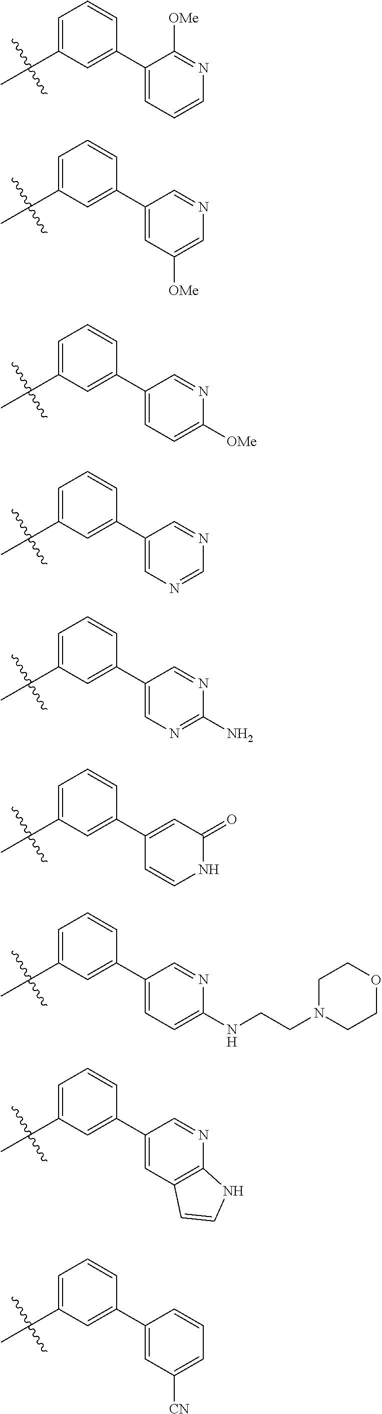

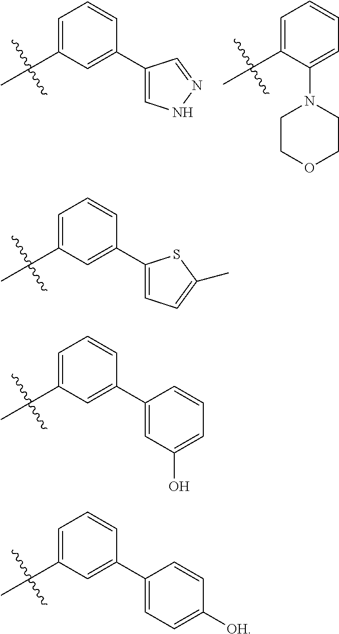 [SMILES: *c1cccc(-c2cn[nH]c2)c1 ; *c1ccccc1N1CCOCC1 ; *c1cccc(-c2ccc(C)s2)c1 ; *c1cccc(-c2cccc(O)c2)c1 ; *c1cccc(-c2ccc(O)cc2)c1]

In some embodiments, Ring C is selected from the group consisting of:

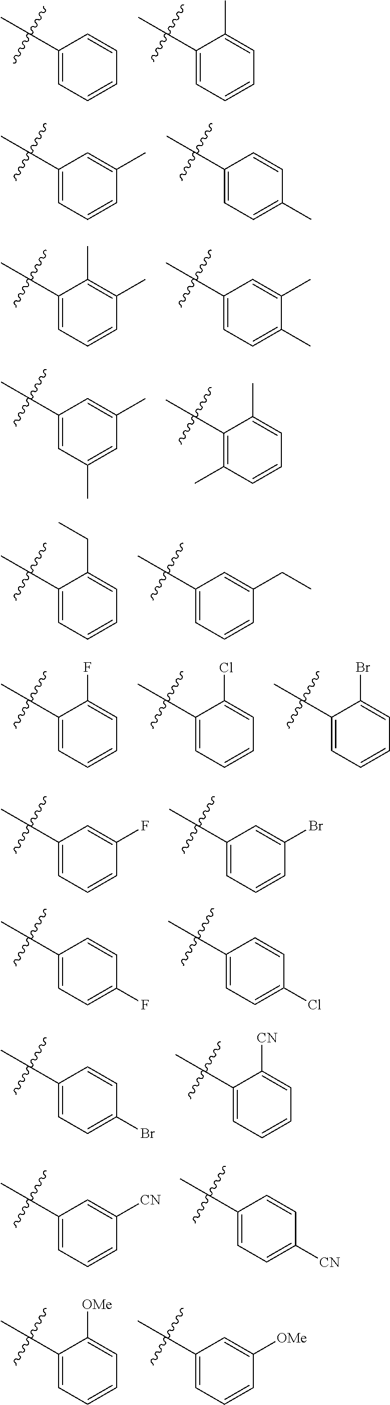

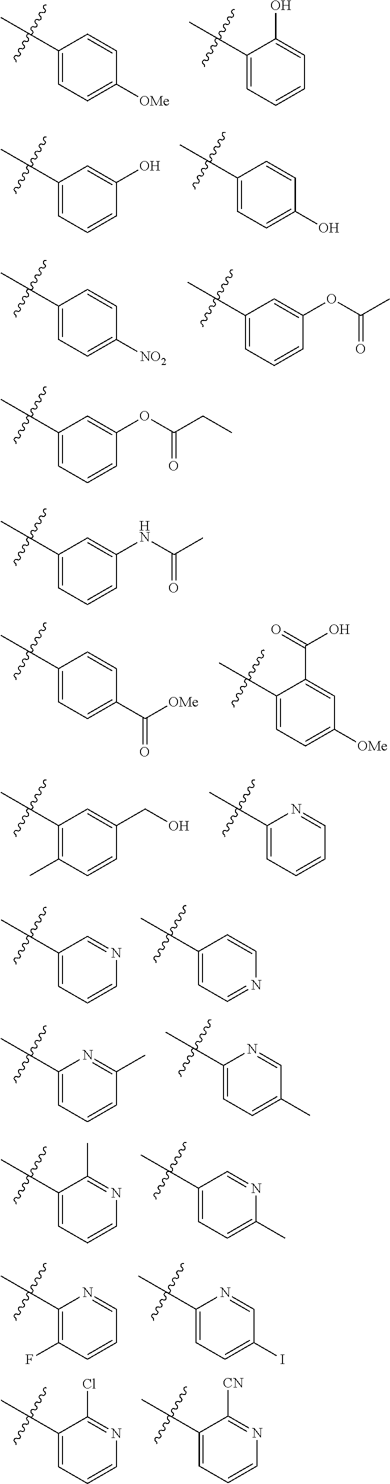

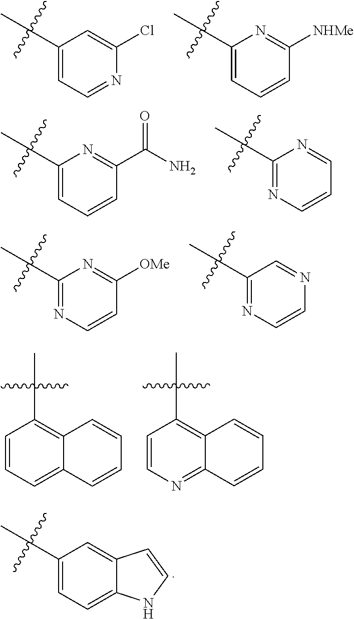

In some embodiments, the present invention provides a compound of formula V-a:

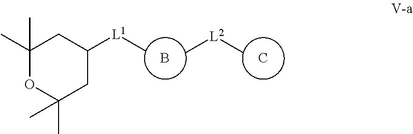

or a pharmaceutically acceptable salt thereof, wherein each of Ring B, Ring C, L¹ and L² is as defined above and described herein.

In some embodiments, the present invention provides a compound of formula V-b:

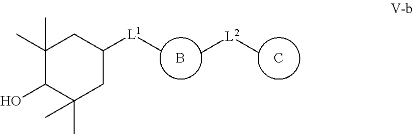

or a pharmaceutically acceptable salt thereof, wherein each of Ring B, Ring C, L¹ and L² is as defined above and described herein.

In some embodiments, the present invention provides a compound of formula V-c:

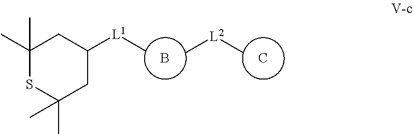

or a pharmaceutically acceptable salt thereof, wherein each of Ring B, Ring C, L¹ and L² is as defined above and described herein.

Exemplary compounds of formula I are set forth in Table 1, below.

TABLE 1
Exemplary Compounds of Formula I:

| | |
|---|---|
| 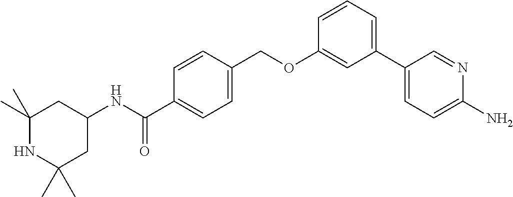 [SMILES: CC1(C)CC(NC(=O)c2ccc(COc3cccc(-c4ccc(N)nc4)c3)cc2)CC(C)(C)N1] | I-1 |
| 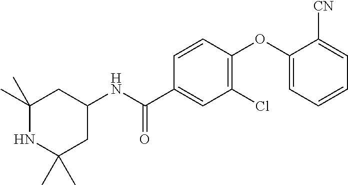 [SMILES: CC1(C)CC(NC(=O)c2ccc(Oc3ccccc3C#N)c(Cl)c2)CC(C)(C)N1] | I-2 |
| 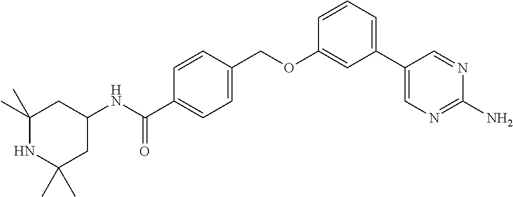 [SMILES: CC1(C)CC(NC(=O)c2ccc(COc3cccc(-c4cnc(N)nc4)c3)cc2)CC(C)(C)N1] | I-3 |
| 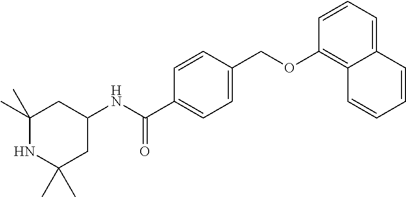 [SMILES: CC1(C)CC(NC(=O)c2ccc(COc3cccc4ccccc34)cc2)CC(C)(C)N1] | I-4 |
| 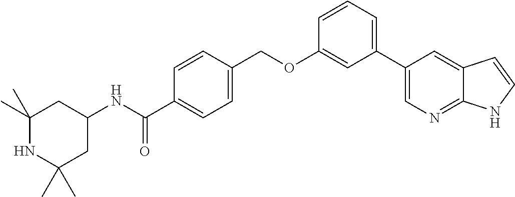 [SMILES: CC1(C)CC(NC(=O)c2ccc(COc3cccc(-c4cnc5[nH]ccc5c4)c3)cc2)CC(C)(C)N1] | I-5 |
| 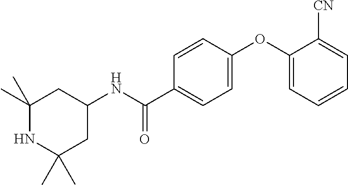 [SMILES: CC1(C)CC(NC(=O)c2ccc(Oc3ccccc3C#N)cc2)CC(C)(C)N1] | I-6 |
| 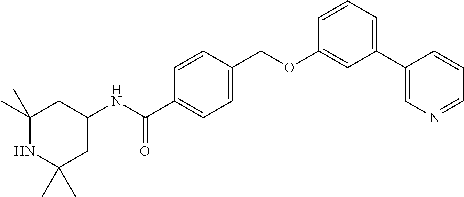 [SMILES: CC1(C)CC(NC(=O)c2ccc(COc3cccc(-c4cccnc4)c3)cc2)CC(C)(C)N1] | I-7 |
| 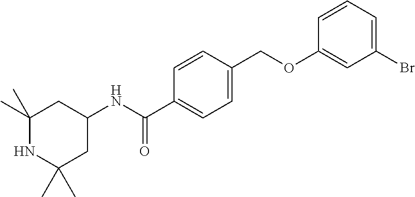 [SMILES: CC1(C)CC(NC(=O)c2ccc(COc3cccc(Br)c3)cc2)CC(C)(C)N1] | I-8 |
| 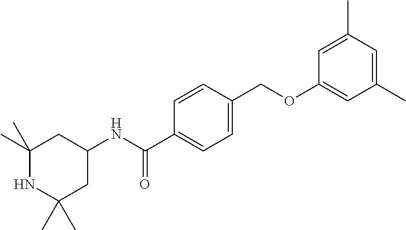 | I-9 |
| 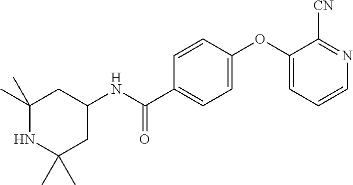 [SMILES: CC1(C)CC(NC(=O)c2ccc(Oc3cccnc3C#N)cc2)CC(C)(C)N1] | I-10 |
| 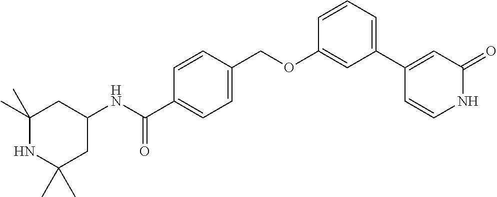 [SMILES: CC1(C)CC(NC(=O)c2ccc(COc3cccc(-c4cc[nH]c(=O)c4)c3)cc2)CC(C)(C)N1] | I-11 |
| 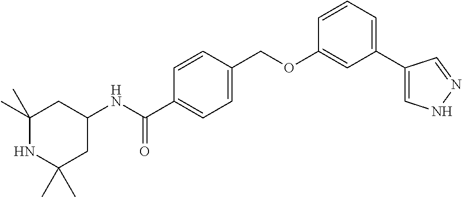 [SMILES: CC1(C)CC(NC(=O)c2ccc(COc3cccc(-c4cn[nH]c4)c3)cc2)CC(C)(C)N1] | I-12 |
| 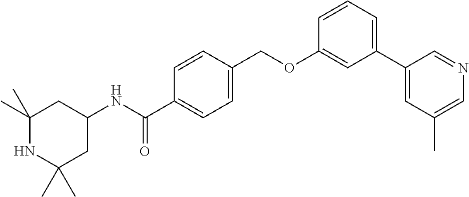 [SMILES: Cc1cncc(-c2cccc(OCc3ccc(C(=O)NC4CC(C)(C)NC(C)(C)C4)cc3)c2)c1] | I-13 |
| 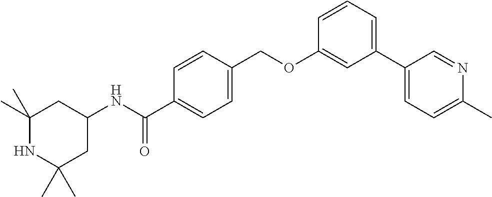 | I-14 |
| 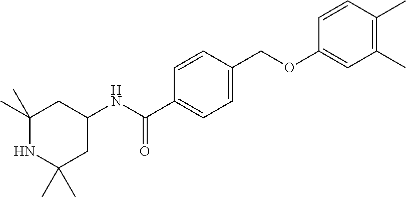 | I-15 |
| 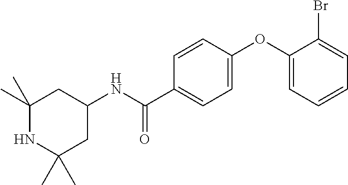 [SMILES: CC1(C)CC(NC(=O)c2ccc(Oc3ccccc3Br)cc2)CC(C)(C)N1] | I-16 |
| 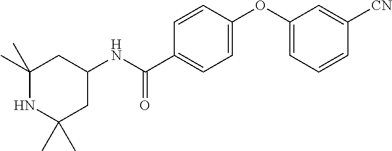 [SMILES: CC1(C)CC(NC(=O)c2ccc(Oc3cccc(C#N)c3)cc2)CC(C)(C)N1] | I-17 |
| 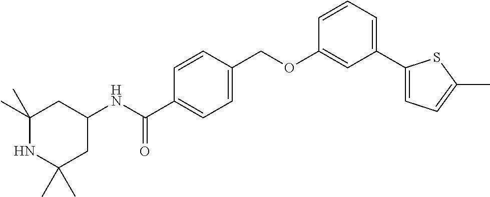 | I-18 |
| 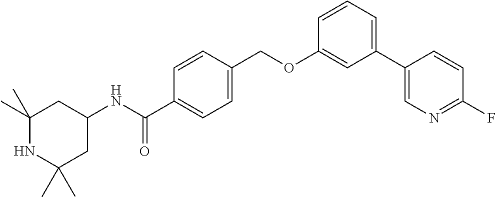 [SMILES: CC1(C)CC(NC(=O)c2ccc(COc3cccc(-c4ccc(F)nc4)c3)cc2)CC(C)(C)N1] | I-19 |
| 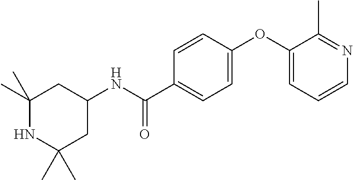 [SMILES: Cc1ncccc1Oc1ccc(C(=O)NC2CC(C)(C)NC(C)(C)C2)cc1] | I-20 |
| 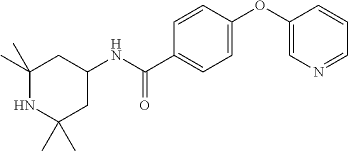 [SMILES: CC1(C)CC(NC(=O)c2ccc(Oc3cccnc3)cc2)CC(C)(C)N1] | I-21 |
| 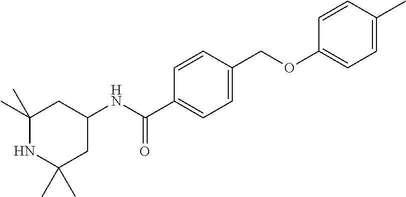 | I-22 |
| 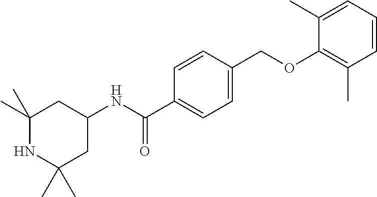 [SMILES: Cc1cccc(C)c1OCc1ccc(C(=O)NC2CC(C)(C)NC(C)(C)C2)cc1] | I-23 |
| 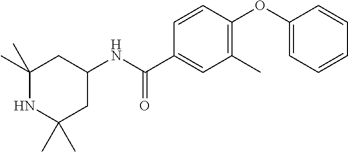 [SMILES: Cc1cc(C(=O)NC2CC(C)(C)NC(C)(C)C2)ccc1Oc1ccccc1] | I-24 |
| 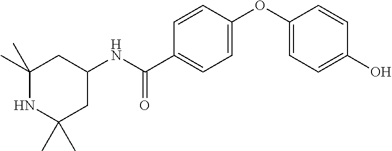 [SMILES: CC1(C)CC(NC(=O)c2ccc(Oc3ccc(O)cc3)cc2)CC(C)(C)N1] | I-25 |
| 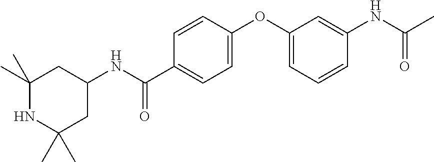 [SMILES: CC(=O)Nc1cccc(Oc2ccc(C(=O)NC3CC(C)(C)NC(C)(C)C3)cc2)c1] | I-26 |
| 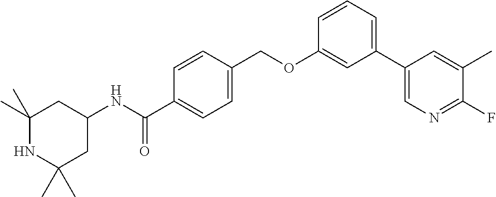 [SMILES: Cc1cc(-c2cccc(OCc3ccc(C(=O)NC4CC(C)(C)NC(C)(C)C4)cc3)c2)cnc1F] | I-27 |
| 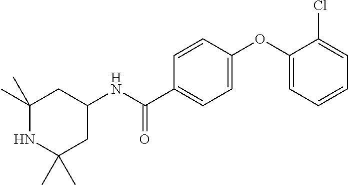 [SMILES: CC1(C)CC(NC(=O)c2ccc(Oc3ccccc3Cl)cc2)CC(C)(C)N1] | I-28 |
| 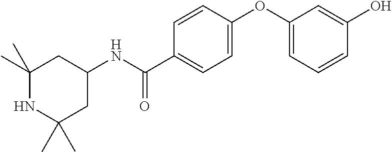 [SMILES: CC1(C)CC(NC(=O)c2ccc(Oc3cccc(O)c3)cc2)CC(C)(C)N1] | I-29 |
| 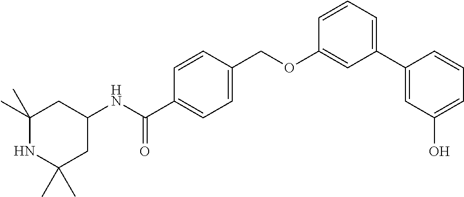 [SMILES: CC1(C)CC(NC(=O)c2ccc(COc3cccc(-c4cccc(O)c4)c3)cc2)CC(C)(C)N1] | I-30 |
| 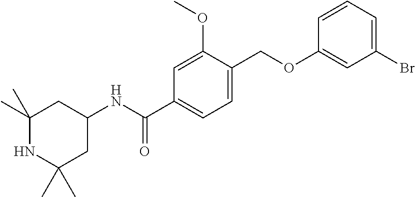 [SMILES: COc1cc(C(=O)NC2CC(C)(C)NC(C)(C)C2)ccc1COc1cccc(Br)c1] | I-31 |
| 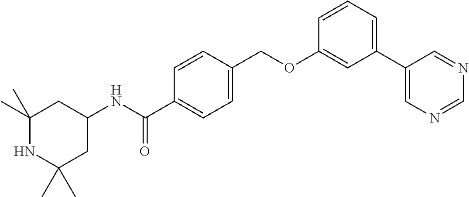 [SMILES: CC1(C)CC(NC(=O)c2ccc(COc3cccc(-c4cncnc4)c3)cc2)CC(C)(C)N1] | I-32 |
| 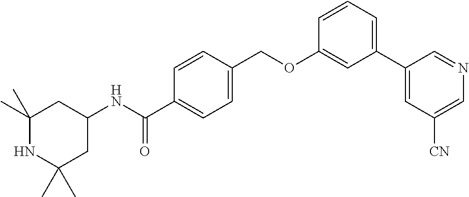 [SMILES: CC1(C)CC(NC(=O)c2ccc(COc3cccc(-c4cncc(C#N)c4)c3)cc2)CC(C)(C)N1] | I-33 |
| 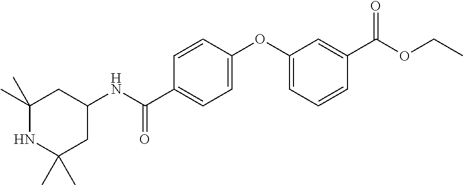 [SMILES: CCOC(=O)c1cccc(Oc2ccc(C(=O)NC3CC(C)(C)NC(C)(C)C3)cc2)c1] | I-34 |
| 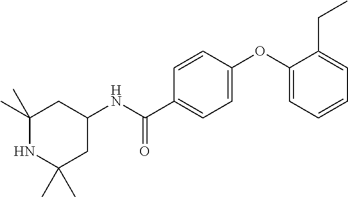 [SMILES: CCc1ccccc1Oc1ccc(C(=O)NC2CC(C)(C)NC(C)(C)C2)cc1] | I-35 |
| 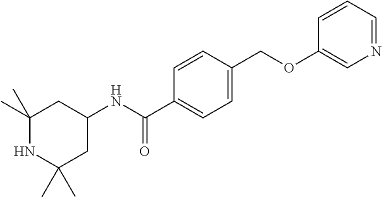 [SMILES: CC1(C)CC(NC(=O)c2ccc(COc3cccnc3)cc2)CC(C)(C)N1] | I-36 |
| 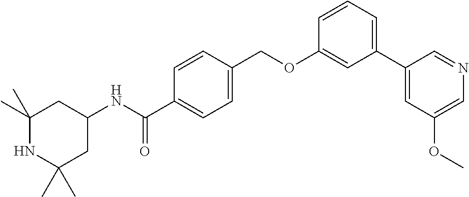 | I-37 |
| 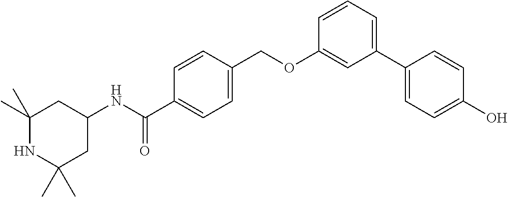 [SMILES: CC1(C)CC(NC(=O)c2ccc(COc3cccc(-c4ccc(O)cc4)c3)cc2)CC(C)(C)N1] | I-38 |
| 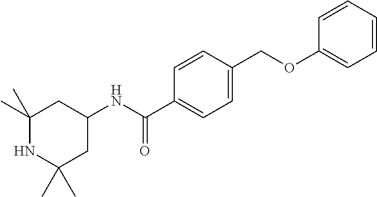 [SMILES: CC1(C)CC(NC(=O)c2ccc(COc3ccccc3)cc2)CC(C)(C)N1] | I-39 |
| 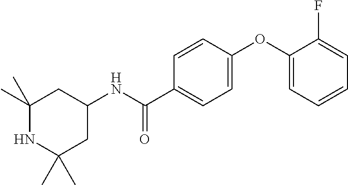 [SMILES: CC1(C)CC(NC(=O)c2ccc(Oc3ccccc3F)cc2)CC(C)(C)N1] | I-40 |
| 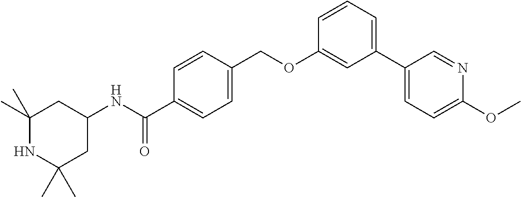 | I-41 |
| 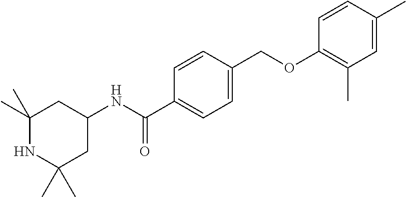 | I-42 |
| 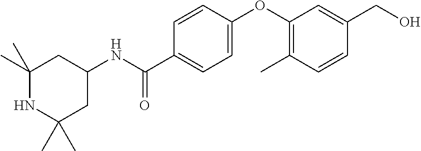 [SMILES: Cc1ccc(CO)cc1Oc1ccc(C(=O)NC2CC(C)(C)NC(C)(C)C2)cc1] | I-43 |
| 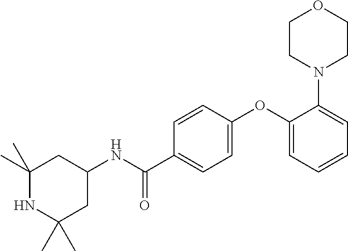 [SMILES: CC1(C)CC(NC(=O)c2ccc(Oc3ccccc3N3CCOCC3)cc2)CC(C)(C)N1] | I-44 |
| 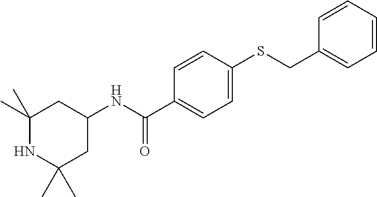 [SMILES: CC1(C)CC(NC(=O)c2ccc(SCc3ccccc3)cc2)CC(C)(C)N1] | I-45 |
| 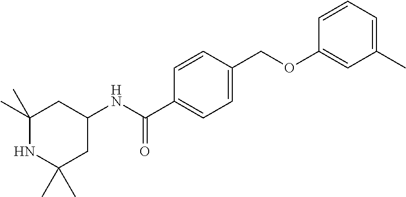 | I-46 |
| 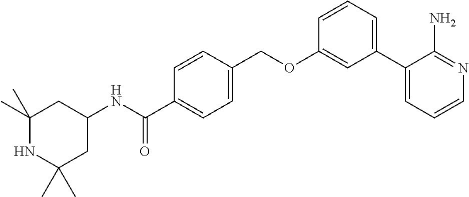 [SMILES: CC1(C)CC(NC(=O)c2ccc(COc3cccc(-c4cccnc4N)c3)cc2)CC(C)(C)N1] | I-47 |
| 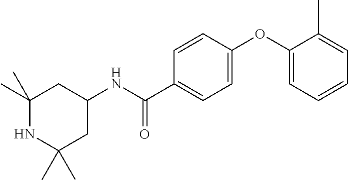 [SMILES: Cc1ccccc1Oc1ccc(C(=O)NC2CC(C)(C)NC(C)(C)C2)cc1] | I-48 |
| 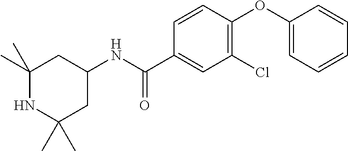 [SMILES: CC1(C)CC(NC(=O)c2ccc(Oc3ccccc3)c(Cl)c2)CC(C)(C)N1] | I-49 |
| 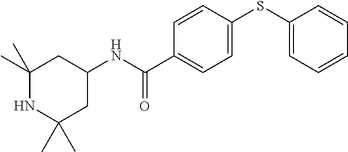 [SMILES: CC1(C)CC(NC(=O)c2ccc(Sc3ccccc3)cc2)CC(C)(C)N1] | I-50 |
| 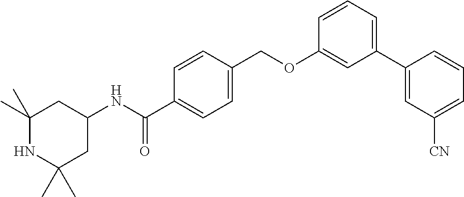 [SMILES: CC1(C)CC(NC(=O)c2ccc(COc3cccc(-c4cccc(C#N)c4)c3)cc2)CC(C)(C)N1] | I-51 |
| 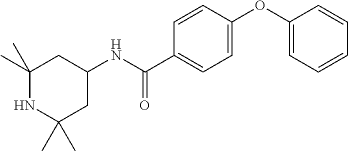 [SMILES: CC1(C)CC(NC(=O)c2ccc(Oc3ccccc3)cc2)CC(C)(C)N1] | I-52 |
| 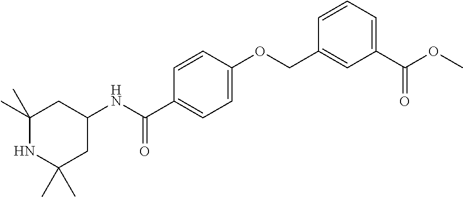 | I-53 |
| 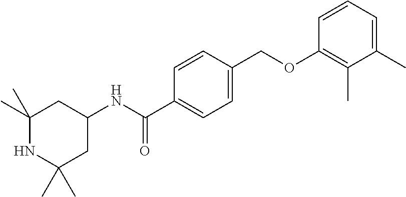 | I-54 |
| 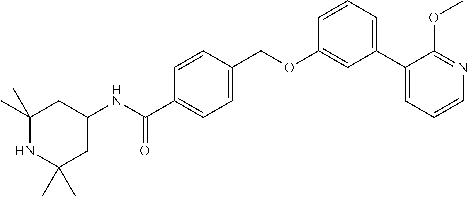 | I-55 |
| 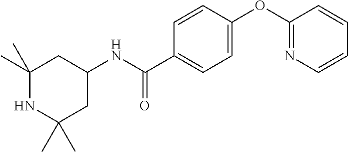 [SMILES: CC1(C)CC(NC(=O)c2ccc(Oc3ccccn3)cc2)CC(C)(C)N1] | I-56 |
| 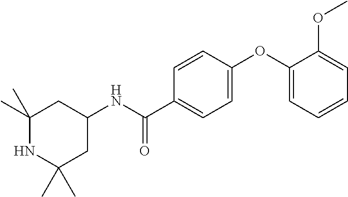 | I-57 |
| 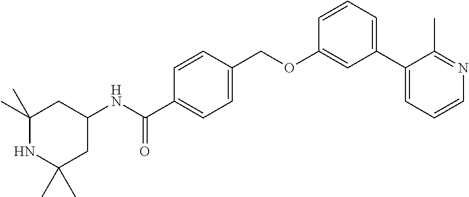 [SMILES: Cc1ncccc1-c1cccc(OCc2ccc(C(=O)NC3CC(C)(C)NC(C)(C)C3)cc2)c1] | I-58 |
| 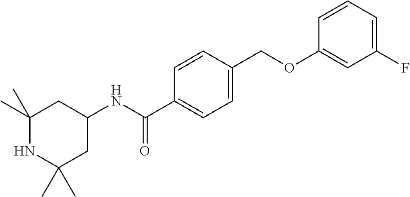 [SMILES: CC1(C)CC(NC(=O)c2ccc(COc3cccc(F)c3)cc2)CC(C)(C)N1] | I-59 |
| 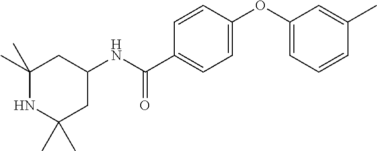 | I-60 |
| 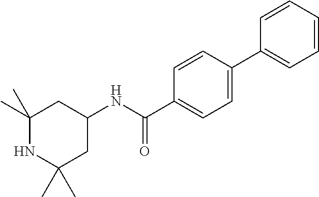 [SMILES: CC1(C)CC(NC(=O)c2ccc(-c3ccccc3)cc2)CC(C)(C)N1] | I-61 |
| 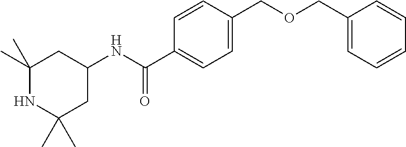 [SMILES: CC1(C)CC(NC(=O)c2ccc(COCc3ccccc3)cc2)CC(C)(C)N1] | I-62 |
| 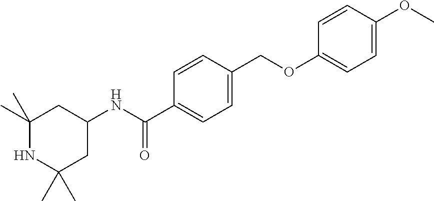 | I-63 |
| 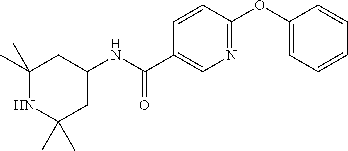 [SMILES: CC1(C)CC(NC(=O)c2ccc(Oc3ccccc3)nc2)CC(C)(C)N1] | I-64 |
| 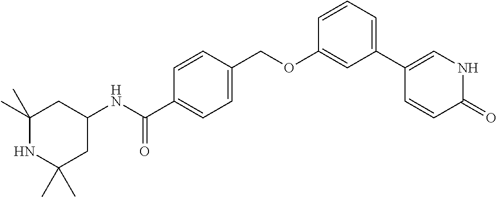 [SMILES: CC1(C)CC(NC(=O)c2ccc(COc3cccc(-c4ccc(=O)[nH]c4)c3)cc2)CC(C)(C)N1] | I-65 |
| 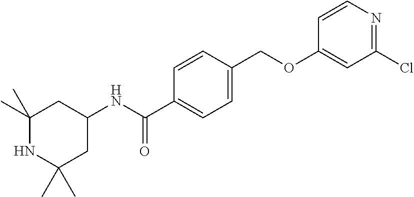 [SMILES: CC1(C)CC(NC(=O)c2ccc(COc3ccnc(Cl)c3)cc2)CC(C)(C)N1] | I-66 |
| 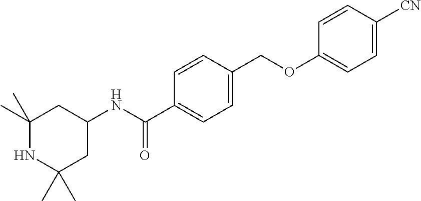 [SMILES: CC1(C)CC(NC(=O)c2ccc(COc3ccc(C#N)cc3)cc2)CC(C)(C)N1] | I-67 |
| 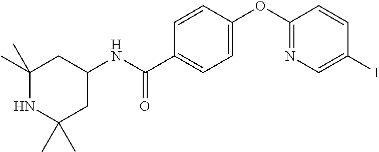 [SMILES: CC1(C)CC(NC(=O)c2ccc(Oc3ccc(I)cn3)cc2)CC(C)(C)N1] | I-68 |
| 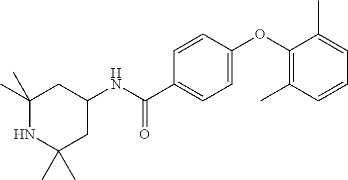 [SMILES: Cc1cccc(C)c1Oc1ccc(C(=O)NC2CC(C)(C)NC(C)(C)C2)cc1] | I-69 |
| 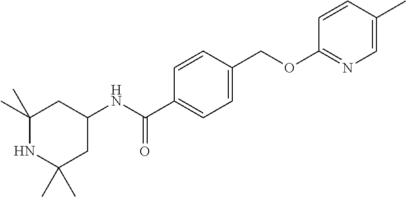 | I-70 |
| 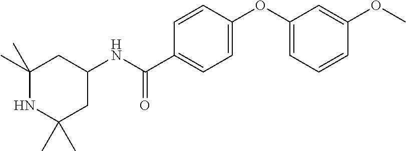 | I-71 |
| 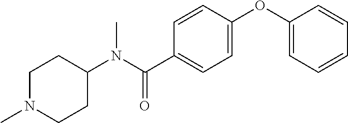 [SMILES: CN1CCC(N(C)C(=O)c2ccc(Oc3ccccc3)cc2)CC1] | I-72 |
| 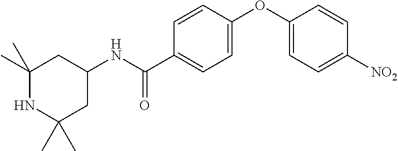 [SMILES: CC1(C)CC(NC(=O)c2ccc(Oc3ccc([N+](=O)[O-])cc3)cc2)CC(C)(C)N1] | I-73 |
| 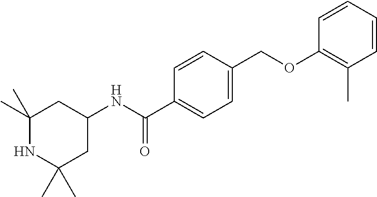 [SMILES: Cc1ccccc1OCc1ccc(C(=O)NC2CC(C)(C)NC(C)(C)C2)cc1] | I-74 |
| 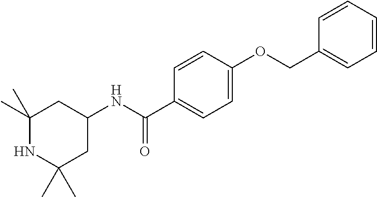 [SMILES: CC1(C)CC(NC(=O)c2ccc(OCc3ccccc3)cc2)CC(C)(C)N1] | I-75 |
| 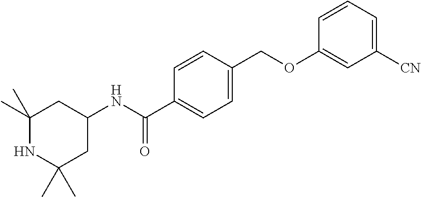 [SMILES: CC1(C)CC(NC(=O)c2ccc(COc3cccc(C#N)c3)cc2)CC(C)(C)N1] | I-76 |
| 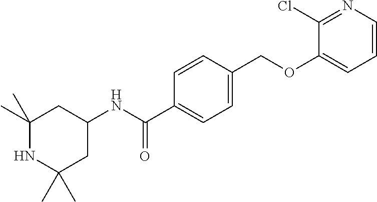 [SMILES: CC1(C)CC(NC(=O)c2ccc(COc3cccnc3Cl)cc2)CC(C)(C)N1] | I-77 |
| 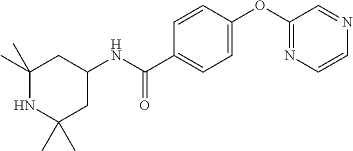 [SMILES: CC1(C)CC(NC(=O)c2ccc(Oc3cnccn3)cc2)CC(C)(C)N1] | I-78 |
| 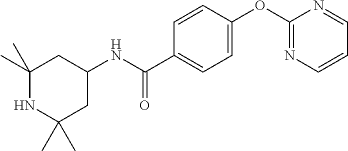 [SMILES: CC1(C)CC(NC(=O)c2ccc(Oc3ncccn3)cc2)CC(C)(C)N1] | I-79 |
| 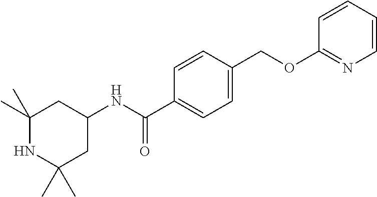 [SMILES: CC1(C)CC(NC(=O)c2ccc(COc3ccccn3)cc2)CC(C)(C)N1] | I-80 |
| 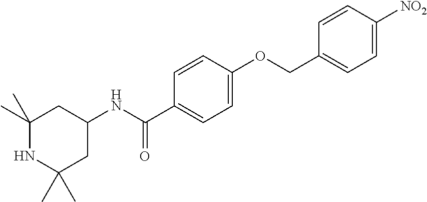 [SMILES: CC1(C)CC(NC(=O)c2ccc(OCc3ccc([N+](=O)[O-])cc3)cc2)CC(C)(C)N1] | I-81 |
| 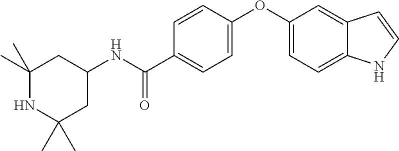 [SMILES: CC1(C)CC(NC(=O)c2ccc(Oc3ccc4[nH]ccc4c3)cc2)CC(C)(C)N1] | I-82 |
| 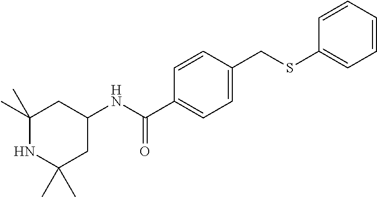 [SMILES: CC1(C)CC(NC(=O)c2ccc(CSc3ccccc3)cc2)CC(C)(C)N1] | I-83 |
| 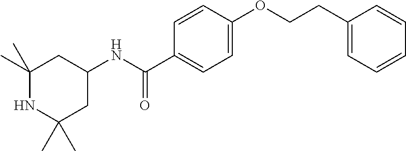 [SMILES: CC1(C)CC(NC(=O)c2ccc(OCCc3ccccc3)cc2)CC(C)(C)N1] | I-84 |
| 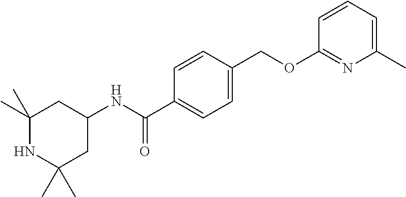 | I-85 |
| 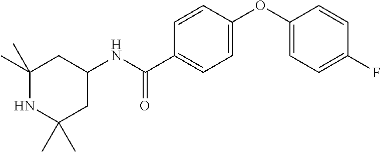 [SMILES: CC1(C)CC(NC(=O)c2ccc(Oc3ccc(F)cc3)cc2)CC(C)(C)N1] | I-86 |
| 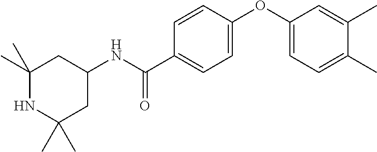 | I-87 |
| 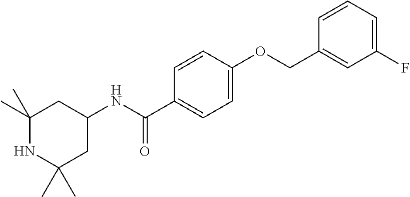 [SMILES: CC1(C)CC(NC(=O)c2ccc(OCc3cccc(F)c3)cc2)CC(C)(C)N1] | I-88 |
| 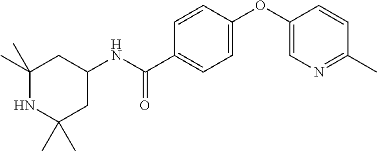 | I-89 |
| 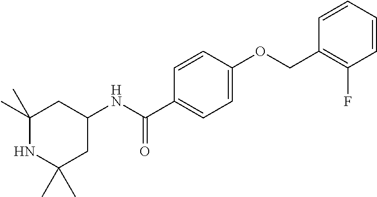 [SMILES: CC1(C)CC(NC(=O)c2ccc(OCc3ccccc3F)cc2)CC(C)(C)N1] | I-90 |
| 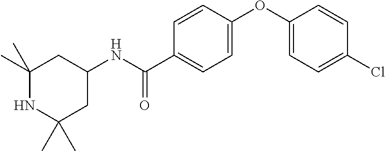 [SMILES: CC1(C)CC(NC(=O)c2ccc(Oc3ccc(Cl)cc3)cc2)CC(C)(C)N1] | I-91 |
| 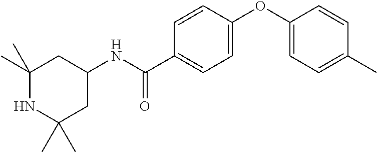 | I-92 |
| 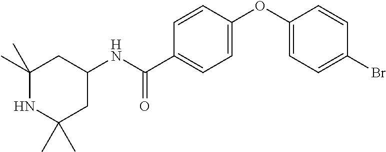 [SMILES: CC1(C)CC(NC(=O)c2ccc(Oc3ccc(Br)cc3)cc2)CC(C)(C)N1] | I-93 |
| 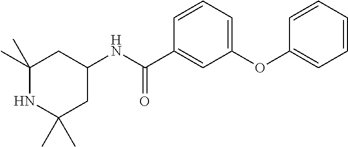 [SMILES: CC1(C)CC(NC(=O)c2cccc(Oc3ccccc3)c2)CC(C)(C)N1] | I-94 |
| 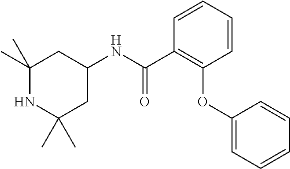 [SMILES: CC1(C)CC(NC(=O)c2ccccc2Oc2ccccc2)CC(C)(C)N1] | I-95 |
| 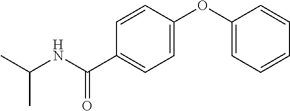 [SMILES: CC(C)NC(=O)c1ccc(Oc2ccccc2)cc1] | I-96 |
| 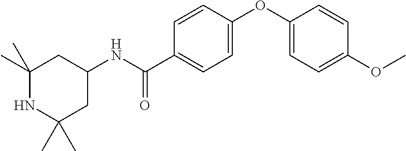 | I-97 |
| 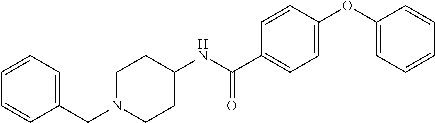 [SMILES: O=C(NC1CCN(Cc2ccccc2)CC1)c1ccc(Oc2ccccc2)cc1] | I-98 |
| 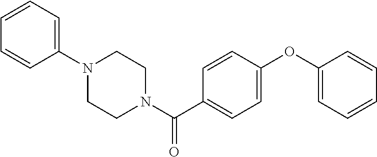 [SMILES: O=C(c1ccc(Oc2ccccc2)cc1)N1CCN(c2ccccc2)CC1] | I-99 |
| 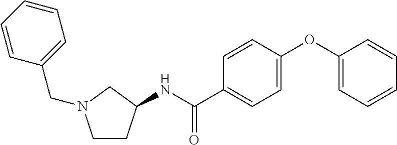 [SMILES: O=C(N[C@H]1CCN(Cc2ccccc2)C1)c1ccc(Oc2ccccc2)cc1] | I-100 |
| 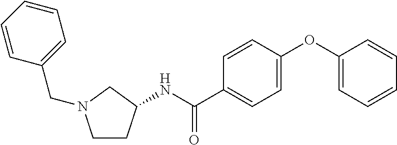 [SMILES: O=C(N[C@@H]1CCN(Cc2ccccc2)C1)c1ccc(Oc2ccccc2)cc1] | I-101 |
| 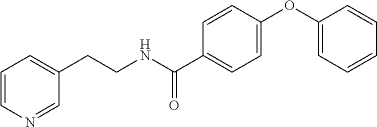 [SMILES: O=C(NCCc1cccnc1)c1ccc(Oc2ccccc2)cc1] | I-102 |
| 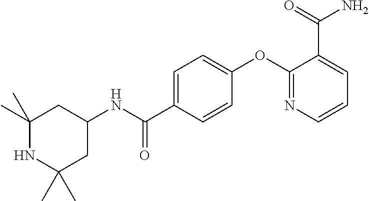 [SMILES: CC1(C)CC(NC(=O)c2ccc(Oc3ncccc3C(N)=O)cc2)CC(C)(C)N1] | I-103 |
| 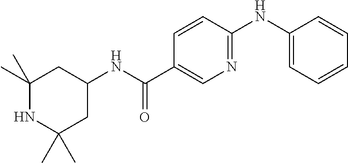 [SMILES: CC1(C)CC(NC(=O)c2ccc(Nc3ccccc3)nc2)CC(C)(C)N1] | I-104 |
| 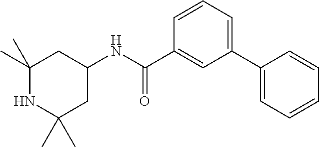 [SMILES: CC1(C)CC(NC(=O)c2cccc(-c3ccccc3)c2)CC(C)(C)N1] | I-105 |
| 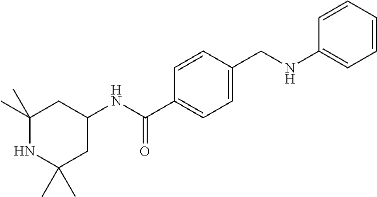 [SMILES: CC1(C)CC(NC(=O)c2ccc(CNc3ccccc3)cc2)CC(C)(C)N1] | I-106 |
| 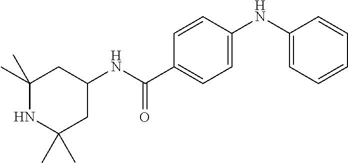 [SMILES: CC1(C)CC(NC(=O)c2ccc(Nc3ccccc3)cc2)CC(C)(C)N1] | I-107 |
| 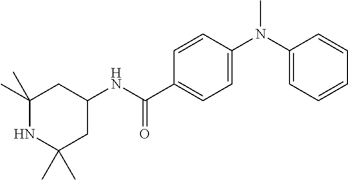 [SMILES: CN(c1ccccc1)c1ccc(C(=O)NC2CC(C)(C)NC(C)(C)C2)cc1] | I-108 |
| 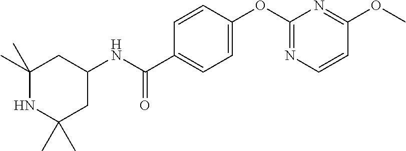 | I-109 |
| 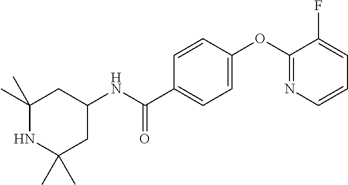 [SMILES: CC1(C)CC(NC(=O)c2ccc(Oc3ncccc3F)cc2)CC(C)(C)N1] | I-110 |
| 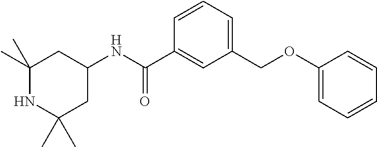 [SMILES: CC1(C)CC(NC(=O)c2cccc(COc3ccccc3)c2)CC(C)(C)N1] | I-111 |
| 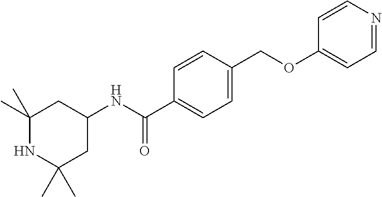 [SMILES: CC1(C)CC(NC(=O)c2ccc(COc3ccncc3)cc2)CC(C)(C)N1] | I-112 |
| 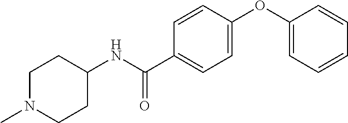 [SMILES: CN1CCC(NC(=O)c2ccc(Oc3ccccc3)cc2)CC1] | I-113 |
| 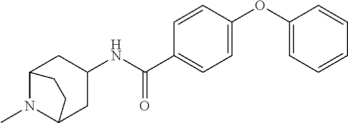 [SMILES: CN1C2CCC1CC(NC(=O)c1ccc(Oc3ccccc3)cc1)C2] | I-114 |
| 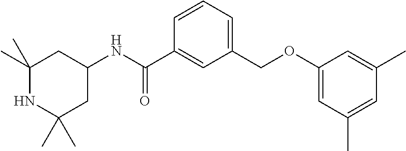 | I-115 |
| 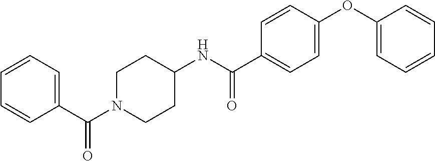 [SMILES: O=C(NC1CCN(C(=O)c2ccccc2)CC1)c1ccc(Oc2ccccc2)cc1] | I-116 |
| 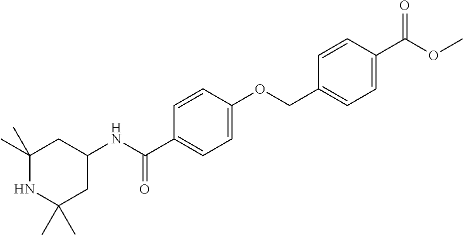 | I-117 |
| 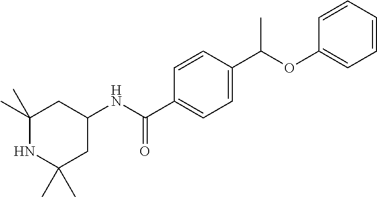 [SMILES: CC(Oc1ccccc1)c1ccc(C(=O)NC2CC(C)(C)NC(C)(C)C2)cc1] | I-118 |
| 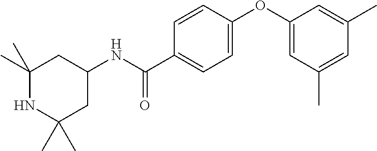 | I-119 |
| 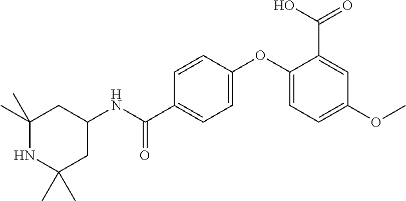 | I-120 |
| 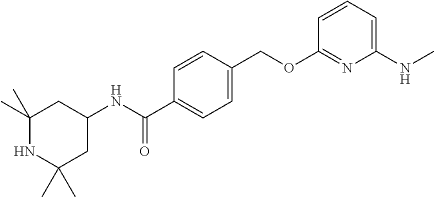 | I-121 |
| 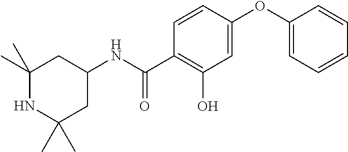 [SMILES: CC1(C)CC(NC(=O)c2ccc(Oc3ccccc3)cc2O)CC(C)(C)N1] | I-122 |
| 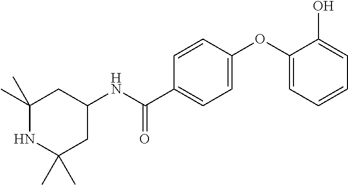 [SMILES: CC1(C)CC(NC(=O)c2ccc(Oc3ccccc3O)cc2)CC(C)(C)N1] | I-123 |
| 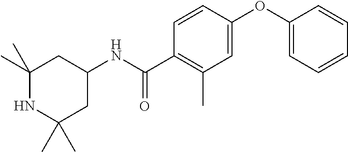 [SMILES: Cc1cc(Oc2ccccc2)ccc1C(=O)NC1CC(C)(C)NC(C)(C)C1] | I-124 |
| 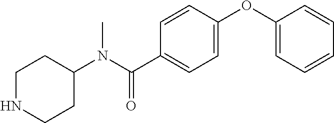 [SMILES: CN(C(=O)c1ccc(Oc2ccccc2)cc1)C1CCNCC1] | I-125 |
| 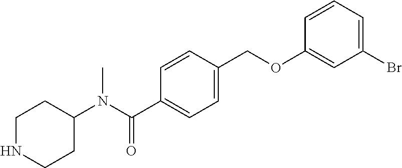 [SMILES: CN(C(=O)c1ccc(COc2cccc(Br)c2)cc1)C1CCNCC1] | I-126 |
| 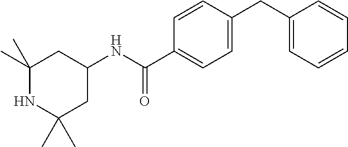 [SMILES: CC1(C)CC(NC(=O)c2ccc(Cc3ccccc3)cc2)CC(C)(C)N1] | I-127 |
| 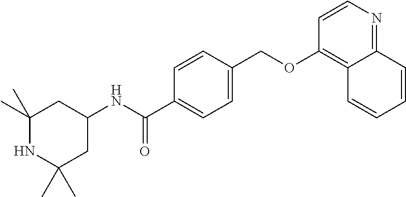 [SMILES: CC1(C)CC(NC(=O)c2ccc(COc3ccnc4ccccc34)cc2)CC(C)(C)N1] | I-128 |
| 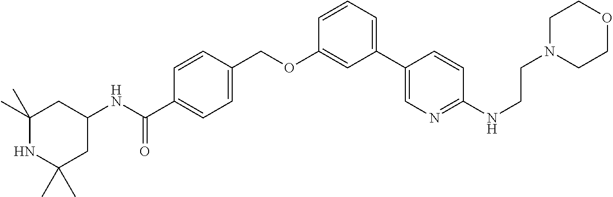 [SMILES: CC1(C)CC(NC(=O)c2ccc(COc3cccc(-c4ccc(NCCN5CCOCC5)nc4)c3)cc2)CC(C)(C)N1] | I-129 |
| 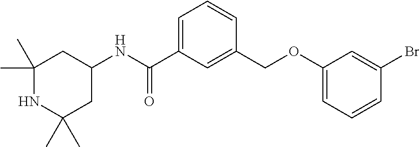 [SMILES: CC1(C)CC(NC(=O)c2cccc(COc3cccc(Br)c3)c2)CC(C)(C)N1] | I-130 |
| 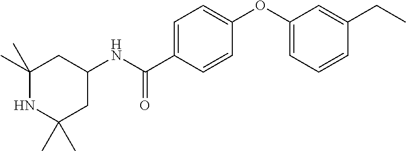 [SMILES: CCc1cccc(Oc2ccc(C(=O)NC3CC(C)(C)NC(C)(C)C3)cc2)c1] | I-131 |
| 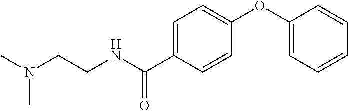 [SMILES: CN(C)CCNC(=O)c1ccc(Oc2ccccc2)cc1] | I-132 |
| 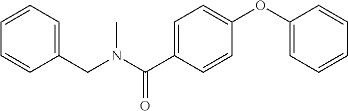 [SMILES: CN(Cc1ccccc1)C(=O)c1ccc(Oc2ccccc2)cc1] | I-133 |
| 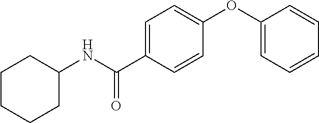 [SMILES: O=C(NC1CCCCC1)c1ccc(Oc2ccccc2)cc1] | I-134 |
| 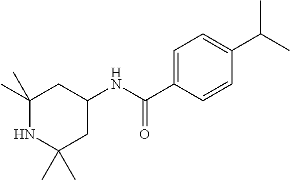 [SMILES: CC(C)c1ccc(C(=O)NC2CC(C)(C)NC(C)(C)C2)cc1] | I-135 |
| 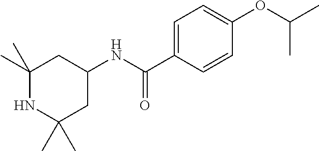 [SMILES: CC(C)Oc1ccc(C(=O)NC2CC(C)(C)NC(C)(C)C2)cc1] | I-136 |
| 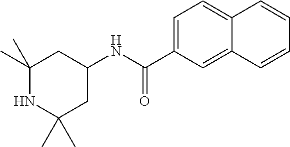 [SMILES: CC1(C)CC(NC(=O)c2ccc3ccccc3c2)CC(C)(C)N1] | I-137 |
| 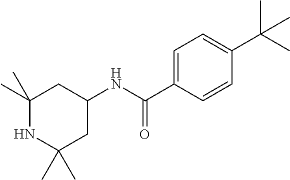 [SMILES: CC1(C)CC(NC(=O)c2ccc(C(C)(C)C)cc2)CC(C)(C)N1] | I-138 |
| 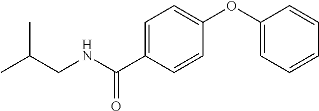 [SMILES: CC(C)CNC(=O)c1ccc(Oc2ccccc2)cc1] | I-139 |
| 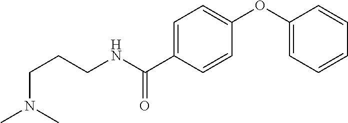 [SMILES: CN(C)CCCNC(=O)c1ccc(Oc2ccccc2)cc1] | I-140 |
| 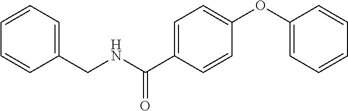 [SMILES: O=C(NCc1ccccc1)c1ccc(Oc2ccccc2)cc1] | I-141 |
| 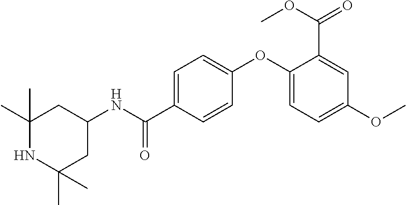 | I-142 |
| 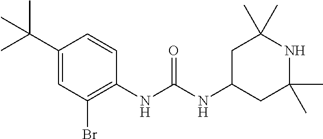 [SMILES: CC1(C)CC(NC(=O)Nc2ccc(C(C)(C)C)cc2Br)CC(C)(C)N1] | I-143 |
| 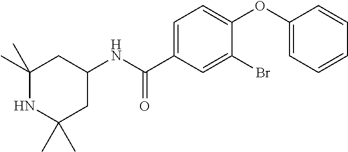 [SMILES: CC1(C)CC(NC(=O)c2ccc(Oc3ccccc3)c(Br)c2)CC(C)(C)N1] | I-144 |
| 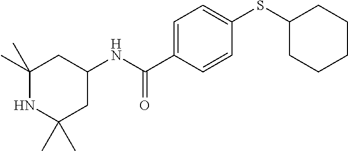 [SMILES: CC1(C)CC(NC(=O)c2ccc(SC3CCCCC3)cc2)CC(C)(C)N1] | I-145 |
| 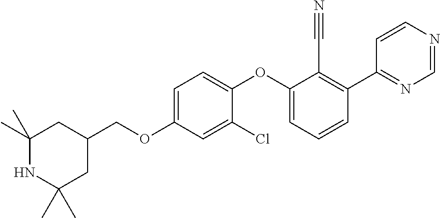 [SMILES: CC1(C)CC(COc2ccc(Oc3cccc(-c4ccncn4)c3C#N)c(Cl)c2)CC(C)(C)N1] | I-146 |
| 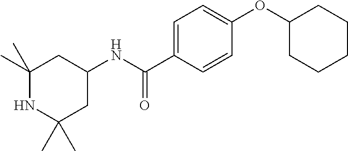 [SMILES: CC1(C)CC(NC(=O)c2ccc(OC3CCCCC3)cc2)CC(C)(C)N1] | I-147 |
| 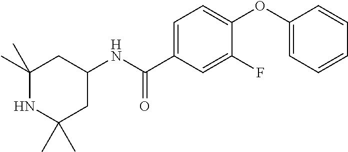 [SMILES: CC1(C)CC(NC(=O)c2ccc(Oc3ccccc3)c(F)c2)CC(C)(C)N1] | I-148 |
| 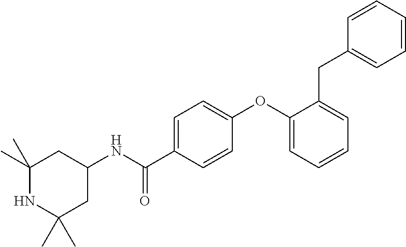 [SMILES: CC1(C)CC(NC(=O)c2ccc(Oc3ccccc3Cc3ccccc3)cc2)CC(C)(C)N1] | I-149 |
| 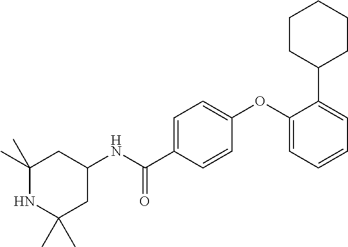 [SMILES: CC1(C)CC(NC(=O)c2ccc(Oc3ccccc3C3CCCCC3)cc2)CC(C)(C)N1] | I-150 |
| 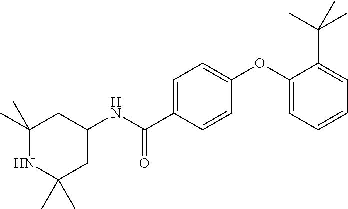 [SMILES: CC1(C)CC(NC(=O)c2ccc(Oc3ccccc3C(C)(C)C)cc2)CC(C)(C)N1] | I-151 |
| 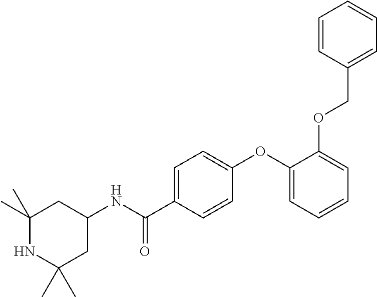 [SMILES: CC1(C)CC(NC(=O)c2ccc(Oc3ccccc3OCc3ccccc3)cc2)CC(C)(C)N1] | I-152 |
| 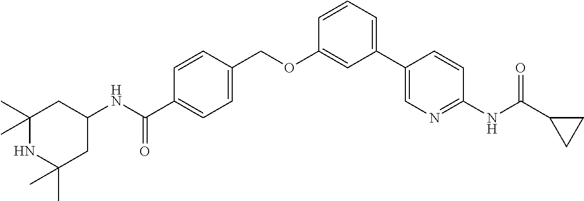 [SMILES: CC1(C)CC(NC(=O)c2ccc(COc3cccc(-c4ccc(NC(=O)C5CC5)nc4)c3)cc2)CC(C)(C)N1] | I-153 |
| 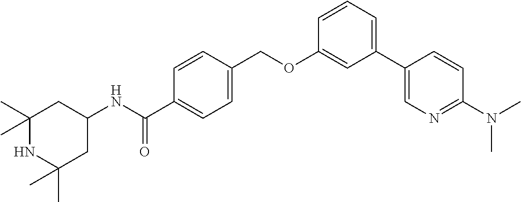 | I-154 |
| 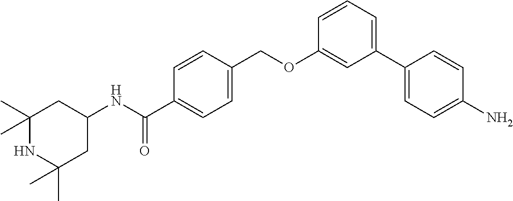 [SMILES: CC1(C)CC(NC(=O)c2ccc(COc3cccc(-c4ccc(N)cc4)c3)cc2)CC(C)(C)N1] | I-155 |
| 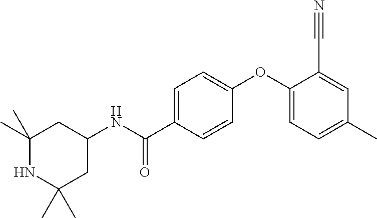 | I-156 |
| 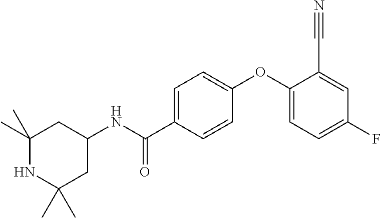 [SMILES: CC1(C)CC(NC(=O)c2ccc(Oc3ccc(F)cc3C#N)cc2)CC(C)(C)N1] | I-157 |
| 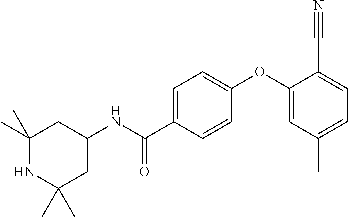 [SMILES: Cc1ccc(C#N)c(Oc2ccc(C(=O)NC3CC(C)(C)NC(C)(C)C3)cc2)c1] | I-158 |
| 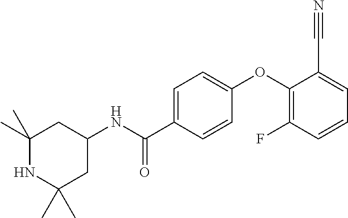 [SMILES: CC1(C)CC(NC(=O)c2ccc(Oc3c(F)cccc3C#N)cc2)CC(C)(C)N1] | I-159 |
| 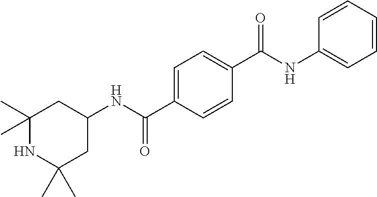 [SMILES: CC1(C)CC(NC(=O)c2ccc(C(=O)Nc3ccccc3)cc2)CC(C)(C)N1] | I-160 |
| 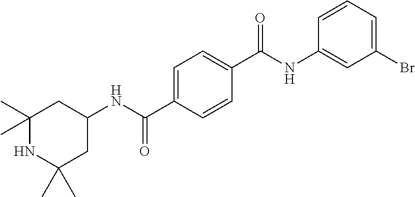 [SMILES: CC1(C)CC(NC(=O)c2ccc(C(=O)Nc3cccc(Br)c3)cc2)CC(C)(C)N1] | I-161 |
| 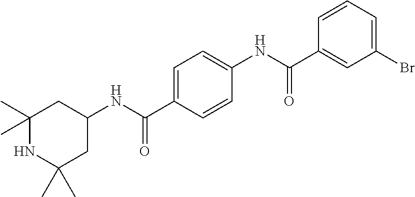 [SMILES: CC1(C)CC(NC(=O)c2ccc(NC(=O)c3cccc(Br)c3)cc2)CC(C)(C)N1] | I-162 |
| 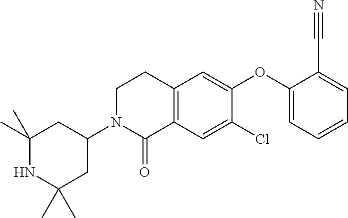 [SMILES: CC1(C)CC(N2CCc3cc(Oc4ccccc4C#N)c(Cl)cc3C2=O)CC(C)(C)N1] | I-163 |
| 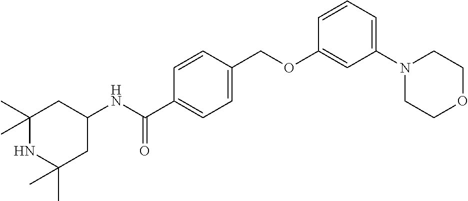 [SMILES: CC1(C)CC(NC(=O)c2ccc(COc3cccc(N4CCOCC4)c3)cc2)CC(C)(C)N1] | I-164 |
| 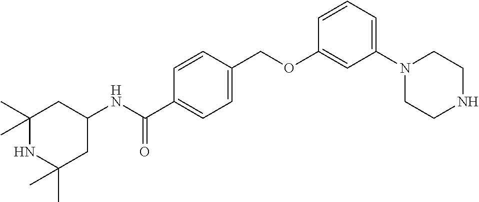 [SMILES: CC1(C)CC(NC(=O)c2ccc(COc3cccc(N4CCNCC4)c3)cc2)CC(C)(C)N1] | I-165 |
| 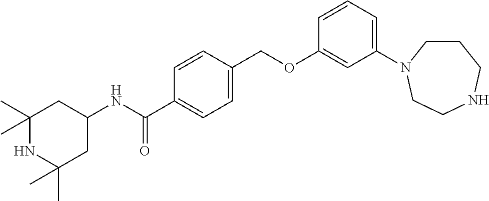 [SMILES: CC1(C)CC(NC(=O)c2ccc(COc3cccc(N4CCCNCC4)c3)cc2)CC(C)(C)N1] | I-166 |
| 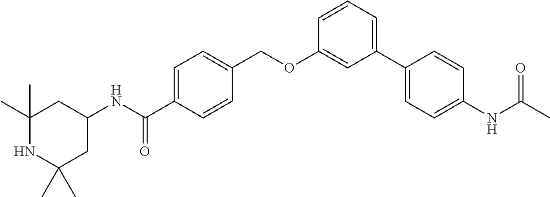 [SMILES: CC(=O)Nc1ccc(-c2cccc(OCc3ccc(C(=O)NC4CC(C)(C)NC(C)(C)C4)cc3)c2)cc1] | I-167 |
| 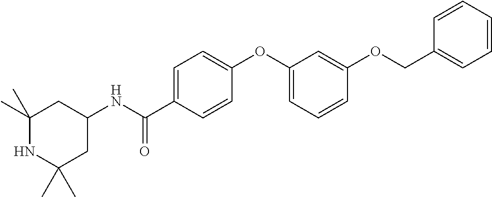 [SMILES: CC1(C)CC(NC(=O)c2ccc(Oc3cccc(OCc4ccccc4)c3)cc2)CC(C)(C)N1] | I-168 |
| 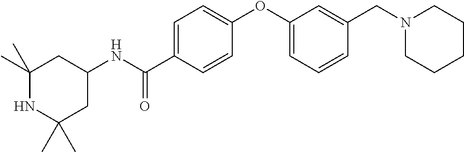 [SMILES: CC1(C)CC(NC(=O)c2ccc(Oc3cccc(CN4CCCCC4)c3)cc2)CC(C)(C)N1] | I-169 |
| 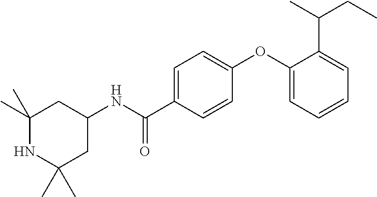 [SMILES: CCC(C)c1ccccc1Oc1ccc(C(=O)NC2CC(C)(C)NC(C)(C)C2)cc1] | I-170 |
| 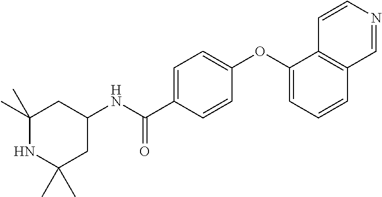 [SMILES: CC1(C)CC(NC(=O)c2ccc(Oc3cccc4cnccc34)cc2)CC(C)(C)N1] | I-171 |
| 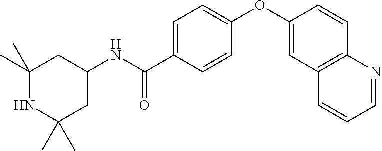 [SMILES: CC1(C)CC(NC(=O)c2ccc(Oc3ccc4ncccc4c3)cc2)CC(C)(C)N1] | I-172 |
| 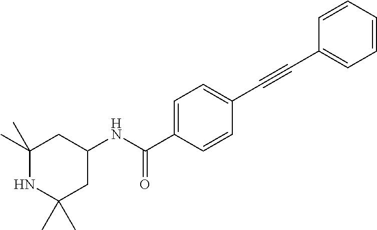 [SMILES: CC1(C)CC(NC(=O)c2ccc(C#Cc3ccccc3)cc2)CC(C)(C)N1] | I-173 |
| 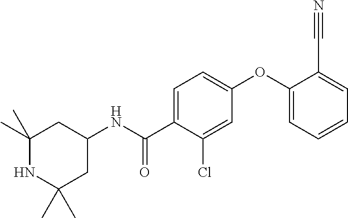 [SMILES: CC1(C)CC(NC(=O)c2ccc(Oc3ccccc3C#N)cc2Cl)CC(C)(C)N1] | I-174 |
| 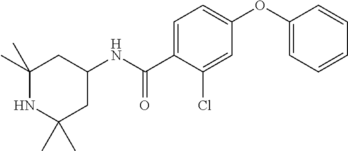 [SMILES: CC1(C)CC(NC(=O)c2ccc(Oc3ccccc3)cc2Cl)CC(C)(C)N1] | I-175 |
| 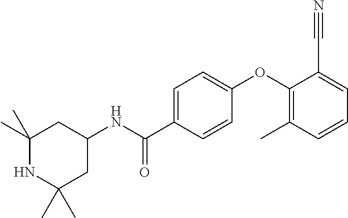 [SMILES: Cc1cccc(C#N)c1Oc1ccc(C(=O)NC2CC(C)(C)NC(C)(C)C2)cc1] | I-176 |
| 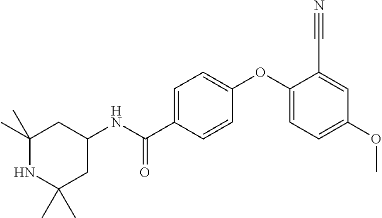 | I-177 |
| 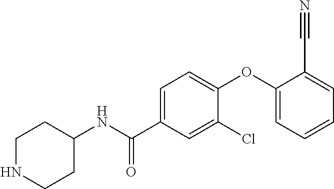 [SMILES: N#Cc1ccccc1Oc1ccc(C(=O)NC2CCNCC2)cc1Cl] | I-178 |
| 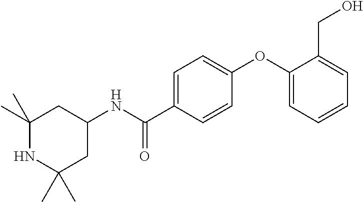 [SMILES: CC1(C)CC(NC(=O)c2ccc(Oc3ccccc3CO)cc2)CC(C)(C)N1] | I-179 |
| 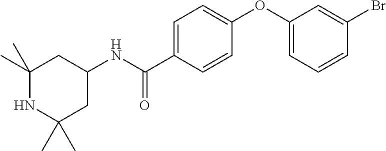 [SMILES: CC1(C)CC(NC(=O)c2ccc(Oc3cccc(Br)c3)cc2)CC(C)(C)N1] | I-180 |
| 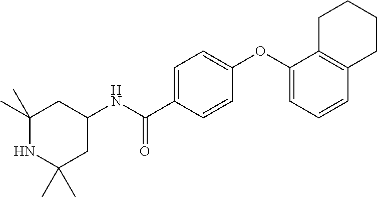 [SMILES: CC1(C)CC(NC(=O)c2ccc(Oc3cccc4c3CCCC4)cc2)CC(C)(C)N1] | I-181 |
| 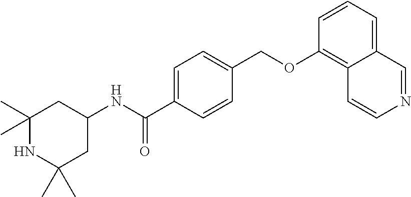 [SMILES: CC1(C)CC(NC(=O)c2ccc(COc3cccc4cnccc34)cc2)CC(C)(C)N1] | I-182 |
| 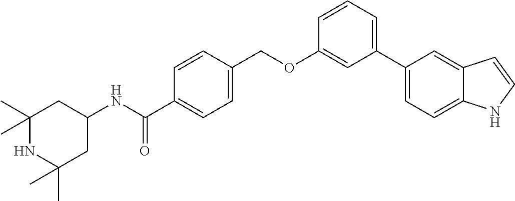 [SMILES: CC1(C)CC(NC(=O)c2ccc(COc3cccc(-c4ccc5[nH]ccc5c4)c3)cc2)CC(C)(C)N1] | I-183 |
| 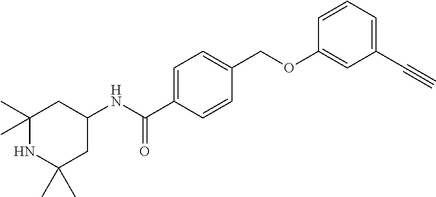 [SMILES: C#Cc1cccc(OCc2ccc(C(=O)NC3CC(C)(C)NC(C)(C)C3)cc2)c1] | I-184 |
| 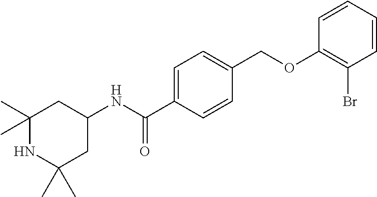 [SMILES: CC1(C)CC(NC(=O)c2ccc(COc3ccccc3Br)cc2)CC(C)(C)N1] | I-185 |
| 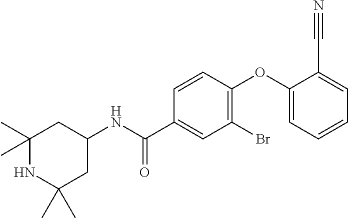 [SMILES: CC1(C)CC(NC(=O)c2ccc(Oc3ccccc3C#N)c(Br)c2)CC(C)(C)N1] | I-186 |
| 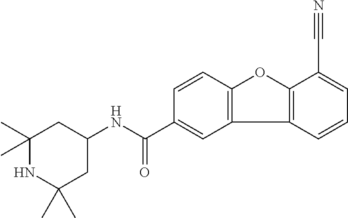 [SMILES: CC1(C)CC(NC(=O)c2ccc3oc4c(C#N)cccc4c3c2)CC(C)(C)N1] | I-187 |
| 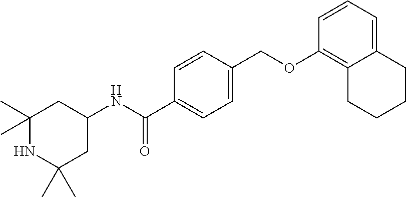 [SMILES: CC1(C)CC(NC(=O)c2ccc(COc3cccc4c3CCCC4)cc2)CC(C)(C)N1] | I-188 |
| 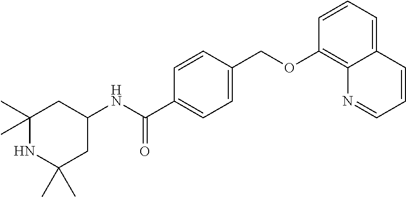 [SMILES: CC1(C)CC(NC(=O)c2ccc(COc3cccc4cccnc34)cc2)CC(C)(C)N1] | I-189 |
| 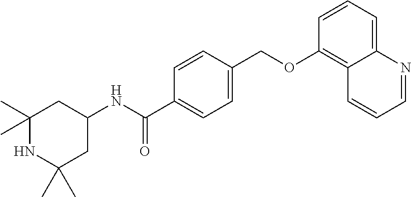 [SMILES: CC1(C)CC(NC(=O)c2ccc(COc3cccc4ncccc34)cc2)CC(C)(C)N1] | I-190 |
| 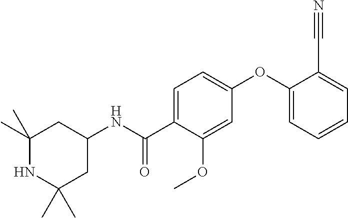 [SMILES: COc1cc(Oc2ccccc2C#N)ccc1C(=O)NC1CC(C)(C)NC(C)(C)C1] | I-191 |
| 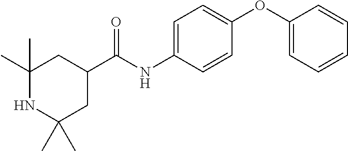 [SMILES: CC1(C)CC(C(=O)Nc2ccc(Oc3ccccc3)cc2)CC(C)(C)N1] | I-192 |
| 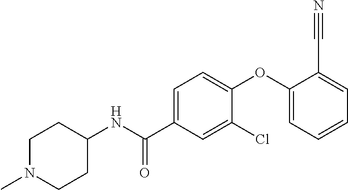 [SMILES: CN1CCC(NC(=O)c2ccc(Oc3ccccc3C#N)c(Cl)c2)CC1] | I-193 |
| 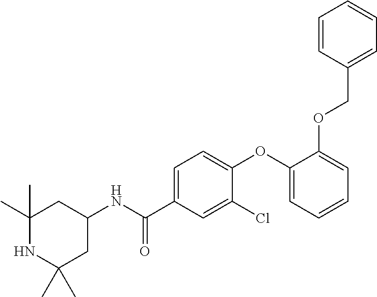 [SMILES: CC1(C)CC(NC(=O)c2ccc(Oc3ccccc3OCc3ccccc3)c(Cl)c2)CC(C)(C)N1] | I-194 |
| 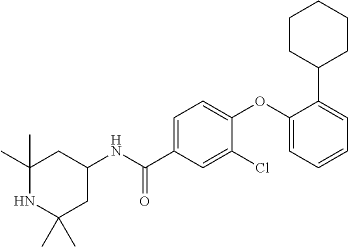 [SMILES: CC1(C)CC(NC(=O)c2ccc(Oc3ccccc3C3CCCCC3)c(Cl)c2)CC(C)(C)N1] | I-195 |
| 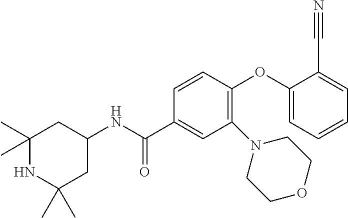 [SMILES: CC1(C)CC(NC(=O)c2ccc(Oc3ccccc3C#N)c(N3CCOCC3)c2)CC(C)(C)N1] | I-196 |
| 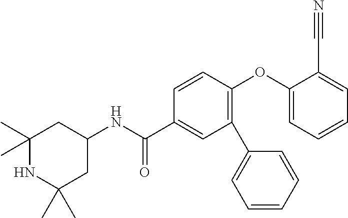 [SMILES: CC1(C)CC(NC(=O)c2ccc(Oc3ccccc3C#N)c(-c3ccccc3)c2)CC(C)(C)N1] | I-197 |
| 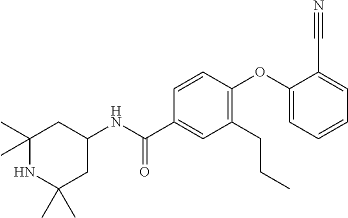 [SMILES: CCCc1cc(C(=O)NC2CC(C)(C)NC(C)(C)C2)ccc1Oc1ccccc1C#N] | I-198 |
| 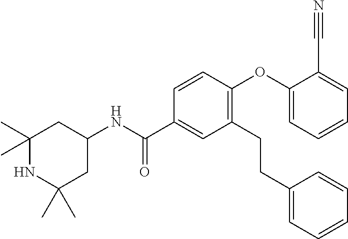 [SMILES: CC1(C)CC(NC(=O)c2ccc(Oc3ccccc3C#N)c(CCc3ccccc3)c2)CC(C)(C)N1] | I-199 |
| 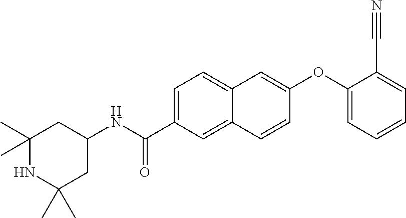 [SMILES: CC1(C)CC(NC(=O)c2ccc3cc(Oc4ccccc4C#N)ccc3c2)CC(C)(C)N1] | I-200 |
| 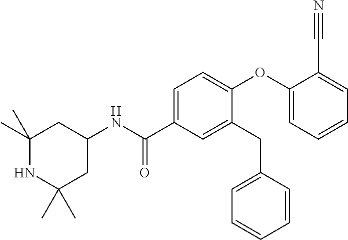 [SMILES: CC1(C)CC(NC(=O)c2ccc(Oc3ccccc3C#N)c(Cc3ccccc3)c2)CC(C)(C)N1] | I-201 |
| 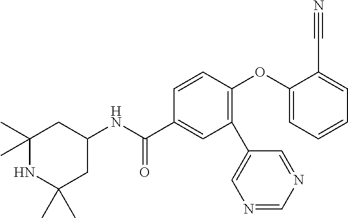 [SMILES: CC1(C)CC(NC(=O)c2ccc(Oc3ccccc3C#N)c(-c3cncnc3)c2)CC(C)(C)N1] | I-202 |
| 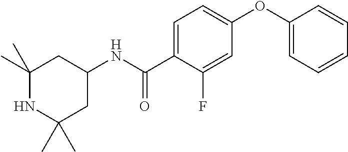 [SMILES: CC1(C)CC(NC(=O)c2ccc(Oc3ccccc3)cc2F)CC(C)(C)N1] | I-203 |
| 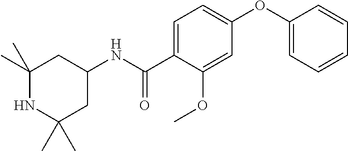 [SMILES: COc1cc(Oc2ccccc2)ccc1C(=O)NC1CC(C)(C)NC(C)(C)C1] | I-204 |
| 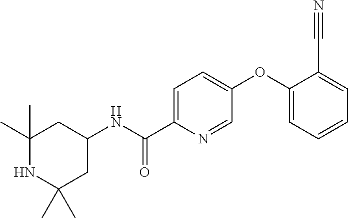 [SMILES: CC1(C)CC(NC(=O)c2ccc(Oc3ccccc3C#N)cn2)CC(C)(C)N1] | I-205 |
| 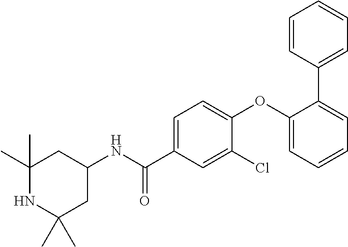 [SMILES: CC1(C)CC(NC(=O)c2ccc(Oc3ccccc3-c3ccccc3)c(Cl)c2)CC(C)(C)N1] | I-206 |
| 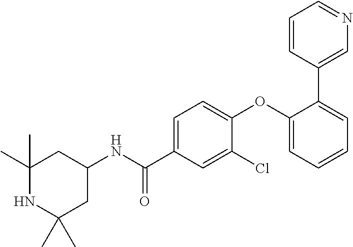 [SMILES: CC1(C)CC(NC(=O)c2ccc(Oc3ccccc3-c3cccnc3)c(Cl)c2)CC(C)(C)N1] | I-207 |
| 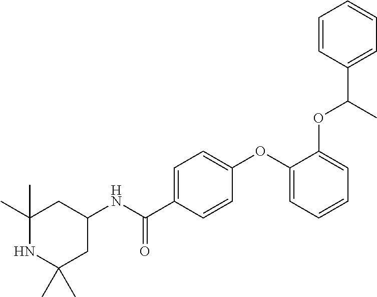 [SMILES: CC(Oc1ccccc1Oc1ccc(C(=O)NC2CC(C)(C)NC(C)(C)C2)cc1)c1ccccc1] | I-208 |
| 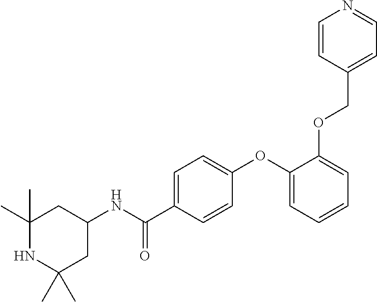 [SMILES: CC1(C)CC(NC(=O)c2ccc(Oc3ccccc3OCc3ccncc3)cc2)CC(C)(C)N1] | I-209 |
| 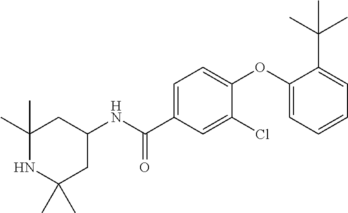 [SMILES: CC1(C)CC(NC(=O)c2ccc(Oc3ccccc3C(C)(C)C)c(Cl)c2)CC(C)(C)N1] | I-210 |
| 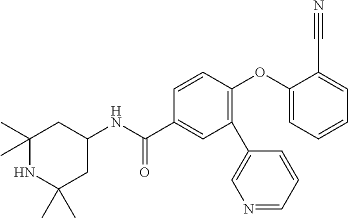 [SMILES: CC1(C)CC(NC(=O)c2ccc(Oc3ccccc3C#N)c(-c3cccnc3)c2)CC(C)(C)N1] | I-211 |
| 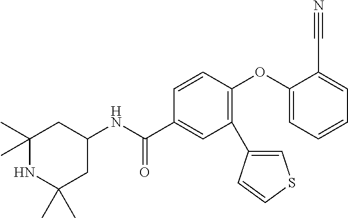 [SMILES: CC1(C)CC(NC(=O)c2ccc(Oc3ccccc3C#N)c(-c3ccsc3)c2)CC(C)(C)N1] | I-212 |
| 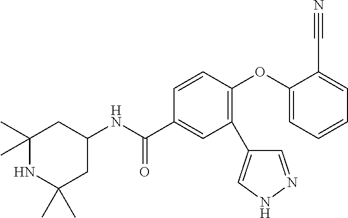 [SMILES: CC1(C)CC(NC(=O)c2ccc(Oc3ccccc3C#N)c(-c3cn[nH]c3)c2)CC(C)(C)N1] | I-213 |
| 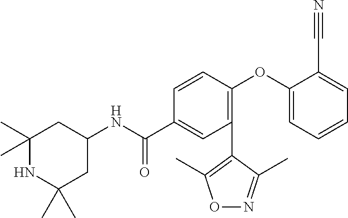 [SMILES: Cc1noc(C)c1-c1cc(C(=O)NC2CC(C)(C)NC(C)(C)C2)ccc1Oc1ccccc1C#N] | I-214 |
| 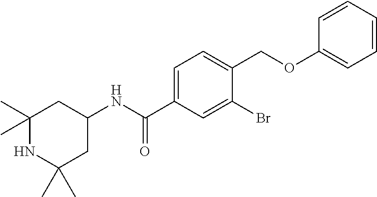 [SMILES: CC1(C)CC(NC(=O)c2ccc(COc3ccccc3)c(Br)c2)CC(C)(C)N1] | I-215 |
| 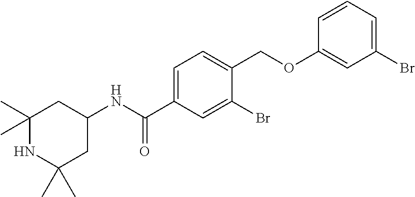 [SMILES: CC1(C)CC(NC(=O)c2ccc(COc3cccc(Br)c3)c(Br)c2)CC(C)(C)N1] | I-216 |
| 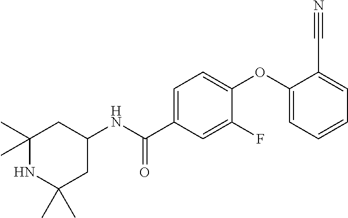 [SMILES: CC1(C)CC(NC(=O)c2ccc(Oc3ccccc3C#N)c(F)c2)CC(C)(C)N1] | I-217 |
| 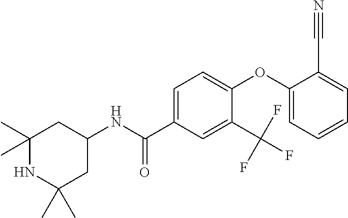 [SMILES: CC1(C)CC(NC(=O)c2ccc(Oc3ccccc3C#N)c(C(F)(F)F)c2)CC(C)(C)N1] | I-218 |
| 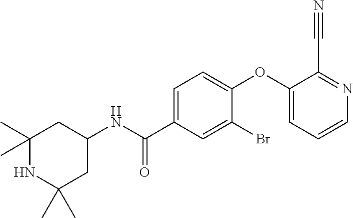 [SMILES: CC1(C)CC(NC(=O)c2ccc(Oc3cccnc3C#N)c(Br)c2)CC(C)(C)N1] | I-219 |
| 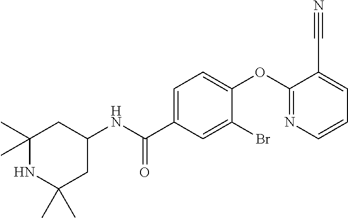 [SMILES: CC1(C)CC(NC(=O)c2ccc(Oc3ncccc3C#N)c(Br)c2)CC(C)(C)N1] | I-220 |
| 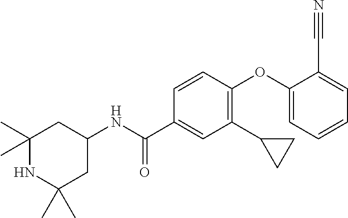 [SMILES: CC1(C)CC(NC(=O)c2ccc(Oc3ccccc3C#N)c(C3CC3)c2)CC(C)(C)N1] | I-221 |
| 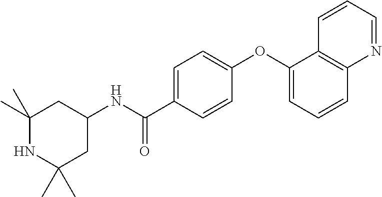 [SMILES: CC1(C)CC(NC(=O)c2ccc(Oc3cccc4ncccc34)cc2)CC(C)(C)N1] | I-222 |
| 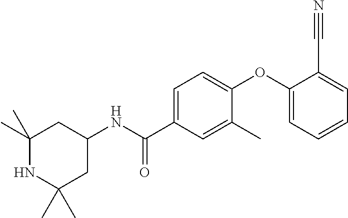 [SMILES: Cc1cc(C(=O)NC2CC(C)(C)NC(C)(C)C2)ccc1Oc1ccccc1C#N] | I-223 |
| 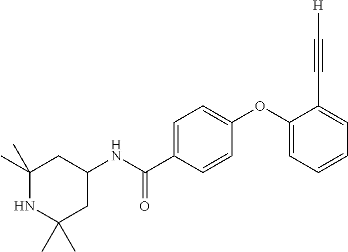 [SMILES: C#Cc1ccccc1Oc1ccc(C(=O)NC2CC(C)(C)NC(C)(C)C2)cc1] | I-224 |
| 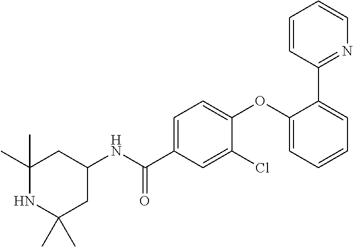 [SMILES: CC1(C)CC(NC(=O)c2ccc(Oc3ccccc3-c3ccccn3)c(Cl)c2)CC(C)(C)N1] | I-225 |
| 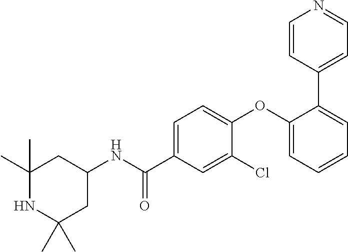 [SMILES: CC1(C)CC(NC(=O)c2ccc(Oc3ccccc3-c3ccncc3)c(Cl)c2)CC(C)(C)N1] | I-226 |
| 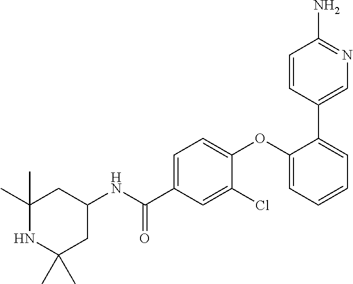 [SMILES: CC1(C)CC(NC(=O)c2ccc(Oc3ccccc3-c3ccc(N)nc3)c(Cl)c2)CC(C)(C)N1] | I-227 |
| 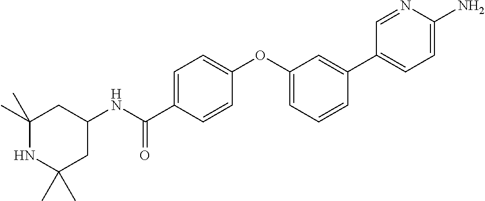 [SMILES: CC1(C)CC(NC(=O)c2ccc(Oc3cccc(-c4ccc(N)nc4)c3)cc2)CC(C)(C)N1] | I-228 |
| 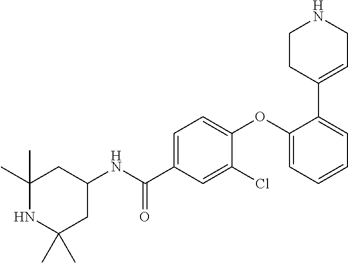 [SMILES: CC1(C)CC(NC(=O)c2ccc(Oc3ccccc3C3=CCNCC3)c(Cl)c2)CC(C)(C)N1] | I-229 |
| 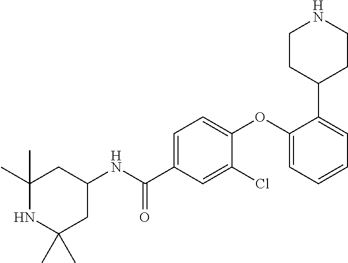 [SMILES: CC1(C)CC(NC(=O)c2ccc(Oc3ccccc3C3CCNCC3)c(Cl)c2)CC(C)(C)N1] | I-230 |
| 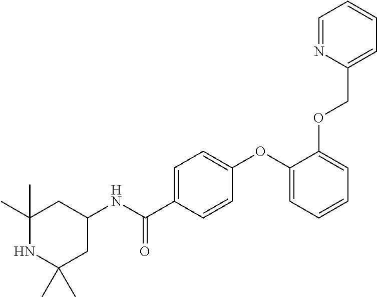 [SMILES: CC1(C)CC(NC(=O)c2ccc(Oc3ccccc3OCc3ccccn3)cc2)CC(C)(C)N1] | I-231 |
| 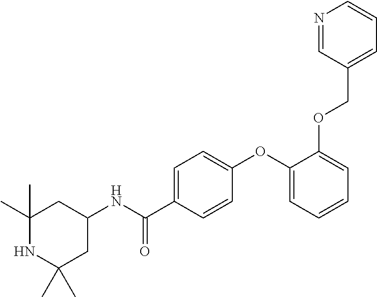 [SMILES: CC1(C)CC(NC(=O)c2ccc(Oc3ccccc3OCc3cccnc3)cc2)CC(C)(C)N1] | I-232 |
| 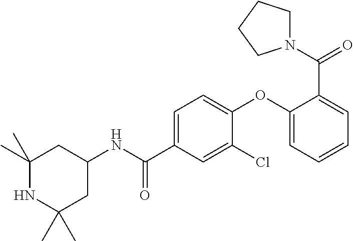 [SMILES: CC1(C)CC(NC(=O)c2ccc(Oc3ccccc3C(=O)N3CCCC3)c(Cl)c2)CC(C)(C)N1] | I-233 |
| 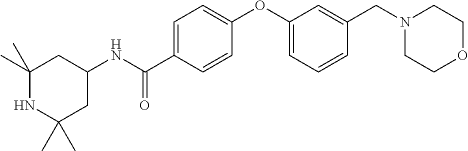 [SMILES: CC1(C)CC(NC(=O)c2ccc(Oc3cccc(CN4CCOCC4)c3)cc2)CC(C)(C)N1] | I-234 |
| 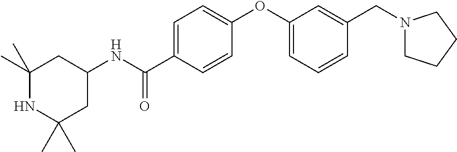 [SMILES: CC1(C)CC(NC(=O)c2ccc(Oc3cccc(CN4CCCC4)c3)cc2)CC(C)(C)N1] | I-235 |
| 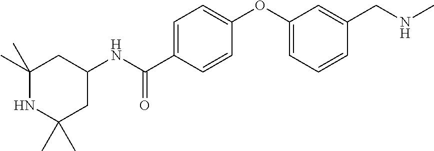 | I-236 |
| 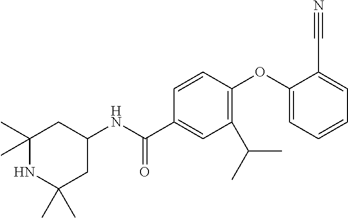 [SMILES: CC(C)c1cc(C(=O)NC2CC(C)(C)NC(C)(C)C2)ccc1Oc1ccccc1C#N] | I-237 |
| 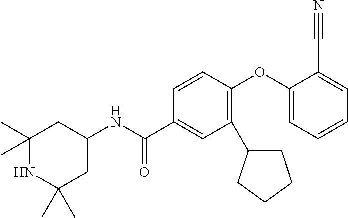 [SMILES: CC1(C)CC(NC(=O)c2ccc(Oc3ccccc3C#N)c(C3CCCC3)c2)CC(C)(C)N1] | I-238 |
| 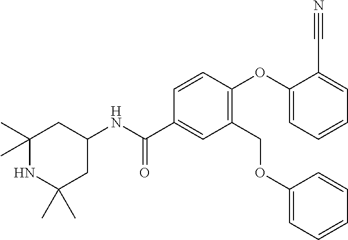 [SMILES: CC1(C)CC(NC(=O)c2ccc(Oc3ccccc3C#N)c(COc3ccccc3)c2)CC(C)(C)N1] | I-239 |
| 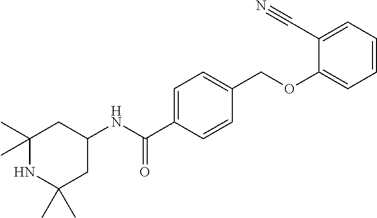 [SMILES: CC1(C)CC(NC(=O)c2ccc(COc3ccccc3C#N)cc2)CC(C)(C)N1] | I-240 |
| 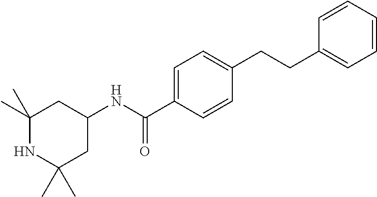 [SMILES: CC1(C)CC(NC(=O)c2ccc(CCc3ccccc3)cc2)CC(C)(C)N1] | I-241 |
| 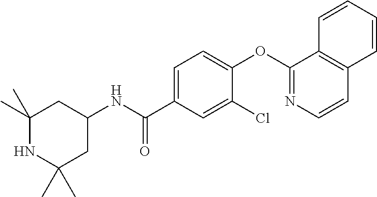 [SMILES: CC1(C)CC(NC(=O)c2ccc(Oc3nccc4ccccc34)c(Cl)c2)CC(C)(C)N1] | I-242 |
| 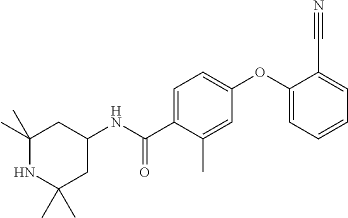 [SMILES: Cc1cc(Oc2ccccc2C#N)ccc1C(=O)NC1CC(C)(C)NC(C)(C)C1] | I-243 |
| 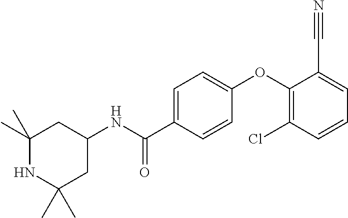 [SMILES: CC1(C)CC(NC(=O)c2ccc(Oc3c(Cl)cccc3C#N)cc2)CC(C)(C)N1] | I-244 |
| 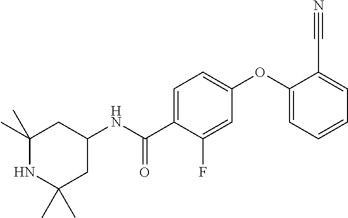 [SMILES: CC1(C)CC(NC(=O)c2ccc(Oc3ccccc3C#N)cc2F)CC(C)(C)N1] | I-245 |
| 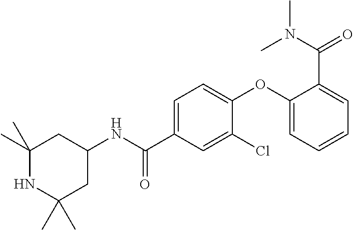 [SMILES: CN(C)C(=O)c1ccccc1Oc1ccc(C(=O)NC2CC(C)(C)NC(C)(C)C2)cc1Cl] | I-246 |
| 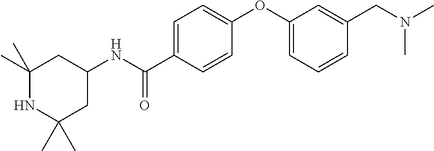 | I-247 |
| 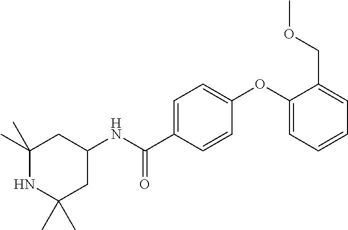 [SMILES: COCc1ccccc1Oc1ccc(C(=O)NC2CC(C)(C)NC(C)(C)C2)cc1] | I-248 |
| 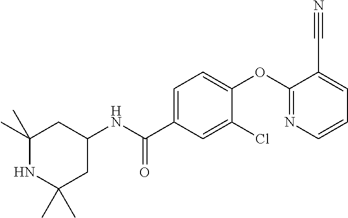 [SMILES: CC1(C)CC(NC(=O)c2ccc(Oc3ncccc3C#N)c(Cl)c2)CC(C)(C)N1] | I-249 |
| 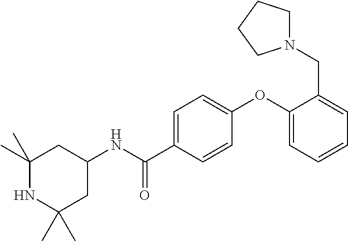 [SMILES: CC1(C)CC(NC(=O)c2ccc(Oc3ccccc3CN3CCCC3)cc2)CC(C)(C)N1] | I-250 |
| 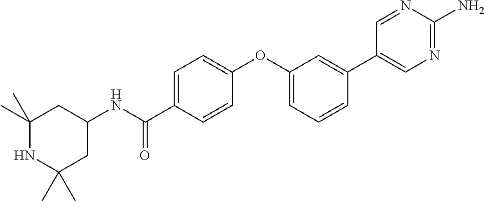 [SMILES: CC1(C)CC(NC(=O)c2ccc(Oc3cccc(-c4cnc(N)nc4)c3)cc2)CC(C)(C)N1] | I-251 |
| 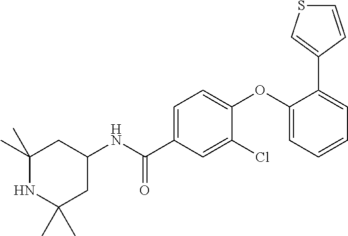 [SMILES: CC1(C)CC(NC(=O)c2ccc(Oc3ccccc3-c3ccsc3)c(Cl)c2)CC(C)(C)N1] | I-252 |
| 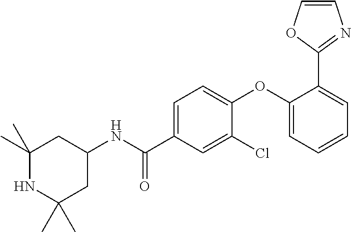 [SMILES: CC1(C)CC(NC(=O)c2ccc(Oc3ccccc3-c3ncco3)c(Cl)c2)CC(C)(C)N1] | I-253 |
| 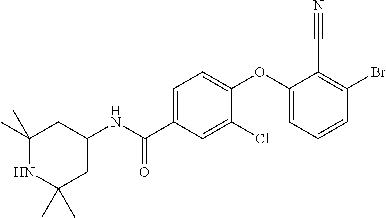 [SMILES: CC1(C)CC(NC(=O)c2ccc(Oc3cccc(Br)c3C#N)c(Cl)c2)CC(C)(C)N1] | I-254 |
| 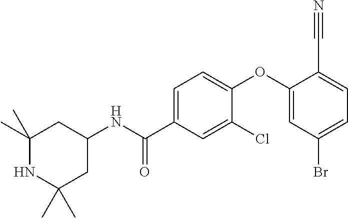 [SMILES: CC1(C)CC(NC(=O)c2ccc(Oc3cc(Br)ccc3C#N)c(Cl)c2)CC(C)(C)N1] | I-255 |
| 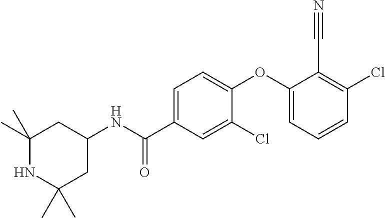 [SMILES: CC1(C)CC(NC(=O)c2ccc(Oc3cccc(Cl)c3C#N)c(Cl)c2)CC(C)(C)N1] | I-256 |
| 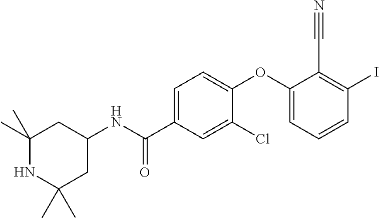 [SMILES: CC1(C)CC(NC(=O)c2ccc(Oc3cccc(I)c3C#N)c(Cl)c2)CC(C)(C)N1] | I-257 |
| 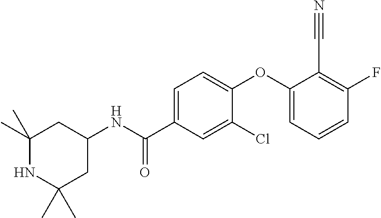 [SMILES: CC1(C)CC(NC(=O)c2ccc(Oc3cccc(F)c3C#N)c(Cl)c2)CC(C)(C)N1] | I-258 |
| 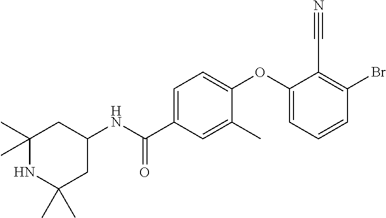 [SMILES: Cc1cc(C(=O)NC2CC(C)(C)NC(C)(C)C2)ccc1Oc1cccc(Br)c1C#N] | I-259 |
| 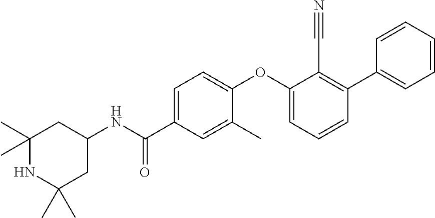 [SMILES: Cc1cc(C(=O)NC2CC(C)(C)NC(C)(C)C2)ccc1Oc1cccc(-c2ccccc2)c1C#N] | I-260 |
| 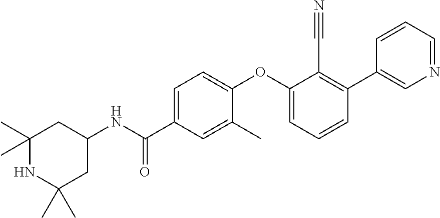 [SMILES: Cc1cc(C(=O)NC2CC(C)(C)NC(C)(C)C2)ccc1Oc1cccc(-c2cccnc2)c1C#N] | I-261 |
| 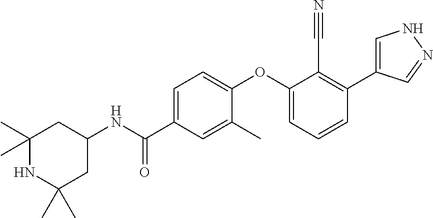 | I-262 |
| 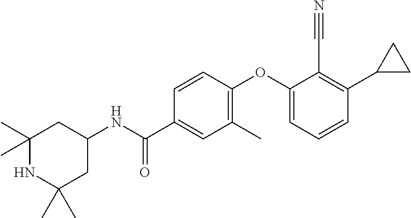 [SMILES: Cc1cc(C(=O)NC2CC(C)(C)NC(C)(C)C2)ccc1Oc1cccc(C2CC2)c1C#N] | I-263 |
| 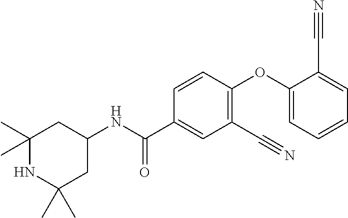 [SMILES: CC1(C)CC(NC(=O)c2ccc(Oc3ccccc3C#N)c(C#N)c2)CC(C)(C)N1] | I-264 |
| 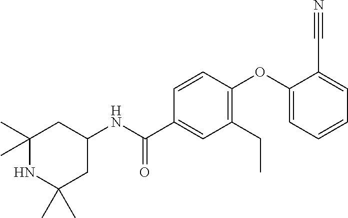 [SMILES: CCc1cc(C(=O)NC2CC(C)(C)NC(C)(C)C2)ccc1Oc1ccccc1C#N] | I-265 |
| 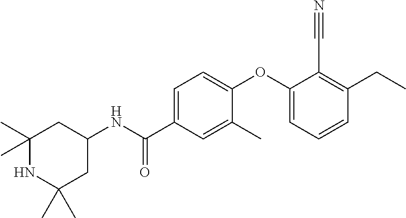 [SMILES: CCc1cccc(Oc2ccc(C(=O)NC3CC(C)(C)NC(C)(C)C3)cc2C)c1C#N] | I-266 |
| 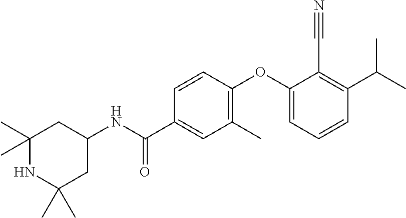 [SMILES: Cc1cc(C(=O)NC2CC(C)(C)NC(C)(C)C2)ccc1Oc1cccc(C(C)C)c1C#N] | I-267 |
| 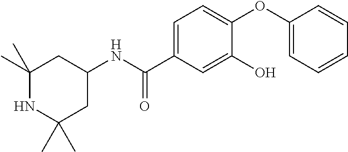 [SMILES: CC1(C)CC(NC(=O)c2ccc(Oc3ccccc3)c(O)c2)CC(C)(C)N1] | I-268 |
| 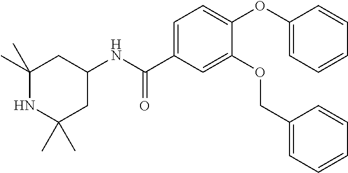 [SMILES: CC1(C)CC(NC(=O)c2ccc(Oc3ccccc3)c(OCc3ccccc3)c2)CC(C)(C)N1] | I-269 |
| 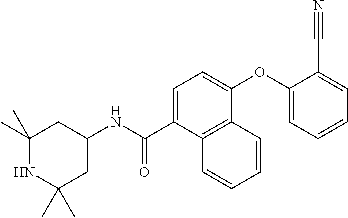 [SMILES: CC1(C)CC(NC(=O)c2ccc(Oc3ccccc3C#N)c3ccccc23)CC(C)(C)N1] | I-270 |
| 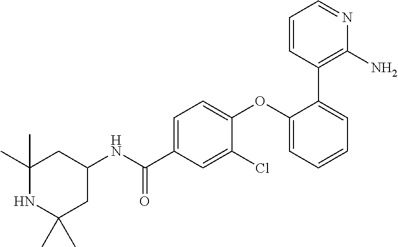 [SMILES: CC1(C)CC(NC(=O)c2ccc(Oc3ccccc3-c3cccnc3N)c(Cl)c2)CC(C)(C)N1] | I-271 |
| 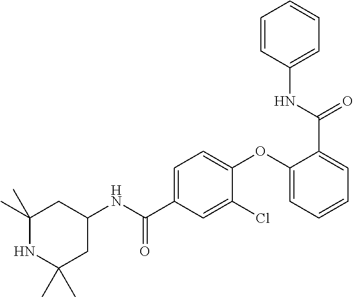 [SMILES: CC1(C)CC(NC(=O)c2ccc(Oc3ccccc3C(=O)Nc3ccccc3)c(Cl)c2)CC(C)(C)N1] | I-272 |
| 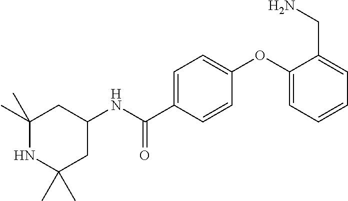 [SMILES: CC1(C)CC(NC(=O)c2ccc(Oc3ccccc3CN)cc2)CC(C)(C)N1] | I-273 |
| 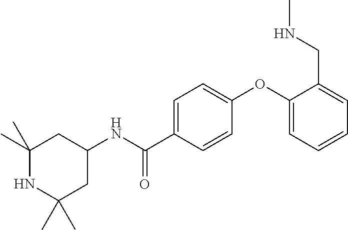 [SMILES: CNCc1ccccc1Oc1ccc(C(=O)NC2CC(C)(C)NC(C)(C)C2)cc1] | I-274 |
| 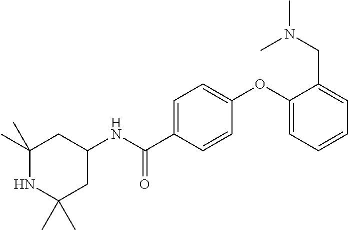 [SMILES: CN(C)Cc1ccccc1Oc1ccc(C(=O)NC2CC(C)(C)NC(C)(C)C2)cc1] | I-275 |
| 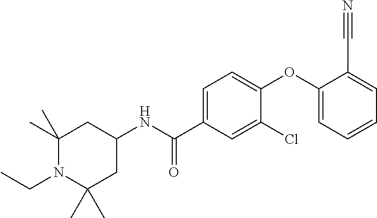 [SMILES: CCN1C(C)(C)CC(NC(=O)c2ccc(Oc3ccccc3C#N)c(Cl)c2)CC1(C)C] | I-276 |
| 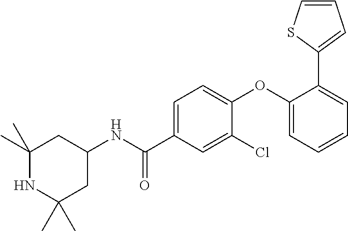 [SMILES: CC1(C)CC(NC(=O)c2ccc(Oc3ccccc3-c3cccs3)c(Cl)c2)CC(C)(C)N1] | I-277 |
| 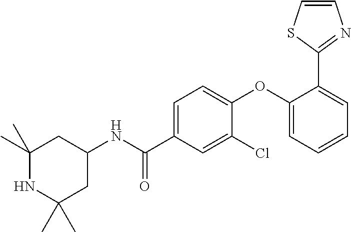 [SMILES: CC1(C)CC(NC(=O)c2ccc(Oc3ccccc3-c3nccs3)c(Cl)c2)CC(C)(C)N1] | I-278 |
| 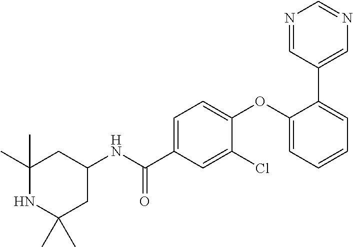 [SMILES: CC1(C)CC(NC(=O)c2ccc(Oc3ccccc3-c3cncnc3)c(Cl)c2)CC(C)(C)N1] | I-279 |
| 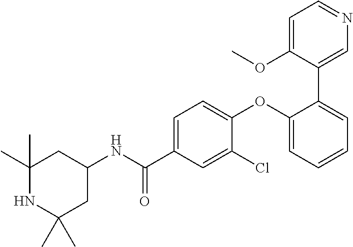 [SMILES: COc1ccncc1-c1ccccc1Oc1ccc(C(=O)NC2CC(C)(C)NC(C)(C)C2)cc1Cl] | I-280 |
| 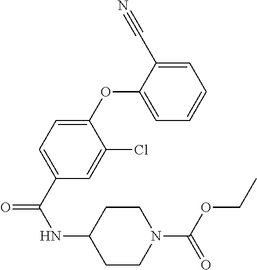 [SMILES: CCOC(=O)N1CCC(NC(=O)c2ccc(Oc3ccccc3C#N)c(Cl)c2)CC1] | I-281 |
| 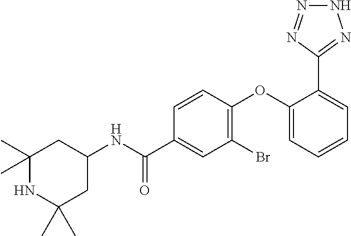 [SMILES: CC1(C)CC(NC(=O)c2ccc(Oc3ccccc3-c3nn[nH]n3)c(Br)c2)CC(C)(C)N1] | I-282 |
| 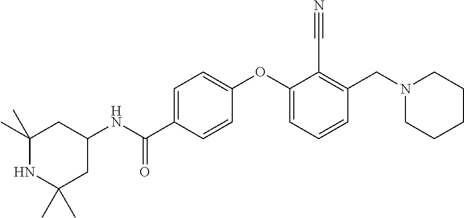 [SMILES: CC1(C)CC(NC(=O)c2ccc(Oc3cccc(CN4CCCCC4)c3C#N)cc2)CC(C)(C)N1] | I-283 |
| 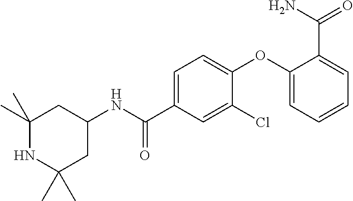 [SMILES: CC1(C)CC(NC(=O)c2ccc(Oc3ccccc3C(N)=O)c(Cl)c2)CC(C)(C)N1] | I-284 |
| 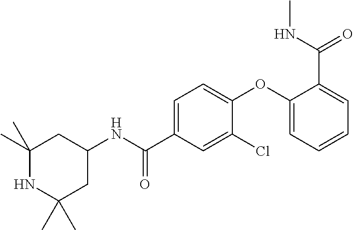 [SMILES: CNC(=O)c1ccccc1Oc1ccc(C(=O)NC2CC(C)(C)NC(C)(C)C2)cc1Cl] | I-285 |
| 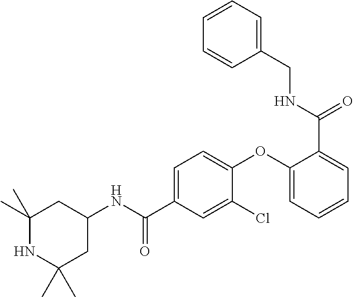 [SMILES: CC1(C)CC(NC(=O)c2ccc(Oc3ccccc3C(=O)NCc3ccccc3)c(Cl)c2)CC(C)(C)N1] | I-286 |
| 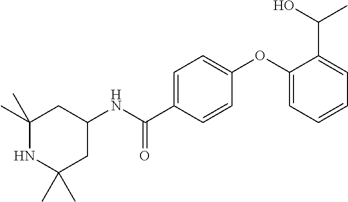 [SMILES: CC(O)c1ccccc1Oc1ccc(C(=O)NC2CC(C)(C)NC(C)(C)C2)cc1] | I-287 |
| 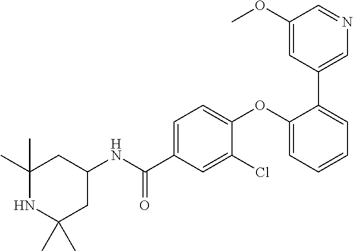 [SMILES: COc1cncc(-c2ccccc2Oc2ccc(C(=O)NC3CC(C)(C)NC(C)(C)C3)cc2Cl)c1] | I-288 |
| 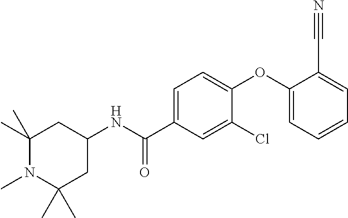 [SMILES: CN1C(C)(C)CC(NC(=O)c2ccc(Oc3ccccc3C#N)c(Cl)c2)CC1(C)C] | I-289 |
| 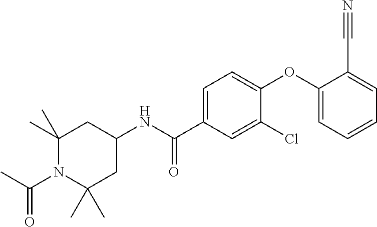 [SMILES: CC(=O)N1C(C)(C)CC(NC(=O)c2ccc(Oc3ccccc3C#N)c(Cl)c2)CC1(C)C] | I-290 |
| 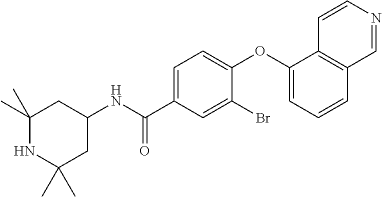 [SMILES: CC1(C)CC(NC(=O)c2ccc(Oc3cccc4cnccc34)c(Br)c2)CC(C)(C)N1] | I-291 |
| 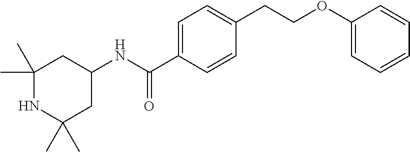 [SMILES: CC1(C)CC(NC(=O)c2ccc(CCOc3ccccc3)cc2)CC(C)(C)N1] | I-292 |
| 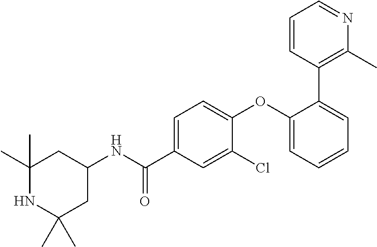 | I-293 |
| 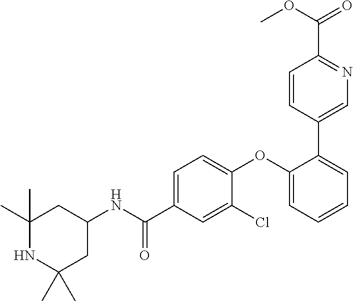 [SMILES: COC(=O)c1ccc(-c2ccccc2Oc2ccc(C(=O)NC3CC(C)(C)NC(C)(C)C3)cc2Cl)cn1] | I-294 |
| 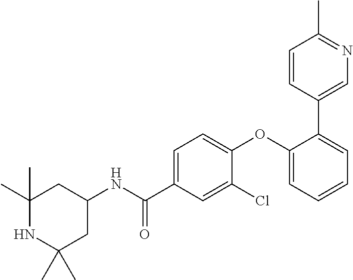 [SMILES: Cc1ccc(-c2ccccc2Oc2ccc(C(=O)NC3CC(C)(C)NC(C)(C)C3)cc2Cl)cn1] | I-295 |
| 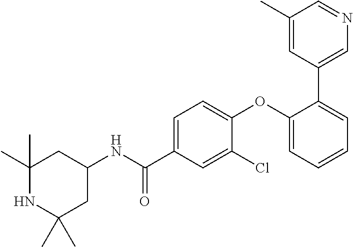 [SMILES: Cc1cncc(-c2ccccc2Oc2ccc(C(=O)NC3CC(C)(C)NC(C)(C)C3)cc2Cl)c1] | I-296 |
| 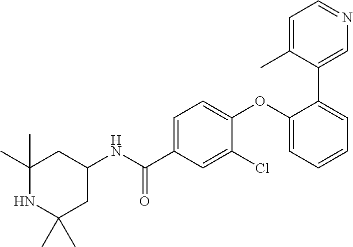 [SMILES: Cc1ccncc1-c1ccccc1Oc1ccc(C(=O)NC2CC(C)(C)NC(C)(C)C2)cc1Cl] | I-297 |
| 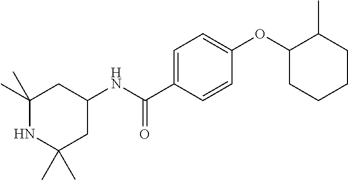 [SMILES: CC1CCCCC1Oc1ccc(C(=O)NC2CC(C)(C)NC(C)(C)C2)cc1] | I-298 |
| 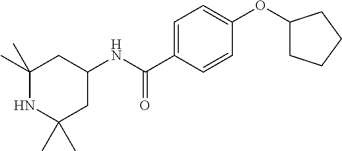 [SMILES: CC1(C)CC(NC(=O)c2ccc(OC3CCCC3)cc2)CC(C)(C)N1] | I-299 |
| 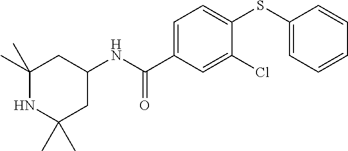 [SMILES: CC1(C)CC(NC(=O)c2ccc(Sc3ccccc3)c(Cl)c2)CC(C)(C)N1] | I-300 |
| 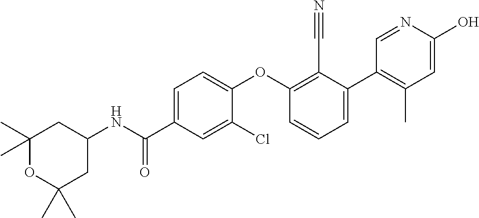 [SMILES: Cc1cc(O)ncc1-c1cccc(Oc2ccc(C(=O)NC3CC(C)(C)OC(C)(C)C3)cc2Cl)c1C#N] | I-301 |
| 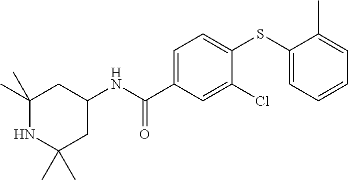 [SMILES: Cc1ccccc1Sc1ccc(C(=O)NC2CC(C)(C)NC(C)(C)C2)cc1Cl] | I-302 |
| 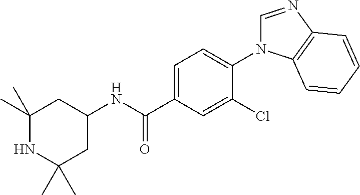 [SMILES: CC1(C)CC(NC(=O)c2ccc(-n3cnc4ccccc43)c(Cl)c2)CC(C)(C)N1] | I-303 |
| 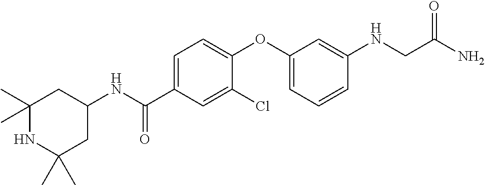 [SMILES: CC1(C)CC(NC(=O)c2ccc(Oc3cccc(NCC(N)=O)c3)c(Cl)c2)CC(C)(C)N1] | I-304 |
| 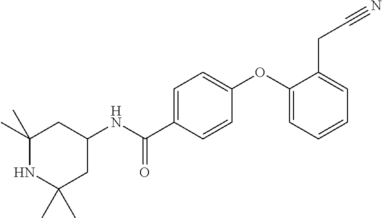 [SMILES: CC1(C)CC(NC(=O)c2ccc(Oc3ccccc3CC#N)cc2)CC(C)(C)N1] | I-305 |
| 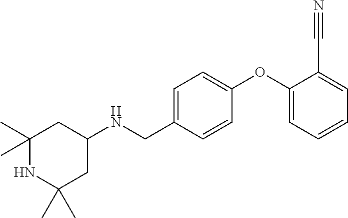 [SMILES: CC1(C)CC(NCc2ccc(Oc3ccccc3C#N)cc2)CC(C)(C)N1] | I-306 |
| 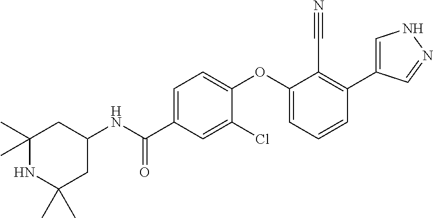 [SMILES: CC1(C)CC(NC(=O)c2ccc(Oc3cccc(-c4cn[nH]c4)c3C#N)c(Cl)c2)CC(C)(C)N1] | I-307 |
| 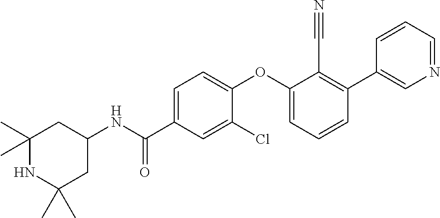 [SMILES: CC1(C)CC(NC(=O)c2ccc(Oc3cccc(-c4cccnc4)c3C#N)c(Cl)c2)CC(C)(C)N1] | I-308 |
| 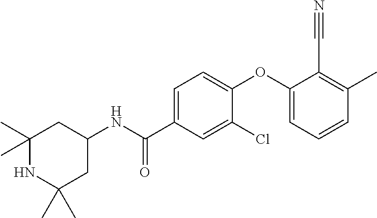 | I-309 |
| 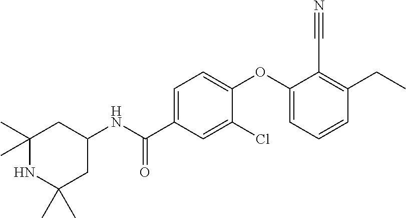 [SMILES: CCc1cccc(Oc2ccc(C(=O)NC3CC(C)(C)NC(C)(C)C3)cc2Cl)c1C#N] | I-310 |
| 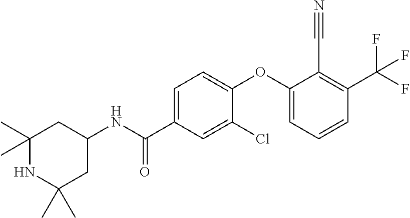 [SMILES: CC1(C)CC(NC(=O)c2ccc(Oc3cccc(C(F)(F)F)c3C#N)c(Cl)c2)CC(C)(C)N1] | I-311 |
| 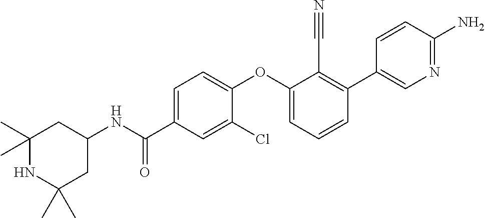 [SMILES: CC1(C)CC(NC(=O)c2ccc(Oc3cccc(-c4ccc(N)nc4)c3C#N)c(Cl)c2)CC(C)(C)N1] | I-312 |
| 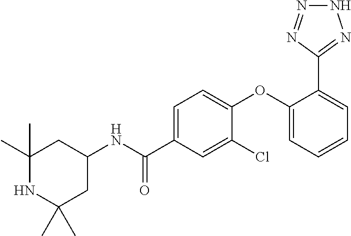 [SMILES: CC1(C)CC(NC(=O)c2ccc(Oc3ccccc3-c3nn[nH]n3)c(Cl)c2)CC(C)(C)N1] | I-313 |
| 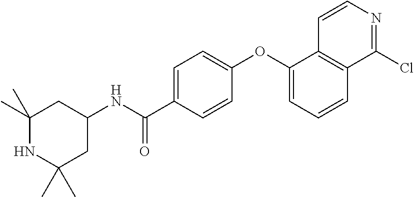 [SMILES: CC1(C)CC(NC(=O)c2ccc(Oc3cccc4c(Cl)nccc34)cc2)CC(C)(C)N1] | I-314 |
| 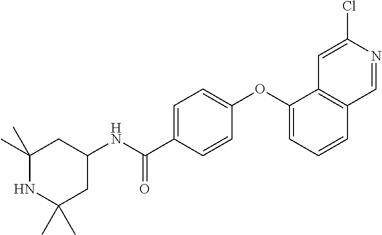 [SMILES: CC1(C)CC(NC(=O)c2ccc(Oc3cccc4cnc(Cl)cc34)cc2)CC(C)(C)N1] | I-315 |
| 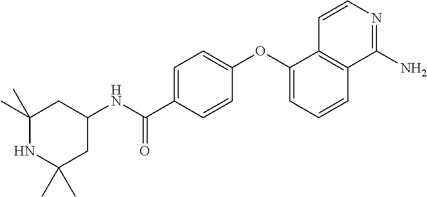 [SMILES: CC1(C)CC(NC(=O)c2ccc(Oc3cccc4c(N)nccc34)cc2)CC(C)(C)N1] | I-316 |
| 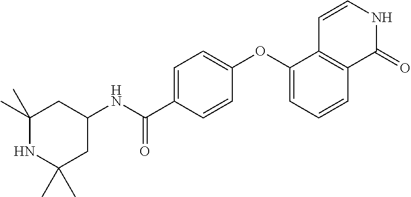 [SMILES: CC1(C)CC(NC(=O)c2ccc(Oc3cccc4c(=O)[nH]ccc34)cc2)CC(C)(C)N1] | I-317 |
| 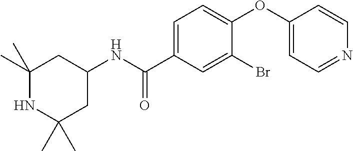 [SMILES: CC1(C)CC(NC(=O)c2ccc(Oc3ccncc3)c(Br)c2)CC(C)(C)N1] | I-318 |
| 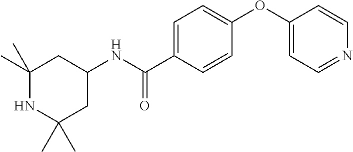 [SMILES: CC1(C)CC(NC(=O)c2ccc(Oc3ccncc3)cc2)CC(C)(C)N1] | I-319 |
| 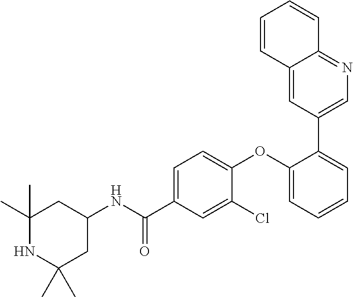 [SMILES: CC1(C)CC(NC(=O)c2ccc(Oc3ccccc3-c3cnc4ccccc4c3)c(Cl)c2)CC(C)(C)N1] | I-320 |
| 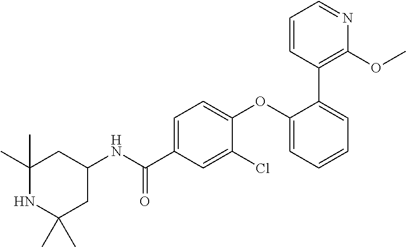 | I-321 |
| 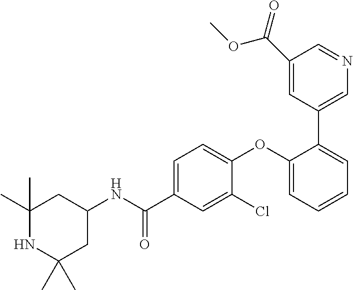 [SMILES: COC(=O)c1cncc(-c2ccccc2Oc2ccc(C(=O)NC3CC(C)(C)NC(C)(C)C3)cc2Cl)c1] | I-322 |
| 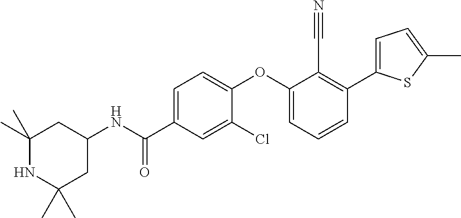 | I-323 |
| 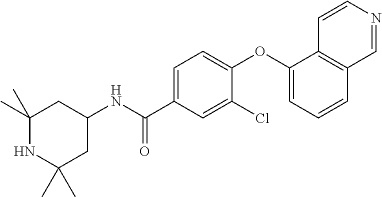 [SMILES: CC1(C)CC(NC(=O)c2ccc(Oc3cccc4cnccc34)c(Cl)c2)CC(C)(C)N1] | I-324 |
| 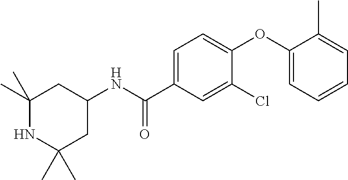 [SMILES: Cc1ccccc1Oc1ccc(C(=O)NC2CC(C)(C)NC(C)(C)C2)cc1Cl] | I-325 |
| 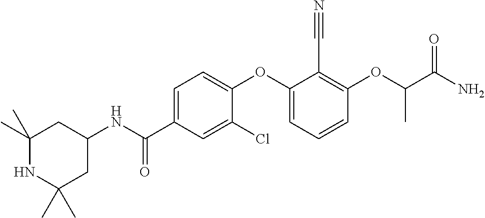 [SMILES: CC(Oc1cccc(Oc2ccc(C(=O)NC3CC(C)(C)NC(C)(C)C3)cc2Cl)c1C#N)C(N)=O] | I-326 |
| 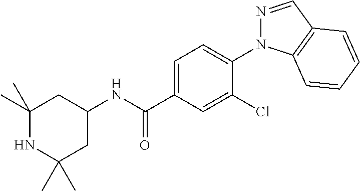 [SMILES: CC1(C)CC(NC(=O)c2ccc(-n3ncc4ccccc43)c(Cl)c2)CC(C)(C)N1] | I-327 |
| 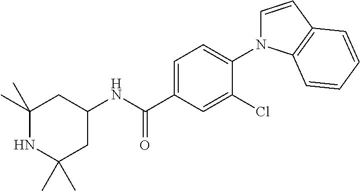 [SMILES: CC1(C)CC(NC(=O)c2ccc(-n3ccc4ccccc43)c(Cl)c2)CC(C)(C)N1] | I-328 |
| 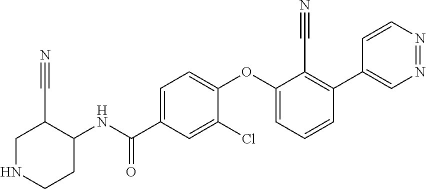 [SMILES: N#Cc1c(Oc2ccc(C(=O)NC3CCNCC3C#N)cc2Cl)cccc1-c1ccnnc1] | I-329 |
| 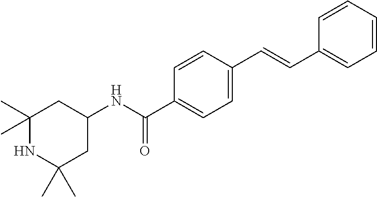 [SMILES: CC1(C)CC(NC(=O)c2ccc(/C=C/c3ccccc3)cc2)CC(C)(C)N1] | I-330 |
| 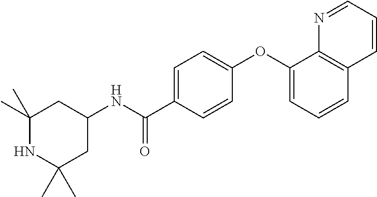 [SMILES: CC1(C)CC(NC(=O)c2ccc(Oc3cccc4cccnc34)cc2)CC(C)(C)N1] | I-331 |
| 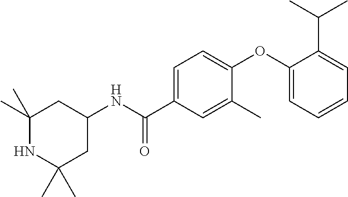 [SMILES: Cc1cc(C(=O)NC2CC(C)(C)NC(C)(C)C2)ccc1Oc1ccccc1C(C)C] | I-332 |
| 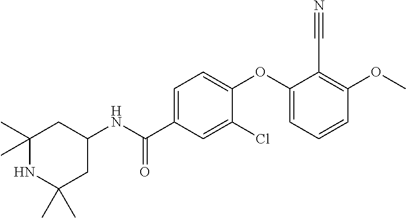 | I-333 |
| 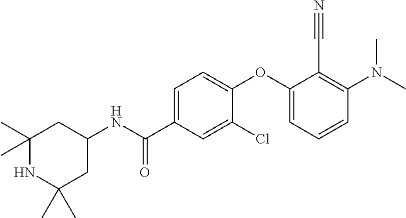 | I-334 |
| 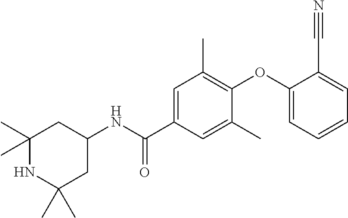 [SMILES: Cc1cc(C(=O)NC2CC(C)(C)NC(C)(C)C2)cc(C)c1Oc1ccccc1C#N] | I-335 |
| 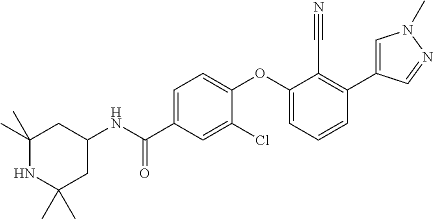 [SMILES: Cn1cc(-c2cccc(Oc3ccc(C(=O)NC4CC(C)(C)NC(C)(C)C4)cc3Cl)c2C#N)cn1] | I-336 |
| 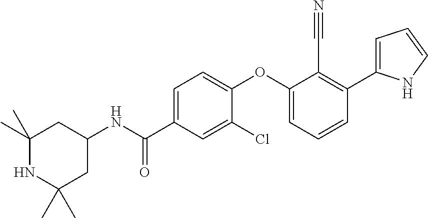 [SMILES: CC1(C)CC(NC(=O)c2ccc(Oc3cccc(-c4ccc[nH]4)c3C#N)c(Cl)c2)CC(C)(C)N1] | I-337 |
| 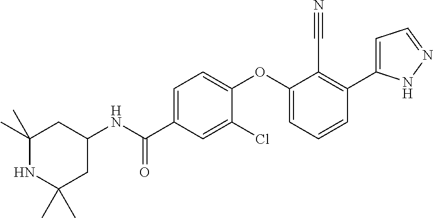 [SMILES: CC1(C)CC(NC(=O)c2ccc(Oc3cccc(-c4ccn[nH]4)c3C#N)c(Cl)c2)CC(C)(C)N1] | I-338 |
| 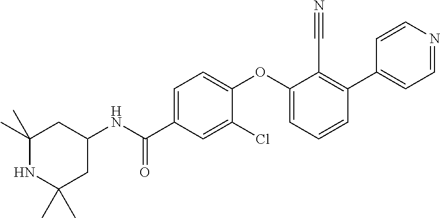 [SMILES: CC1(C)CC(NC(=O)c2ccc(Oc3cccc(-c4ccncc4)c3C#N)c(Cl)c2)CC(C)(C)N1] | I-339 |
| 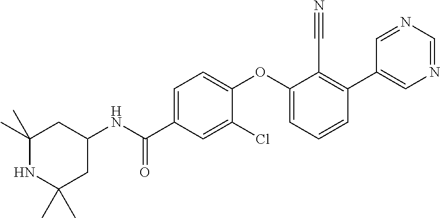 [SMILES: CC1(C)CC(NC(=O)c2ccc(Oc3cccc(-c4cncnc4)c3C#N)c(Cl)c2)CC(C)(C)N1] | I-340 |
| 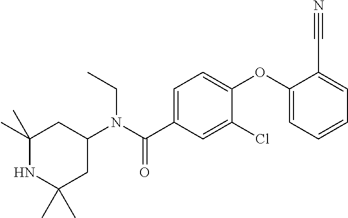 [SMILES: CCN(C(=O)c1ccc(Oc2ccccc2C#N)c(Cl)c1)C1CC(C)(C)NC(C)(C)C1] | I-341 |
| 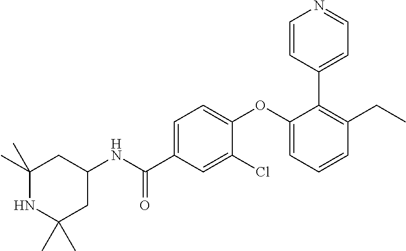 [SMILES: CCc1cccc(Oc2ccc(C(=O)NC3CC(C)(C)NC(C)(C)C3)cc2Cl)c1-c1ccncc1] | I-342 |
| 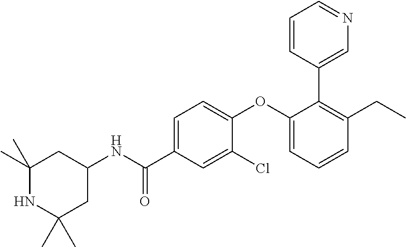 [SMILES: CCc1cccc(Oc2ccc(C(=O)NC3CC(C)(C)NC(C)(C)C3)cc2Cl)c1-c1cccnc1] | I-343 |
| 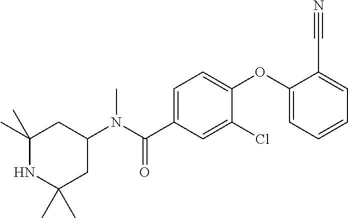 [SMILES: CN(C(=O)c1ccc(Oc2ccccc2C#N)c(Cl)c1)C1CC(C)(C)NC(C)(C)C1] | I-344 |
| 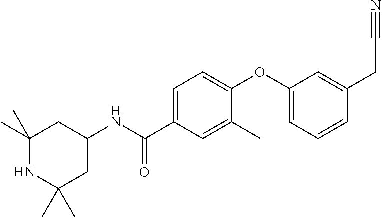 [SMILES: Cc1cc(C(=O)NC2CC(C)(C)NC(C)(C)C2)ccc1Oc1cccc(CC#N)c1] | I-345 |
| 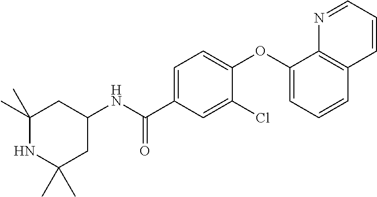 [SMILES: CC1(C)CC(NC(=O)c2ccc(Oc3cccc4cccnc34)c(Cl)c2)CC(C)(C)N1] | I-346 |
| 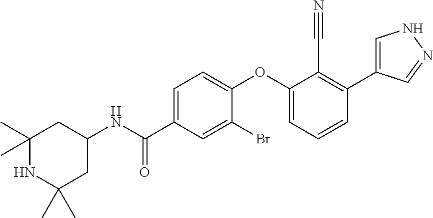 [SMILES: CC1(C)CC(NC(=O)c2ccc(Oc3cccc(-c4cn[nH]c4)c3C#N)c(Br)c2)CC(C)(C)N1] | I-347 |
| 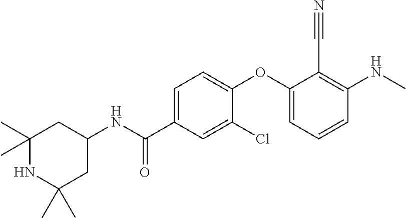 | I-348 |
| 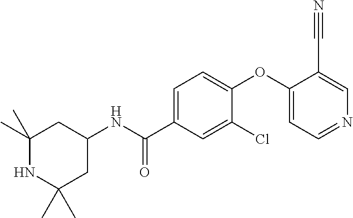 [SMILES: CC1(C)CC(NC(=O)c2ccc(Oc3ccncc3C#N)c(Cl)c2)CC(C)(C)N1] | I-349 |
| 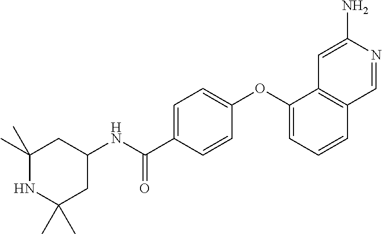 [SMILES: CC1(C)CC(NC(=O)c2ccc(Oc3cccc4cnc(N)cc34)cc2)CC(C)(C)N1] | I-350 |
| 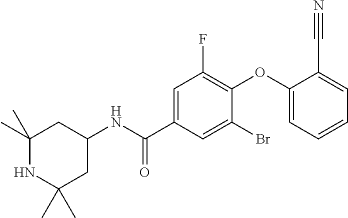 [SMILES: CC1(C)CC(NC(=O)c2cc(F)c(Oc3ccccc3C#N)c(Br)c2)CC(C)(C)N1] | I-351 |
| 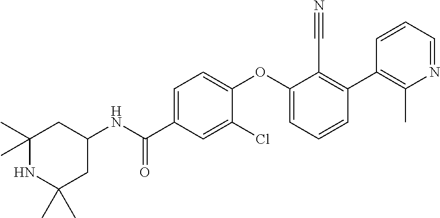 [SMILES: Cc1ncccc1-c1cccc(Oc2ccc(C(=O)NC3CC(C)(C)NC(C)(C)C3)cc2Cl)c1C#N] | I-352 |
| 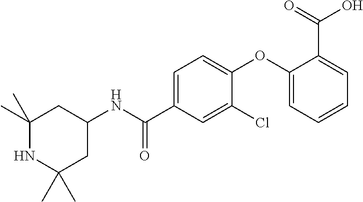 [SMILES: CC1(C)CC(NC(=O)c2ccc(Oc3ccccc3C(=O)O)c(Cl)c2)CC(C)(C)N1] | I-353 |
| 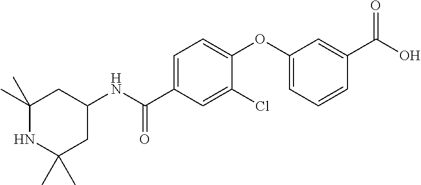 [SMILES: CC1(C)CC(NC(=O)c2ccc(Oc3cccc(C(=O)O)c3)c(Cl)c2)CC(C)(C)N1] | I-354 |
| 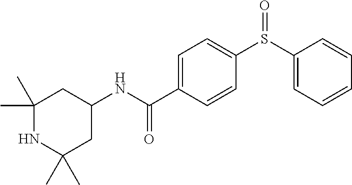 [SMILES: CC1(C)CC(NC(=O)c2ccc(S(=O)c3ccccc3)cc2)CC(C)(C)N1] | I-355 |
| 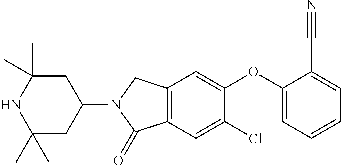 [SMILES: CC1(C)CC(N2Cc3cc(Oc4ccccc4C#N)c(Cl)cc3C2=O)CC(C)(C)N1] | I-356 |
| 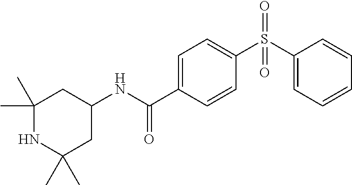 [SMILES: CC1(C)CC(NC(=O)c2ccc(S(=O)(=O)c3ccccc3)cc2)CC(C)(C)N1] | I-357 |
| 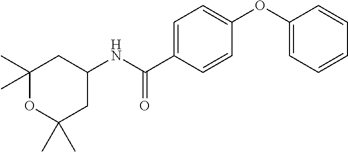 [SMILES: CC1(C)CC(NC(=O)c2ccc(Oc3ccccc3)cc2)CC(C)(C)O1] | I-358 |
| 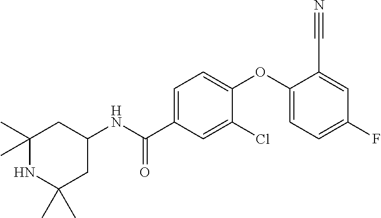 [SMILES: CC1(C)CC(NC(=O)c2ccc(Oc3ccc(F)cc3C#N)c(Cl)c2)CC(C)(C)N1] | I-359 |
| 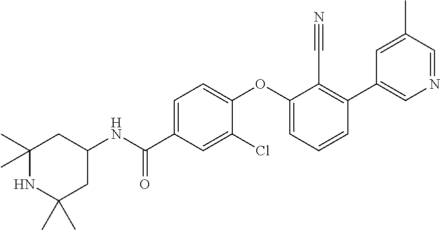 [SMILES: Cc1cncc(-c2cccc(Oc3ccc(C(=O)NC4CC(C)(C)NC(C)(C)C4)cc3Cl)c2C#N)c1] | I-360 |
| 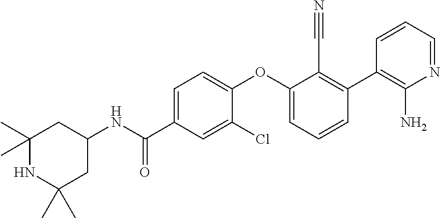 [SMILES: CC1(C)CC(NC(=O)c2ccc(Oc3cccc(-c4cccnc4N)c3C#N)c(Cl)c2)CC(C)(C)N1] | I-361 |
| 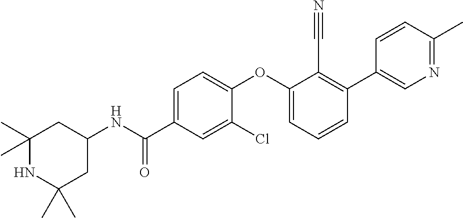 | I-362 |
| 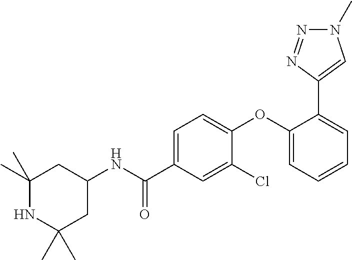 | I-363 |
| 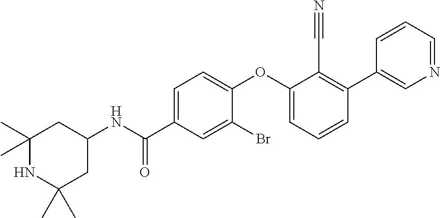 [SMILES: CC1(C)CC(NC(=O)c2ccc(Oc3cccc(-c4cccnc4)c3C#N)c(Br)c2)CC(C)(C)N1] | I-364 |
| 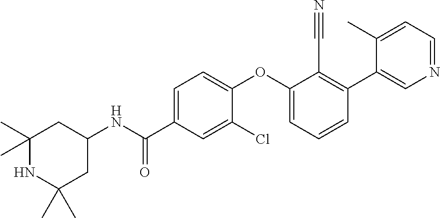 [SMILES: Cc1ccncc1-c1cccc(Oc2ccc(C(=O)NC3CC(C)(C)NC(C)(C)C3)cc2Cl)c1C#N] | I-365 |
| 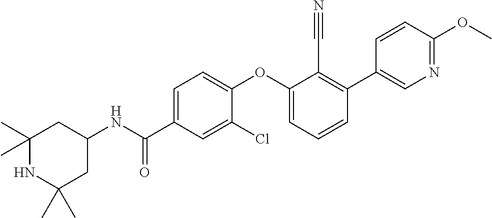 | I-366 |
| 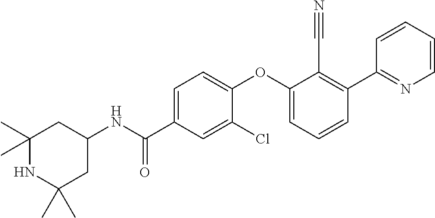 [SMILES: CC1(C)CC(NC(=O)c2ccc(Oc3cccc(-c4ccccn4)c3C#N)c(Cl)c2)CC(C)(C)N1] | I-367 |
| 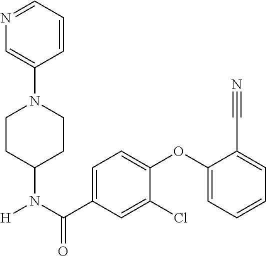 [SMILES: N#Cc1ccccc1Oc1ccc(C(=O)NC2CCN(c3cccnc3)CC2)cc1Cl] | I-368 |
| 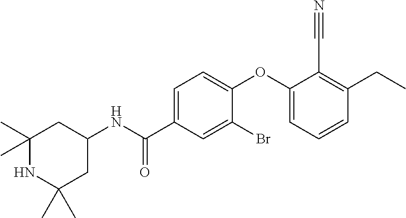 [SMILES: CCc1cccc(Oc2ccc(C(=O)NC3CC(C)(C)NC(C)(C)C3)cc2Br)c1C#N] | I-369 |
| 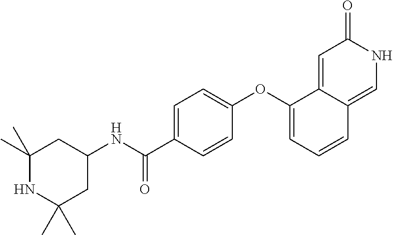 [SMILES: CC1(C)CC(NC(=O)c2ccc(Oc3cccc4c[nH]c(=O)cc34)cc2)CC(C)(C)N1] | I-370 |
| 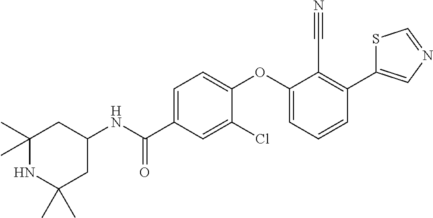 [SMILES: CC1(C)CC(NC(=O)c2ccc(Oc3cccc(-c4cncs4)c3C#N)c(Cl)c2)CC(C)(C)N1] | I-371 |
| 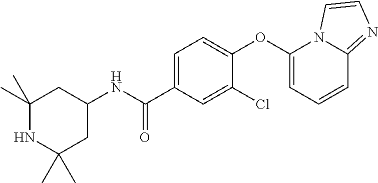 [SMILES: CC1(C)CC(NC(=O)c2ccc(Oc3cccc4nccn34)c(Cl)c2)CC(C)(C)N1] | I-372 |
| 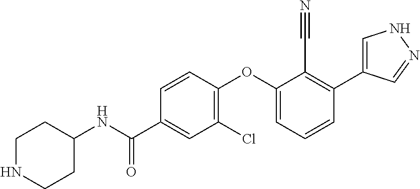 [SMILES: N#Cc1c(Oc2ccc(C(=O)NC3CCNCC3)cc2Cl)cccc1-c1cn[nH]c1] | I-373 |
| 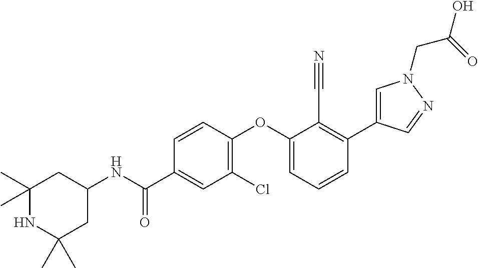 [SMILES: CC1(C)CC(NC(=O)c2ccc(Oc3cccc(-c4cnn(CC(=O)O)c4)c3C#N)c(Cl)c2)CC(C)(C)N1] | I-374 |
| 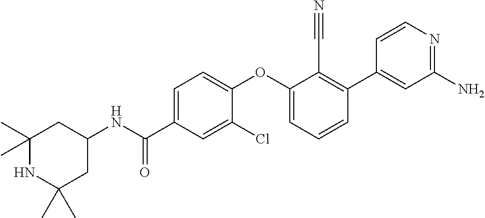 [SMILES: CC1(C)CC(NC(=O)c2ccc(Oc3cccc(-c4ccnc(N)c4)c3C#N)c(Cl)c2)CC(C)(C)N1] | I-375 |
| 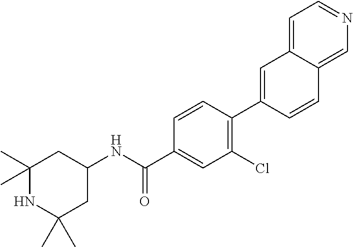 [SMILES: CC1(C)CC(NC(=O)c2ccc(-c3ccc4cnccc4c3)c(Cl)c2)CC(C)(C)N1] | I-376 |
| 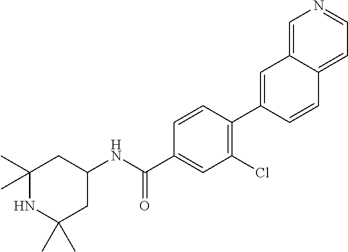 [SMILES: CC1(C)CC(NC(=O)c2ccc(-c3ccc4ccncc4c3)c(Cl)c2)CC(C)(C)N1] | I-377 |
| 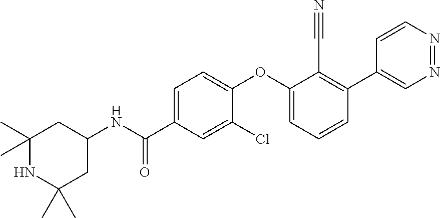 [SMILES: CC1(C)CC(NC(=O)c2ccc(Oc3cccc(-c4ccnnc4)c3C#N)c(Cl)c2)CC(C)(C)N1] | I-378 |
| 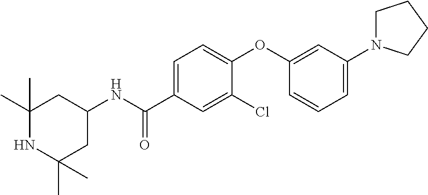 [SMILES: CC1(C)CC(NC(=O)c2ccc(Oc3cccc(N4CCCC4)c3)c(Cl)c2)CC(C)(C)N1] | I-379 |
| 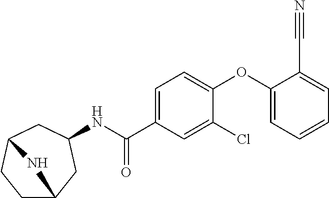 [SMILES: N#Cc1ccccc1Oc1ccc(C(=O)N[C@@H]2C[C@H]3CC[C@@H](C2)N3)cc1Cl] | I-380 |
| 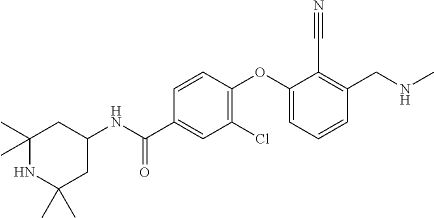 | I-381 |
| 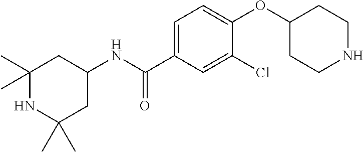 [SMILES: CC1(C)CC(NC(=O)c2ccc(OC3CCNCC3)c(Cl)c2)CC(C)(C)N1] | I-382 |
| 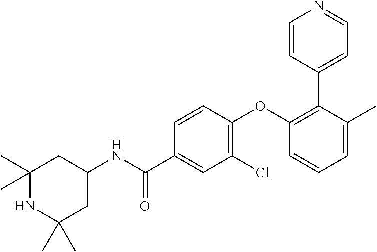 | I-383 |
| 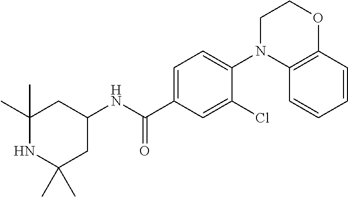 [SMILES: CC1(C)CC(NC(=O)c2ccc(N3CCOc4ccccc43)c(Cl)c2)CC(C)(C)N1] | I-384 |
| 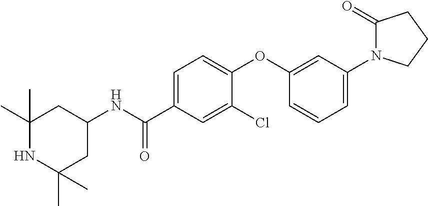 [SMILES: CC1(C)CC(NC(=O)c2ccc(Oc3cccc(N4CCCC4=O)c3)c(Cl)c2)CC(C)(C)N1] | I-385 |
| 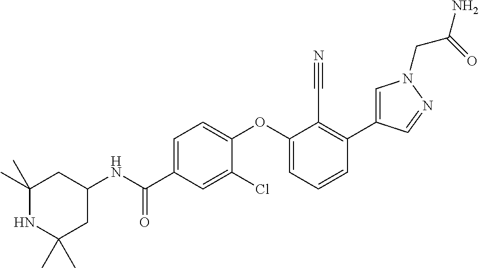 [SMILES: CC1(C)CC(NC(=O)c2ccc(Oc3cccc(-c4cnn(CC(N)=O)c4)c3C#N)c(Cl)c2)CC(C)(C)N1] | I-386 |
| 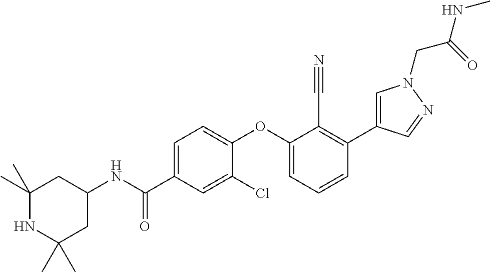 | I-387 |
| 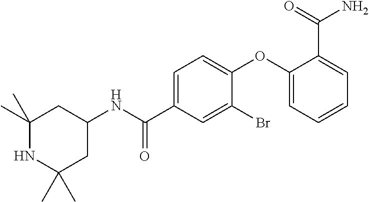 [SMILES: CC1(C)CC(NC(=O)c2ccc(Oc3ccccc3C(N)=O)c(Br)c2)CC(C)(C)N1] | I-388 |
| 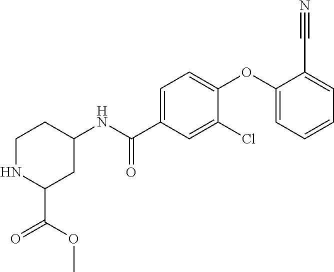 [SMILES: COC(=O)C1CC(NC(=O)c2ccc(Oc3ccccc3C#N)c(Cl)c2)CCN1] | I-389 |
| 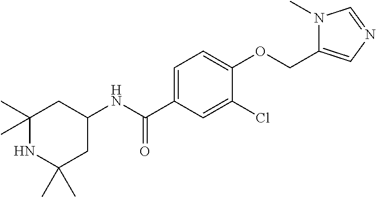 [SMILES: Cn1cncc1COc1ccc(C(=O)NC2CC(C)(C)NC(C)(C)C2)cc1Cl] | I-390 |
| 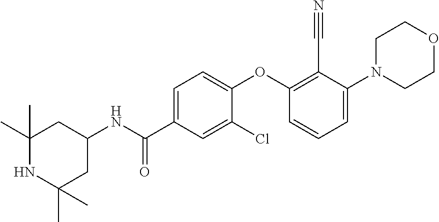 [SMILES: CC1(C)CC(NC(=O)c2ccc(Oc3cccc(N4CCOCC4)c3C#N)c(Cl)c2)CC(C)(C)N1] | I-391 |
| 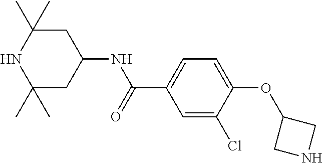 [SMILES: CC1(C)CC(NC(=O)c2ccc(OC3CNC3)c(Cl)c2)CC(C)(C)N1] | I-392 |
| 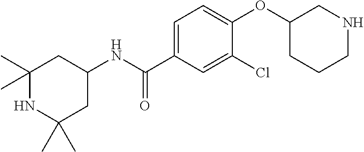 [SMILES: CC1(C)CC(NC(=O)c2ccc(OC3CCCNC3)c(Cl)c2)CC(C)(C)N1] | I-393 |
| 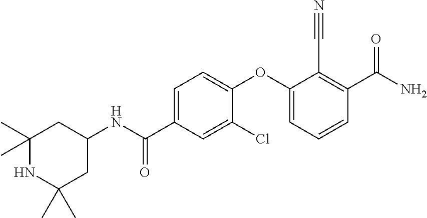 [SMILES: CC1(C)CC(NC(=O)c2ccc(Oc3cccc(C(N)=O)c3C#N)c(Cl)c2)CC(C)(C)N1] | I-394 |
| 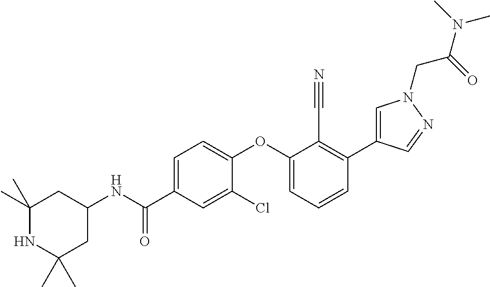 | I-395 |
| 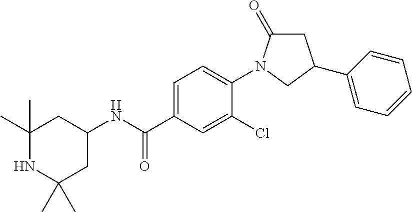 [SMILES: CC1(C)CC(NC(=O)c2ccc(N3CC(c4ccccc4)CC3=O)c(Cl)c2)CC(C)(C)N1] | I-396 |
| 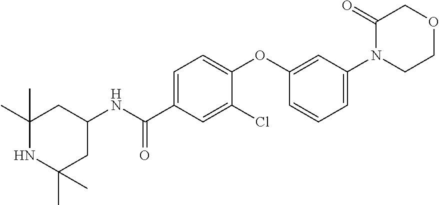 [SMILES: CC1(C)CC(NC(=O)c2ccc(Oc3cccc(N4CCOCC4=O)c3)c(Cl)c2)CC(C)(C)N1] | I-397 |
| 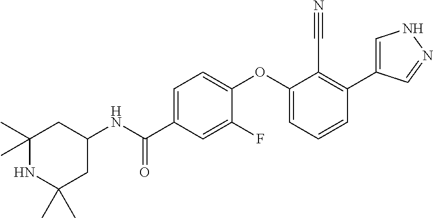 [SMILES: CC1(C)CC(NC(=O)c2ccc(Oc3cccc(-c4cn[nH]c4)c3C#N)c(F)c2)CC(C)(C)N1] | I-398 |
| 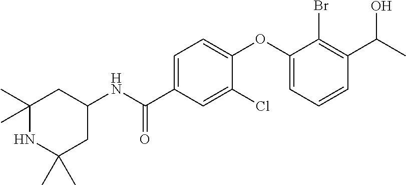 [SMILES: CC(O)c1cccc(Oc2ccc(C(=O)NC3CC(C)(C)NC(C)(C)C3)cc2Cl)c1Br] | I-399 |
| 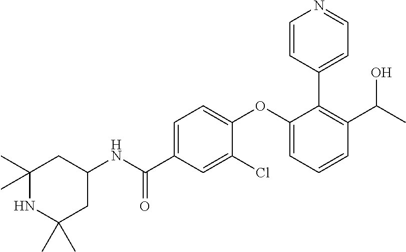 [SMILES: CC(O)c1cccc(Oc2ccc(C(=O)NC3CC(C)(C)NC(C)(C)C3)cc2Cl)c1-c1ccncc1] | I-400 |
| 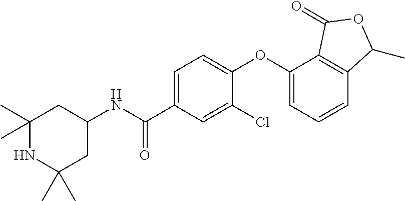 | I-401 |
| 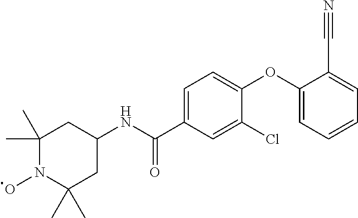 | I-402 |
| 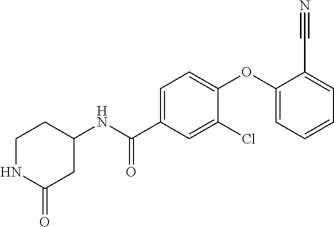 [SMILES: N#Cc1ccccc1Oc1ccc(C(=O)NC2CCNC(=O)C2)cc1Cl] | I-403 |
| 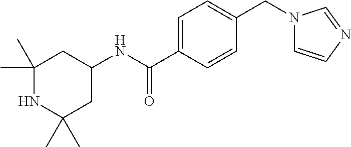 [SMILES: CC1(C)CC(NC(=O)c2ccc(Cn3ccnc3)cc2)CC(C)(C)N1] | I-404 |
| 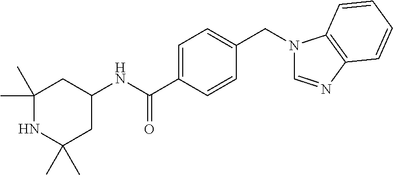 [SMILES: CC1(C)CC(NC(=O)c2ccc(Cn3cnc4ccccc43)cc2)CC(C)(C)N1] | I-405 |
| 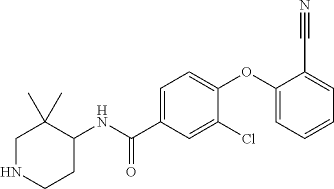 [SMILES: CC1(C)CNCCC1NC(=O)c1ccc(Oc2ccccc2C#N)c(Cl)c1] | I-406 |
| 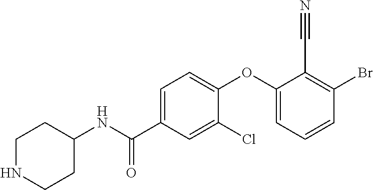 [SMILES: N#Cc1c(Br)cccc1Oc1ccc(C(=O)NC2CCNCC2)cc1Cl] | I-407 |
| 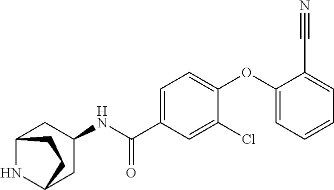 | I-408 |
| 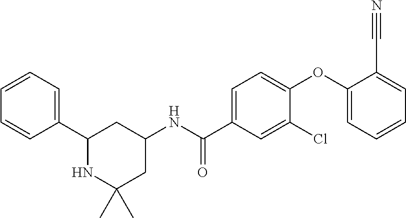 [SMILES: CC1(C)CC(NC(=O)c2ccc(Oc3ccccc3C#N)c(Cl)c2)CC(c2ccccc2)N1] | I-409 |
| 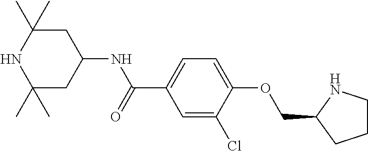 [SMILES: CC1(C)CC(NC(=O)c2ccc(OC[C@@H]3CCCN3)c(Cl)c2)CC(C)(C)N1] | I-410 |
| 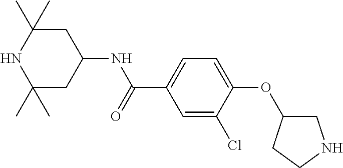 [SMILES: CC1(C)CC(NC(=O)c2ccc(OC3CCNC3)c(Cl)c2)CC(C)(C)N1] | I-411 |
| 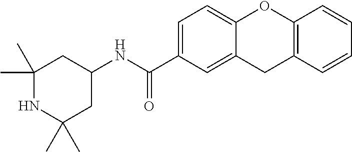 [SMILES: CC1(C)CC(NC(=O)c2ccc3c(c2)Cc2ccccc2O3)CC(C)(C)N1] | I-412 |
| 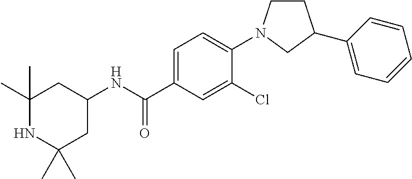 [SMILES: CC1(C)CC(NC(=O)c2ccc(N3CCC(c4ccccc4)C3)c(Cl)c2)CC(C)(C)N1] | I-413 |
| 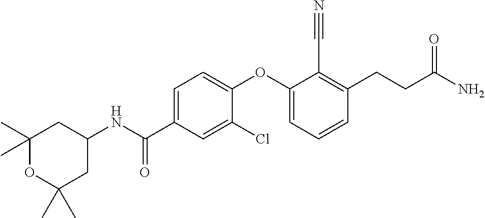 [SMILES: CC1(C)CC(NC(=O)c2ccc(Oc3cccc(CCC(N)=O)c3C#N)c(Cl)c2)CC(C)(C)O1] | I-414 |
| 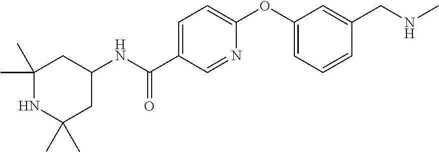 | I-415 |
| 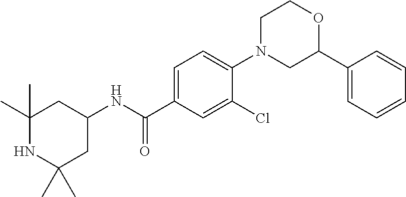 [SMILES: CC1(C)CC(NC(=O)c2ccc(N3CCOC(c4ccccc4)C3)c(Cl)c2)CC(C)(C)N1] | I-416 |
| 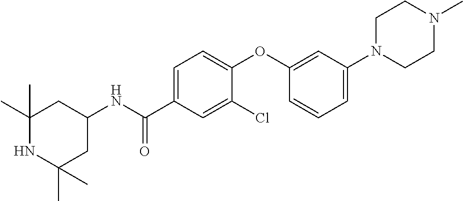 | I-417 |
| 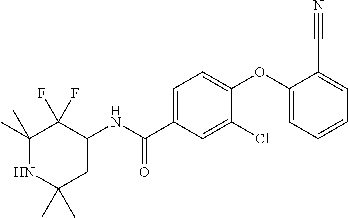 [SMILES: CC1(C)CC(NC(=O)c2ccc(Oc3ccccc3C#N)c(Cl)c2)C(F)(F)C(C)(C)N1] | I-418 |
| 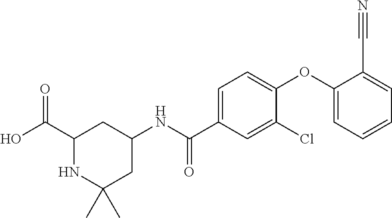 [SMILES: CC1(C)CC(NC(=O)c2ccc(Oc3ccccc3C#N)c(Cl)c2)CC(C(=O)O)N1] | I-419 |
| 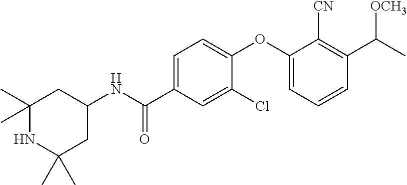 [SMILES: COC(C)c1cccc(Oc2ccc(C(=O)NC3CC(C)(C)NC(C)(C)C3)cc2Cl)c1C#N] | I-420 |
| 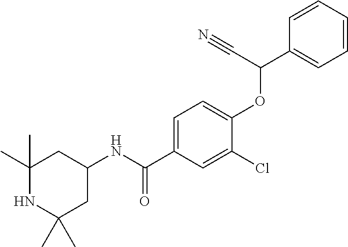 [SMILES: CC1(C)CC(NC(=O)c2ccc(OC(C#N)c3ccccc3)c(Cl)c2)CC(C)(C)N1] | I-421 |
| 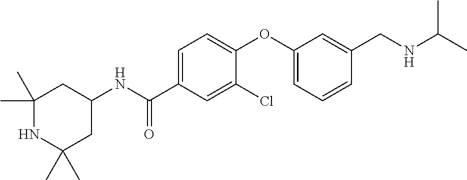 [SMILES: CC(C)NCc1cccc(Oc2ccc(C(=O)NC3CC(C)(C)NC(C)(C)C3)cc2Cl)c1] | I-422 |
| 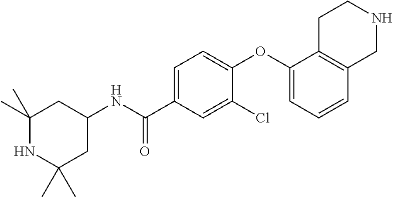 [SMILES: CC1(C)CC(NC(=O)c2ccc(Oc3cccc4c3CCNC4)c(Cl)c2)CC(C)(C)N1] | I-423 |
| 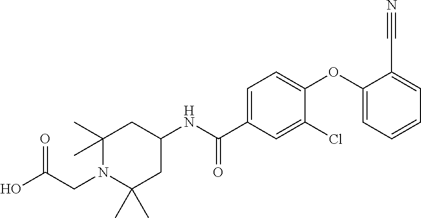 [SMILES: CC1(C)CC(NC(=O)c2ccc(Oc3ccccc3C#N)c(Cl)c2)CC(C)(C)N1CC(=O)O] | I-424 |
| 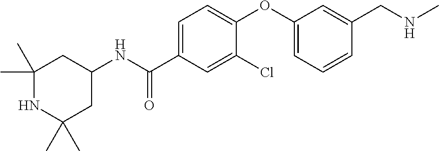 | I-425 |
| 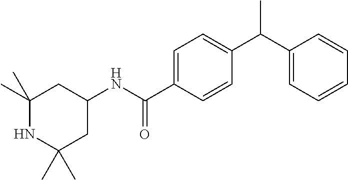 [SMILES: CC(c1ccccc1)c1ccc(C(=O)NC2CC(C)(C)NC(C)(C)C2)cc1] | I-426 |
| 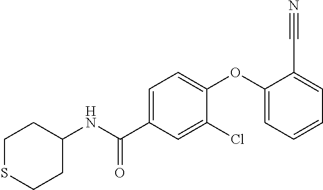 [SMILES: N#Cc1ccccc1Oc1ccc(C(=O)NC2CCSCC2)cc1Cl] | I-427 |
| 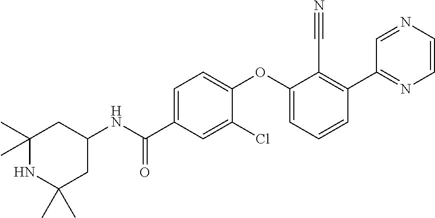 [SMILES: CC1(C)CC(NC(=O)c2ccc(Oc3cccc(-c4cnccn4)c3C#N)c(Cl)c2)CC(C)(C)N1] | I-428 |
| 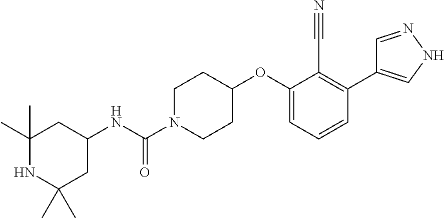 [SMILES: CC1(C)CC(NC(=O)N2CCC(Oc3cccc(-c4cn[nH]c4)c3C#N)CC2)CC(C)(C)N1] | I-429 |
| 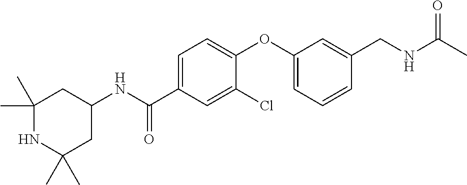 [SMILES: CC(=O)NCc1cccc(Oc2ccc(C(=O)NC3CC(C)(C)NC(C)(C)C3)cc2Cl)c1] | I-430 |
| 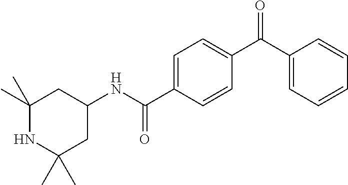 [SMILES: CC1(C)CC(NC(=O)c2ccc(C(=O)c3ccccc3)cc2)CC(C)(C)N1] | I-431 |
| 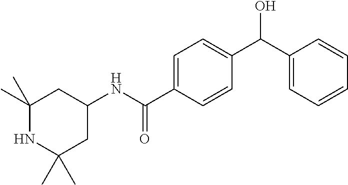 [SMILES: CC1(C)CC(NC(=O)c2ccc(C(O)c3ccccc3)cc2)CC(C)(C)N1] | I-432 |
| 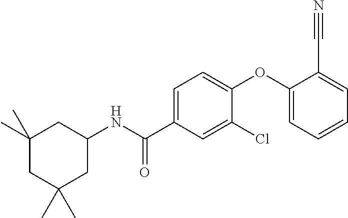 [SMILES: CC1(C)CC(NC(=O)c2ccc(Oc3ccccc3C#N)c(Cl)c2)CC(C)(C)C1] | I-433 |
| 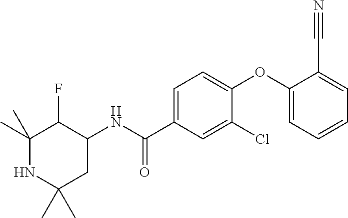 [SMILES: CC1(C)CC(NC(=O)c2ccc(Oc3ccccc3C#N)c(Cl)c2)C(F)C(C)(C)N1] | I-434 |
| 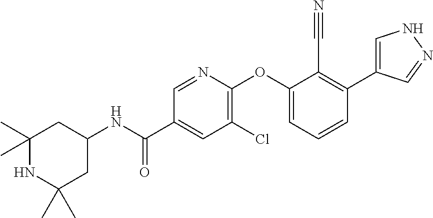 [SMILES: CC1(C)CC(NC(=O)c2cnc(Oc3cccc(-c4cn[nH]c4)c3C#N)c(Cl)c2)CC(C)(C)N1] | I-435 |
| 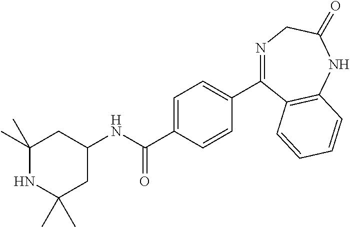 [SMILES: CC1(C)CC(NC(=O)c2ccc(C3=NCC(=O)Nc4ccccc43)cc2)CC(C)(C)N1] | I-436 |
| 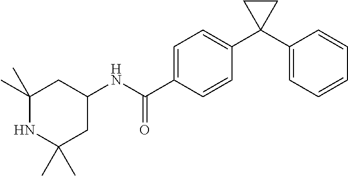 [SMILES: CC1(C)CC(NC(=O)c2ccc(C3(c4ccccc4)CC3)cc2)CC(C)(C)N1] | I-437 |
| 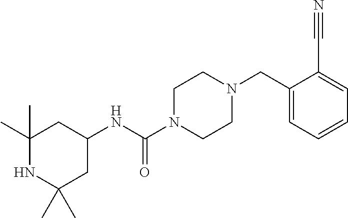 [SMILES: CC1(C)CC(NC(=O)N2CCN(Cc3ccccc3C#N)CC2)CC(C)(C)N1] | I-438 |
| 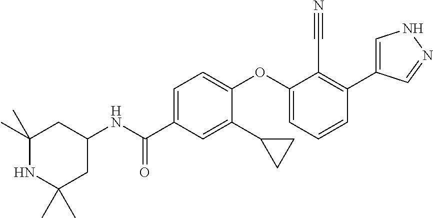 [SMILES: CC1(C)CC(NC(=O)c2ccc(Oc3cccc(-c4cn[nH]c4)c3C#N)c(C3CC3)c2)CC(C)(C)N1] | I-439 |
| 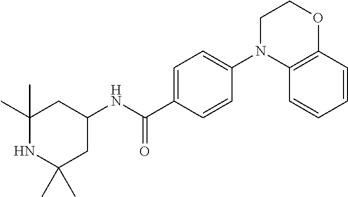 [SMILES: CC1(C)CC(NC(=O)c2ccc(N3CCOc4ccccc43)cc2)CC(C)(C)N1] | I-440 |
| 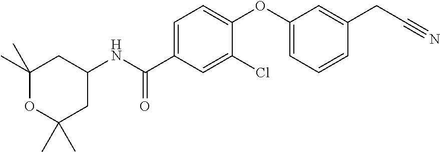 [SMILES: CC1(C)CC(NC(=O)c2ccc(Oc3cccc(CC#N)c3)c(Cl)c2)CC(C)(C)O1] | I-441 |
| 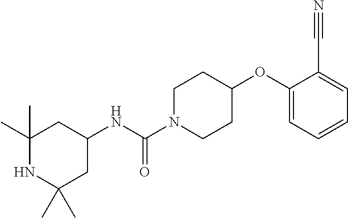 [SMILES: CC1(C)CC(NC(=O)N2CCC(Oc3ccccc3C#N)CC2)CC(C)(C)N1] | I-442 |
| 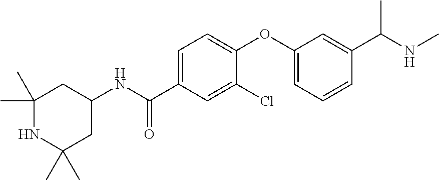 | I-443 |
| 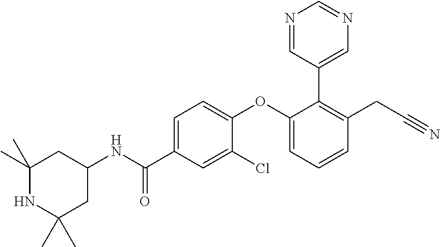 [SMILES: CC1(C)CC(NC(=O)c2ccc(Oc3cccc(CC#N)c3-c3cncnc3)c(Cl)c2)CC(C)(C)N1] | I-444 |
| 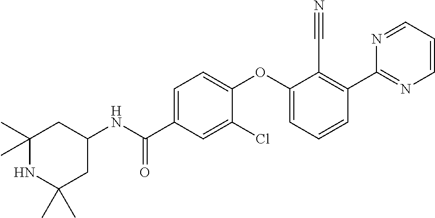 [SMILES: CC1(C)CC(NC(=O)c2ccc(Oc3cccc(-c4ncccn4)c3C#N)c(Cl)c2)CC(C)(C)N1] | I-445 |
| 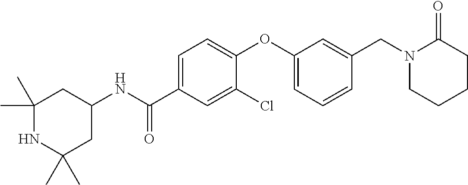 [SMILES: CC1(C)CC(NC(=O)c2ccc(Oc3cccc(CN4CCCCC4=O)c3)c(Cl)c2)CC(C)(C)N1] | I-446 |
| 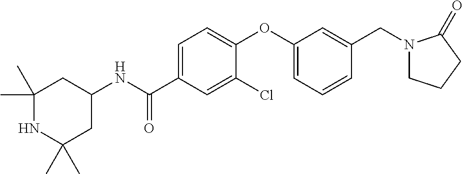 [SMILES: CC1(C)CC(NC(=O)c2ccc(Oc3cccc(CN4CCCC4=O)c3)c(Cl)c2)CC(C)(C)N1] | I-447 |
| 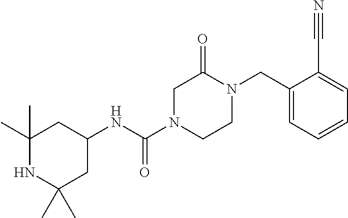 [SMILES: CC1(C)CC(NC(=O)N2CCN(Cc3ccccc3C#N)C(=O)C2)CC(C)(C)N1] | I-448 |
| 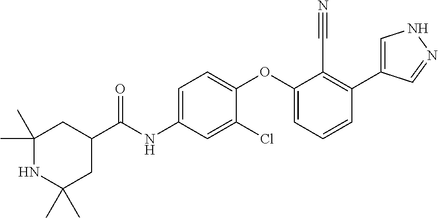 [SMILES: CC1(C)CC(C(=O)Nc2ccc(Oc3cccc(-c4cn[nH]c4)c3C#N)c(Cl)c2)CC(C)(C)N1] | I-449 |
| 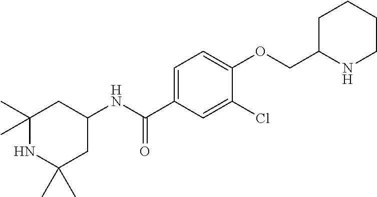 [SMILES: CC1(C)CC(NC(=O)c2ccc(OCC3CCCCN3)c(Cl)c2)CC(C)(C)N1] | I-450 |
| 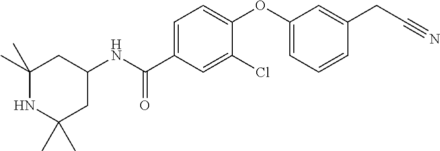 [SMILES: CC1(C)CC(NC(=O)c2ccc(Oc3cccc(CC#N)c3)c(Cl)c2)CC(C)(C)N1] | I-451 |
| 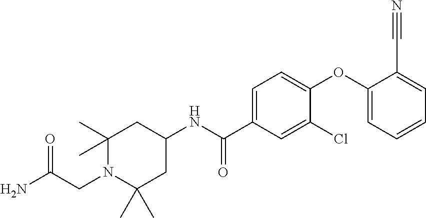 [SMILES: CC1(C)CC(NC(=O)c2ccc(Oc3ccccc3C#N)c(Cl)c2)CC(C)(C)N1CC(N)=O] | I-452 |
| 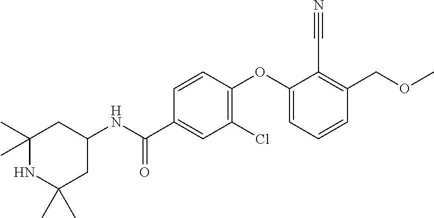 | I-453 |
| 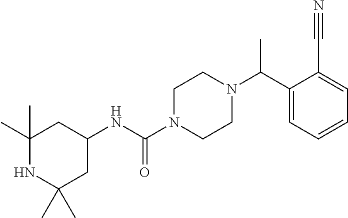 [SMILES: CC(c1ccccc1C#N)N1CCN(C(=O)NC2CC(C)(C)NC(C)(C)C2)CC1] | I-454 |
| 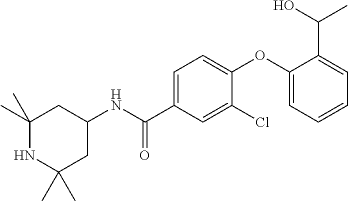 [SMILES: CC(O)c1ccccc1Oc1ccc(C(=O)NC2CC(C)(C)NC(C)(C)C2)cc1Cl] | I-455 |
| 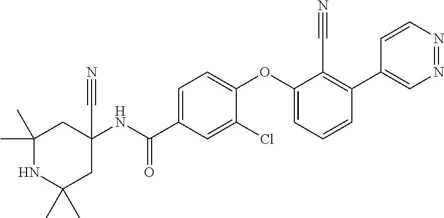 [SMILES: CC1(C)CC(C#N)(NC(=O)c2ccc(Oc3cccc(-c4ccnnc4)c3C#N)c(Cl)c2)CC(C)(C)N1] | I-456 |
| 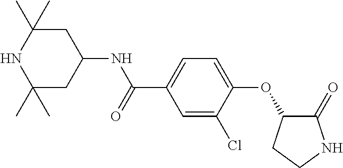 | I-457 |
| 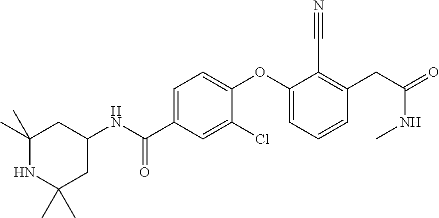 [SMILES: CNC(=O)Cc1cccc(Oc2ccc(C(=O)NC3CC(C)(C)NC(C)(C)C3)cc2Cl)c1C#N] | I-458 |
| 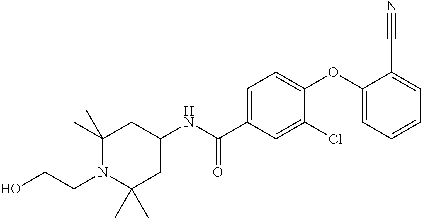 [SMILES: CC1(C)CC(NC(=O)c2ccc(Oc3ccccc3C#N)c(Cl)c2)CC(C)(C)N1CCO] | I-459 |
| 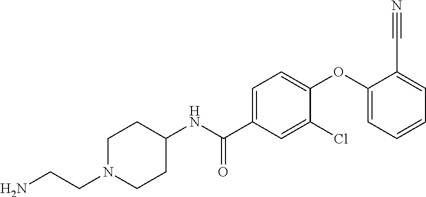 [SMILES: N#Cc1ccccc1Oc1ccc(C(=O)NC2CCN(CCN)CC2)cc1Cl] | I-460 |
| 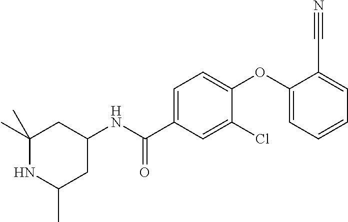 [SMILES: CC1CC(NC(=O)c2ccc(Oc3ccccc3C#N)c(Cl)c2)CC(C)(C)N1] | I-461 |
| 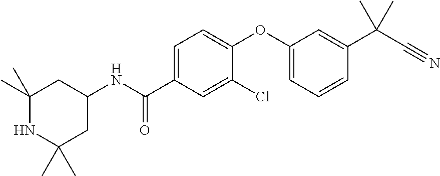 [SMILES: CC1(C)CC(NC(=O)c2ccc(Oc3cccc(C(C)(C)C#N)c3)c(Cl)c2)CC(C)(C)N1] | I-462 |
| 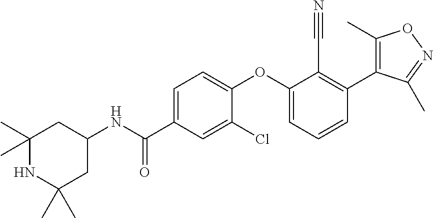 [SMILES: Cc1noc(C)c1-c1cccc(Oc2ccc(C(=O)NC3CC(C)(C)NC(C)(C)C3)cc2Cl)c1C#N] | I-463 |
| 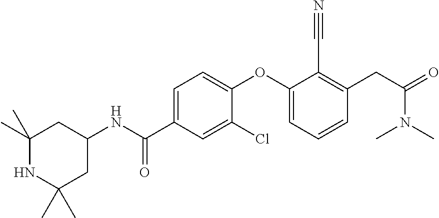 | I-464 |
| 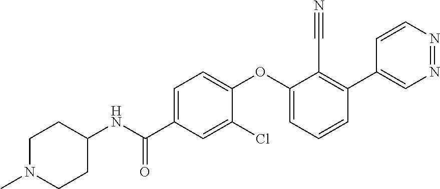 [SMILES: CN1CCC(NC(=O)c2ccc(Oc3cccc(-c4ccnnc4)c3C#N)c(Cl)c2)CC1] | I-465 |
| 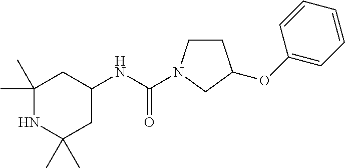 [SMILES: CC1(C)CC(NC(=O)N2CCC(Oc3ccccc3)C2)CC(C)(C)N1] | I-466 |
| 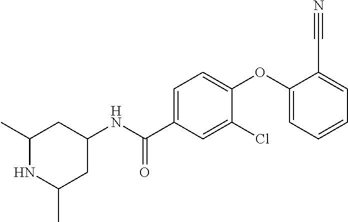 [SMILES: CC1CC(NC(=O)c2ccc(Oc3ccccc3C#N)c(Cl)c2)CC(C)N1] | I-467 |
| 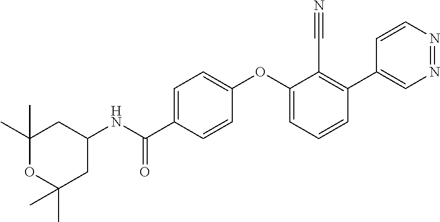 [SMILES: CC1(C)CC(NC(=O)c2ccc(Oc3cccc(-c4ccnnc4)c3C#N)cc2)CC(C)(C)O1] | I-468 |
| 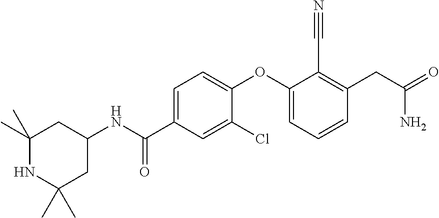 [SMILES: CC1(C)CC(NC(=O)c2ccc(Oc3cccc(CC(N)=O)c3C#N)c(Cl)c2)CC(C)(C)N1] | I-469 |
| 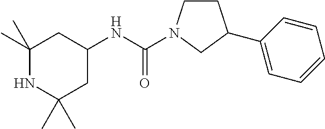 [SMILES: CC1(C)CC(NC(=O)N2CCC(c3ccccc3)C2)CC(C)(C)N1] | I-470 |
| 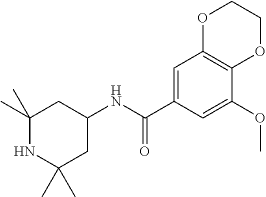 [SMILES: COc1cc(C(=O)NC2CC(C)(C)NC(C)(C)C2)cc2c1OCCO2] | I-471 |
| 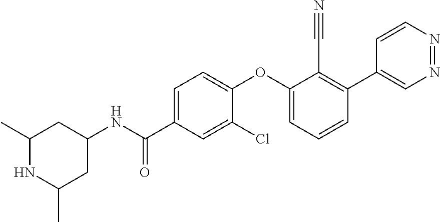 [SMILES: CC1CC(NC(=O)c2ccc(Oc3cccc(-c4ccnnc4)c3C#N)c(Cl)c2)CC(C)N1] | I-472 |
| 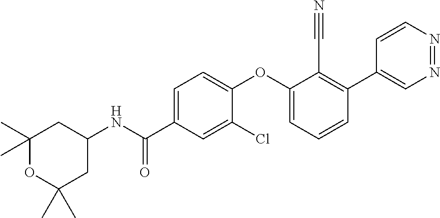 [SMILES: CC1(C)CC(NC(=O)c2ccc(Oc3cccc(-c4ccnnc4)c3C#N)c(Cl)c2)CC(C)(C)O1] | I-473 |
| 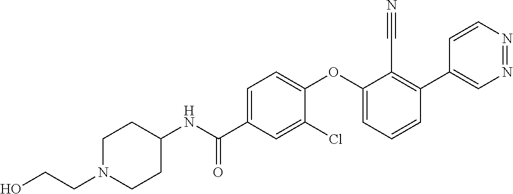 [SMILES: N#Cc1c(Oc2ccc(C(=O)NC3CCN(CCO)CC3)cc2Cl)cccc1-c1ccnnc1] | I-474 |
| 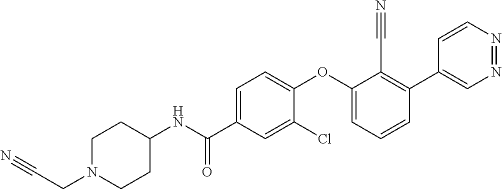 [SMILES: N#CCN1CCC(NC(=O)c2ccc(Oc3cccc(-c4ccnnc4)c3C#N)c(Cl)c2)CC1] | I-475 |
| 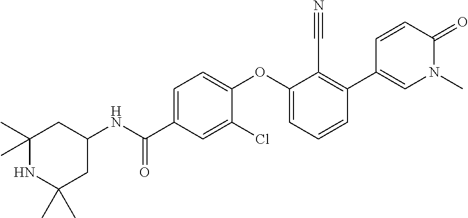 [SMILES: Cn1cc(-c2cccc(Oc3ccc(C(=O)NC4CC(C)(C)NC(C)(C)C4)cc3Cl)c2C#N)ccc1=O] | I-476 |
| 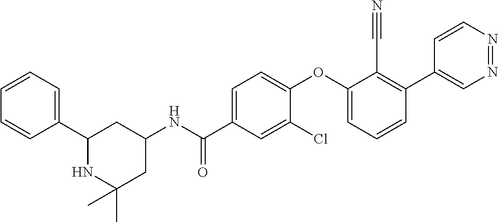 [SMILES: CC1(C)CC(NC(=O)c2ccc(Oc3cccc(-c4ccnnc4)c3C#N)c(Cl)c2)CC(c2ccccc2)N1] | I-477 |
| 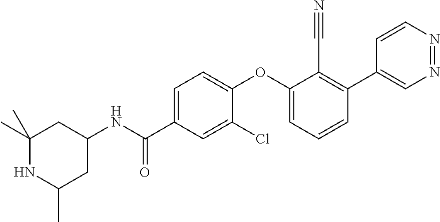 [SMILES: CC1CC(NC(=O)c2ccc(Oc3cccc(-c4ccnnc4)c3C#N)c(Cl)c2)CC(C)(C)N1] | I-478 |
| 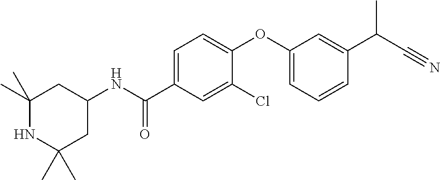 [SMILES: CC(C#N)c1cccc(Oc2ccc(C(=O)NC3CC(C)(C)NC(C)(C)C3)cc2Cl)c1] | I-479 |
| 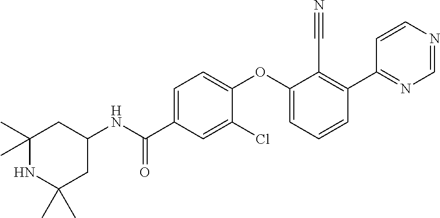 [SMILES: CC1(C)CC(NC(=O)c2ccc(Oc3cccc(-c4ccncn4)c3C#N)c(Cl)c2)CC(C)(C)N1] | I-480 |
| 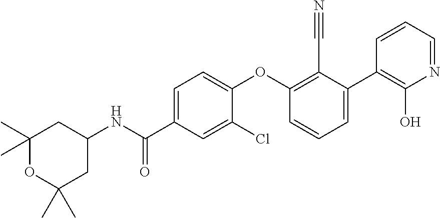 [SMILES: CC1(C)CC(NC(=O)c2ccc(Oc3cccc(-c4cccnc4O)c3C#N)c(Cl)c2)CC(C)(C)O1] | I-481 |
| 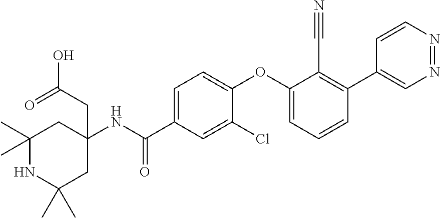 [SMILES: CC1(C)CC(CC(=O)O)(NC(=O)c2ccc(Oc3cccc(-c4ccnnc4)c3C#N)c(Cl)c2)CC(C)(C)N1] | I-482 |
| 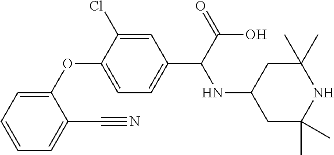 [SMILES: CC1(C)CC(NC(C(=O)O)c2ccc(Oc3ccccc3C#N)c(Cl)c2)CC(C)(C)N1] | I-483 |
| 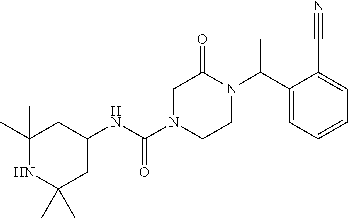 [SMILES: CC(c1ccccc1C#N)N1CCN(C(=O)NC2CC(C)(C)NC(C)(C)C2)CC1=O] | I-484 |
| 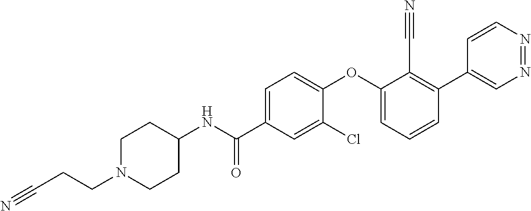 [SMILES: N#CCCN1CCC(NC(=O)c2ccc(Oc3cccc(-c4ccnnc4)c3C#N)c(Cl)c2)CC1] | I-485 |
| 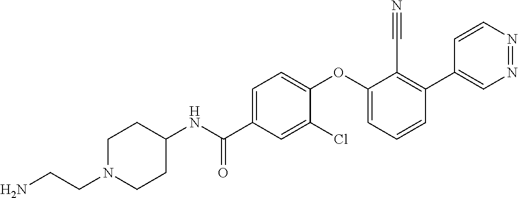 [SMILES: N#Cc1c(Oc2ccc(C(=O)NC3CCN(CCN)CC3)cc2Cl)cccc1-c1ccnnc1] | I-486 |
| 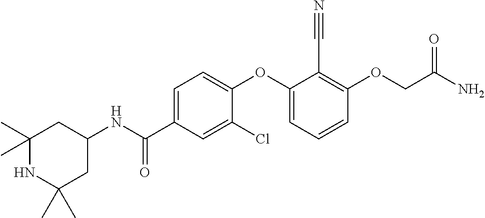 [SMILES: CC1(C)CC(NC(=O)c2ccc(Oc3cccc(OCC(N)=O)c3C#N)c(Cl)c2)CC(C)(C)N1] | I-487 |
| 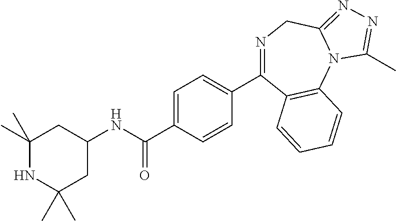 | I-488 |
| 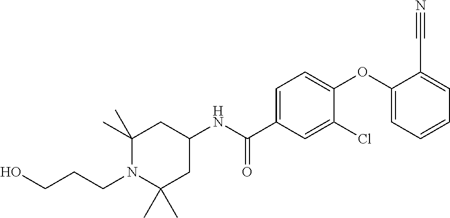 [SMILES: CC1(C)CC(NC(=O)c2ccc(Oc3ccccc3C#N)c(Cl)c2)CC(C)(C)N1CCCO] | I-489 |
| 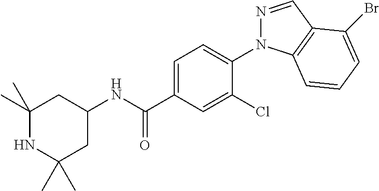 [SMILES: CC1(C)CC(NC(=O)c2ccc(-n3ncc4c(Br)cccc43)c(Cl)c2)CC(C)(C)N1] | I-490 |
| 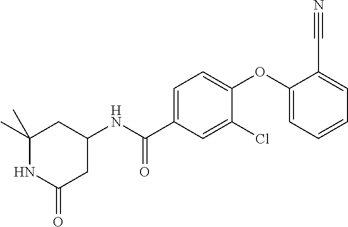 [SMILES: CC1(C)CC(NC(=O)c2ccc(Oc3ccccc3C#N)c(Cl)c2)CC(=O)N1] | I-491 |
| 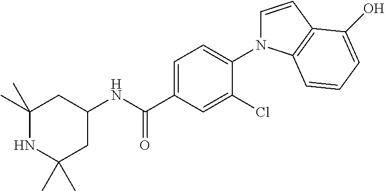 [SMILES: CC1(C)CC(NC(=O)c2ccc(-n3ccc4c(O)cccc43)c(Cl)c2)CC(C)(C)N1] | I-492 |
| 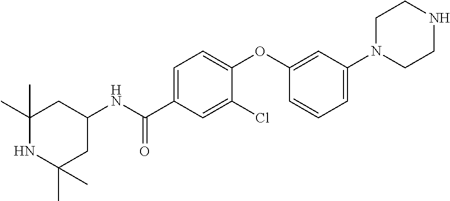 [SMILES: CC1(C)CC(NC(=O)c2ccc(Oc3cccc(N4CCNCC4)c3)c(Cl)c2)CC(C)(C)N1] | I-493 |
| 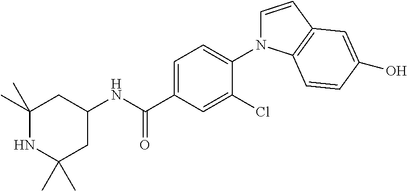 [SMILES: CC1(C)CC(NC(=O)c2ccc(-n3ccc4cc(O)ccc43)c(Cl)c2)CC(C)(C)N1] | I-494 |
| 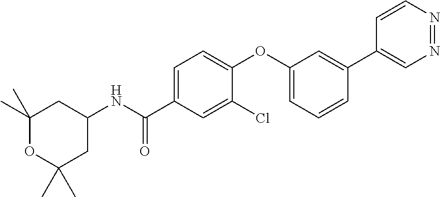 [SMILES: CC1(C)CC(NC(=O)c2ccc(Oc3cccc(-c4ccnnc4)c3)c(Cl)c2)CC(C)(C)O1] | I-495 |
| 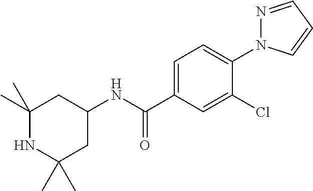 [SMILES: CC1(C)CC(NC(=O)c2ccc(-n3cccn3)c(Cl)c2)CC(C)(C)N1] | I-496 |
| 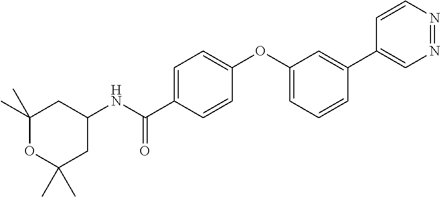 [SMILES: CC1(C)CC(NC(=O)c2ccc(Oc3cccc(-c4ccnnc4)c3)cc2)CC(C)(C)O1] | I-497 |
| 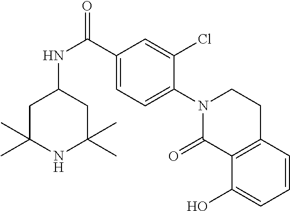 [SMILES: CC1(C)CC(NC(=O)c2ccc(N3CCc4cccc(O)c4C3=O)c(Cl)c2)CC(C)(C)N1] | I-498 |
| 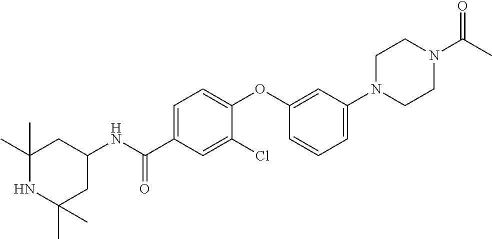 [SMILES: CC(=O)N1CCN(c2cccc(Oc3ccc(C(=O)NC4CC(C)(C)NC(C)(C)C4)cc3Cl)c2)CC1] | I-499 |
| 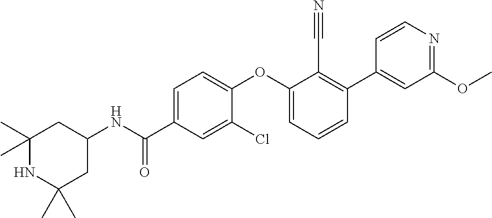 | I-500 |
| 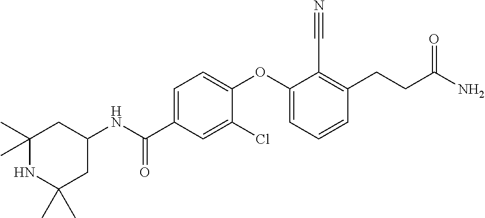 [SMILES: CC1(C)CC(NC(=O)c2ccc(Oc3cccc(CCC(N)=O)c3C#N)c(Cl)c2)CC(C)(C)N1] | I-501 |
| 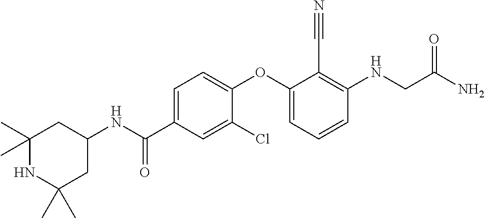 [SMILES: CC1(C)CC(NC(=O)c2ccc(Oc3cccc(NCC(N)=O)c3C#N)c(Cl)c2)CC(C)(C)N1] | I-502 |
| 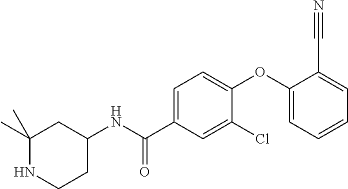 [SMILES: CC1(C)CC(NC(=O)c2ccc(Oc3ccccc3C#N)c(Cl)c2)CCN1] | I-503 |
| 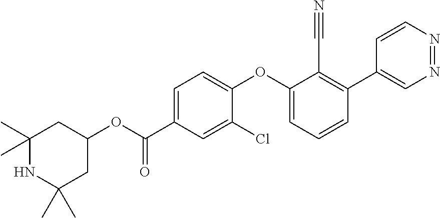 [SMILES: CC1(C)CC(OC(=O)c2ccc(Oc3cccc(-c4ccnnc4)c3C#N)c(Cl)c2)CC(C)(C)N1] | I-504 |
| 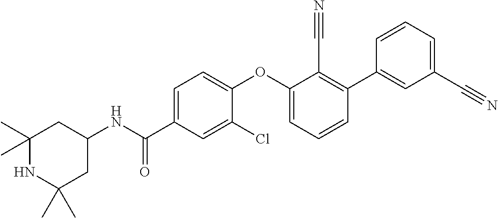 [SMILES: CC1(C)CC(NC(=O)c2ccc(Oc3cccc(-c4cccc(C#N)c4)c3C#N)c(Cl)c2)CC(C)(C)N1] | I-505 |
| 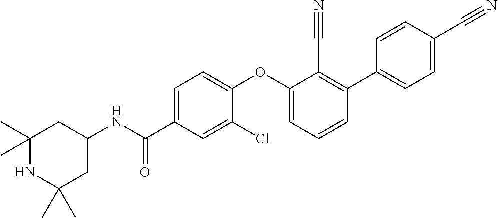 [SMILES: CC1(C)CC(NC(=O)c2ccc(Oc3cccc(-c4ccc(C#N)cc4)c3C#N)c(Cl)c2)CC(C)(C)N1] | I-506 |
| 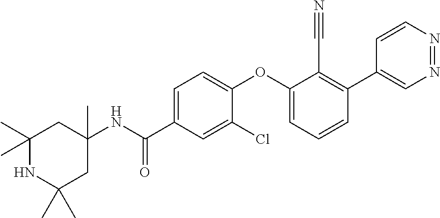 [SMILES: CC1(C)CC(C)(NC(=O)c2ccc(Oc3cccc(-c4ccnnc4)c3C#N)c(Cl)c2)CC(C)(C)N1] | I-507 |
| 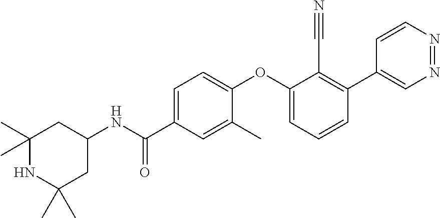 [SMILES: Cc1cc(C(=O)NC2CC(C)(C)NC(C)(C)C2)ccc1Oc1cccc(-c2ccnnc2)c1C#N] | I-508 |
| 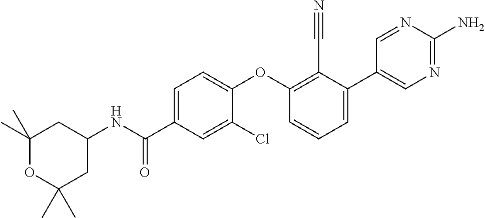 [SMILES: CC1(C)CC(NC(=O)c2ccc(Oc3cccc(-c4cnc(N)nc4)c3C#N)c(Cl)c2)CC(C)(C)O1] | I-509 |
| 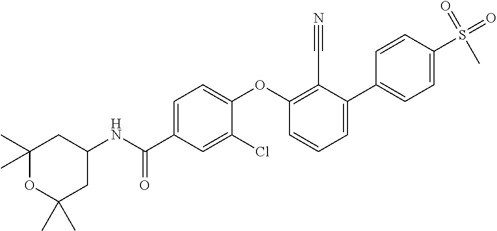 [SMILES: CC1(C)CC(NC(=O)c2ccc(Oc3cccc(-c4ccc(S(C)(=O)=O)cc4)c3C#N)c(Cl)c2)CC(C)(C)O1] | I-510 |
| 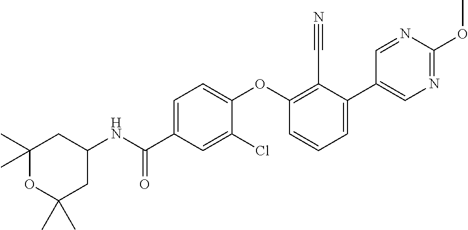 | I-511 |
| 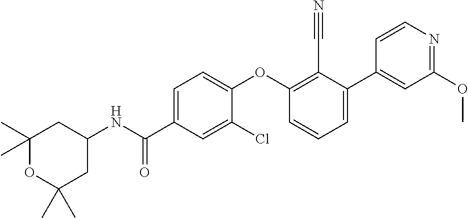 | I-512 |
| 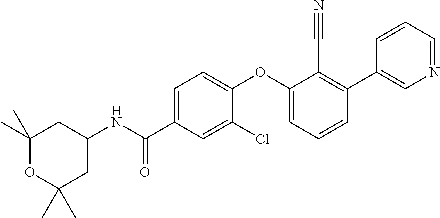 [SMILES: CC1(C)CC(NC(=O)c2ccc(Oc3cccc(-c4cccnc4)c3C#N)c(Cl)c2)CC(C)(C)O1] | I-513 |
| 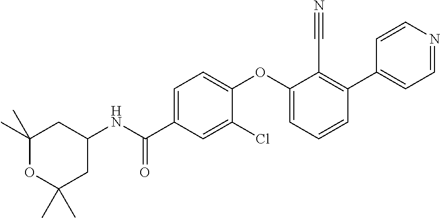 [SMILES: CC1(C)CC(NC(=O)c2ccc(Oc3cccc(-c4ccncc4)c3C#N)c(Cl)c2)CC(C)(C)O1] | I-514 |
| 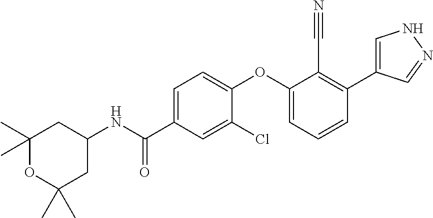 [SMILES: CC1(C)CC(NC(=O)c2ccc(Oc3cccc(-c4cn[nH]c4)c3C#N)c(Cl)c2)CC(C)(C)O1] | I-515 |
| 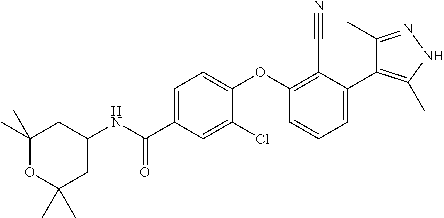 [SMILES: Cc1n[nH]c(C)c1-c1cccc(Oc2ccc(C(=O)NC3CC(C)(C)OC(C)(C)C3)cc2Cl)c1C#N] | I-516 |
| 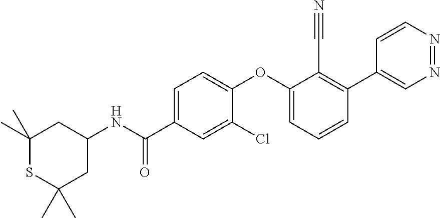 [SMILES: CC1(C)CC(NC(=O)c2ccc(Oc3cccc(-c4ccnnc4)c3C#N)c(Cl)c2)CC(C)(C)S1] | I-517 |
| 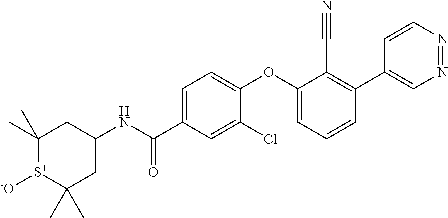 | I-518 |
| 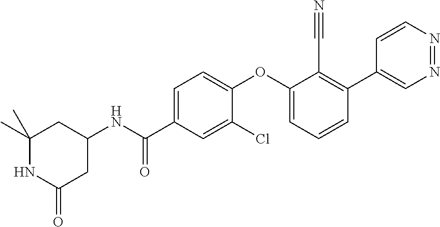 [SMILES: CC1(C)CC(NC(=O)c2ccc(Oc3cccc(-c4ccnnc4)c3C#N)c(Cl)c2)CC(=O)N1] | I-519 |
| 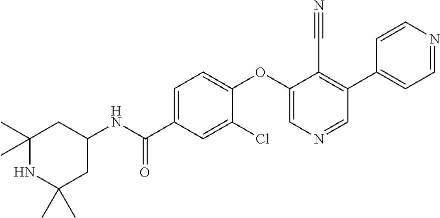 [SMILES: CC1(C)CC(NC(=O)c2ccc(Oc3cncc(-c4ccncc4)c3C#N)c(Cl)c2)CC(C)(C)N1] | I-520 |
| 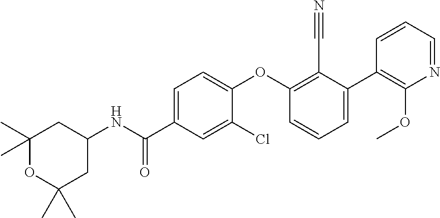 [SMILES: COc1ncccc1-c1cccc(Oc2ccc(C(=O)NC3CC(C)(C)OC(C)(C)C3)cc2Cl)c1C#N] | I-521 |
| 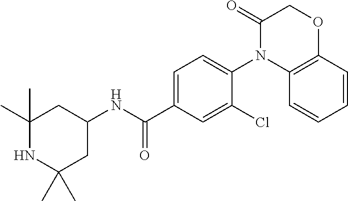 [SMILES: CC1(C)CC(NC(=O)c2ccc(N3C(=O)COc4ccccc43)c(Cl)c2)CC(C)(C)N1] | I-522 |
| 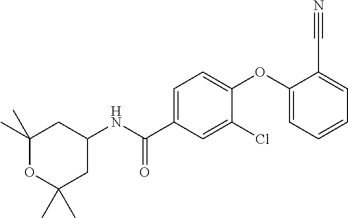 [SMILES: CC1(C)CC(NC(=O)c2ccc(Oc3ccccc3C#N)c(Cl)c2)CC(C)(C)O1] | I-523 |
| 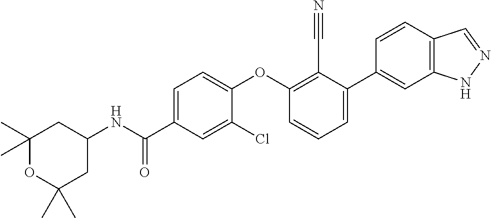 [SMILES: CC1(C)CC(NC(=O)c2ccc(Oc3cccc(-c4ccc5cn[nH]c5c4)c3C#N)c(Cl)c2)CC(C)(C)O1] | I-524 |
| 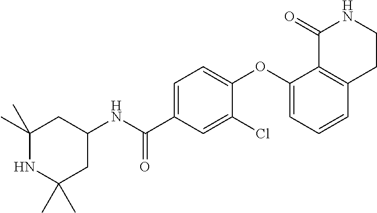 [SMILES: CC1(C)CC(NC(=O)c2ccc(Oc3cccc4c3C(=O)NCC4)c(Cl)c2)CC(C)(C)N1] | I-525 |
| 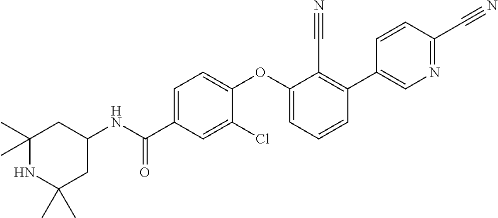 [SMILES: CC1(C)CC(NC(=O)c2ccc(Oc3cccc(-c4ccc(C#N)nc4)c3C#N)c(Cl)c2)CC(C)(C)N1] | I-526 |
| 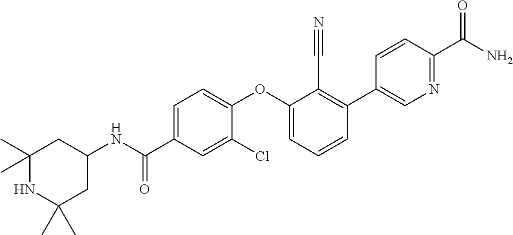 [SMILES: CC1(C)CC(NC(=O)c2ccc(Oc3cccc(-c4ccc(C(N)=O)nc4)c3C#N)c(Cl)c2)CC(C)(C)N1] | I-527 |
| 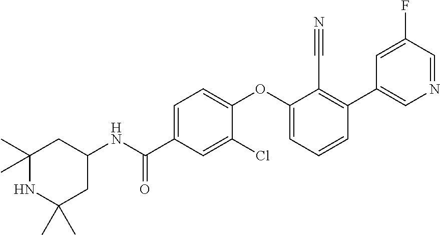 [SMILES: CC1(C)CC(NC(=O)c2ccc(Oc3cccc(-c4cncc(F)c4)c3C#N)c(Cl)c2)CC(C)(C)N1] | I-528 |
| 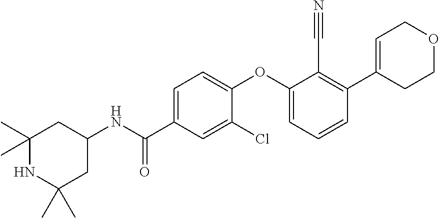 [SMILES: CC1(C)CC(NC(=O)c2ccc(Oc3cccc(C4=CCOCC4)c3C#N)c(Cl)c2)CC(C)(C)N1] | I-529 |
| 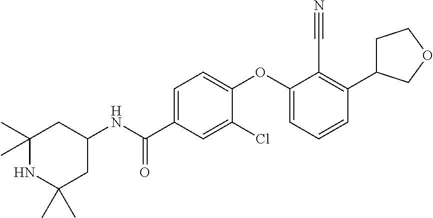 [SMILES: CC1(C)CC(NC(=O)c2ccc(Oc3cccc(C4CCOC4)c3C#N)c(Cl)c2)CC(C)(C)N1] | I-530 |
| 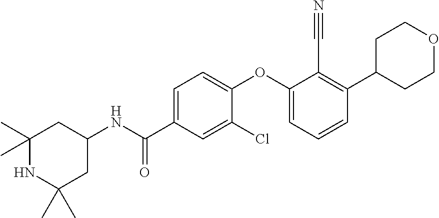 [SMILES: CC1(C)CC(NC(=O)c2ccc(Oc3cccc(C4CCOCC4)c3C#N)c(Cl)c2)CC(C)(C)N1] | I-531 |
| 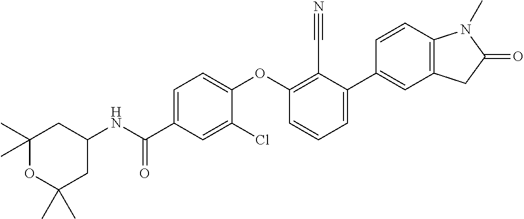 [SMILES: CN1C(=O)Cc2cc(-c3cccc(Oc4ccc(C(=O)NC5CC(C)(C)OC(C)(C)C5)cc4Cl)c3C#N)ccc21] | I-532 |
| 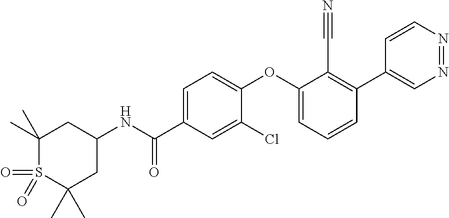 [SMILES: CC1(C)CC(NC(=O)c2ccc(Oc3cccc(-c4ccnnc4)c3C#N)c(Cl)c2)CC(C)(C)S1(=O)=O] | I-533 |
| 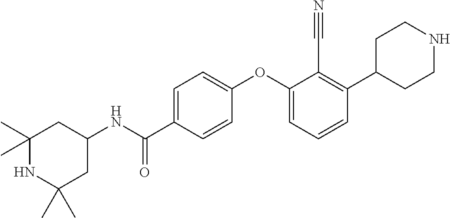 [SMILES: CC1(C)CC(NC(=O)c2ccc(Oc3cccc(C4CCNCC4)c3C#N)cc2)CC(C)(C)N1] | I-534 |
| 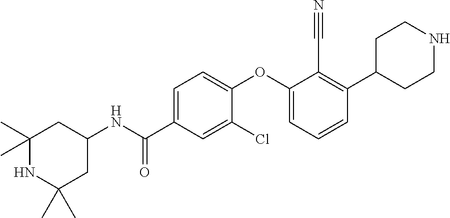 [SMILES: CC1(C)CC(NC(=O)c2ccc(Oc3cccc(C4CCNCC4)c3C#N)c(Cl)c2)CC(C)(C)N1] | I-535 |
| 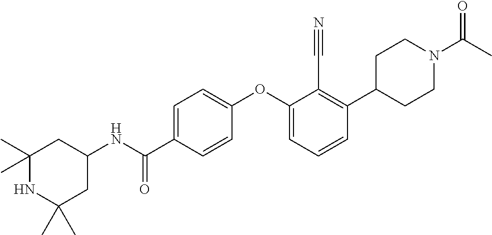 [SMILES: CC(=O)N1CCC(c2cccc(Oc3ccc(C(=O)NC4CC(C)(C)NC(C)(C)C4)cc3)c2C#N)CC1] | I-536 |
| 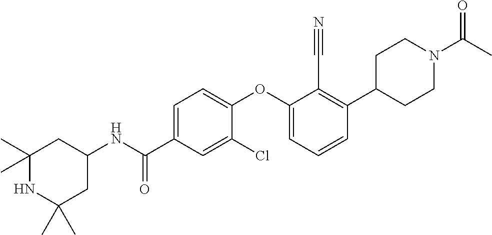 [SMILES: CC(=O)N1CCC(c2cccc(Oc3ccc(C(=O)NC4CC(C)(C)NC(C)(C)C4)cc3Cl)c2C#N)CC1] | I-537 |
| 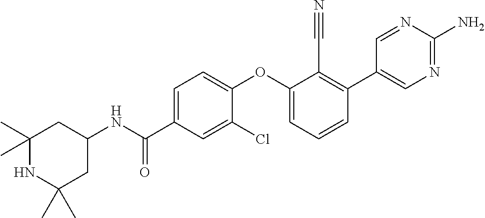 [SMILES: CC1(C)CC(NC(=O)c2ccc(Oc3cccc(-c4cnc(N)nc4)c3C#N)c(Cl)c2)CC(C)(C)N1] | I-538 |
| 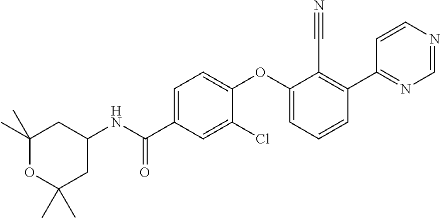 [SMILES: CC1(C)CC(NC(=O)c2ccc(Oc3cccc(-c4ccncn4)c3C#N)c(Cl)c2)CC(C)(C)O1] | I-539 |
| 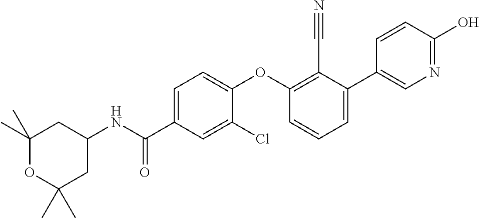 [SMILES: CC1(C)CC(NC(=O)c2ccc(Oc3cccc(-c4ccc(O)nc4)c3C#N)c(Cl)c2)CC(C)(C)O1] | I-540 |
| 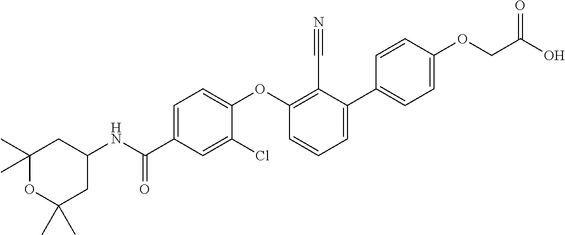 [SMILES: CC1(C)CC(NC(=O)c2ccc(Oc3cccc(-c4ccc(OCC(=O)O)cc4)c3C#N)c(Cl)c2)CC(C)(C)O1] | I-541 |
| 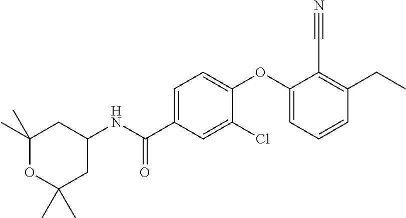 [SMILES: CCc1cccc(Oc2ccc(C(=O)NC3CC(C)(C)OC(C)(C)C3)cc2Cl)c1C#N] | I-542 |
| 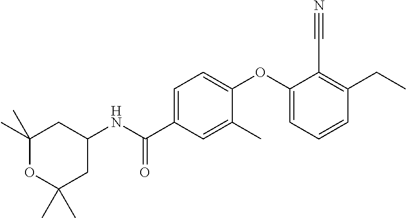 [SMILES: CCc1cccc(Oc2ccc(C(=O)NC3CC(C)(C)OC(C)(C)C3)cc2C)c1C#N] | I-543 |
| 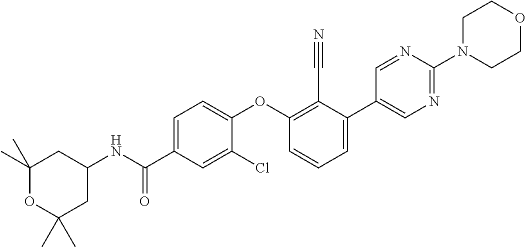 [SMILES: CC1(C)CC(NC(=O)c2ccc(Oc3cccc(-c4cnc(N5CCOCC5)nc4)c3C#N)c(Cl)c2)CC(C)(C)O1] | I-544 |
| 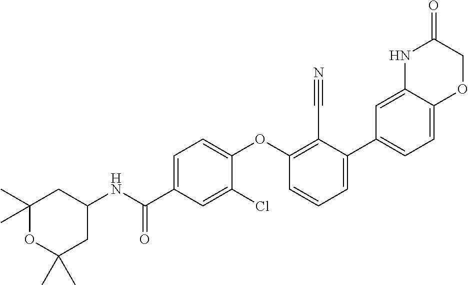 [SMILES: CC1(C)CC(NC(=O)c2ccc(Oc3cccc(-c4ccc5c(c4)NC(=O)CO5)c3C#N)c(Cl)c2)CC(C)(C)O1] | I-545 |
| 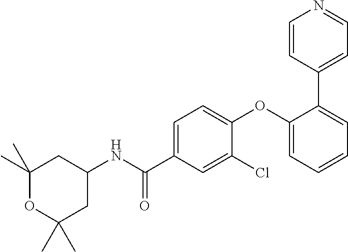 [SMILES: CC1(C)CC(NC(=O)c2ccc(Oc3ccccc3-c3ccncc3)c(Cl)c2)CC(C)(C)O1] | I-546 |
| 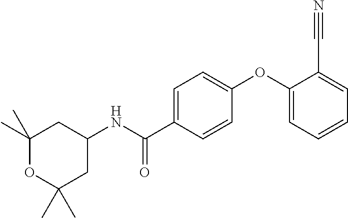 [SMILES: CC1(C)CC(NC(=O)c2ccc(Oc3ccccc3C#N)cc2)CC(C)(C)O1] | I-547 |
| 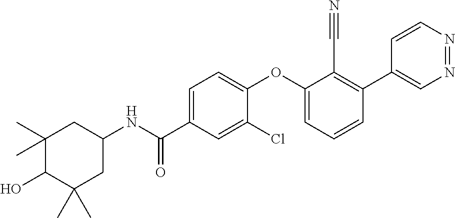 [SMILES: CC1(C)CC(NC(=O)c2ccc(Oc3cccc(-c4ccnnc4)c3C#N)c(Cl)c2)CC(C)(C)C1O] | I-548 |
| 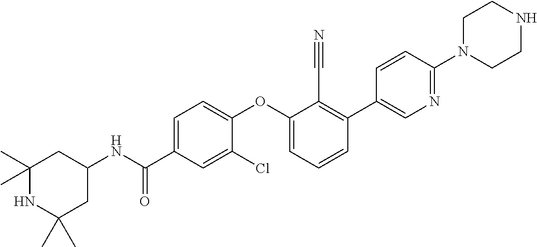 [SMILES: CC1(C)CC(NC(=O)c2ccc(Oc3cccc(-c4ccc(N5CCNCC5)nc4)c3C#N)c(Cl)c2)CC(C)(C)N1] | I-549 |
| 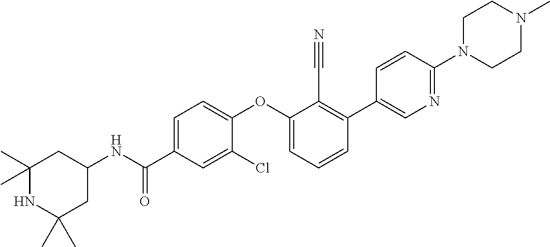 | I-550 |
| 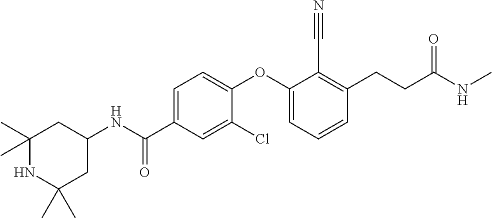 | I-551 |
| 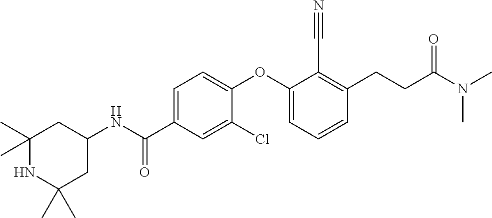 | I-552 |
| 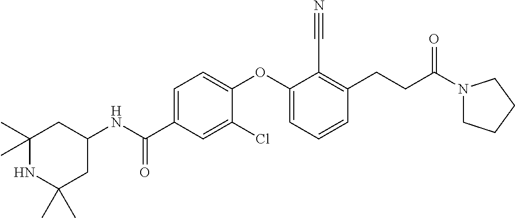 [SMILES: CC1(C)CC(NC(=O)c2ccc(Oc3cccc(CCC(=O)N4CCCC4)c3C#N)c(Cl)c2)CC(C)(C)N1] | I-553 |
| 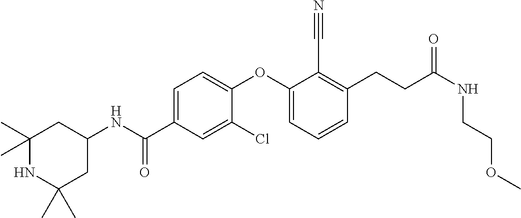 | I-554 |
| 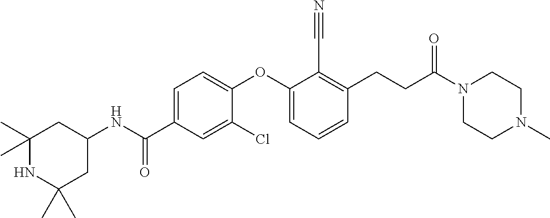 | I-555 |
| 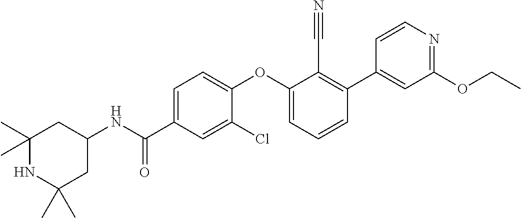 [SMILES: CCOc1cc(-c2cccc(Oc3ccc(C(=O)NC4CC(C)(C)NC(C)(C)C4)cc3Cl)c2C#N)ccn1] | I-556 |
| 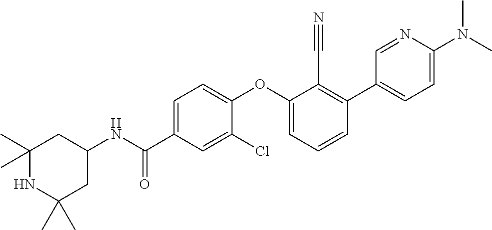 | I-557 |
| 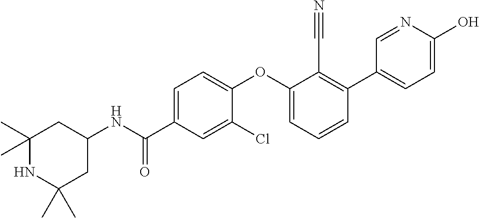 [SMILES: CC1(C)CC(NC(=O)c2ccc(Oc3cccc(-c4ccc(O)nc4)c3C#N)c(Cl)c2)CC(C)(C)N1] | I-558 |
| 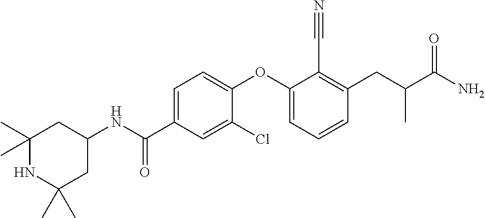 [SMILES: CC(Cc1cccc(Oc2ccc(C(=O)NC3CC(C)(C)NC(C)(C)C3)cc2Cl)c1C#N)C(N)=O] | I-559 |
| 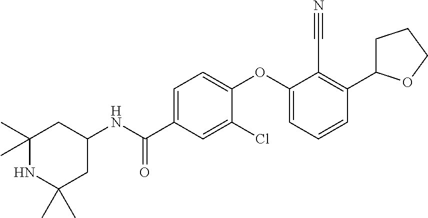 [SMILES: CC1(C)CC(NC(=O)c2ccc(Oc3cccc(C4CCCO4)c3C#N)c(Cl)c2)CC(C)(C)N1] | I-560 |
| 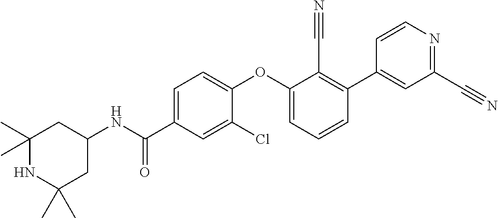 [SMILES: CC1(C)CC(NC(=O)c2ccc(Oc3cccc(-c4ccnc(C#N)c4)c3C#N)c(Cl)c2)CC(C)(C)N1] | I-561 |
| 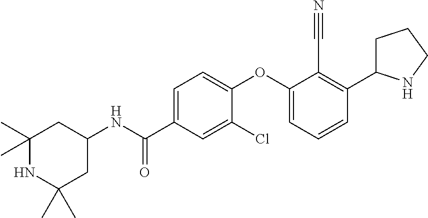 [SMILES: CC1(C)CC(NC(=O)c2ccc(Oc3cccc(C4CCCN4)c3C#N)c(Cl)c2)CC(C)(C)N1] | I-562 |
| 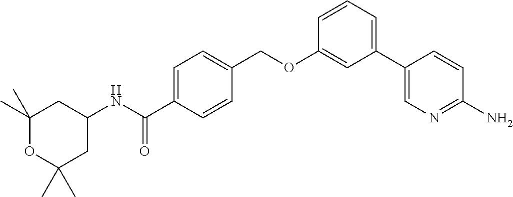 [SMILES: CC1(C)CC(NC(=O)c2ccc(COc3cccc(-c4ccc(N)nc4)c3)cc2)CC(C)(C)O1] | I-563 |
| 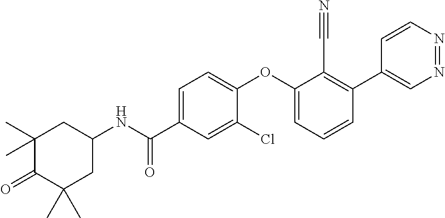 [SMILES: CC1(C)CC(NC(=O)c2ccc(Oc3cccc(-c4ccnnc4)c3C#N)c(Cl)c2)CC(C)(C)C1=O] | I-564 |
| 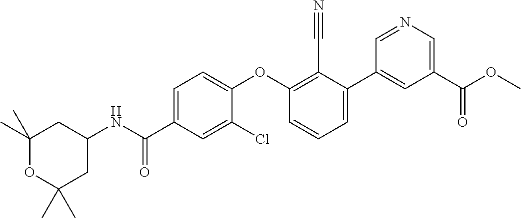 | I-565 |
| 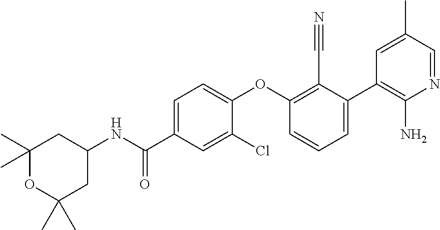 [SMILES: Cc1cnc(N)c(-c2cccc(Oc3ccc(C(=O)NC4CC(C)(C)OC(C)(C)C4)cc3Cl)c2C#N)c1] | I-566 |
| 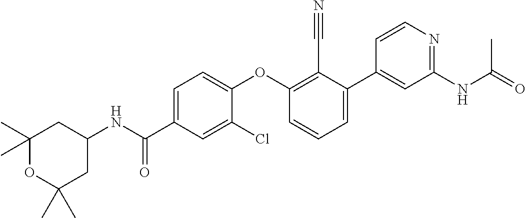 [SMILES: CC(=O)Nc1cc(-c2cccc(Oc3ccc(C(=O)NC4CC(C)(C)OC(C)(C)C4)cc3Cl)c2C#N)ccn1] | I-567 |
| 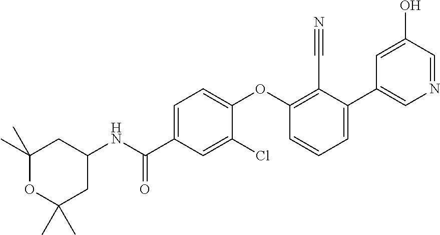 [SMILES: CC1(C)CC(NC(=O)c2ccc(Oc3cccc(-c4cncc(O)c4)c3C#N)c(Cl)c2)CC(C)(C)O1] | I-568 |
| 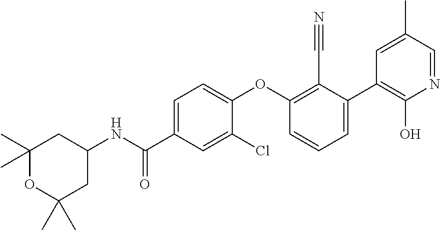 [SMILES: Cc1cnc(O)c(-c2cccc(Oc3ccc(C(=O)NC4CC(C)(C)OC(C)(C)C4)cc3Cl)c2C#N)c1] | I-569 |
| 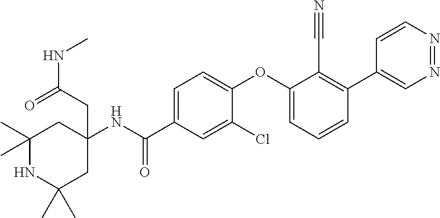 [SMILES: CNC(=O)CC1(NC(=O)c2ccc(Oc3cccc(-c4ccnnc4)c3C#N)c(Cl)c2)CC(C)(C)NC(C)(C)C1] | I-570 |
| 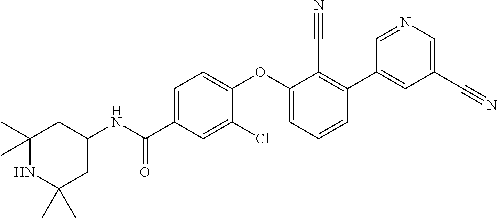 [SMILES: CC1(C)CC(NC(=O)c2ccc(Oc3cccc(-c4cncc(C#N)c4)c3C#N)c(Cl)c2)CC(C)(C)N1] | I-571 |
| 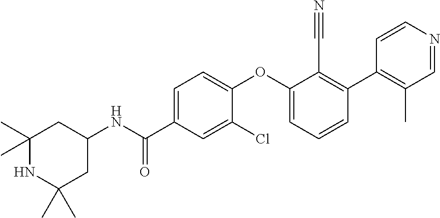 [SMILES: Cc1cnccc1-c1cccc(Oc2ccc(C(=O)NC3CC(C)(C)NC(C)(C)C3)cc2Cl)c1C#N] | I-572 |
| 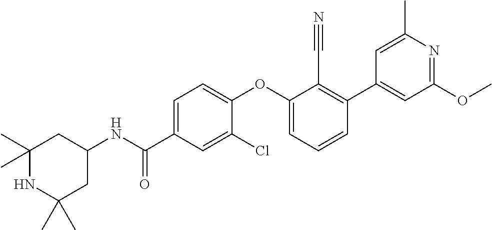 | I-573 |
| 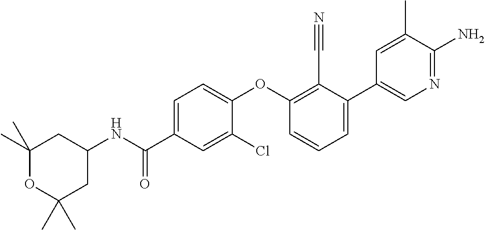 [SMILES: Cc1cc(-c2cccc(Oc3ccc(C(=O)NC4CC(C)(C)OC(C)(C)C4)cc3Cl)c2C#N)cnc1N] | I-574 |
| 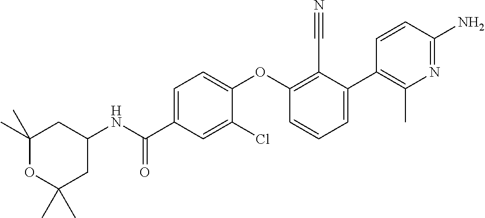 [SMILES: Cc1nc(N)ccc1-c1cccc(Oc2ccc(C(=O)NC3CC(C)(C)OC(C)(C)C3)cc2Cl)c1C#N] | I-575 |
| 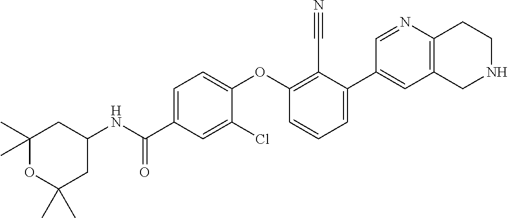 [SMILES: CC1(C)CC(NC(=O)c2ccc(Oc3cccc(-c4cnc5c(c4)CNCC5)c3C#N)c(Cl)c2)CC(C)(C)O1] | I-576 |
| 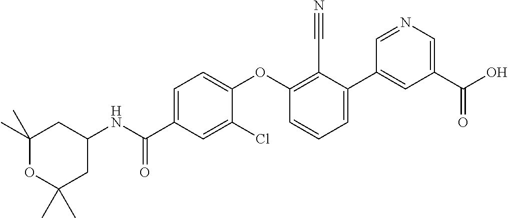 [SMILES: CC1(C)CC(NC(=O)c2ccc(Oc3cccc(-c4cncc(C(=O)O)c4)c3C#N)c(Cl)c2)CC(C)(C)O1] | I-577 |
| 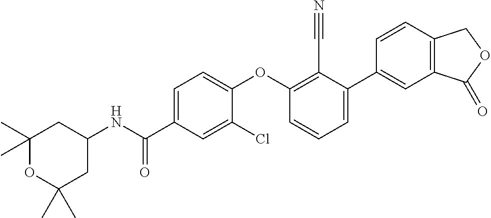 [SMILES: CC1(C)CC(NC(=O)c2ccc(Oc3cccc(-c4ccc5c(c4)C(=O)OC5)c3C#N)c(Cl)c2)CC(C)(C)O1] | I-578 |
| 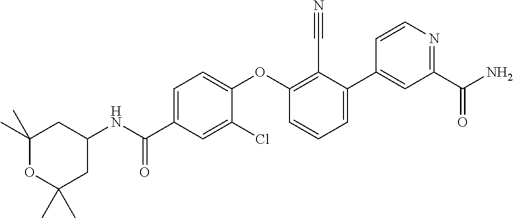 [SMILES: CC1(C)CC(NC(=O)c2ccc(Oc3cccc(-c4ccnc(C(N)=O)c4)c3C#N)c(Cl)c2)CC(C)(C)O1] | I-579 |
| 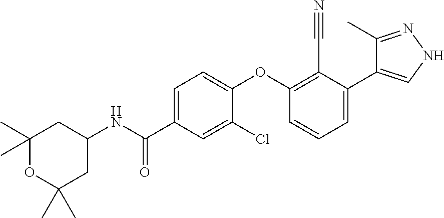 [SMILES: Cc1n[nH]cc1-c1cccc(Oc2ccc(C(=O)NC3CC(C)(C)OC(C)(C)C3)cc2Cl)c1C#N] | I-580 |
| 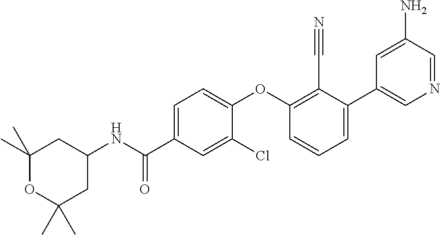 [SMILES: CC1(C)CC(NC(=O)c2ccc(Oc3cccc(-c4cncc(N)c4)c3C#N)c(Cl)c2)CC(C)(C)O1] | I-581 |
| 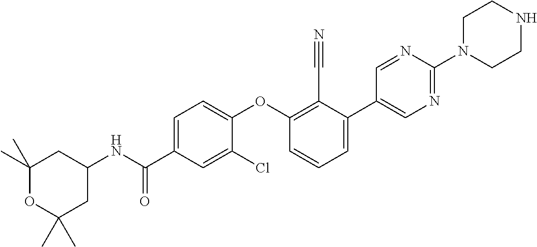 [SMILES: CC1(C)CC(NC(=O)c2ccc(Oc3cccc(-c4cnc(N5CCNCC5)nc4)c3C#N)c(Cl)c2)CC(C)(C)O1] | I-582 |
| 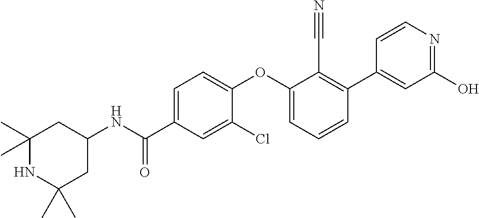 [SMILES: CC1(C)CC(NC(=O)c2ccc(Oc3cccc(-c4ccnc(O)c4)c3C#N)c(Cl)c2)CC(C)(C)N1] | I-583 |
| 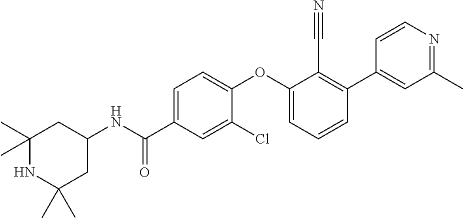 | I-584 |
| 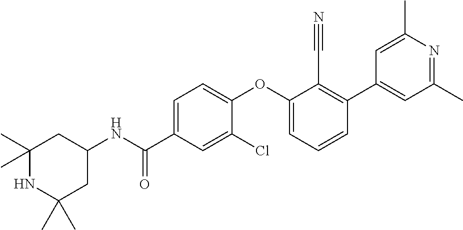 | I-585 |
| 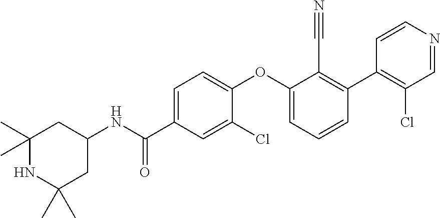 [SMILES: CC1(C)CC(NC(=O)c2ccc(Oc3cccc(-c4ccncc4Cl)c3C#N)c(Cl)c2)CC(C)(C)N1] | I-586 |
| 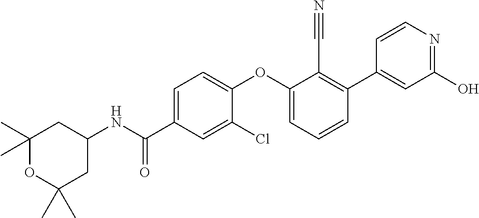 [SMILES: CC1(C)CC(NC(=O)c2ccc(Oc3cccc(-c4ccnc(O)c4)c3C#N)c(Cl)c2)CC(C)(C)O1] | I-587 |
| 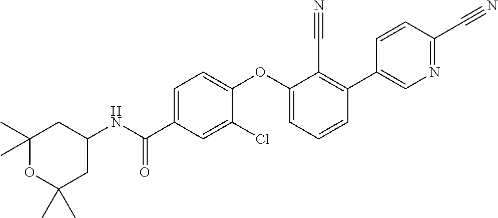 [SMILES: CC1(C)CC(NC(=O)c2ccc(Oc3cccc(-c4ccc(C#N)nc4)c3C#N)c(Cl)c2)CC(C)(C)O1] | I-588 |
| 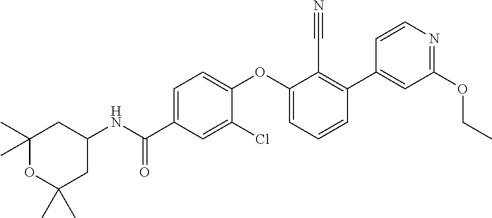 [SMILES: CCOc1cc(-c2cccc(Oc3ccc(C(=O)NC4CC(C)(C)OC(C)(C)C4)cc3Cl)c2C#N)ccn1] | I-589 |
| 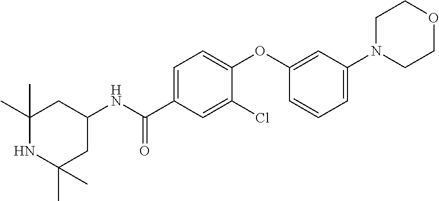 [SMILES: CC1(C)CC(NC(=O)c2ccc(Oc3cccc(N4CCOCC4)c3)c(Cl)c2)CC(C)(C)N1] | I-590 |
| 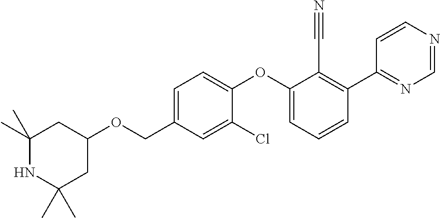 [SMILES: CC1(C)CC(OCc2ccc(Oc3cccc(-c4ccncn4)c3C#N)c(Cl)c2)CC(C)(C)N1] | I-591 |
| 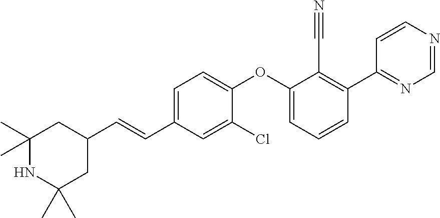 [SMILES: CC1(C)CC(/C=C/c2ccc(Oc3cccc(-c4ccncn4)c3C#N)c(Cl)c2)CC(C)(C)N1] | I-592 |
| 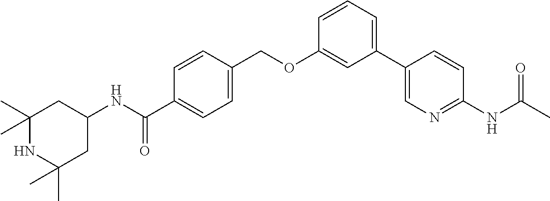 [SMILES: CC(=O)Nc1ccc(-c2cccc(OCc3ccc(C(=O)NC4CC(C)(C)NC(C)(C)C4)cc3)c2)cn1] | I-593 |
| 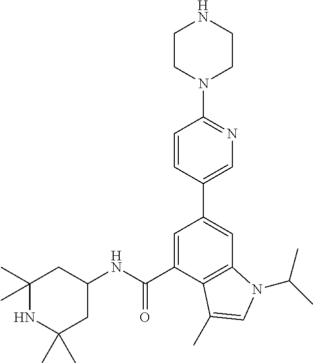 [SMILES: Cc1cn(C(C)C)c2cc(-c3ccc(N4CCNCC4)nc3)cc(C(=O)NC3CC(C)(C)NC(C)(C)C3)c12] | I-594 |
| 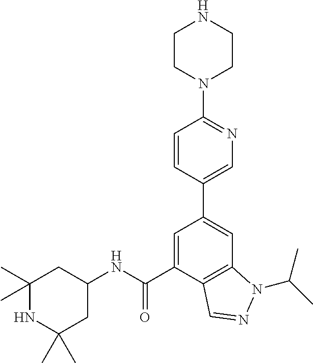 [SMILES: CC(C)n1ncc2c(C(=O)NC3CC(C)(C)NC(C)(C)C3)cc(-c3ccc(N4CCNCC4)nc3)cc21] | I-595 |

In some embodiments, the present invention provides a compound depicted in Table 1, above, or a pharmaceutically acceptable salt thereof.

### 4. Uses, Formulation and Administration

Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably modulate a histone methyl modifying enzyme, or a mutant thereof, in a biological sample or in a patient. In certain embodiments, the amount of compound in compositions of this invention is such that is effective to measurably modulate a histone methyl modifying enzyme, or a mutant thereof, in a biological sample or in a patient.

In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated and the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Compounds and compositions described herein are generally useful for the modulating of activity of one or more enzymes involved in epigenetic regulation.

Epigenetics is the study of heritable changes in gene expression caused by mechanisms other than changes in the underlying DNA sequence. Molecular mechanisms that play a role in epigenetic regulation include DNA methylation and chromatin/histone modifications. Histone methylation, in particular, is critical in many epigenetic phenomena.

Chromatin, the organized assemblage of nuclear DNA and histone proteins, is the basis for a multitude of vital nuclear processes including regulation of transcription, replication, DNA-damage repair and progression through the cell cycle. A number of factors, such as chromatin-modifying enzymes, have been identified that play an important role in maintaining the dynamic equilibrium of chromatin (Margueron, et al. (2005) Curr. Opin. Genet. Dev. 15:163-176).

Histones are the chief protein components of chromatin. They act as spools around which DNA winds, and they play a role in gene regulation. There are a total of six classes of histones (H1, H2A, H2B, H3, H4, and H5) organized into two super classes: core histones (H2A, H2B, H3, and H4) and linker histones (H1 and H5). The basic unit of chromatin is the nucleosome, which consists of about 147 base pairs of DNA wrapped around the histone octamer, consisting of two copies each of the core histones H2A, H2B, H3, and H4 (Luger, et al. (1997) Nature 389:251-260).

Histones, particularly residues of the amino termini of histones H3 and H4 and the amino and carboxyl termini of histones H2A, H2B and H1, are susceptible to a variety of post-translational modifications including acetylation, methylation, phosphorylation, ribosylation, sumoylation, ubiquitination, citrullination, deimination, and biotinylation. The core of histones H2A and H3 can also be modified. Histone modifications are integral to diverse biological processes such as gene regulation, DNA repair, and chromosome condensation.

The present disclosure provides compounds and compositions for modulating activity of histone methyl modifying enzymes. Histone methyl modifying enzymes are key regulators of cellular and developmental processes. Histone methyl modifying enzymes may be characterized as either histone methyl transferases or histone demethylases. Histone demethylase enzymes have modules that mediate binding to methylated residues. For example, multiple demethylases contain a Tudor domain (e.g., JMJD2C/GASC1) or a PHD domain (e.g., JARID1C/SMCX, PHF8).

The lysine specificities of many histone methyltransferases have been characterized. For example SET7/9, SMYD3, and MLL1-5 are specific for H3K4. SUV39H1, DIM-5, and G9a are specific for H3K9. SET8 is specific for H4K20.

DOT1 is an example of a non-SET domain containing histone methylase. DOT1 methylates H3 on lysine 79.

Just as histone methylases have been shown to regulate transcriptional activity, chromatin structure, and gene silencing, demethylases have also been discovered which impact gene expression. LSD1 was the first histone lysine demethylase to be characterized. This enzyme displays homology to FAD-dependent amine oxidases and acts as a transcriptional corepressor of neuronal genes (Shi et al., Cell 119:941-953, 2004). Additional demethylases defining separate demethylase families have been discovered, including JHDM1 (or KDM2), JHDM2 (or KDM3), JMJD2 (or KDM4), JARID (or KDM5), JMJD3 (or KDM6), and JMJD6 families (Lan et al., Curr. Opin. Cell Biol. 20(3):316-325, 2008).

Demethylases act on specific lysine residues within substrate sequences and discriminate between the degree of methylation present on a given residue. For example, LSD1 removes mono- or dimethyl-groups from H3K4. Members of the JARID1A-D family remove trimethyl groups from H3K4. UTX and JMJD3 demethylate H3K27, counteracting effects of EZH2 methylase activity. Substrate specificities of other demethylases have been characterized (see Shi, Nat. Rev. 8:829-833, 2007).

One class of histone methylases is characterized by the presence of a SET domain, named after proteins that share the domain, Su(var)3-9, enhancer of zeste [E(Z)], and trithorax. A SET domain includes about 130 amino acids. SET domain-containing methylase families include SUV39H1, SET1, SET2, EZH2, RIZ1, SMYD3, SUV4-20H1, SET7/9, and PR-SET7/SET8 families (reviewed in Dillon et al., Genome Biol. 6:227, 2005). Members of a family typically include similar sequence motifs in the vicinity of and within the SET domain. The human genome encodes over 50 SET domain-containing histone protein methylases, any of which can be used in an assay described herein.

EZH2 is an example of a human SET-domain containing methylase. EZH2 associates with EED (Embryonic Ectoderm Development) and SUZ12 (suppressor of zeste 12 homolog) to form a complex known as PRC2 (Polycomb Group Repressive Complex 2) having the ability to tri-methylate histone H3 at lysine 27 (Cao and Zhang, Mol. Cell. 15:57-67, 2004). PRC2 complexes can also include RBAP46 and RBAP48 subunits.

The oncogenic activities of EZH2 have been shown by a number of studies. In cell line experiments, over-expression of EZH2 induces cell invasion, growth in soft agar, and motility while knockdown of EZH2 inhibits cell proliferation and cell invasion (Kleer et al., 2003, Proc. Nat. Acad. Sci. USA 100:11606-11611; Varambally et al., (2002), “The polycomb group protein EZH2 is involved in progression of prostate cancer,” Nature 419, 624-629). It has been shown that EZH2 represses the expression of several tumor supressors, including E-cadherin, DAB2IP and RUNX3 among others. In xenograft models, EZH2 knockdown inhibits tumor growth and metastasis. Recently, it has been shown that down modulation of EZH2 in murine models blocks prostate cancer metastasis (Min et al., “An oncogene-tumor suppressor cascade drives metastatic prostate cancer by coordinately activating Ras and nuclear factor-kappaB,” Nat. Med. 2010 March; 16(3):286-94). EZH2 overexpression is associated with aggressiveness of certain cancers such as breast cancer (Kleer et al., Proc. Nat. Acad. Sci. USA 100:11606-11611, 2003). Recent studies also suggest that prostate cancer specific oncogenic fusion gene TMPRSS2-ERG induces repressive epigenetic programs via direct activation of EZH2 (Yu et al., “An Integrated Network of Androgen Receptor, Polycomb, and TMPRSS2-ERG Gene Fusions in Prostate Cancer Progression,” Cancer Cell. 2010 May 18; 17(5):443-454).

In some embodiments, compounds of the present invention modulate the activity of one or more enzymes involved in epigenetic regulation. In some embodiments, compounds of the present invention modulate the activity of a histone methyl modifying enzyme, or a mutant thereof. In some embodiments, compounds of the present invention modulate EZH2 activity. In some embodiments, compounds of the present invention down-regulate or suppress the activity of EZH2. In some embodiments, compounds of the present invention are antagonists of EZH2 activity.

In some embodiments, compounds and compositions of the present invention are useful in treating diseases and/or disorders associated with a histone methyl modifying enzyme. Accordingly, in some embodiments, the present invention provides a method of modulating a disease and/or disorder associated with a histone methyl modifying enzyme. In some embodiments, the present invention provides a method of treating a subject suffering from a disease and/or disorder associated with a histone methyl modifying enzyme comprising the step of administering a compound or composition of formula I.

In some embodiments, compounds and compositions of the present invention are useful in treating diseases and/or disorders associated with overexpression of EZH2. In some embodiments, the present invention provides a method of treating a subject suffering from a disease and/or disorder associated with overexpression of EZH2 comprising the step of administering a compound or composition of formula I. In some embodiments, the above method additionally comprises the preliminary step of determining if the subject is overexpressing EZH2.

In some embodiments, compounds and compositions of the present invention are useful in treating diseases and/or disorders associated with cellular proliferation. In some embodiments, compounds and compositions of the present invention are useful in treating diseases and/or disorders associated with misregulation of cell cycle or DNA repair. In some embodiments, compounds and compositions of the present invention are useful in treating cancer. Exemplary types of cancer include breast cancer, prostate cancer, colon cancer, renal cell carcinoma, glioblastoma multiforme cancer, bladder cancer, melanoma, bronchial cancer, lymphoma and liver cancer.

The study of EZH2 deletions, missense and frameshift mutations suggest that EZH2 functions as a tumor suppressor in blood disorders such as myelodysplastic syndromes (MDS) and myeloid malignancies (Ernst et al., Nat Genet. 2010 August; 42(8):722-6; Nikoloski et al., Nat Genet. 2010 August; 42(8):665-7). Accordingly, in some embodiments, compounds and compositions of the present invention are useful in treating diseases and/or disorders associated with the presence of a mutant form of EZH2. In some embodiments, compounds and compositions of the present invention are useful in treating diseases and/or disorders associated with the presence of Y641N EZH2. In some embodiment, the disease or disorder associated with the presence of a mutant form of EZH2 is a human B cell lymphoma. In some embodiments, the disease and/or disorder associated with the presence of Y641N EZH2 is follicular lymphoma or diffuse large-B-cell lymphoma. In some embodiments, compounds or compositions of the present invention are useful in treating blood disorders, such as myelodysplastic syndromes, leukemia, anemia and cytopenia. Sneeringer et al., “Coordinated activities of wild-type plus mutant EZH2 drive tumor-associated hypertrimethylation of lysine 27 on histone H3 (H3K27) in human B-cell lymphomas,” Proceedings of the National Academy of Sciences, PNAS Early Edition published ahead of print on Nov. 15, 2010.

In some embodiments, the present invention provides a method of reducing the activity of a mutant form of EZH2, such as Y641N EZH2, in a subject in need thereof comprising the step of administering a compound or composition of formula I. In some embodiments, the present invention provides a method of treating a subject suffering from a disease and/or disorder associated with a mutant form of EZH2 comprising the step of administering a compound or composition of formula I. In some embodiments, the above method additionally comprises the preliminary step of determining if the subject is expressing a mutant form of EZH2, such as Y641N EZH2. In some embodiments, that determination is made by determining if the subject has increased levels of histone H3 Lys-27-specific trimethylation (H3K27me3), as compared to a subject known not to express a mutant form of EZH2.

### EQUIVALENTS

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples that follow and the references to the scientific and patent literature cited herein. It should further be appreciated that the contents of those cited references are incorporated herein by reference to help illustrate the state of the art.

It will be appreciated that for compound preparations described herein, when reverse phase HPLC is used to purify a compound, a compound may exist as an acid addition salt. In some embodiments, a compound may exist as a formic acid or mono-, di-, or tri-trifluoroacetic acid salt.

It will further be appreciated that the present invention contemplates individual compounds described herein. Where individual compounds exemplified are isolated and/or characterized as a salt, for example, as a trifluoroacetic acid salt, the present invention contemplates a free base of the salt, as well as other pharmaceutically acceptable salts of the free base.

The following examples contain important additional information, exemplification and guidance that can be adapted to the practice of this invention in its various embodiments and the equivalents thereof

### EXAMPLES

As depicted in the Examples below, in certain exemplary embodiments, compounds are prepared according to the following general procedures. It will be appreciated that, although the synthetic methods and Schemes depict the synthesis of certain compounds of the present invention, the following methods and other methods known to one of ordinary skill in the art can be applied to all compounds and subclasses and species of each of these compounds, as described herein.

### Example 1

### Synthesis of N-(2,2,6,6-tetramethylpiperidin-4-yl)-4-(m-tolyloxymethyl)benzamide (I-46)

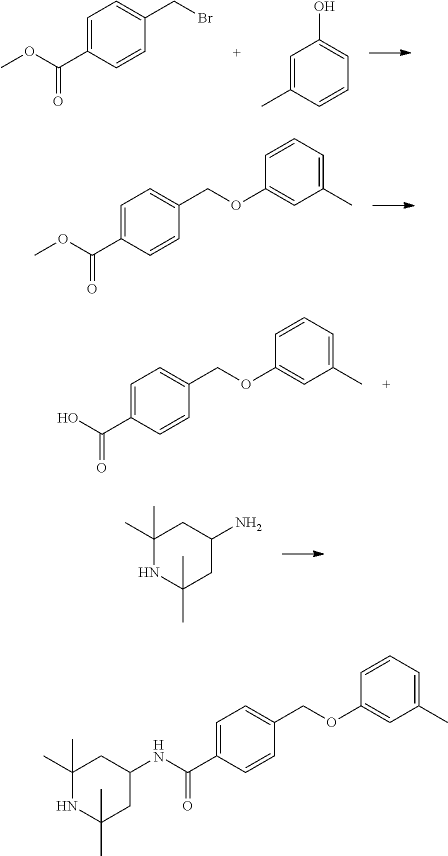

### Synthesis of methyl 4-(m-tolyloxymethyl)benzoate

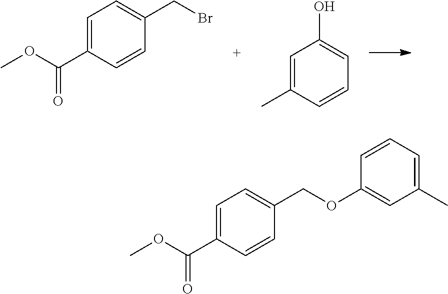

To a suspension of K₂CO₃ (902 mg, 6.5 mmol) in acetone (50 mL) was added 2-cresol (473 mg, 4.4 mmol) at room temperature. To the above suspension was added a solution of methyl 4-(bromomethyl)benzoate (1 g, 4.4 mmol) in 5 mL of acetone at room temperature. The mixture was stirred at reflux for about 5 h. The mixture was concentrated and subjected to column chromatography purification to afford methyl 4-(m-tolyloxymethyl)benzoate (1.1 g, 90%) as a white solid. LRMS [M+H]⁺ m/z: calcd 256.11. found 256.

### Synthesis of 4-(m-tolyloxymethyl)benzoic acid

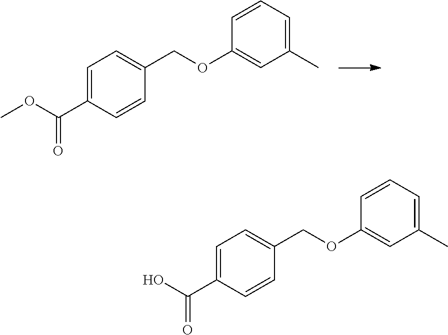

To a solution of methyl 4-(m-tolyloxymethyl)benzoate (500 mg, 1.95 mmol) in EtOH (6 mL) was added a solution of NaOH (390 mg, 9.76 mmol) in water (3 mL) dropwise at room temperature. The mixture was stirred at 80° C. for 0.5 h. After the mixture was cooled to room temperature, 1N HCl (5 mL) was dropped to the above solution, a precipitate was formed. The precipitate was collected and dried to afford 4-(m-tolyloxymethyl)benzoic acid (417 mg, 88%) as a white solid.

### Synthesis of N-(2,2,6,6-tetramethylpiperidin-4-yl)-4-(m-tolyloxymethyl)-benzamide (I-46)

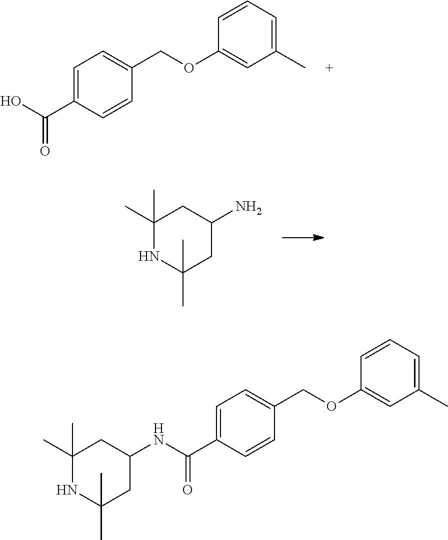

To a solution of 4-(m-tolyloxymethyl)benzoic acid (100 mg, 0.41 mmol) in DMF (4 mL) was added HATU (188 mg, 0.50 mmol) and 2,2,6,6-tetramethylpiperidin-4-amine (71 mg, 0.45 mmol) at room temperature. The mixture was stirred for about half an hour. Then to the above solution was added DIPEA (133 mg, 1.03 mmol). The mixture was stirred at room temperature for 12 hours, diluted with water (10 mL), extracted with EA (10 mL×3). The combined organic layers were dried over sodium sulfate, filtered, concentrated and subjected to the prep-HPLC to afford N-(2,2,6,6-tetramethylpiperidin-4-yl)-4-(m-tolyloxymethyl)-benzamide (15 mg, 9% as a white solid. LRMS [M+H]⁺ m/z: calcd: 380.25. found 380. ¹H NMR (300 MHz, CD₃OD): δ 7.86 (d, J=8.3 Hz, 2H), 7.56 (d, J=8.2 Hz, 2H), 7.15 (t, J=7.8 Hz, 1H), 6.91-6.73 (m, 3H), 5.15 (s, 2H), 4.54 (t, J=12.0 Hz, 1H), 2.32 (s, 3H), 2.18 (dd, J=13.9, 3.3 Hz, 2H), 1.69 (t, J=13.0 Hz, 2H), 1.60 (s, 6H), 1.50 (s, 6H).

By a similar method as Example 1, the following compounds were prepared and isolated unless where noted below.

| Compound | Structure | Data |
|---|---|---|
| I-4 | 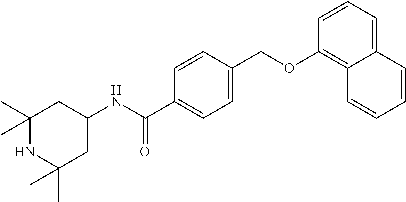 [SMILES: CC1(C)CC(NC(=O)c2ccc(COc3cccc4ccccc34)cc2)CC(C)(C)N1] | [M + H]⁺ = 417 ¹H NMR (400 MHz, DMSO-d₆) δ = 8.24-8.20 (m, 1H), 8.16 (d, J = 8.0 Hz, 1H), 7.90-7.84 (m, 3H), 7.61 (d, J = 8.2 Hz, 2H), 7.56-7.47 (m, 3H), 7.44-7.37 (m, 1H), 7.05 (d, J = 7.1 Hz, 1H), 5.36 (s, 2H), 4.33-4.21 (m, 1H), 1.68 (d, J = 9.2 Hz, 2H), 1.20-1.15 (m, 6H), 1.15-1.09 (m, 2H), 1.04 (s, 6H). |
| I-8 | 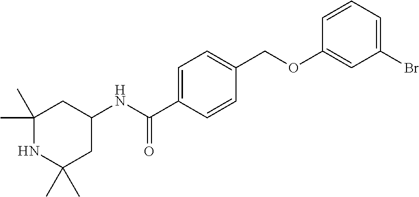 [SMILES: CC1(C)CC(NC(=O)c2ccc(COc3cccc(Br)c3)cc2)CC(C)(C)N1] | [M + H]⁺ = 445/447 |
| I-9 | 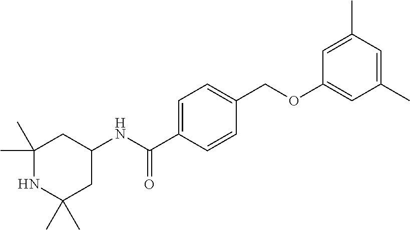 | [M + H]⁺ = 395 ¹H NMR (400 MHz, DMSO-d₆) δ: 8.57 (d, J = 12.6 Hz, 1H), 8.43 (d, J = 7.3 Hz, 1H), 7.81 (d, J = 8.2 Hz, 2H), 7.76 (d, J = 13.5 Hz, 1H), 7.49 (d, J = 8.5 Hz, 2H), 6.60 (s, 2H), 6.55 (s, 1H), 5.09 (s, 2H), 4.24-4.38 (m, 1H), 2.19 (s, 6H), 1.95 (d, J = 10.5 Hz, 2H), 1.52 (t, J = 12.8 Hz, 2H), 1.42 (s, 6H), 1.35 (s, 6H). |
| I-15 | 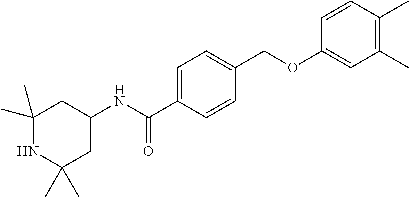 | LRMS [M + H⁺] m/z: calcd 394.26; found 394. ¹H NMR (300 MHz, CD₃OD): δ 7.83 (d, J = 8.2 Hz, 2H), 7.52 (d, J = 8.2 Hz, 2H), 6.99 (d, J = 8.4 Hz, 1H), 6.78 (d, J = 2.2 Hz, 1H), 6.68 (dd, J = 2.2, 8.4 Hz, 1H), 5.08 (s, 2H), 4.52-4.50 (m, 1H), 2.20 (s, 3H), 2.16 (s, 3H), 2.11 (d, J = 3.0, 2H), 1.66 (t, J = 13.0 Hz, 2H), 1.57 (s, 6H), 1.48 (s, 6H). |
| I-22 | 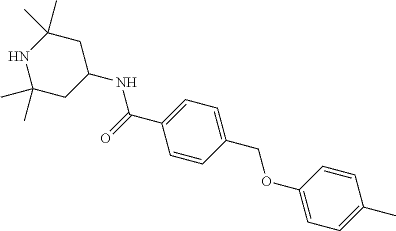 | LRMS [M + H⁺] m/z: calcd 380.25; found 380. ¹H NMR (300 MHz, CD₃OD): δ 7.82 (d, J = 8.1 Hz, 2H), 7.52 (d, J = 8.1 Hz, 2H), 7.06 (d, J = 8.7 Hz, 2H), 6.85 (d, J = 8.7 Hz, 2H), 5.11 (s, 2H), 4.46-4.56 (m, 1H), 2.25 (s, 3H), 2.15 (dd, J = 3.6, 13.8 Hz, 2H), 1.65 (t, J = 13.2 Hz, 2H), 1.58 (s, 6H), 1.48 (s, 6H). |
| I-23 | 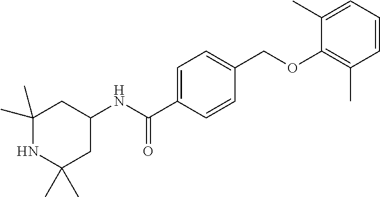 [SMILES: Cc1cccc(C)c1OCc1ccc(C(=O)NC2CC(C)(C)NC(C)(C)C2)cc1] | LRMS [M + H⁺] m/z: calcd 394.26; found 394. ¹H NMR (300 MHz, CD₃OD): δ 7.86 (d, J = 8.1 Hz, 2H), 7.60 (d, J = 8.1 Hz, 2H), 7.04 (m, 2H), 6.95-6.90 (m, 1H), 4.89 (s, 2H), 4.60-4.48 (m, 1H), 2.27 (s, 6H), 2.18 (dd, J = 3.5, 14.0 Hz, 2H), 1.66 (t, J = 12.9 Hz, 2H), 1.59 (s, 6H), 1.49 (s, 6H). |
| I-31 | 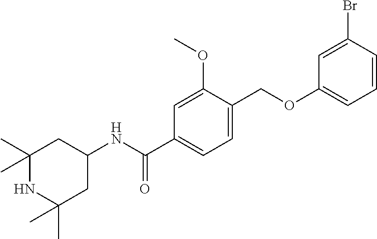 [SMILES: COc1cc(C(=O)NC2CC(C)(C)NC(C)(C)C2)ccc1COc1cccc(Br)c1] | [M + H]⁺ = 475/478 ¹H NMR (400 MHz, DMSO-d₆) δ = 8.16 (d, J = 7.6 Hz, 1H), 7.42-7.47 (m, 3H), 7.20-7.27 (m, 2H), 7.11-7.15 (m, 1H), 6.98-7.02 (m, 1H), 5.10 (s, 2H), 4.21-4.33 (m, 1H), 3.88 (s, 3H), 1.69 (dd, J = 12.1, 3.4 Hz, 2H), 1.17 (s, 6H), 1.10-1.16 (m, 2H), 1.04 ppm (s, 6H). |
| I-36 | 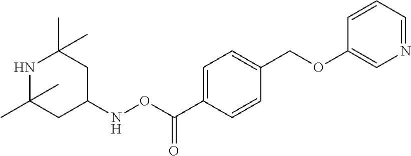 | LRMS [M + H⁺] m/z: calcd 367.23; found 367. ¹H NMR (300 MHz, CD₃OD): δ 8.37 (s, 2H), 8.18 (s, 2H), 7.90 (d, J = 4.8 Hz, 2H), 7.69 (s, 2H), 7.54 (d, J = 4.8 Hz, 2H), 5.70 (s, 2H), 4.47- 4.55 (m, 1H), 2.10 (d, J = 13.2 Hz, 2H), 1.71 (t, J = 12.9 Hz, 2H), 1.57 (s, 6H), 1.48 (s, 6H). |
| I-42 | 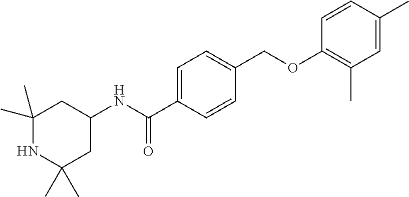 | LRMS [M + H⁺] m/z: calcd 394.26; found 394. ¹H NMR (300 MHz, CD₃OD): δ 7.86 (d, J = 8.2 Hz, 2H), 7.58 (d, J = 8.2 Hz, 2H), 6.97 (s, 1H), 6.94-6.91 (m, 1H), 6.82 (d, J = 8.1 Hz, 1H), 5.15 (s, 2H), 4.60-4.49 (m, 1H), 2.24 (s, 6H), 2.18 (dd, J = 3.7, 14.2 Hz, 2H), 1.66 (t, J = 12.9 Hz, 2H), 1.60 (s, 6H), 1.50 (s, 6H). |
| I-54 | 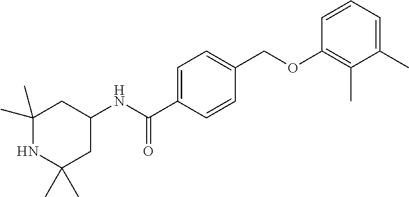 | LRMS [M + H⁺] m/z: calcd 394.26: found 394. ¹H NMR (300 MHz, DMSO-d₆): δ 8.34 (s, 1H), 8.27 (d, J = 7.8 Hz, 1H), 7.86 (s, 1H), 7.84 (s, 1H), 7.54 (s, 1H), 7.51 (s, 1H), 7.02 (t, J = 8.4 Hz, 1H), 6.85 (d, J = 7.8 Hz, H), 6.77 (d, J = 7.8 Hz, 1H), 5.15 (s, 2H), 4.30 (m, 1H), 2.22 (s, 3H), 2.13 (s, 3H), 1.80(d, J = 3.3 Hz, 1H), 1.76 (d, J = 3.6 Hz, 1H), 1.34 (s, 1H), 1.3 (s, 1H), 1.27 (s, 6H), 1.17 (s, 6H). |
| I-59 | 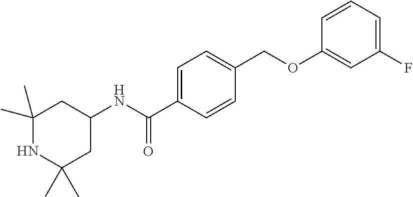 [SMILES: CC1(C)CC(NC(=O)c2ccc(COc3cccc(F)c3)cc2)CC(C)(C)N1] | LRMS [M + H⁺] m/z: calcd 384.22; found 384. ¹H NMR (300 MHz, CD₃OD): δ 7.86 (d, J = 8.4 Hz, 2H), 7.57 (d, J = 8.4 Hz, 2H), 7.29 (dd, J = 15.0, 8.0 Hz, 1H), 6.86-6.67 (m, 3H), 5.19 (s, 2H), 4.55-4.50 (m, 1H), 2.18 (dd, J = 3.6, 13.8 Hz, 2H), 1.66 (d, J = 12.9 Hz, 2H), 1.60 (s, 6H), 1.50 (s, 6H). |
| I-62 | 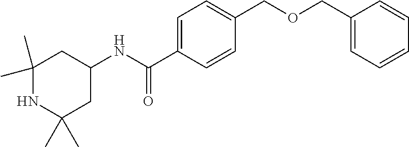 [SMILES: CC1(C)CC(NC(=O)c2ccc(COCc3ccccc3)cc2)CC(C)(C)N1] | LRMS [M + H⁺] m/z: calcd 380.25; found 380. ¹H NMR (300 MHz, CD₃OD): δ 8.55 (s, 1H), 7.82 (d, J = 8.4 Hz, 2H), 7.47 (d, J = 8.4 Hz, 2H), 7.25-7.36 (m, 4H), 4.61 (s, 2H), 4.46-4.58 (m, 1H), 2.11 (d, J = 13.8 Hz, 2H), 1.66 (t, J = 12.9 Hz, 2H), 1.56 (s, 6H), 1.46 (s, 6H). |
| I-63 | 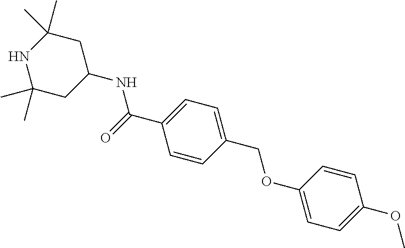 | LRMS [M + H⁺] m/z: calcd 396.24; found 396. ¹H NMR (300 MHz, CD₃OD): δ 7.82 (d, J = 8.4 Hz, 2H), 7.52 (d, J = 8.4 Hz, 2H), 6.80-6.92 (m, 4H), 5.09 (s, 2H), 4.46-4.56 (m, 1H), 3.73 (s, 3H), 2.16 (dd, J = 3.6, 14.1 Hz, 2H), 1.65 (t, J = 12.9 Hz, 2H), 1.58 (s, 6H), 1.48 (s, 6H). |
| I-66 | 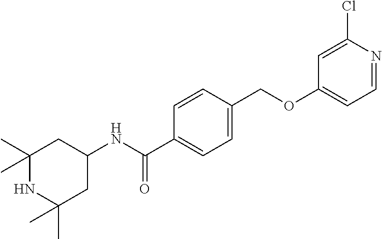 [SMILES: CC1(C)CC(NC(=O)c2ccc(COc3ccnc(Cl)c3)cc2)CC(C)(C)N1] | [M + H]⁺ = 402 ¹H NMR (400 MHz, DMSO-d₆) δ = 8.22 (d, J = 5.7 Hz, 1H), 8.16 (d, J = 7.8 Hz, 1H), 7.85 (d, J = 8.2 Hz, 2H), 7.51 (d, J = 8.0 Hz, 2H), 7.19 (d, J = 2.3 Hz, 1H), 7.07 (dd, J = 2.2, 5.8 Hz, 1H), 5.29 (s, 2H), 4.33-4.20 (m, 1H), 1.67 (dd, J = 3.7, 12.4 Hz, 2H), 1.16 (s, 6H), 1.14-1.09 (m, 2H), 1.03 (s, 6H). |
| I-67 | 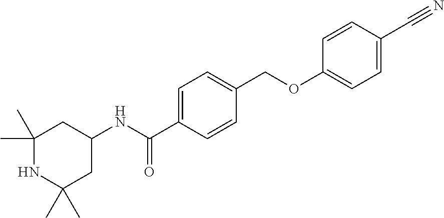 [SMILES: CC1(C)CC(NC(=O)c2ccc(COc3ccc(C#N)cc3)cc2)CC(C)(C)N1] | [M + H]⁺ = 392 |
| I-74 | 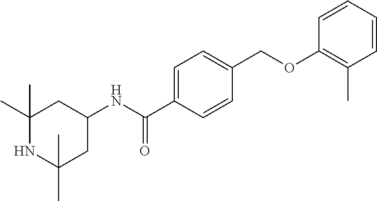 | LRMS [M + H⁺] m/z: calcd 380.25; found 380. ¹H NMR (300 MHz, CD₃OD): δ 7.85 (d, J = 8.2 Hz, 2H), 7.57 (d, J = 8.2 Hz, 2H), 7.11 (dd, J = 7.6, 11.8 Hz, 2H), 6.92 (d, J = 8.0 Hz, 1H), 6.84 (t, J = 7.4 Hz, 1H), 5.18 (s, 2H), 4.52 (t, J = 12.4 Hz, 1H), 2.26 (s, 3H), 2.17 (dd, J = 3.6, 13.9 Hz, 2H), 1.64 (t, J = 13.0 Hz, 2H), 1.58 (s, 7H), 1.48 (s, 7H). |
| I-76 | 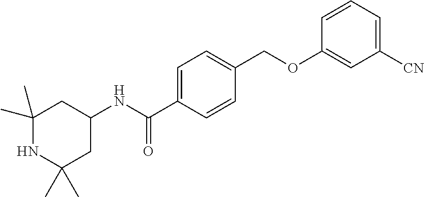 [SMILES: CC1(C)CC(NC(=O)c2ccc(COc3cccc(C#N)c3)cc2)CC(C)(C)N1] | LRMS [M + H⁺] m/z: calcd 391.23; found 391. ¹H NMR (300 MHz, CD₃OD): δ 7.87 (d, J = 8.4 Hz, 2H), 7.59 (d, J = 8.4 Hz, 2H), 7.51-7.46 (m, 1H), 7.38-7.32 (m, 3H), 5.25 (s, 2H), 4.55-4.50 (m, 1H), 2.18 (dd, J = 3.6, 13.8 Hz, 2H), 1.67 (d, J = 12.6 Hz, 2H), 1.60 (s, 6H), 1.49 (s, 6H). |
| I-77 | 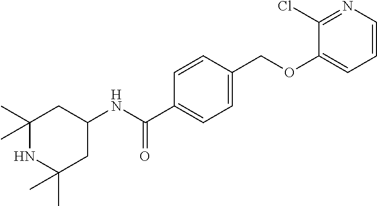 [SMILES: CC1(C)CC(NC(=O)c2ccc(COc3cccnc3Cl)cc2)CC(C)(C)N1] | [M + H]⁺ = 402 ¹H NMR (400 MHz, DMSO-d₆) δ = 8.16 (d, J = 8.0 Hz, 1H), 7.98 (dd, J = 1.5, 4.7 Hz, 1H), 7.85 (d, J = 8.2 Hz, 2H), 7.65 (dd, J = 1.6, 8.2 Hz, 1H), 7.52 (d, J = 8.2 Hz, 2H), 7.39 (dd, J = 4.6, 8.2 Hz, 1H), 5.31 (s, 2H), 4.33-4.19 (m, 1H), 1.67 (dd, J = 3.5, 12.2 Hz, 2H), 1.16 (s, 6H), 1.14-1.09 (m, 2H), 1.03 (s, 6H). |
| I-83 | 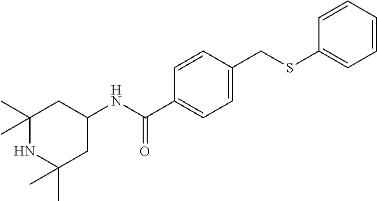 [SMILES: CC1(C)CC(NC(=O)c2ccc(CSc3ccccc3)cc2)CC(C)(C)N1] | [M + H]⁺ = 383; ¹H NMR (400 MHz, DMSO-d₆) δ = 8.09 (d, J = 7.6 Hz, 1H), 7.75-7.71 (m, 2H), 7.39 (d, J = 8.2 Hz, 2H), 7.34-7.24 (m, 4H), 7.19-7.14 (m, 1H), 4.27 (s, 2H), 4.26-4.18 (m, 1H), 1.65 (dd, J = 3.4, 12.4 Hz, 2H), 1.15 (s, 6H), 1.13-1.08 (m, 2H), 1.03 (s, 6H) |
| I-84 | 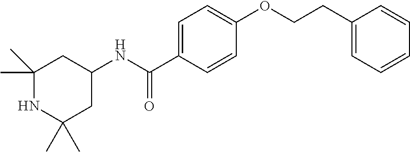 [SMILES: CC1(C)CC(NC(=O)c2ccc(OCCc3ccccc3)cc2)CC(C)(C)N1] | LRMS [M + H⁺] m/z: calcd 380.25; found 380. 1H NMR (300 MHz, CD₃OD): δ 7.78 (d, J = 8.9 Hz, 2H), 7.20-7.30 (m, 5H), 6.97 (d, J = 8.6 Hz, 2H), 4.49 (t, J = 12.2 Hz, 1H), 4.25 (t, J = 6.7 Hz, 2H), 3.09 (t, J = 6.7 Hz, 1H), 2.13 (dd, J = 2.9, 13.6 Hz, 2H), 1.65 (t, J= 13.0 Hz, 2H), 1.57 (s, 6H), 1.47 (s, 6H). |
| I-111 | 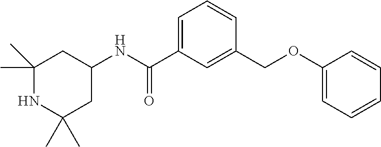 [SMILES: CC1(C)CC(NC(=O)c2cccc(COc3ccccc3)c2)CC(C)(C)N1] | [M + H]⁺ = 367 ¹H NMR (400 MHz, DMSO-d₆) δ: 8.68 (d, J = 12.4 Hz, 1H), 8.49 (d, J = 7.3 Hz, 1H), 7.90 (s, 1H), 7.84 (d, J = 12.4 Hz, 1H), 7.78 (d, J = 7.8 Hz, 1H), 7.59 (d, J = 7.6 Hz, 1H), 7.44-7.50 (m, 1H), 7.24- 7.31 (m, 2H), 6.97-7.02 (m, 2H), 6.93 (t, J = 7.3 Hz, 1H), 5.12 (s, 2H), 4.27-4.38 (m, 1H), 1.95 (dd, J = 13.4, 3.1 Hz, 2H), 1.55 (t, J = 12.8 Hz, 2H), 1.43 (s, 6H), 1.36 (s, 6H). |
| I-112 | 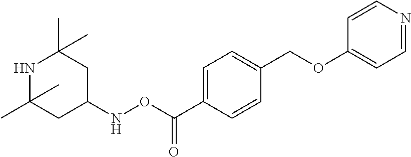 | LRMS [M + H⁺] m/z: calcd 367.23; found 367. ¹H NMR (300 MHz, CD₃OD): δ 7.88 (m, J = 7.5 Hz, 4H), 7.40 (d, J = 8.1 Hz, 2H), 7.46 (d, J = 7.5 Hz, 2H), 5.25 (s, 2H), 4.47-4.57 (m, 1H), 2.11 (dd, J = 3.0, 13.5 Hz, 2H), 1.69 (t, J = 11.1 Hz, 2H), 1.58 (s, 6H), 1.48 (s, 6H). |
| I-115 | 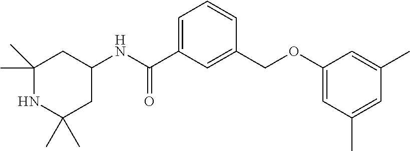 | [M + H]⁺ = 395 1H NMR (400 MHz, DMSO-d₆) δ: 8.66-8.79 (m, 1H), 8.50 (d, J = 7.6 Hz, 1H), 7.88 (s, 2H), 7.77 (d, J = 7.8 Hz, 1H), 7.57 (d, J = 7.6 Hz, 1H), 7.42- 7.51 (m, 1H), 6.62 (s, 2H), 6.57 (s, 1H), 4.23- 4.40 (m, 1H), 2.20 (s, 6H), 1.95 (dd, J = 13.5, 3.0 Hz, 2H), 1.56 (t, J = 12.9 Hz, 2H), 1.43 (s, 6H), 1.37 (s, 6H). |
| I-128 | 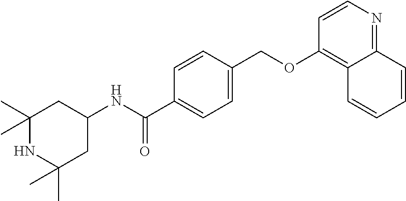 [SMILES: CC1(C)CC(NC(=O)c2ccc(COc3ccnc4ccccc34)cc2)CC(C)(C)N1] | [M + H]⁺ = 418 ¹H NMR (400 MHz, DMSO-d₆) δ = 8.22 (d, J = 7.8 Hz, 1H), 8.17 (dd, J = 1.6, 8.0 Hz, 1H), 8.09 (d, J = 7.6 Hz, 1H), 7.76 (d, J = 8.2 Hz, 2H), 7.62- 7.56 (m, 1H), 7.53-7.49 (m, 1H), 7.35-7.30 (m, 1H), 7.27 (d, J = 8.2 Hz, 2H), 6.15 (d, J = 7.8 Hz, 1H), 5.57 (s, 2H), 4.28-4.16 (m, 1H), 1.63 (dd, J = 3.5, 12.2 Hz, 2H), 1.14 (s, 6H), 1.09 (t, J = 12.2 Hz, 2H), 1.01 (s, 6H). |
| I-130 | 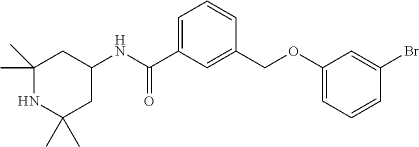 [SMILES: CC1(C)CC(NC(=O)c2cccc(COc3cccc(Br)c3)c2)CC(C)(C)N1] | [M + H]⁺ = 445/446 ¹H NMR (400 MHz, DMSO-d₆) δ: 8.65 (d, J = 11.4 Hz, 1H), 8.49 (d, J = 7.3 Hz, 1H), 7.89 (s, 1H), 7.76-7.85 (m, 2H), 7.59 (d, J = 7.8 Hz, 1H), 7.45- 7.51 (m, 1H), 7.21-7.25 (m, 2H), 7.13 (ddd, J = 8.0, 1.7, 0.7 Hz, 1H), 7.02 (ddd, J = 8.3, 2.5, 0.9 Hz, 1H), 5.16 (s, 2H), 4.26-4.38 (m, 1H), 1.96 (dd, J = 13.2, 2.9 Hz, 2H), 1.55 (t, J = 13.0 Hz, 2H), 1.43 (s, 6H), 1.36 (s, 6H). |

### Example 2

### Synthesis of 2-methyl-4-phenoxy-N-(2,2,6,6-tetramethylpiperidin-4-yl)-benzamide (I-124)

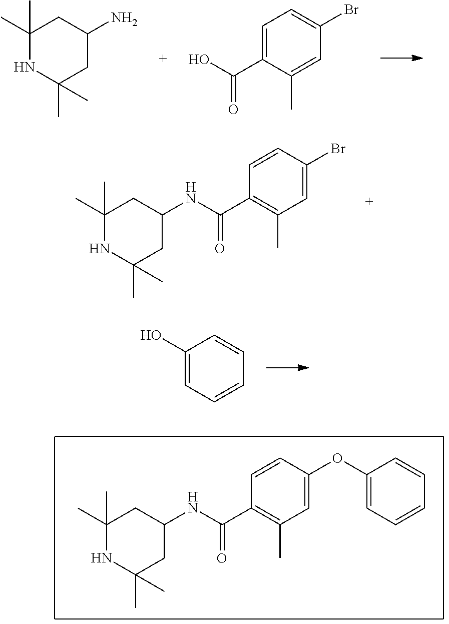

### Synthesis of 4-bromo-2-methyl-N-(2,2,6,6-tetramethylpiperidin-4-yl)benzamide

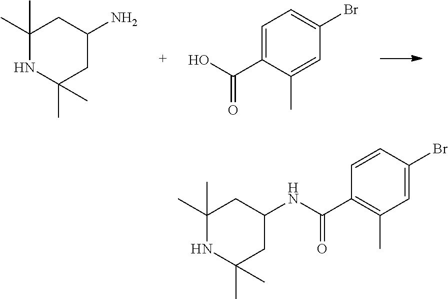 [SMILES: CC1(C)CC(N)CC(C)(C)N1 ; Cc1cc(Br)ccc1C(=O)O ; Cc1cc(Br)ccc1C(=O)NC1CC(C)(C)NC(C)(C)C1]

To a solution of 4-bromo-2-methylbenzoic acid (1 g, 4.65 mmol) in DMF (20 mL) HATU (2.12 g, 5.58 mmol) and 2,2,6,6-tetramethylpiperidin-4-amine (0.8 mg, 5.1 mmol) DIPEA (1.5 g, 11.6 mmol) were added. The mixture was stirred at room temperature for 12 hours, diluted with water (50 mL), extracted with CH₂Cl₂ (50 mL×3). The combined organic layers were dried over sodium sulfate, filtered, concentrated and subjected to column chromatography purification to afford 4-bromo-2-methyl-N-(2,2,6,6-tetramethylpiperidin-4-yl)benzamide (1.1 g, 67% as a white solid. LRMS [M+H]⁺ m/z: calcd 352.12. found 352.

### Synthesis of 2-methyl-4-phenoxy-N-(2,2,6,6-tetramethylpiperidin-4-yl)benzamide (I-124)

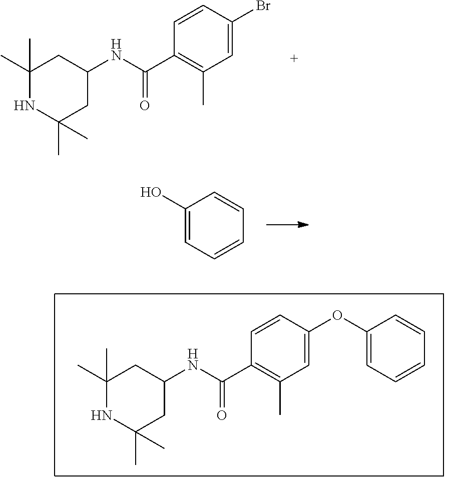 [SMILES: Cc1cc(Br)ccc1C(=O)NC1CC(C)(C)NC(C)(C)C1 ; Oc1ccccc1 ; Cc1cc(Oc2ccccc2)ccc1C(=O)NC1CC(C)(C)NC(C)(C)C1]

A mixture of 4-bromo-2-methyl-N-(2,2,6,6-tetramethylpiperidin-4-yl)benzamide (100 mg, 0.28 mmol), phenol (32 mg, 0.34 mmol), CuI (2.7 mg, 0.014 mmol), Cs₂CO₃ (120 mg, 0.56 mmol) and picolinic acid (3.69 mg, 0.03 mmol) in DMF (20 mL) was stirred at 150° C. overnight under nitrogen atmosphere. The reaction mixture was diluted with water (10 mL), extracted with CH₂Cl₂ (10 mL×3). The combined organic layers were dried over sodium sulfate, filtered, concentrated and subjected to prep-HPLC to afford 2-methyl-4-phenoxy-N-(2,2,6,6-tetramethylpiperidin-4-yl)-benzamide (50 mg, 46%) as a white solid. LRMS [M+H]⁺ m/z: calcd 366.23. found 366. ¹H NMR (300 MHz, CD₃OD): δ 8.43 (s, 1H), 7.34-7.21 (m, 3H), 7.05 (t, J=7.4 Hz, 1H), 6.90 (d, J=8.5 Hz, 2H), 6.75 (s, 1H), 6.70 (dd, J=2.2, 8.5 Hz, 1H), 4.36 (t, J=12.2 Hz, 1H), 2.28 (s, 3H), 2.05 (dd, J=3.2, 13.7 Hz, 1H), 1.57-1.38 (m, 8H), 1.35 (s, 6H).

By a similar method as Example 2, using the appropriate starting materials, the following compounds were prepared and isolated unless where noted below.

| Compound | Structure | Data |
|---|---|---|
| I-6 | 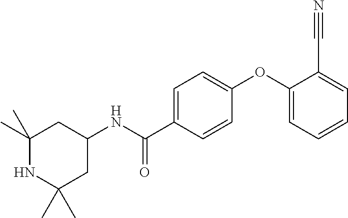 [SMILES: CC1(C)CC(NC(=O)c2ccc(Oc3ccccc3C#N)cc2)CC(C)(C)N1] | m/z (ESI) 378 [M + H]⁺. |
| I-16 | 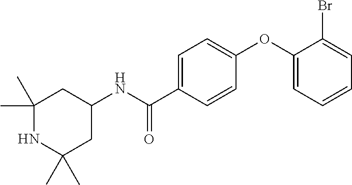 [SMILES: CC1(C)CC(NC(=O)c2ccc(Oc3ccccc3Br)cc2)CC(C)(C)N1] | LRMS [M + H]⁺ m/z: calcd 430.13; found 430. ¹H NMR (300 MHz, CD₃OD): δ 1.49 (s, 6H), 1.58 (s, 6H), 1.64-1.73 (m, 2H), 2.09-2.15 (m, 2H), 4.47-4.55 (m, 1H), 6.92-6.95 (m, 2H), 7.13-7.21 (m, 2H), 7.39-7.42 (m, 1H), 7.70- 7.73 (m, 1H), 7.83-7.86 (m, 2H). |
| I-17 | 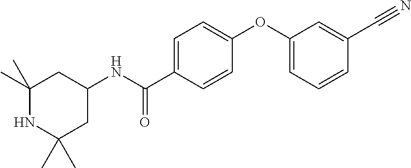 [SMILES: CC1(C)CC(NC(=O)c2ccc(Oc3cccc(C#N)c3)cc2)CC(C)(C)N1] | [M + H]⁺ = 378 ¹H NMR (DMSO-d₆) δ: 8.61 (d, J = 11.0 Hz, 1H), 8.42 (d, J = 7.6 Hz, 1H), 7.89 (d, J = 8.7 Hz, 2H), 7.78 (d, J = 12.4 Hz, 1H), 7.57-7.67 (m, 2H), 7.56 (s, 1H), 7.39 (dt, J = 8.0, 1.3 Hz, 1H), 7.12 (d, J = 8.7 Hz, 2H), 4.25-4.39 (m, 1H), 1.95 (d, J = 10.8 Hz, 2H), 1.54 (t, J = 12.9 Hz, 2H), 1.34-1.45 (m, 12H). |
| I-20 | 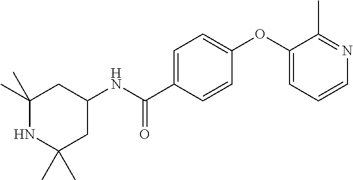 [SMILES: Cc1ncccc1Oc1ccc(C(=O)NC2CC(C)(C)NC(C)(C)C2)cc1] | [M + H]⁺ = 368 ¹H NMR (DMSO-d₆) δ: 8.80 (d, J = 12.1 Hz, 1H), 8.34-8.48 (m, 2H), 7.83-7.98 (m, 3H), 7.53 (dd, J = 8.2, 1.1 Hz, 1H), 7.41 (dd, J = 8.2, 5.0 Hz, 1H), 6.96-7.07 (m, 2H), 4.22- 4.41 (m, 1H), 2.41 (s, 3H), 1.93 (dd, J = 13.5, 3.0 Hz, 2H), 1.56 (t, J = 12.9 Hz, 2H), 1.33- 1.46 (m, 12H). |
| I-21 | 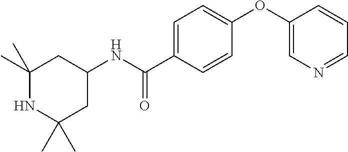 [SMILES: CC1(C)CC(NC(=O)c2ccc(Oc3cccnc3)cc2)CC(C)(C)N1] | [M + H]⁺ = 354.2 ¹H NMR (DMSO-d₆) δ: 8.75 (d, J = 11.7 Hz, 1H), 8.44 (d, J = 6.9 Hz, 3H), 7.84-7.94 (m, 3H), 7.53-7.59 (m, 1H), 7.46-7.52 (m, 1H), 7.07-7.14 (m, 2H), 4.25-4.38 (m, 1H), 1.94 (dd, J = 13.4, 2.9 Hz, 2H), 1.56 (t, J = 12.9 Hz, 2H), 1.42 (s, 6H), 1.37 (s, 6H). |
| I-24 | 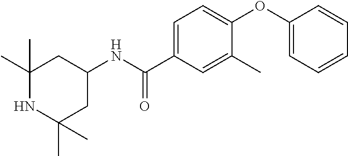 [SMILES: Cc1cc(C(=O)NC2CC(C)(C)NC(C)(C)C2)ccc1Oc1ccccc1] | LRMS [M + H]⁺ m/z: calcd 366.23; found 366. ¹H NMR (300 MHz, CD₃OD): δ 8.59 (s, 1H), 7.80 (s, 1H), 7.66 (dd, J = 1.6, 8.4 Hz, 1H), 7.36-7.42 (m, 2H), 7.13-7.19 (m, 1H), 6.96- 6.99 (m, 2H), 6.85 (d, J = 8.4 Hz, 1H), 4.47- 4.57 (m, 1H), 2.33 (s, 3H), 2.12 (dd, J = 3.9, 13.8 Hz, 2H), 1.64 (t, J = 13.2 Hz, 2H), 1.57 (s, 6H), 1.47 (s, 6H). |
| I-28 | 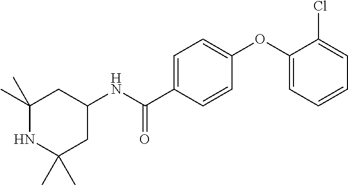 [SMILES: CC1(C)CC(NC(=O)c2ccc(Oc3ccccc3Cl)cc2)CC(C)(C)N1] | LRMS [M + H]⁺ m/z: calcd 386.18; found 386. ¹H NMR (300 MHz, CD₃OD): δ 1.49 (s, 6H), 1.58 (s, 6H), 1.63-1.72 (m, 2H), 2.10-2.16 (m, 2H), 4.47-4.55 (m, 1H), 6.92-6.96 (m, 2H), 7.17 (dd, J = 1.5, 8.1 Hz, 1H), 7.23-7.28 (m, 1H), 7.35-7.40 (m, 1H), 7.55 (dd, J = 1.5, 8.1 Hz, 1H), 7.82-7.86 (m, 2H). |
| I-35 | 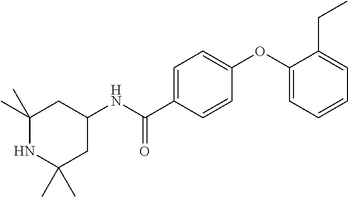 [SMILES: CCc1ccccc1Oc1ccc(C(=O)NC2CC(C)(C)NC(C)(C)C2)cc1] | [M + H]⁺ = 381.2 ¹H NMR (DMSO-d₆) δ: 8.68 (d, J = 11.9 Hz, 1H), 8.34 (d, J = 7.6 Hz, 1H), 7.78-7.88 (m, 3H), 7.33-7.38 (m, 1H), 7.22-7.27 (m, 1H), 7.15-7.20 (m, 1H), 6.94 (d, J = 8.0 Hz, 1H), 6.91 (d, J = 8.7 Hz, 2H), 4.21-4.40 (m, 1H), 2.52 (q, J = 4.0 Hz, 2H), 1.93 (dd, J = 13.4, 2.6 Hz, 2H), 1.54 (t, J = 12.9 Hz, 2H), 1.42 (s, 6H), 1.36 (s, 6H), 1.08 (t, J = 7.4 Hz, 3H). |
| I-40 | 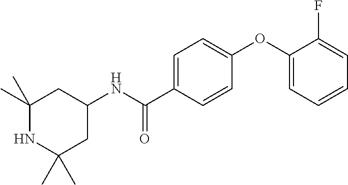 [SMILES: CC1(C)CC(NC(=O)c2ccc(Oc3ccccc3F)cc2)CC(C)(C)N1] | [M + H]⁺ =371.2 ¹H NMR (DMSO-d₆) δ: 8.52-8.61 (m, 1H), 8.37 (d, J = 7.6 Hz, 1H), 7.80-7.88 (m, 2H), 7.71-7.79 (m, 1H), 7.41 (s, 1H), 7.21-7.32 (m, 3H), 7.00 (d, J = 8.7 Hz, 2H), 4.24-4.37 (m, 1H), 1.89-2.01 (m, 2H), 1.52 (t, J = 12.9 Hz, 2H), 1.42 (s, 6H), 1.36 (s, 6H). |
| I-44 | 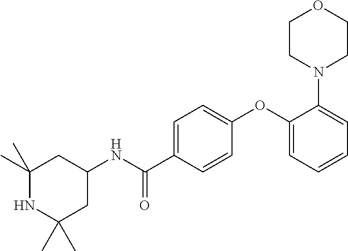 [SMILES: CC1(C)CC(NC(=O)c2ccc(Oc3ccccc3N3CCOCC3)cc2)CC(C)(C)N1] | LRMS [M + H]⁺ m/z: calcd 437.27; found 437. ¹H NMR (300 MHz, CD₃OD): δ 1.48 (s, 6H), 1.57 (s, 6H), 1.68 (t, J= 12.9 Hz, 2H), 2.10- 2.16 (m, 2H), 3.00-3.03 ( m, 4H), 3.48-3.51 (m, 4H), 4.47-4.55 (m, 1H), 6.88 (d, J = 8.7 Hz, 2H), 7.06-7.12 (m, 3H), 7.20-7.26 (m, 1H), 7.80 (d, J = 8.7 Hz, 2H). |
| I-48 | 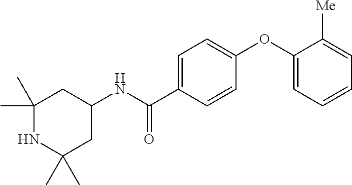 [SMILES: Cc1ccccc1Oc1ccc(C(=O)NC2CC(C)(C)NC(C)(C)C2)cc1] | LRMS [M + H]⁺ m/z: calcd 366.23; found 366. ¹H NMR (300 MHz, CD₃OD): δ 1.46 (s, 6H), 1.55 (s, 6H), 1.66 (t, J = 13.2 Hz, 2H), 2.05- 2.10 (m, 2H), 2.15 (s, 3H), 4.45-4.53 (m, 1H), 6.86-6.89 (m, 2H), 6.94-6.96 (m, 1H), 7.11- 7.16 (m, 1H), 7.20-7.25 (m, 2H), 7.29-7.31 (m, 1H), 7.79-7.82 (m, 1H). |
| I-57 | 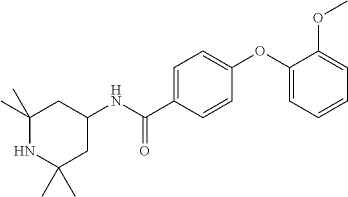 | LRMS [M + H]⁺ m/z: calcd 382.23; found 382. ¹H NMR (300 MHz, CD₃Cl): δ 1.24 (s, 6H), 1.36 (s, 6H), 1.40-1.44 (d, J = 11.1 Hz, 2H), 1.93-1.98 (d, J = 13.2 Hz, 2H), 3.74 (s, 3H), 4.45 (m, 1H), 6.83-6.86 (m, 2H), 6.96-7.26 (m, 4H), 7.75-7.78 (m, 2H). |
| I-60 | 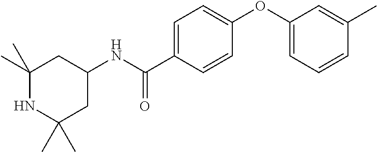 | [M + H]⁺ = 367.2 ¹H NMR (DMSO-d₆) δ = 8.61 (d, J = 12.4 Hz, 1 H), 8.36 (d, J = 7.3 Hz, 1 H), 7.88-7.74 (m, 3 H), 7.28 (t, J = 7.9 Hz, 1 H), 7.05-6.97 (m, 3 H), 6.89-6.80 (m, 2 H), 4.31 (dd, J = 3.7, 7.6 Hz, 1 H), 2.28 (s, 3 H), 1.94 (dd, J = 2.7, 13.3 Hz, 2 H), 1.53 (t, J = 12.8 Hz, 2 H), 1.42 (s, 6 H), 1.36 (s, 6 H). |
| I-69 | 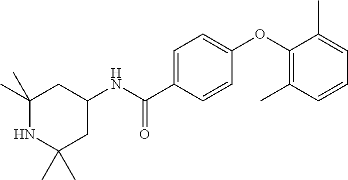 [SMILES: Cc1cccc(C)c1Oc1ccc(C(=O)NC2CC(C)(C)NC(C)(C)C2)cc1] | [M + H]⁺ = 381.2 ¹H NMR (DMSO-d₆) δ = 8.59-8.47 (m, 1 H), 8.32-8.21 (m, 1 H), 7.84-7.66 (m, 2 H), 7.22-7.06 (m, 3 H), 6.82-6.73 (m, 2 H), 4.36-4.22 (m, 1 H), 2.03 (s, 6 H), 1.97-1.89 (m, 2 H), 1.57-1.46 (m, 2 H), 1.41 (s, 6 H), 1.35 (s, 6H). |
| I-82 | 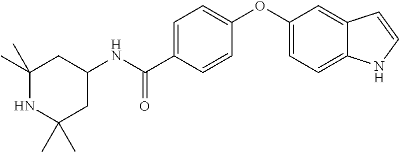 [SMILES: CC1(C)CC(NC(=O)c2ccc(Oc3ccc4[nH]ccc4c3)cc2)CC(C)(C)N1] | [M + H]⁺ = 392 ¹H NMR (DMSO-d₆) δ = 9.08-8.89 (m, 1 H), 8.69 (d, J = 11.9 Hz, 1 H), 8.53 (d, J = 7.6 Hz, 1 H), 8.01 (d, J = 8.5 Hz, 2 H), 7.85 (d, J = 12.6 Hz, 1 H), 7.67 (d, J = 8.5 Hz, 2 H), 7.62 (d, J = 3.2 Hz, 1 H), 7.46 (d, J = 8.7 Hz, 1 H), 6.96 (d, J = 2.3 Hz, 1 H), 6.72 (dd, J = 2.2, 8.8 Hz, 1 H), 6.56 (d, J = 3.4 Hz, 1 H), 4.44-4.30 (m, 1 H), 2.04-1.94 (m, 2 H), 1.59 (t, J = 12.9 Hz, 2 H), 1.46 (s, 6 H), 1.39 (s, 6 H). |
| I-87 | 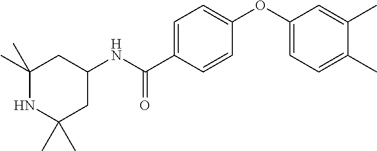 | [M + H]⁺ = 381 |
| I-89 | 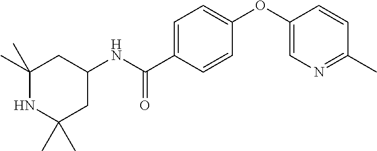 | [M + H]⁺ = 368 ¹H NMR (DMSO-d₆) δ = 8.76 (d, J = 12.4 Hz, 1 H), 8.43 (d, J = 7.3 Hz, 1 H), 8.36 (d, J = 2.7 Hz, 1 H), 7.95-7.83 (m, 3 H), 7.57 (dd, J = 2.4, 8.6 Hz, 1 H), 7.42 (d, J = 8.7 Hz, 1 H), 7.07 (d, J = 8.7 Hz, 2 H), 4.38-4.24 (m, 1 H), 2.50 (s, 3 H), 1.93 (dd, J = 2.7, 13.3 Hz, 2 H), 1.56 (t, J = 12.9 Hz, 2 H), 1.42 (s, 6 H), 1.36 (s, 6 H). |
| I-119 | 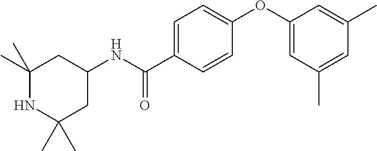 | [M + H]⁺ = 381 ¹H NMR (DMSO-d₆) δ: 8.91-9.23 (m, 1H), 8.59-8.74 (m, 1H), 8.37 (d, J = 7.6 Hz, 1H), 7.74-7.90 (m, 2H), 6.95-7.05 (m, 1H), 6.81 (s, 1H), 6.65 (s, 1H), 6.29-6.42 (m, 2H), 4.23- 4.38 (m, 1H), 2.23 (s, 3H), 2.13 (s, 3H), 1.94 (dd, J = 13.3, 2.7 Hz, 2H), 1.54 (t, J = 12.9 Hz, 2H), 1.32-1.46 (m, 12H). |
| I-122 | 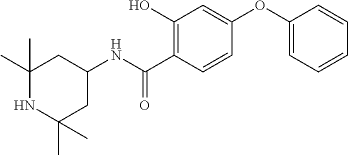 [SMILES: CC1(C)CC(NC(=O)c2ccc(Oc3ccccc3)cc2O)CC(C)(C)N1] | LRMS [M + H]⁺ m/z: calcd 368.47; found 368. ¹H NMR (300 MHz, CD₃OD): δ 7.77 (d, J = 8.7, 1H), 7.40 (m, J = 8.7, 2H), 7.19 (m, J = 7.5, 2H), 7.50 (d, J = 7.8, 2H), 6.43-6.34 (m, 2H), 4.52 (s, 1H), 2.16-2.115 (m, 2H), 1.72- 1.41 (m, 14H). |
| I-123 | 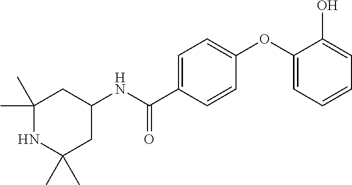 [SMILES: CC1(C)CC(NC(=O)c2ccc(Oc3ccccc3O)cc2)CC(C)(C)N1] | LRMS [M + H]⁺ m/z: calcd 368.21; found 368. ¹H NMR (300 MHz, DMSO-d₆): δ 1.26 (s, 12H), 1.52 (m, 2H), 1.60-1.51 (t, 7= 12.9 Hz, 2H), 1.89-1.85 (m, 2H), 4.33-4.28 (m, 1H), 6.85-6.81 (m, 3H), 7.07-6.81 (m, 3H), 7.83- 7.80 (m, 2H), 8.38 (d, J = 7.5 Hz, 1H), 9.72 (s, 1H). |
| I-131 | 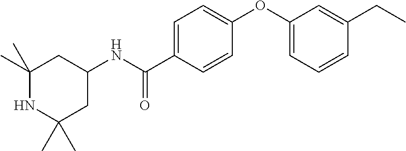 [SMILES: CCc1cccc(Oc2ccc(C(=O)NC3CC(C)(C)NC(C)(C)C3)cc2)c1] | M + H]⁺ = 381.2 ¹H NMR (DMSO-d₆) δ: 9.19 (br. s., 1H), 8.67 (d, J = 12.1 Hz, 1H), 8.37 (d, J = 7.3 Hz, 1H), 7.79-7.89 (m, 2H), 7.31 (t, J = 7.8 Hz, 1H), 6.99-7.05 (m, 2H), 6.82-6.91 (m, 1H), 6.51- 6.60 (m, 1H), 4.25-4.37 (m, 1H), 2.58 (q, J = 7.6 Hz, 2H), 1.94 (dd, J = 13.2, 2.6 Hz, 2H), 1.54 (t, J = 12.9 Hz, 2H), 1.42 (s, 6H), 1.36 (s, 6H). [ |
| I-142 | 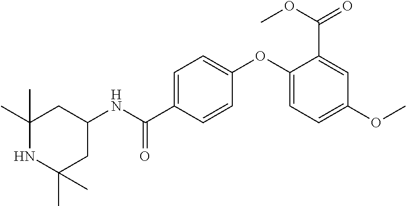 | LRMS [M + H]⁺ m/z: calcd 440.23; found 440. |

### Example 3

### Synthesis of 3-chloro-N-(2,2,6,6-tetramethylpiperidin-4-yl)-4-phenoxybenzamide (I-49)

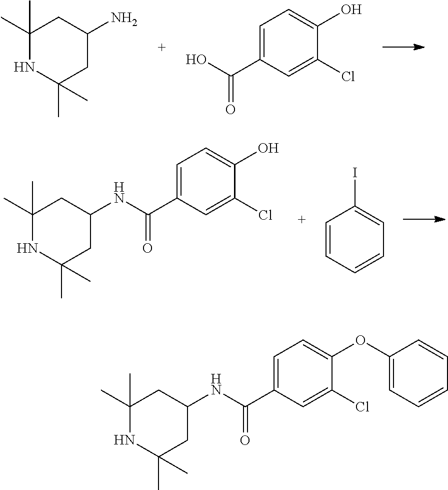

### Synthesis of 3-chloro-4-hydroxy-N-(2,2,6,6-tetramethylpiperidin-4-yl)benzamide

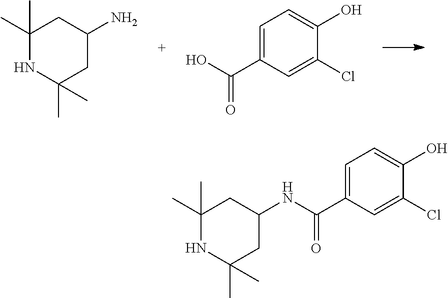 [SMILES: CC1(C)CC(N)CC(C)(C)N1 ; O=C(O)c1ccc(O)c(Cl)c1 ; CC1(C)CC(NC(=O)c2ccc(O)c(Cl)c2)CC(C)(C)N1]

3-Chloro-4-hydroxybenzoic acid (2 g, 11.6 mmol), HOBt (2.3 g, 17.4 mmol) and EDCI (3.3 g, 17.4 mmol) were dissolved in dry DCM (50 mL) and stirred for 30 ml at rt. 2,2,6,6-tetramethylpiperidin-4-amine (1.8 g, 11.6 mmol) was added and the reaction mixture was stirred at room temperature over night. Water (30 mL) was added the phases were separated. The organic phase was washed with brine (10 mL), dried with Na₂SO₄. Organic solvent was removed under reduced pressure to afford 3-chloro-4-hydroxy-N-(2,2,6,6-tetramethylpiperidin-4-yl)benzamide (2 g, 55%). LRMS [M+H]⁺ m/z: calcd 310.14. found 310.

### Synthesis of 3-chloro-N-(2,2,6,6-tetramethylpiperidin-4-yl)-4-phenoxybenzamide (I-49)

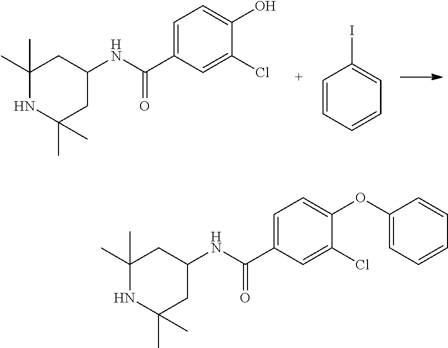 [SMILES: CC1(C)CC(NC(=O)c2ccc(O)c(Cl)c2)CC(C)(C)N1 ; Ic1ccccc1 ; CC1(C)CC(NC(=O)c2ccc(Oc3ccccc3)c(Cl)c2)CC(C)(C)N1]

3-chloro-4-hydroxy-N-(2,2,6,6-tetramethylpiperidin-4-yl)benzamide (200 mg, 0.64 mmol), iodobenzene (261 mg, 1.28 mmol) and pyridine (2 drops) were dissolved in DMF (3 mL). K₂CO₃ (265 mg, 1.92 mmol) and Cu₂O (51 mg, 0.64 mmol) were added and heated to 150° C. by microwave. Then DMF was removed under reduced pressure and water (20 mL) was added and extracted with EA (30 mL×3), dried with Na₂SO₄. The solvent was removed under reduced pressure, the residue was purified by prep-HPLC to give 3-chloro-N-(2,2,6,6-tetramethylpiperidin-4-yl)-4-phenoxybenzamide (40 mg, 18%) as a white solid. LRMS [M+H]⁺ m/z: calcd 386.18. found 386. ¹H NMR (300 MHz, CD₃OD): δ 8.44 (s, 2H), 8.00 (dd, J=2.1, 8.7 Hz, 2H), 7.75 (d, J=2.4 Hz, 2H), 7.41 (t, J=7.5 Hz, 2H), 7.20 (t, J=7.5 Hz, 2H), 6.95-7.03 (m, 3H), 4.46-4.52 (m, 1H), 2.14 (d, J=3.6, 14.1 Hz, 2H), 1.61-1.69 (m, 2H), 1.57 (s, 6H), 1.48 (s, 6H).

By a similar method as Example 3, using the appropriate starting materials, the following compounds were prepared and isolated unless where noted below.

| Compound | Structure | Data |
|---|---|---|
| I-2 | 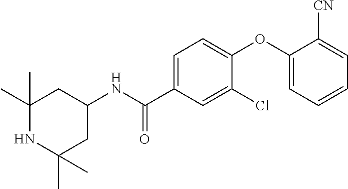 [SMILES: CC1(C)CC(NC(=O)c2ccc(Oc3ccccc3C#N)c(Cl)c2)CC(C)(C)N1] | LRMS [M + H]⁺ m/z: calcd 411.17; found 411. ¹H NMR (300 MHz, CD₃OD): δ 8.05 (d, J = 1.8 Hz, 1H), 7.79-1.86 (m, 2H), 7.63 (m, 1H), 7.31 (t, J = 7.3 Hz, 1H), 7.21 (d, J = 8.6 Hz, 1H), 6.91 (d, J = 8.5 Hz, 1H), 4.54-4.46 (m, 1H), 2.15 (dd, J = 3.3, 13.4 Hz, 2H), 1.59-1.67 (m, 2H), 1.57 (s, 6H), 1.47 (s, 6H). |
| I-10 | 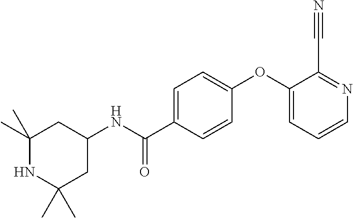 [SMILES: CC1(C)CC(NC(=O)c2ccc(Oc3cccnc3C#N)cc2)CC(C)(C)N1] | [M + H]⁺ = 379 ¹H NMR (DMSO-d₆) δ: 8.62 (d, J = 11.4 Hz, 1H), 8.55 (dd, J = 4.6, 1.1 Hz, 1H), 8.50 (d, J = 7.3 Hz, 1H), 7.93-7.98 (m, 2H), 7.80 (d, J = 12.8 Hz, 1H), 7.75 (dd, J = 8.8, 4.5 Hz, 1H), 7.60 (dd, J = 8.7, 1.1 Hz, 1H), 7.27-7.33 (m, 2H), 4.27-4.43 (m, 1H), 1.98 (dd, J = 13.3, 3.0 Hz, 2H), 1.50- 1.63 (m, 3H), 1.33-1.48 (m, 14H). |
| I-34 | 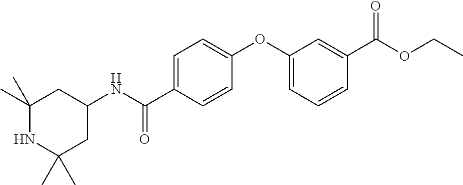 [SMILES: CCOC(=O)c1cccc(Oc2ccc(C(=O)NC3CC(C)(C)NC(C)(C)C3)cc2)c1] | [M + H]⁺ = 425 ¹H NMR (DMSO-d₆) δ: 8.56 (d, J = 11.7 Hz, 1H), 8.41 (d, J = 7.3 Hz, 2H), 7.84-7.92 (m, 2H), 7.71- 7.79 (m, 2H), 7.53-7.60 (m, 1H), 7.49 (dd, J = 2.5, 1.6 Hz, 1H), 7.36 (ddd, J = 8.1, 2.6, 1.0 Hz, 1H), 7.06-7.14 (m, 2H), 4.22-4.39 (m, 3H), 1.96 (dd, J = 13.5, 3.0 Hz, 2H), 1.53 (t, J = 12.8 Hz, 2H), 1.42 (s, 6H), 1.36 (s, 6H), 1.27 (t, J = 7.1 Hz, 3H). |
| I-43 | 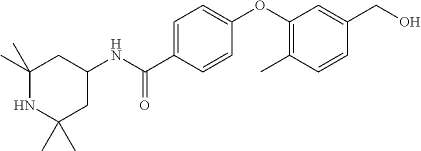 [SMILES: Cc1ccc(CO)cc1Oc1ccc(C(=O)NC2CC(C)(C)NC(C)(C)C2)cc1] | [M + H]⁺ = 397 ¹H NMR (DMSO-d₆) δ: 7.98 -8.11 (m, 1H), 7.76- 7.84 (m, 2H), 7.26 (d, J = 7.8 Hz, 1H), 7.06 (d, J = 7.6 Hz, 1H), 6.87 (d, J = 8.9 Hz, 3H), 5.15 (t, J = 5.7 Hz, 1H), 4.42 (d, J = 5.7 Hz, 2H), 4.18-4.30 (m, 1H), 2.48-2.52 (m, 2H), 2.10 (s, 3H), 1.65 (br. s., 2H), 0.97-1.23 (m, 12H). |

### Example 4

### Synthesis of 4-((3-(5-methylpyridin-3-yl)phenoxy)methyl)-N-(2,2,6,6-tetramethylpiperidin-4-yl)benzamide (I-13)

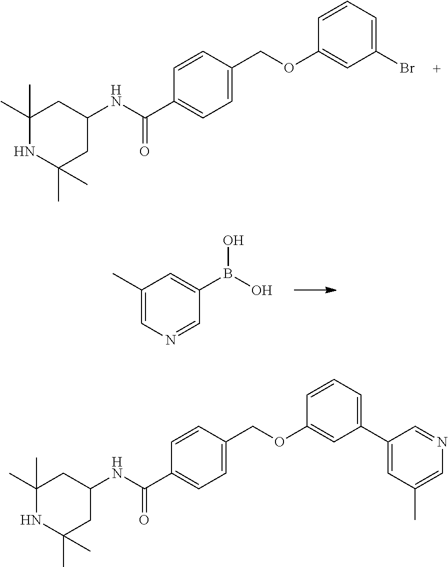

A mixture of 4-((3-bromophenoxy)methyl)-N-(2,2,6,6-tetramethylpiperidin-4-yl)benzamide (100 mg, 0.225 mmol), 5-methylpyridin-3-ylboronic acid (34 mg, 0.247 mmol), Na₂CO₃ (47.7 mg, 0.45 mmol) and Pd(PPh₃)₄ (26 mg, 0.022 mmol) in CH₃CN (4 mL) and H₂O (1 mL) was subjected to microwave heating at 130° C. for 30 min., after cooling, the mixture was concentrated. The residue was purified by column chromatography (CH₂Cl₂:MeOH=15:1) to afford 4-((3-(5-methylpyridin-3-yl)phenoxy)methyl)-N-(2,2,6,6-tetramethylpiperidin-4-yl)benzamide (40 mg, 40%) as a white solid. LRMS [M+H]⁺ m/z: calcd 457.27. found 457. ¹H NMR (300 MHz, DMSO-d₆): δ 8.69 (s, 1H), 8.42-8.46 (m, 2H), 7.88 (t, J=8.1 Hz, 3H), 7.58 (d, J=8.1 Hz, 2H), 7.29-7.44 (m, 3H), 7.06 (dd, J=1.8, 8.1 Hz, 1H), 5.28 (s, 2H), 4.29-4.40 (m, 1H), 2.37 (s, 3H), 1.98 (d, J=13.2 Hz, 2H), 1.55 (t, J=12.9 Hz, 2H), 1.44 (s, 6H), 1.38 (s, 6H).

By a similar method as Example 4, the following compounds were prepared and isolated unless where noted below.

| Compound | Structure | Data |
|---|---|---|
| I-1 | 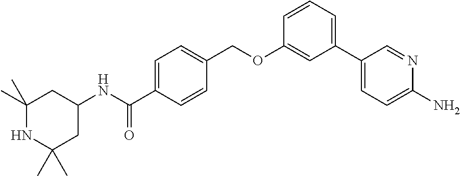 [SMILES: CC1(C)CC(NC(=O)c2ccc(COc3cccc(-c4ccc(N)nc4)c3)cc2)CC(C)(C)N1] | LRMS [M + H]⁺ m/z: calcd 458; found 458. ¹H NMR (300 MHz, CD₃OD): δ 8.40 (s, 1H), 8.10 (s, 1H), 7.84 (d, J = 8.1 Hz, 2H), 7.79 (d, J = 8.7 Hz, 1H), 7.57 (d, J = 8.1 Hz, 2H), 7.35 (t, J = 8.1 Hz, 1H), 7.15 (d, J = 9.0 Hz, 2H), 6.95 (dd, J = 2.4, 8.4Hz , 1H), 6.73 (dd, J = 4.2, 9.0 Hz, 1H), 5.22 (s, 2H), 4.52 (m, 1H), 2.13 (dd, J = 3.3, 13.8 Hz, 2H), 1.66 (t, J = 12.9 Hz, 2H), 1.57 (s, 6H), 1.47 (s, 6H). |
| I-3 | 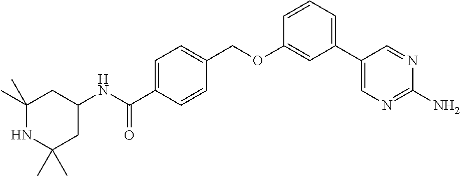 [SMILES: CC1(C)CC(NC(=O)c2ccc(COc3cccc(-c4cnc(N)nc4)c3)cc2)CC(C)(C)N1] | LRMS [M + H]⁺ m/z: calcd 459.26; found 459. ¹H NMR (300 MHz, CD₃OD): δ 1.48 (s, 6H), 1.58 (s, 6H), 1.62-1.70 (m, 2H), 2.12-2.17 (m, 2H), 4.48-4.59 (m, 1H), 7.00-7.03 (m, 1H), 7.14-7.19 (m, 2H), 7.38 (t, J = 8.1 Hz, 1H), 7.60 (d, J = 8.4 Hz, 2H), 7.87 (d, J = 8.4 Hz, 2H), 8.53 (s, 2H). |
| I-5 | 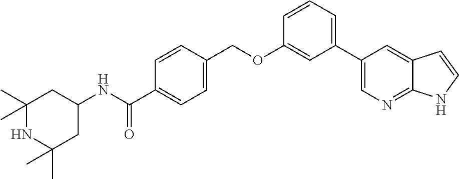 [SMILES: CC1(C)CC(NC(=O)c2ccc(COc3cccc(-c4cnc5[nH]ccc5c4)c3)cc2)CC(C)(C)N1] | [M + H]⁺ = 483; ¹H NMR (400 MHz , DMSO-d₆) δ = 11.74 (br. s., 1 H), 8.65 (d, J = 12.1 Hz, 1 H), 8.51 (d, J = 2.3 Hz, 1 H), 8.45 (d, J = 7.3 Hz, 1 H), 8.22 (d, J = 2.3 Hz, 1 H), 7.90-7.77 (m, 3 H), 7.58 (d, J = 8.5 Hz, 2 H), 7.53-7.48 (m, 1 H), 7.41- 7.36 (m, 1 H), 7.35-7.33 (m, 1 H), 7.31- 7.26 (m, 1 H), 6.99 (dd, J = 2.1, 7.8 Hz, 1 H), 6.50 (dd, J = 1.9, 3.3 Hz, 1 H), 5.28 (s, 2 H), 4.40-4.28 (m, 1 H), 1.97 (dd, J = 3.0, 13.3 Hz, 2 H), 1.56 (t, J = 12.9 Hz, 2 H), 1.44 (s, 6 H), 1.37 (s, 6H) |
| I-7 | 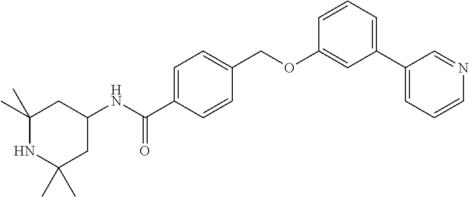 [SMILES: CC1(C)CC(NC(=O)c2ccc(COc3cccc(-c4cccnc4)c3)cc2)CC(C)(C)N1] | LRMS [M + H]⁺ m/z: calcd 443.26; found 222. ¹H NMR (300 MHz, CD₃OD): δ 1.51 (s, 6H), 1.60 (s, 6H), 1.65-1.74 (m, 2H), 2.14-2.20 (m, 2H), 4.50-4.87 (m, 1H), 7.09-7.12 (m, 1H), 7.27-7.30 (m, 2H), 7.44 (t, J = 7.8 Hz, 1H), 7.52-7.56 (m, 1H), 7.62 (d, J = 8.4 Hz, 2H), 7.88 (d, J = 8.4 Hz, 2H), 8.11-8.12 (m, 1H), 8.83-8.55 (m, 1H), 8.78-8.79 (m, 1H). |
| I-11 | 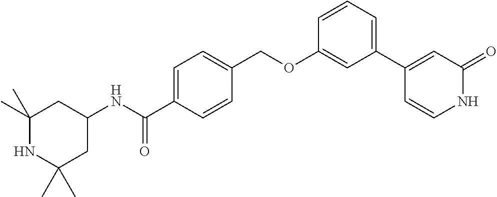 [SMILES: CC1(C)CC(NC(=O)c2ccc(COc3cccc(-c4cc[nH]c(=O)c4)c3)cc2)CC(C)(C)N1] | LRMS [M + H]⁺ m/z: calcd 459.25, found 459. ¹H NMR (300 MHz, CD₃OD): δ 7.86 (d, J = 7.8 Hz, 2H), 7.58 (d, J = 8.1 Hz, 2H), 7.49 (d, J = 7.5 Hz, 1H), 7.40 (t, J = 8.4 Hz,lH), 7.25 (d, J = 6.6 Hz, 2H), 7.11 (d, J = 6.3 Hz, 2H), 6.70 (d, J = 5.7 Hz, 2H), 5.25 (s, 2H), 4.52 (m, 1H), 2.15 (dd, J = 3.6 , 15 Hz, 2H), 1.68- 1.62 (m, 2H), 1.58 (s, 6H), 1.48 (s, 6H). |
| I-12 | 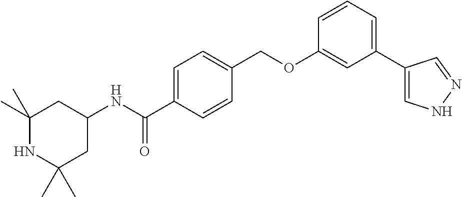 [SMILES: CC1(C)CC(NC(=O)c2ccc(COc3cccc(-c4cn[nH]c4)c3)cc2)CC(C)(C)N1] | LRMS [M + H]⁺ m/z: calcd 432.24; found 432. ¹H NMR (300 MHz, CD₃OD): δ 1.51 (s, 6H), 1.58 (s, 6H), 1.7 (t, J = 12.9 Hz, 2H,), 2.09- 2.15 (m, 2H), 4.86-4.48 (m, 1H), 5.20 (s, 2H), 6.84-6.87 (m, 1H), 7.15-7.28 (m, 3H), 7.57 (d, J = 8.1 Hz, 2H), 7.85-7.87 (m, 2H), 7.90 (s, 2H). |
| I-14 | 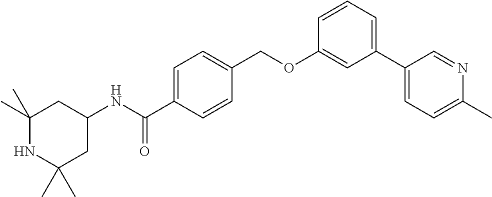 | LRMS [M+H]⁺ m/z: calcd 457.27; found 457. ¹H NMR (300 MHz, CD₃OD): δ 8.57 (d, J = 19.2 Hz, 2H), 8.54 (s, 1H), 7.95 (d, J = 5.7 Hz, 1H), 7.85(d, J = 8.4 Hz, 2H), 7.58 (d, J = 8.8 Hz, 2H), 7.39-7.37 (m, 2H), 7.24-7.23 (m, 2H), 7.04 (d, J = 6.0 Hz, 1H), 5.25 (s, 2H), 4.51 (m, 1H), 2.56 (s, 3H), 2.13 (d, J = 13.5 Hz, 2H), 1.64 (t, J = 11.7 Hz, 2H), 1.56 (s, 611), 1.46 (s, 6H). |
| I-18 | 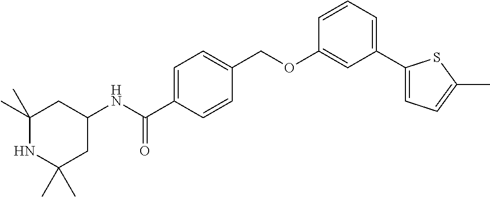 | LRMS [M + H]⁺ m/z: calcd 462.23; found 462. ¹H NMR (300 MHz, CD₃OD): δ 1.51 (s, 6H), 1.60 (s, 6H), 1.66-1.74 (m, 2H), 2.13-2.19 (m, 2H), 2.50 (s, 2H), 4.49-4.58 (m, 1H), 5.23 (s, 2H), 6.75-6.76 (m, 1H), 6.89-6.92 (m, 1H), 7.15-7.18 (m, 3H), 7.25-7.30 (m, 1H), 7.60 (d, J= 8.4 Hz, 2H), 7.88 (d, J= 8.1 Hz, 2H). |
| I-19 | 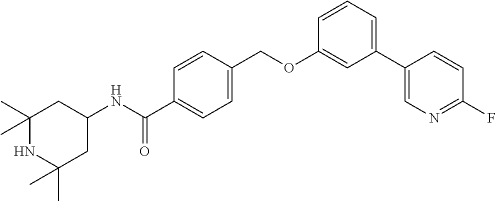 [SMILES: CC1(C)CC(NC(=O)c2ccc(COc3cccc(-c4ccc(F)nc4)c3)cc2)CC(C)(C)N1] | [M + H]⁺ = 462; ¹H NMR (400 MHz, DMSO-d₆) δ = 8.67 (d, J = 12.6 Hz, 1 H), 8.55 (d, J = 2.5 Hz, 1 H), 8.46 (d, J = 7.3 Hz, 1 H), 8.29 (td, J = 8.2, 2.7 Hz, 1 H), 7.81-7.89 (m, 3 H), 7.56 (d, J = 8.2 Hz, 2 H), 7.38-7.44 (m, 1 H), 7.37 (s, 1 H), 7.25- 7.31 (m, 2 H), 7.07 (dd, J = 8.2, 2.5 Hz, 1 H), 5.27 (s, 2 H), 4.28-4.39 (m, 1 H), 1.93-2.00 (m, 2 H), 1.56 (t, J = 12.8 Hz, 2 H), 1.44 (s, 6 H), 1.38 ppm (s, 6 H) |
| I-27 | 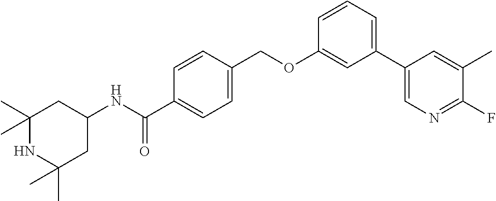 | [M + H]⁺ = 476; ¹H NMR (400 MHz, DMSO-d₆) δ = 8.65 (d, J = 11.7 Hz, 1 H), 8.46 (d, J = 7.6 Hz, 1 H), 8.34 (s, 1 H), 8.16 (dd, J = 9.6, 2.1 Hz, 1 H), 7.78- 7.89 (m, 3 H), 7.56 (d, J = 8.2 Hz, 2 H), 7.37 - 7.43 (m, 1 H), 7.35 (t, J = 1.9 Hz, 1 H), 7.28 (d, J = 7.8 Hz, 1 H), 7.05 (dd, J = 8.2, 2.5 Hz, 1 H), 5.26 (s, 2 H), 4.27-4.40 (m, 1 H), 2.31 (s, 3 H), 1.97 (dd, J = 13.5, 2.7 Hz, 2 H), 1.56 (t, J = 12.9 Hz, 2 H), 1.44 (s, 6 H), 1.38 ppm (s, 6 H) |
| I-30 | 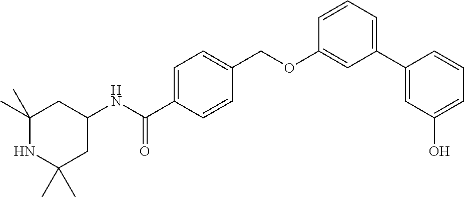 [SMILES: CC1(C)CC(NC(=O)c2ccc(COc3cccc(-c4cccc(O)c4)c3)cc2)CC(C)(C)N1] | LRMS [M + H]⁺ m/z: calcd 458.26; found 458. ¹H NMR (300 MHz, CDCl₃): δ 1.47 (s, 6H), 1.59 (s, 6H), 1.64-1.73 (m, 2H), 2.11-2.17 (m, 2H), 4.53-4.59 (m, 1H), 5.25 (s, 2H), 6.76-6.80 (m, 1H), 6.98-7.07 (m, 3H), 7.17- 7.27 (m, 4H), 7.37-7.62 (m, 2H), 7.86-7.89 (m, 2H). |
| I-32 | 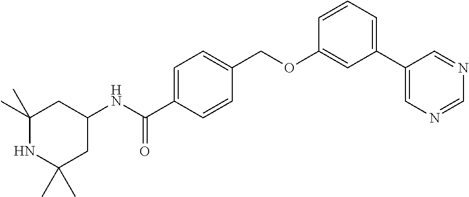 [SMILES: CC1(C)CC(NC(=O)c2ccc(COc3cccc(-c4cncnc4)c3)cc2)CC(C)(C)N1] | LRMS [M + H]⁺ m/z: calcd 444.25; found 444. ¹H NMR (300 MHz, CDCl₃): δ 1.53 (s, 6H), 1.58 (s, 6H), 1.68-1.76 (m, 2H), 2.08-2.14 (m, 2H), 4.48-4.56 (m, 1H), 5.27 (s, 2H), 7.12-7.15 (m, 1H), 7.31-7.65 (m, 2H), 7.44- 7.49 (m, 1H), 7.58-7.61 (m, 2H), 7.86-7.89 (m, 2H), 9.05 (s, 2H), 9.13 (s, 1H). |
| I-33 | 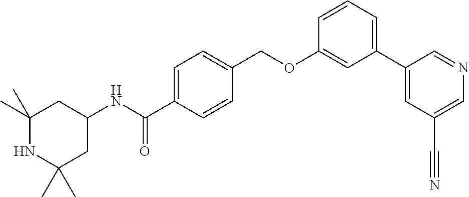 [SMILES: CC1(C)CC(NC(=O)c2ccc(COc3cccc(-c4cncc(C#N)c4)c3)cc2)CC(C)(C)N1] | LRMS [M + H]⁺ m/z: calcd 468.25; found 468. ¹H NMR (300 MHz, CD₃OD): δ 1.50 (s, 6H), 1.58 (s, 6H), 1.68-1.77 (m, 2H), 2.12-2.20 (m, 2H), 4.49-4.59 (m, 1H), 5.29 (s, 2H), 7.13-7.17 (m, 1H), 7.31-7.36 (m, 2H), 7.45- 7.50 (m, 1H), 7.62 (d, J = 8.1 Hz, 2H), 7.88 (d, J = 8.1 Hz, 2H), 8.89-8.90 (m, 1H), 9.07 (s, 1H), 9.08 (m, 1H). |
| I-37 | 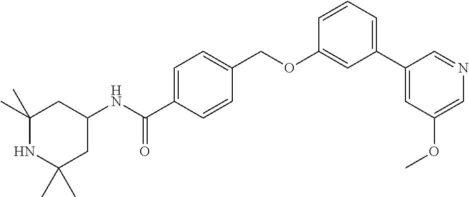 [SMILES: COc1cncc(-c2cccc(OCc3ccc(C(=O)NC4CC(C)(C)NC(C)(C)C4)cc3)c2)c1] | LRMS [M + H]⁺ m/z: calcd 473.27; found 473. ¹H NMR (300 MHz, CD₃OD): δ 1.37 (s, 6H), 1.42 (s, 6H), 1.55-1.63 (m, 2H), 1.87-1.89 (m, 2H), 3.89 (s, 3H), 4.31-4.34 (m, 1H) 5.27 (s, 2H), 7.05-7.08 (m, 1H), 7.30-7.35 (m, 3H), 7.39-7.44 (m, 3H), 7.57-7.59 (m, 3H), 7.85 (d, J = 8.1 Hz, 2H), 8.26 (s, 1H), 8.45 (s, 1H). |
| I-38 | 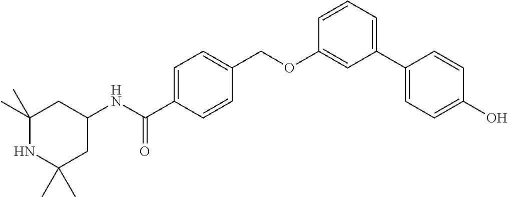 [SMILES: CC1(C)CC(NC(=O)c2ccc(COc3cccc(-c4ccc(O)cc4)c3)cc2)CC(C)(C)N1] | LRMS [M + H]⁺ m/z: calcd 458.26; found 458. ¹H NMR (300 MHz, CDCl₃): δ 1.52 (s, 6H), 1.59 (s, 6H), 1.70-1.79 (m, 2H), 2.10-2.15 (m, 2H), 4.49-4.60 (m, 1H), 5.23 (s, 2H), 6.84- 6.87 (m, 3H), 7.13-7.16 (m, 2H), 7.28-7.30 (m, 1H), 7.42-7.45 (m, 2H), 7.58-7.61 (m, 2H), 7.91-7.93 (m, 2H). |
| I-41 | 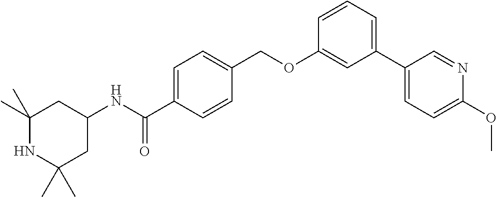 | LRMS [M + H]⁺ m/z: calcd 473.27; found 473. ¹H NMR (300 MHz, CD₃OD): δ 1.26 (s, 6H), 1.37 (s, 8H), 1.91-1.97 (m, 2H), 3.93 (s, 3H), 4.41-4.49 (m, 1H), 5.20 (s, 2H), 6.84 (d, J = 8.7 Hz, 1H), 6.96-6.99 (m, 1H), 7.13-7.17 (m, 2H), 7.31-7.37 (m, 1H), 7.54-7.57 (m, 2H), 7.82-7.90 (m, 3H), 8.31-8.32 (m, 1H). |
| I-47 | 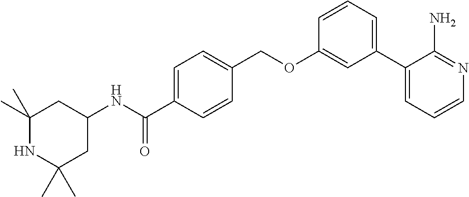 [SMILES: CC1(C)CC(NC(=O)c2ccc(COc3cccc(-c4cccnc4N)c3)cc2)CC(C)(C)N1] | LRMS [M + H]⁺ m/z: calcd 458.27; found 458. ¹H NMR (300 MHz, CD₃OD): δ 7.92 (d, J = 5.1 Hz, 1H), 7.85 (d, J = 8.1 Hz, 2H), 7.57 (d, J = 5.1 Hz, 2H), 7.49 (d, J = 8.7 Hz, 1H), 7.41 (t, J = 9.0 Hz, 1H), 7.05 (t, J = 7.8 Hz, 3H), 5.22 (s, 2H), 4.52 (m, 1H), 2.14 (dd, J = 5.1, 12.9 Hz, 2H), 1.69 (t, J = 12.9 Hz, 2H), 1.58 (s, 6H), 1.49 (s, 6H). |
| I-51 | 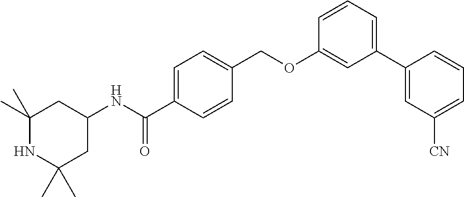 [SMILES: CC1(C)CC(NC(=O)c2ccc(COc3cccc(-c4cccc(C#N)c4)c3)cc2)CC(C)(C)N1] | LRMS [M + H]⁺ m/z: calcd 467.26; found 467. ¹H NMR (300 MHz, CDCl₃): δ 1.49-1.57 (m, 12H), 1.66-1.74 (m, 2H), 2.09-2.15 (m, 2H), 4.51-4.53 (m, 1H), 5.25 (s, 2H), 7.04-7.06 (m, 1H), 7.22-7.26 (m, 2H), 7.36-7.42 (m, 1H), 7.57-7.71 (m, 4H), 7.84-7.94 (m, 4H). |
| I-55 | 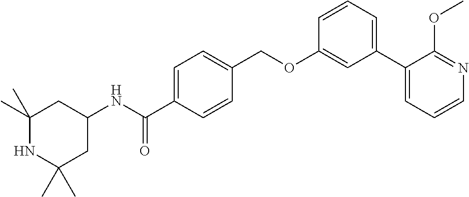 | LRMS [M + H]⁺ m/z: calcd 473.27; found 473. ¹H NMR (300 MHz, CD₃OD): δ 1.50 (s, 6H), 1.58 (s, 6H), 1.68-1.77 (m, 2H), 2.12-2.20 (m, 2H), 3.93 (s, 3H), 4.50-4.58 (m, 1H), 5.23 (s, 2H), 6.99-7.07 (m, 2H), 7.12-7.15 (m, 1H), 7.32-7.37 (m, 1H), 7.60 (d, J = 8.4 Hz, 2H), 7.70 (dd, J = 1.8, 7.2 Hz, 1H), 7.86- 7.89 (m, 2H), 8.12-8.14 (m, 1H). |
| I-58 | 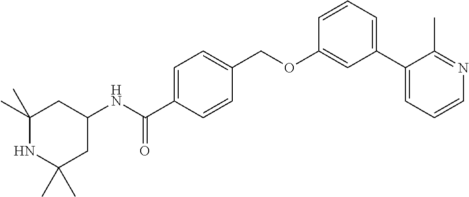 [SMILES: Cc1ncccc1-c1cccc(OCc2ccc(C(=O)NC3CC(C)(C)NC(C)(C)C3)cc2)c1] | LRMS [M + H]⁺ m/z: calcd 457.27; found 457. ¹H NMR (300 MHz, CD₃OD): δ 8.54 (s, 1H), 8.39 (dd, J = 1.8, 5.1Hz, 1H), 7.85 (d, J = 14.4 Hz, 2H), 7.64 (dd, J = 1.5, 7.5Hz, 1H), 7.57 (d, J = 8.1 Hz, 2H), 7.41-7.30 (m, 2H), 7.06 (dd, J = 2.4 , 9.0 Hz, 2H), 6.96-6.92 (m, 1H), 5.22 (s, 2H), 4.51 (m, 1H), 2.41 (s, 3H), 2.13 (d, J = 14.1 Hz, 2H), 1.65 (t, J = 12.6 Hz, 2H), 1.57 (s, 6H), 1.47 (s, 6H). |
| I-65 | 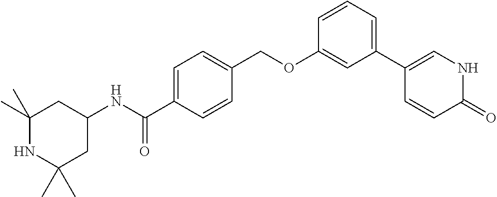 [SMILES: CC1(C)CC(NC(=O)c2ccc(COc3cccc(-c4ccc(=O)[nH]c4)c3)cc2)CC(C)(C)N1] | LRMS [M + H]⁺ m/z: calcd 459.25; found 459. ¹H NMR (300 MHz, CD₃OD): δ 1.50 (s, 6H), 1.58 (s, 6H), 1.68-1.77 (m, 2H), 2.09-2.15 (m, 2H), 4.48-4.58 (m, 1H), 6.63 (d, J = 9.6 Hz, 1H), 6.96-6.99 (m, 1H), 7.09-7.15 (m, 2H), 7.34 (t, J = 8.1 Hz, 1H),7.58 (d, J = 8.4 Hz, 2H), 7.67-7.68 (m, 1H), 7.87 (d, J = 8.4 Hz, 2H), 7.92 (dd, J = 2.7, 9.3 Hz, 1H). |
| I-129 | 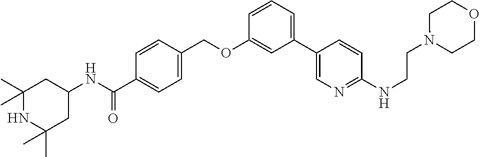 [SMILES: CC1(C)CC(NC(=O)c2ccc(COc3cccc(-c4ccc(NCCN5CCOCC5)nc4)c3)cc2)CC(C)(C)N1] | [M + H]⁺ = 572; ¹H NMR (400 MHz, DMSO-d₆) δ = 8.72 (d, J = 11.4 Hz, 1 H), 8.47 (d, J = 7.6 Hz, 1 H), 8.37 (d, J = 2.3 Hz, 1 H), 7.94-7.83 (m, 4 H), 7.55 (d, J = 8.2 Hz, 2 H), 7.50-7.42 (m, 1 H), 7.38-7.32 (m, 1 H), 7.24 (d, J = 1.8 Hz, 1 H), 7.20 (d, J = 7.8 Hz, 1 H), 6.97 (dd, J = 2.2, 8.1 Hz, 1 H), 6.76 (d, J = 8.7 Hz, 1 H), 5.24 (s, 2 H), 4.41-4.26 (m, 1 H), 3.85 (br. s., 4 H), 3.69 (br. s., 2 H), 1.96 (dd, J = 2.9, 13.2 Hz, 2 H), 1.57 (t, J = 12.9 Hz, 2 H), 1.44 (s, 6 H), 1.38 (s, 6H) |

### Example 5

### Synthesis of 4-((6-methylpyridin-2-yloxy)methyl)-N-(2,2,6,6-tetramethylpiperidin-4-yl)benzamide (I-85)

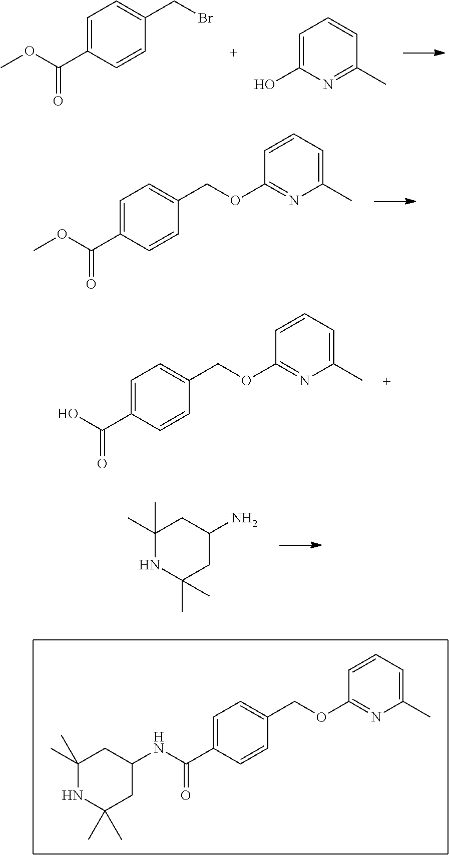

### Synthesis of methyl 4-((6-methylpyridin-2-yloxy)methyl)benzoate

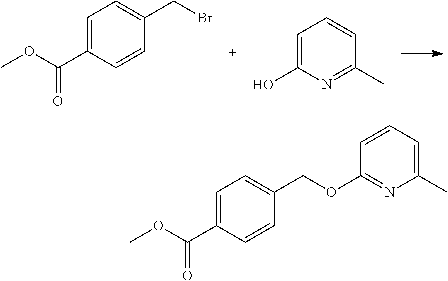

Methyl 4-(bromomethyl)benzoate (500 mg, 2.18 mmol), 6-methylpyridin-2-ol (262 mg, 2.40 mmol), Ag₂CO₃ (600 mg, 2.18 mmol) and n-hexane (6 mL) were treated in a 10 mL microwave tube. Then the mixture was reacted at 150° C. for 10 min. The mixture was filtrated and the filtrate was purified by prep-TLC to give methyl 4-((6-methylpyridin-2-yloxy)methyl)benzoate (350 mg, 62.5%) as a white solid.

### Synthesis of 4-((6-methylpyridin-2-yloxy)methyl)benzoic acid

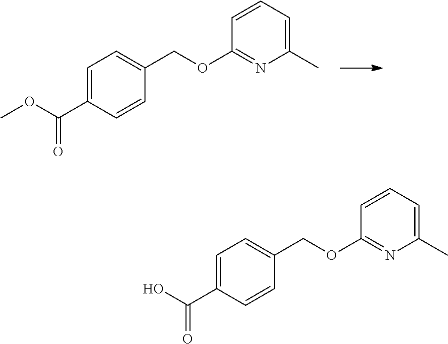

To a solution of methyl 4-((6-methylpyridin-2-yloxy)methyl)benzoate (47-26-b) (350 mg, 1.36 mmol) in THF (12 mL), water (4 mL) and MeOH (4 mL) was added LiOH (172 mg, 4.09 mmol). The reaction solution was stirred at 60° C. for 2 h. The solvent was evaporated. To the residue, water was added and the pH value of the resulting solution was adjusted to 1˜2 by addition of dilute HCl (1N). 4-((6-methylpyridin-2-yloxy)methyl)benzoic acid (120 mg, 36.4%) was obtained as white solid by filtration and further washed with water.

### Synthesis of 4-((6-methylpyridin-2-yloxy)methyl)-N-(2,2,6,6-tetramethylpiperidin-4-yl)benzamide (I-85)

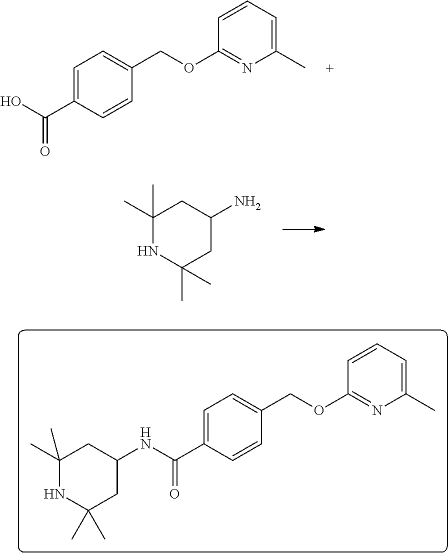

To a solution of 4-((6-methylpyridin-2-yloxy)methyl)benzoic acid (120 mg, 0.49 mmol) in DCM (10 mL) was added HOSu (68 mg, 0.59 mmol), EDCI.HCl (113 mg, 0.59 mmol) and Na₂CO₃ (114 mg, 1.08 mmol). Then the mixture was stirred at rt for overnight. 2,2,6,6-tetramethylpiperidin-4-amine (154 mg, 0.99 mmol) was added to the reaction mixture. After 1 h, the solvent was removed and the residue was purified by prep-TLC. 4-((6-methylpyridin-2-yloxy)methyl)-N-(2,2,6,6-tetramethylpiperidin-4-yl)benzamide (150 mg, 80.2%) was obtained as a white solid. LRMS [M+H]⁺ m/z: calcd 381.24. found 381. ¹H NMR (300 MHz, CD₃OD): δ 7.84 (d, J=8.1 Hz, 2H), 7.54-7.60 (m, 3H), 6.83 (d, J=7.5 Hz, 1H), 6.67 (d, J=8.1 Hz, 1H), 5.42 (s, 2H), 4.44-4.52 (m, 1H), 2.43 (s, 3H), 1.96-2.01 (m, 2H), 1.37-1.46 (m, 8H), 1.31 (s, 6H).

By a similar method to Example 5, using the appropriate starting materials, the following compounds were prepared and isolated unless where noted below.

| Compound | Structure | Data |
|---|---|---|
| I-70 | 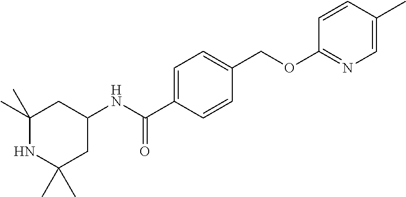 | LRMS [M + H]⁺ m/z: calcd 381.24; found 381. ¹H NMR (300 MHz, CD₃OD): δ 7.93- 7.92 (m, 1H), 7.85-7.82 (m, 2H), 7.54- 7.51 (m, 3H), 6.80 (t, J = 8.4 Hz, 1H,), 5.37 ( s, 2H), 4.59-4.48 (m, 1H), 2.25 (s, 3H), 2.13-2.08 (m, 2H), 1.79-1.70 (m, 2H), 1.59 (s, 6H), 1.52 (s, 6H). |
| I-80 | 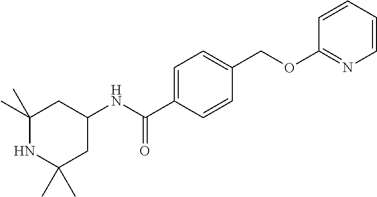 [SMILES: CC1(C)CC(NC(=O)c2ccc(COc3ccccn3)cc2)CC(C)(C)N1] | LRMS [M + H]⁺ m/z: calcd 367.23; found 367. ¹H NMR (300 MHz, CD₃OD): δ 8.12- 8.10 (m, 1H), 7.83 (d, J = 8.1 Hz, 2H), 7.66-7.69 ( m, 1H), 7.53 (t, J = 8.1 Hz, 2H), 6.97-6.86 (m, 2H), 5.41 (s, 2H), 4.56-4.48 (m, 1H), 2.14-2.10 ( m, 2H), 1.68-1.76 ( m, 2H), 1.58 ( s, 6H), 1.50 (s, 6H). |

### Example 6

### 4-(((6-(methylamino)pyridin-2-yl)oxy)methyl)-N-(2,2,6,6-tetramethylpiperidin-4-yl)benzamide (I-121)

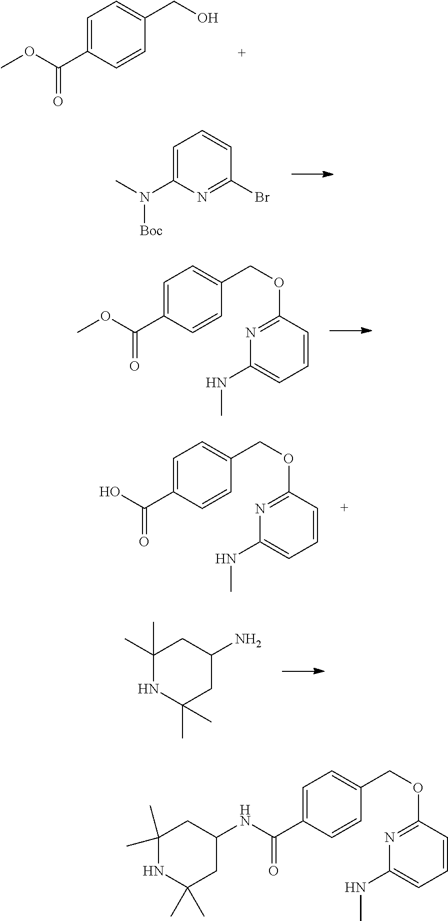

### Synthesis of methyl 4-(6-(methylamino)pyridin-2-yloxy)benzoate

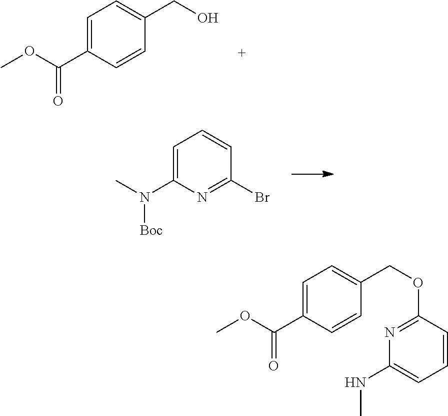 [SMILES: COC(=O)c1ccc(CO)cc1 ; CN(C(=O)OC(C)(C)C)c1cccc(Br)n1 ; CNc1cccc(OCc2ccc(C(=O)OC)cc2)n1]

To a solution of methyl 4-(hydroxymethyl)benzoate (200 mg, 1.32 mmol) and tert-butyl 6-bromopyridin-2-yl(methyl)carbamate (377 mg, 1.32 mmol) in DMF (10 mL) was added K₂CO₃ (200 mg, 1.447 mmol). The mixture was stirred at 150° C. overnight. The solvent was removed and the residue was dissolved in DCM (30 mL) and washed with water (10 mL) and brine (10 mL), The organic solvent was removed under reduced pressure and the residue was purified by column chromatography to give methyl 4-(6-(methylamino)pyridin-2-yloxy)benzoate (0.25 g, 73.3%) as white solid. LRMS [M+H]⁺ m/z: calcd 272.12. found 272.

### Synthesis of 4-(((6-(methylamino)pyridin-2-yl)oxy)methyl)benzoic acid

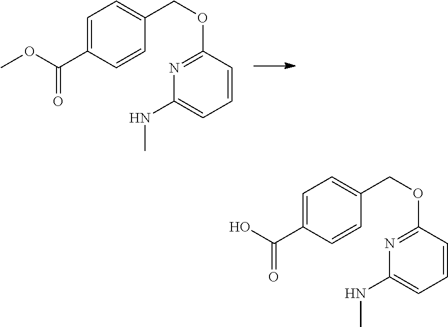 [SMILES: CNc1cccc(OCc2ccc(C(=O)OC)cc2)n1 ; CNc1cccc(OCc2ccc(C(=O)O)cc2)n1]

To a solution of methyl 4-(pyridin-2-yloxy)benzoate (250 mg, 0.97 mmol) in THF (12 mL), water (4 mL) and MeOH (4 mL) was added LiOH (110 mg, 2.90 mmol). The reaction solution was stirred at 60° C. for 2 h. The solvent was evaporated. To the residue, water was added and the pH value of the resulting solution was adjusted to 1˜2 by addition of dilute HCl (1N). 4-(((6-(methylamino)pyridin-2-yl)oxy)methyl)benzoic acid (200 mg, 76%) was obtained as white solid by filtration and further washed with water.

### 4-(((6-(methylamino)pyridin-2-yl)oxy)methyl)-N-(2,2,6,6-tetramethylpiperidin-4-yl)benzamide (I-121)

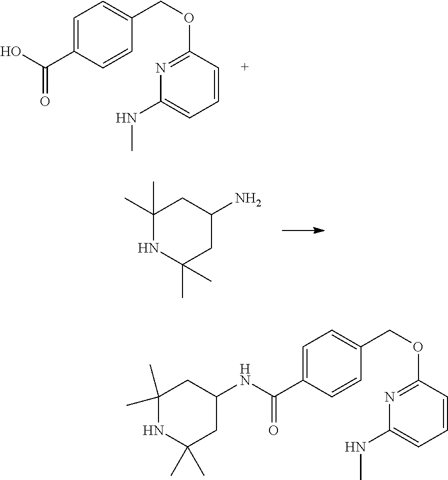

To a solution of 4-(pyridin-2-yloxy)benzoic acid (200 mg, 0.82 mmol) in DCM (10 mL) was added HOSu (128 mg, 1.12 mmol), EDCI.HCl (215 mg, 1.12 mmol) and Na₂CO₃ (217 mg, 2.05 mmol). Then the mixture was stirred at rt overnight. 2,2,6,6-tetramethylpiperidin-4-amine (290 mg, 1.86 mmol) was added to the reaction mixture. After 1 h, the solvent was removed and the residue was purified by prep-TLC. 4-(((6-(methylamino)pyridin-2-yl)oxy)methyl)-N-(2,2,6,6-tetramethylpiperidin-4-yl)benzamide (150 mg, 39%) was obtained as a white solid. LRMS [M+H]⁺ m/z: calcd 396.25. found 396. ¹H NMR (300 MHz, CD₃OD): δ 7.81 (d, J=8.4 Hz, 2H), 7.40-7.31 (m, 3H), 5.81-5.74 (m, 2H), 4.75 (s, 2H), 4.55-4.46 (m, 1H), 3.06 (s, 3H), 2.15-2.09 (m, 2H), 1.69 (m, J=12.9 Hz, 2H), 1.57 (s, 6H), 1.49 (s, 6H).

By a similar method to Example 6, using the appropriate starting materials, the following compounds were prepared and isolated unless where noted below.

| Compound | Structure | Data |
|---|---|---|
| I-56 | 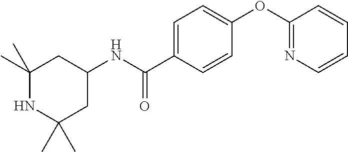 [SMILES: CC1(C)CC(NC(=O)c2ccc(Oc3ccccn3)cc2)CC(C)(C)N1] | LRMS [M + H]⁺ m/z: calcd 353.21; found 353. ¹H NMR (300 MHz, CD3OD): δ 8.18- 8.21 (m, 1H), 7.90-7.94 (m, 3H), 7.18- 7.22 (m, 3H), 7.06 (d, J = 7.5 Hz, 1H), 4.49-4.58 (m, 1H), 2.12-2.17 (m, 2H), 1.56-1.63 (m, 2H), 1.57 (s, 6H), 1.47 (s, 6H). |

### Example 7

### Synthesis of 4-(1-phenoxyethyl)-N-(2,2,6,6-tetramethylpiperidin-4-yl)benzamide (I-118)

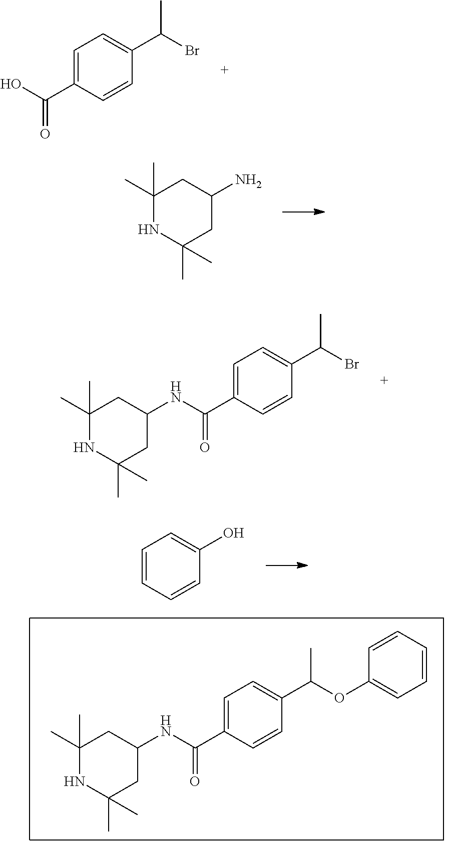 [SMILES: CC(Br)c1ccc(C(=O)O)cc1 ; CC1(C)CC(N)CC(C)(C)N1 ; CC(Br)c1ccc(C(=O)NC2CC(C)(C)NC(C)(C)C2)cc1 ; Oc1ccccc1 ; CC(Oc1ccccc1)c1ccc(C(=O)NC2CC(C)(C)NC(C)(C)C2)cc1]

### Synthesis of 4-(1-bromoethyl)-N-(2,2,6,6-tetramethylpiperidin-4-yl)benzamide

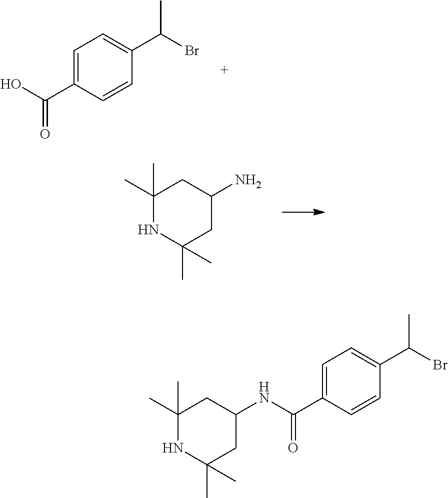 [SMILES: CC(Br)c1ccc(C(=O)O)cc1 ; CC1(C)CC(N)CC(C)(C)N1 ; CC(Br)c1ccc(C(=O)NC2CC(C)(C)NC(C)(C)C2)cc1]

A mixture of 4-(1-bromoethyl)benzoic acid (400 mg, 1.75 mmol), HOSU (210 mg, 1.83 mmol), EDCI (352 mg, 1.83 mmol), Na₂CO₃ (555 mg, 5.24 mmol) in DCM (10 mL) was stirred at room temperature for 15 hours. Then 2,2,6,6-tetramethylpiperidin-4-amine (273 mg, 1.747 mmol) was added and the mixture was stirred further for 2 hours. The mixture was purified by column chromatography (DCM: MeOH=40:1) to give 4-(1-bromoethyl)-N-(2,2,6,6-tetramethylpiperidin-4-yl)benzamide as a white solid (100 mg, 23%).

### Synthesis of 4-(1-phenoxyethyl)-N-(2,2,6,6-tetramethylpiperidin-4-yl)-benzamide (I-118)

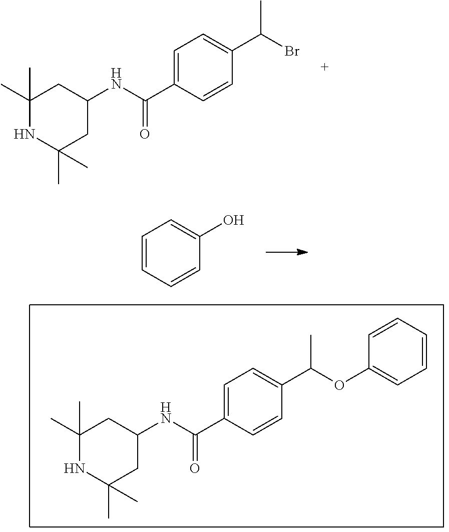 [SMILES: CC(Br)c1ccc(C(=O)NC2CC(C)(C)NC(C)(C)C2)cc1 ; Oc1ccccc1 ; CC(Oc1ccccc1)c1ccc(C(=O)NC2CC(C)(C)NC(C)(C)C2)cc1]

A suspension of 4-(1-bromoethyl)-N-(2,2,6,6-tetramethylpiperidin-4-yl)-benzamide (50 mg, 0.14 mmol), phenol (25.7 mg, 0.274 mmol), K₂CO₃ (56.7 mg, 0.41 mmol) in acetonitrile (10 mL) was stirred at 60° C. for 3 hours. The mixture was concentrated and purified by prep-TLC (DCM: MeOH=20:1) to give the 4-(1-phenoxyethyl)-N-(2,2,6,6-tetramethylpiperidin-4-yl)-benzamide as a white solid (20 mg, 38%). LRMS [M+H]⁺ m/z: calcd 380.25. found 380. ¹H NMR (300 MHz, CD₃OD): δ 7.78 (d, J=8.4 Hz, 2H), 7.50 (d, J=8.4 Hz, 2H), 7.18-7.13 (m, 2H), 6.86-6.81 (m, 3H), 5.46 (q, J=6.6 Hz, 1H), 4.55-4.44 (m, 1H), 2.15-2.09 (m, 2H), 1.68-1.60 (m, 2H), 1.56 (s, 6H), 1.48 (s, 6H).

### Example 8

### Synthesis of 2-(4-(2,2,6,6-tetramethylpiperidin-4-ylcarbamoyl)phenoxy)-5-methoxy benzoic acid (I-120)

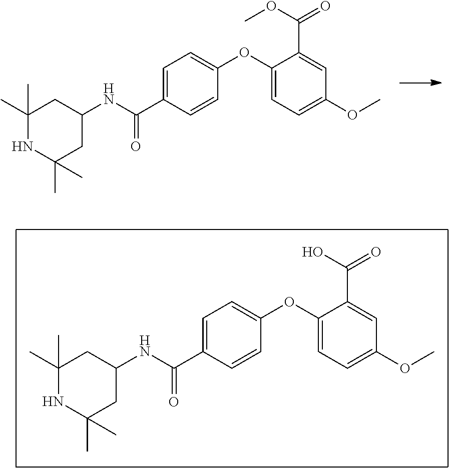

The crude product from the previous step was dissolved in 20 mL of LiOH (1M) solution and MeOH (20 mL). The resulting mixture was stirred at rt. for 1 hour. Then MeOH was removed under reduced pressure and the water phase was washed with DCM (10 mL) and the pH value was adjusted to 6. 2-(4-(2,2,6,6-Tetramethylpiperidin-4-ylcarbamoyl)phenoxy)-5-methoxy benzoic acid was obtained (20 mg, 3.2%) by prep-HPLC. LRMS [M+H]⁺ m/z: calcd 426.51. found 426. ¹H NMR (600 MHz, F₃CCOOD): δ 8.28-8.26 (m, 3H), 7.79-7.77 (s, 1H), 7.53 (m, J=8.4 Hz, 3H), 7.11 (d, J=8.4 Hz, 1H), 5.19 (d, J=5.7 Hz, 1H), 4.45 (d, 3H), 2.76 (d, J=6.6 Hz, 2H), 2.70 (s, 1H), 2.16 (s, 6H), 2.04 (s, 6H).

### Example 9

### 4-Phenoxy-N-(2,2,6,6-tetramethylpiperidin-4-yl)benzamide (I-53)

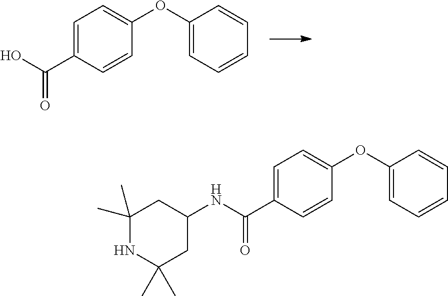 [SMILES: O=C(O)c1ccc(Oc2ccccc2)cc1 ; CC1(C)CC(NC(=O)c2ccc(Oc3ccccc3)cc2)CC(C)(C)N1]

4-Phenoxybenzoic acid (75 mg, 0.35 mmol) was dissolved in dry DMF (1 mL) and the solution was cooled to 0° C. 2,2,6,6-Tetramethylpiperidin-4-amine (64 μL, 0.37 mmol), HATU (160 mg, 0.420 mmol), and N-ethyl-N-isopropylpropan-2-amine (152 μL, 0.875 mmol) were added and the reaction mixture was stirred at r.t. until complete disappearance of the starting material. The mixture was filtered and purified by preparative HPLC to afford 4-phenoxy-N-(2,2,6,6-tetramethylpiperidin-4-yl)benzamide 2,2,2-trifluoroacetate (112 mg, 69%) as a white solid. LRMS [M+H]⁺ m/z: calcd 352. found 353. ¹H NMR (400 MHz, DMSO-d₆) δ=8.60 (d, J=12.1 Hz, 1H), 8.38 (d, J=7.3 Hz, 1H), 7.84 (d, J=8.7 Hz, 2H), 7.77 (d, J=12.1 Hz, 1H), 7.41 (t, J=7.9 Hz, 2H), 7.22-7.14 (m, 1H), 7.03 (dd, J=8.5, 11.7 Hz, 4H), 4.37-4.24 (m, 1H), 1.94 (d, J=11.4 Hz, 2H), 1.53 (t, J=12.9 Hz, 2H), 1.41 (s, 6H), 1.35 (s, 6H).

By a similar method to Example 9, using the appropriate starting materials, the following compounds were prepared and isolated unless where noted below.

| Compound | Structure | Data |
|---|---|---|
| I-25 | 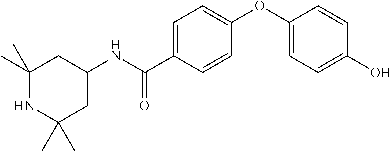 [SMILES: CC1(C)CC(NC(=O)c2ccc(Oc3ccc(O)cc3)cc2)CC(C)(C)N1] | [[M + H]⁺ = 369 ¹H NMR (DMSO-d₆) δ: 9.39-9.50 (m, 1H), 8.65 (d, J = 12.1 Hz, 1H), 8.32 (d, J = 7.3 Hz, 1H), 7.75-7.86 (m, 3H), 6.85- 6.95 (m, 4H), 6.73-6.82 (m, 2H), 4.22- 4.37 (m, 1H), 1.93 (dd, J = 13.3, 3.0 Hz, 2H), 1.53 (t, J = 12.9 Hz, 2H), 1.42 (s, 6H), 1.36 (s, 6H). |
| I-61 | 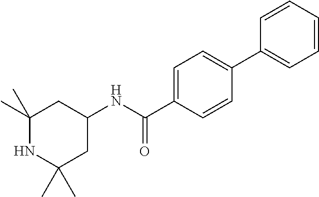 [SMILES: CC1(C)CC(NC(=O)c2ccc(-c3ccccc3)cc2)CC(C)(C)N1] | [M + H]⁺ = 337 ¹H NMR (DMSO-d₆) δ: 8.65 (d, J = 11.4 Hz, 1H), 8.49 (d, J = 7.3 Hz, 1H), 7.89 (s, 1H), 7.76-7.85 (m, 2H), 7.59 (d, J = 7.8 Hz, 1H), 7.45-7.51 (m, 1H), 7.20-7.25 (m, 2H), 7.13 (ddd, J = 8.0, 1.7, 0.7 Hz, 1H), 7.02 (ddd, J = 8.3, 2.5, 0.9 Hz, 1H), 4.26-4.40 (m, 1H), 1.96 (dd, J = 13.2, 2.9 Hz, 2H), 1.55 (t, J = 13.0 Hz, 2H), 1.43 (s, 6H), 1.36 (s, 6H). |
| I-72 | 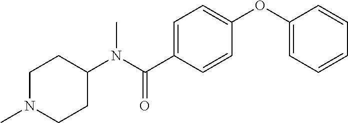 [SMILES: CN1CCC(N(C)C(=O)c2ccc(Oc3ccccc3)cc2)CC1] | [M + H]⁺ = 325 ¹H NMR (400 MHz DMSO-d₆) δ = 9.26- 9.15 (m, 1 H), 7.46-7.36 (m, 4 H), 7.23- 7.14 (m, 1 H), 7.10-7.03 (m, 2 H), 6.99 (d, J = 8.7 Hz, 2 H), 6.56-6.49 (m, 1 H), 4.52-4.35 (m, 1 H), 3.50 - 3.39 (m, 2 H), 3.13-2.95 (m, 2 H), 2.78 (s, 3 H), 2.75- 2.70 (m, 3 H), 2.03-1.90 (m, 2 H), 1.89- 1.81 (m, 2H) |
| I-73 | 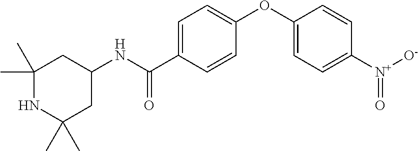 [SMILES: CC1(C)CC(NC(=O)c2ccc(Oc3ccc([N+](=O)[O-])cc3)cc2)CC(C)(C)N1] | [M + H]⁺ = 398 |
| I-75 | 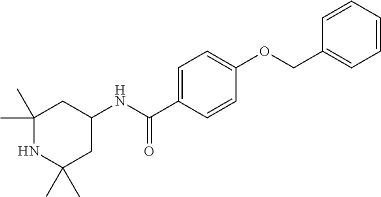 [SMILES: CC1(C)CC(NC(=O)c2ccc(OCc3ccccc3)cc2)CC(C)(C)N1] | [M + H]⁺ = 367 |
| I-86 | 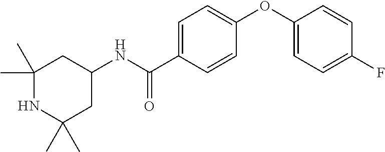 [SMILES: CC1(C)CC(NC(=O)c2ccc(Oc3ccc(F)cc3)cc2)CC(C)(C)N1] | [M + H]⁺ = 371 ¹H NMR (400 MHz DMSO-d₆) δ = 8.56 (d, J = 12.1 Hz, 1 H), 8.36 (d, J = 7.6 Hz, 1 H), 7.83 (d, J = 8.7 Hz, 2 H), 7.74 (d, J = 11.9 Hz, 1 H), 7.29-7.21 (m, 2 H), 7.14- 7.07 (m, 2 H), 7.00 (d, J = 8.7 Hz, 2 H), 4.36-4.23 (m, 1 H), 1.94 (d, J = 10.8 Hz, 2 H), 1.51 (t, J = 12.8 Hz, 2 H), 1.41 (s, 6 H), 1.35 (s, 6H). |
| I-91 | 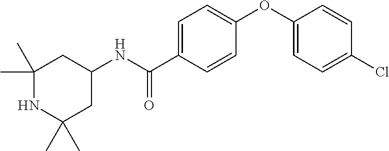 [SMILES: CC1(C)CC(NC(=O)c2ccc(Oc3ccc(Cl)cc3)cc2)CC(C)(C)N1] | [M + H]⁺ = 387 ¹H NMR (400 MHz DMSO-d₆) δ = 8.65 (d, J = 12.4 Hz, 1 H), 8.40 (d, J = 7.3 Hz, 1 H), 7.88-7.78 (m, 3 H), 7.48-7.42 (m, 2 H), 7.10-7.01 (m, 4 H), 4.37-4.24 (m, 1 H), 1.93 (dd, J = 3.0, 13.3 Hz, 2 H), 1.53 (t, J = 12.9 Hz, 2 H), 1.41 (s, 6 H), 1.35 (s, 6H). |
| I-92 | 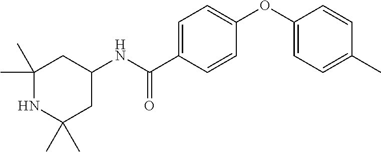 | [M + H]⁺ = 367 ¹H NMR (400 MHz DMSO-d6) δ = 8.58 (d, J = 12.4 Hz, 1 H), 8.35 (d, J = 7.3 Hz, 1 H), 7.85-7.80 (m, 2 H), 7.76 (d, J = 13.0 Hz, 1 H), 7.21 (d, J = 8.5 Hz, 2 H), 7.00-6.91 (m, 4 H), 4.29 (dd, J = 4.0, 7.9 Hz, 1 H), 2.28 (s, 3 H), 1.93 (dd, J = 2.7, 13.7 Hz, 2 H), 1.52 (t, J = 12.9 Hz, 2 H), 1.41 (s, 6H), 1.35 (s, 6H). |
| I-93 | 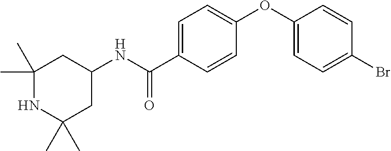 [SMILES: CC1(C)CC(NC(=O)c2ccc(Oc3ccc(Br)cc3)cc2)CC(C)(C)N1] | [M + H]⁺ = 431.1/433.1 |
| I-94 | 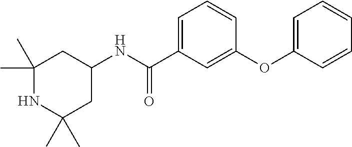 [SMILES: CC1(C)CC(NC(=O)c2cccc(Oc3ccccc3)c2)CC(C)(C)N1] | [M + H]⁺ = 353.2 |
| I-95 | 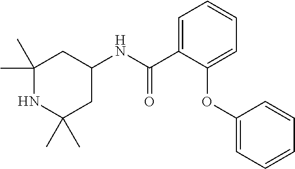 [SMILES: CC1(C)CC(NC(=O)c2ccccc2Oc2ccccc2)CC(C)(C)N1] | [M + H]⁺ = 353.2 |
| I-96 | 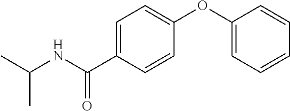 [SMILES: CC(C)NC(=O)c1ccc(Oc2ccccc2)cc1] | [M + H]⁺ = 256 ¹H NMR (DMSO-d₆) δ: 8.13 (d, J = 8.0 Hz, 1H), 7.81-7.87 (m, 2H), 7.37-7.43 (m, 2H), 7.14-7.20 (m, 1H), 7.02-7.06 (m, 1H), 6.96-7.01 (m, 2H), 3.99-4.11 (m, 1H), 1.12 (d, J = 6.6 Hz, 6H). |
| I-97 | 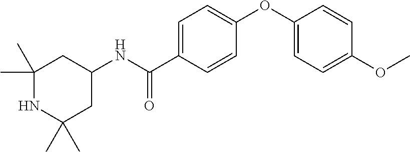 | [M + H]⁺ = 383 |
| I-98 | 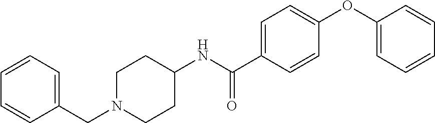 [SMILES: O=C(NC1CCN(Cc2ccccc2)CC1)c1ccc(Oc2ccccc2)cc1] | [M + H]⁺ = 387 |
| I-99 | 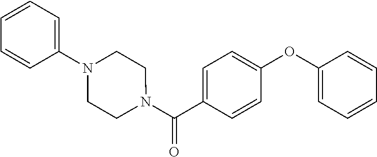 [SMILES: O=C(c1ccc(Oc2ccccc2)cc1)N1CCN(c2ccccc2)CC1] | [M + H]⁺ = 359 |
| I-100 | 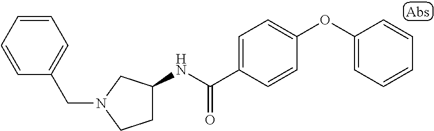 [SMILES: O=C(N[C@H]1CCN(Cc2ccccc2)C1)c1ccc(Oc2ccccc2)cc1] | [M + H]⁺ = 373; ¹H NMR (400 MHz DMSO-d₆) δ = 10.38 (br. s., 1 H), 8.74-8.54 (m, 1 H), 7.90- 7.80 (m, 2 H), 7.50 (br. s., 2 H), 7.47- 7.38 (m, 5 H), 7.22-7.16 (m, 1 H), 7.08- 6.98 (m, 4 H), 4.62-4.32 (m, 3 H), 3.76- 3.54 (m, 1 H), 3.34 (br. s., 1 H), 3.26- 3.07 (m, 1 H), 2.43 (br. s., 1 H), 2.27- 1.98 (m, 2H) |
| I-101 | 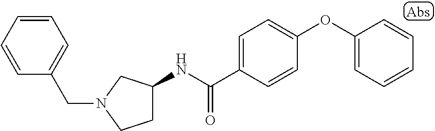 [SMILES: O=C(N[C@H]1CCN(Cc2ccccc2)C1)c1ccc(Oc2ccccc2)cc1] | [M + H]⁺ = 373 |
| I-102 | 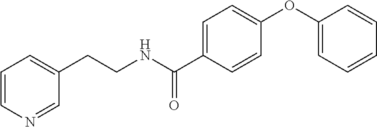 [SMILES: O=C(NCCc1cccnc1)c1ccc(Oc2ccccc2)cc1] | [M + H]⁺ = 319 |
| I-105 | 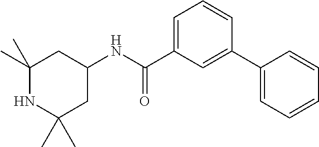 [SMILES: CC1(C)CC(NC(=O)c2cccc(-c3ccccc3)c2)CC(C)(C)N1] | [M + H]⁺ = 337 |
| I-114 | 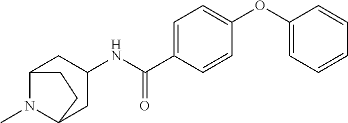 [SMILES: CN1C2CCC1CC(NC(=O)c1ccc(Oc3ccccc3)cc1)C2] | [M + H]⁺ = 337; |
| I-116 | 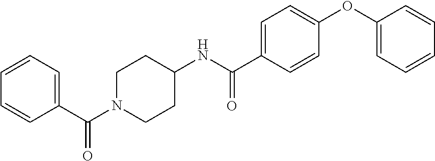 [SMILES: O=C(NC1CCN(C(=O)c2ccccc2)CC1)c1ccc(Oc2ccccc2)cc1] | [M + H]⁺ = 401; ¹H NMR (DMSO-d₆) δ: 8.23 (d, J = 7.6 Hz, 1H), 7.81-7.88 (m, 2H), 7.33-7.46 (m, 7H), 7.15-7.21 (m, 1H), 6.97-7.07 (m, 4H), 4.42 (br. s., 1H), 3.98-4.12 (m, 1H), 3.59 (br. s., 1H), 3.14 (br. s, 1H), 2.92 (br. s., 1H), 1.69-1.95 (m, 2H), 1.48 (br. s., 2H) |
| I-132 | 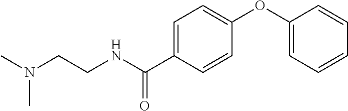 [SMILES: CN(C)CCNC(=O)c1ccc(Oc2ccccc2)cc1] | [M + H]⁺ = 285 |
| I-133 | 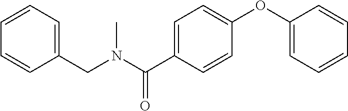 [SMILES: CN(Cc1ccccc1)C(=O)c1ccc(Oc2ccccc2)cc1] | [M + H]⁺ = 318; ¹H NMR (400 MHz DMSO-d₆) δ = 7.52- 7.23 (m, 8 H), 7.20-7.13 (m, 2 H), 7.05 (d, J = 7.8 Hz, 2 H), 6.99 (d, J = 6.4 Hz, 2 H), 4.63 (br. s., 2 H), 2.84 (s, 3 H). |
| I-134 | 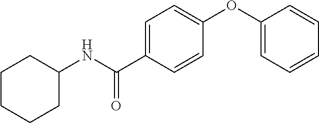 [SMILES: O=C(NC1CCCCC1)c1ccc(Oc2ccccc2)cc1] | [M + H]⁺ = 296; ¹H NMR (400 MHz DMSO-d₆) δ = 8.12 (d, J = 7.8 Hz, 1 H), 7.88-7.80 (m, 2 H), 7.45-7.35 (m, 2 H), 7.22-7.14 (m, 1 H), 7.07-7.02 (m, 2 H), 7.01-6.96 (m, 2 H), 3.78-3.66 (m, 1 H), 1.81-1.65 (m, 4 H), 1.61-1.54 (m, 1 H), 1.33-1.18 (m, 4 H), 1.15-1.01 (m, 1 H) |
| I-135 | 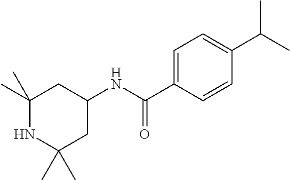 [SMILES: CC(C)c1ccc(C(=O)NC2CC(C)(C)NC(C)(C)C2)cc1] | [M + H]⁺ = 303 |
| I-136 | 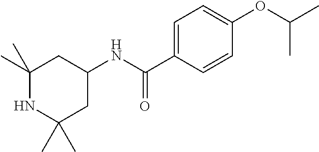 [SMILES: CC(C)Oc1ccc(C(=O)NC2CC(C)(C)NC(C)(C)C2)cc1] | [M + H]⁺ = 319 |
| I-137 | 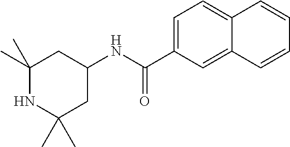 [SMILES: CC1(C)CC(NC(=O)c2ccc3ccccc3c2)CC(C)(C)N1] | [M + H]⁺ = 311 |
| I-138 | 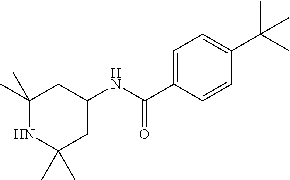 [SMILES: CC1(C)CC(NC(=O)c2ccc(C(C)(C)C)cc2)CC(C)(C)N1] | [M + H]⁺ = 317 |
| I-139 | 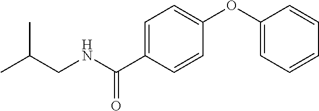 [SMILES: CC(C)CNC(=O)c1ccc(Oc2ccccc2)cc1] | [M + H]⁺ = 270 |
| I-140 | 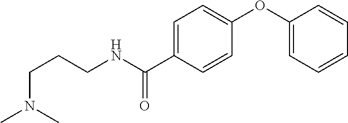 [SMILES: CN(C)CCCNC(=O)c1ccc(Oc2ccccc2)cc1] | [M + H]⁺ = 299 |
| I-141 | 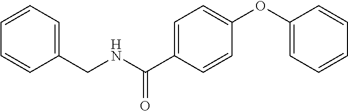 [SMILES: O=C(NCc1ccccc1)c1ccc(Oc2ccccc2)cc1] | [M + H]⁺ = 304 |

### Example 10

### 4-(3-Acetamidophenoxy)-N-(2,2,6,6-tetramethylpiperidin-4-yl)benzamide (I-26)

### Methyl 4-(3-acetamidophenoxy)benzamide

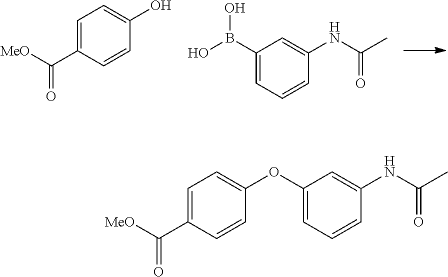 [SMILES: COC(=O)c1ccc(O)cc1 ; CC(=O)Nc1cccc(B(O)O)c1 ; COC(=O)c1ccc(Oc2cccc(NC(C)=O)c2)cc1]

Methyl 4-hydroxybenzoate (1.00 g, 6.57 mmol) was dissolved in DCM (10 mL). 3-Acetamidophenylboronic acid (1.18 g, 6.57 mmol), copper (II) acetate (1.19 g, 6.57 mmol), and triethylamine (4.6 mL, 33 mmol) were added. The reaction mixture was stirred o.n. under air, filtered over celite, and washed with ethyl acetate (200 mL). The resulting mixture was washed with aq. sat. sodium bicarbonate (100 mL), brine (100 mL), dried over sodium sulfate, filtered, and concentrated under reduced pressure. The residue was purified by flash chromatography (10% to 80% EtOAc/Hexanes) to afford methyl 4-(3-acetamidophenoxy)benzamide (1.0 g, 53%).

### 4-(3-Acetamidophenoxy)benzoic acid

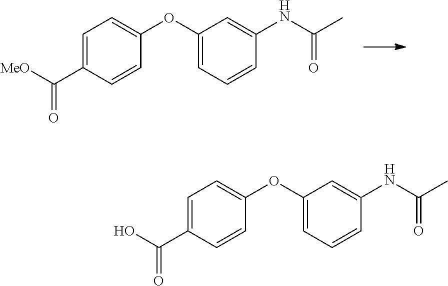 [SMILES: COC(=O)c1ccc(Oc2cccc(NC(C)=O)c2)cc1 ; CC(=O)Nc1cccc(Oc2ccc(C(=O)O)cc2)c1]

Methyl 4-(3-acetamidophenoxy)benzamide (1.0 g, 3.5 mmol) was dissolved in THF/methanol (3:1, 4 mL). The solution was cooled to 0° C. and 1N aq. sodium hydroxide (5.26 mL, 5.26 mmol) was added dropwise. The solution was warmed to r.t. and stirred until complete disappearance of the starting material. The reaction mixture was acidified with 1N aq. HCl, and extracted with DCM (2×100 mL). The combined organic phases were dried over sodium sulfate, filtered, concentrated under reduced pressure to afford 4-(3-acetamidophenoxy)benzoic acid (0.95 g, 45%) as a white solid.

### 4-(3-Acetamidophenoxy)-N-(2,2,6,6-tetramethylpiperidin-4-yl)benzamide (I-26)

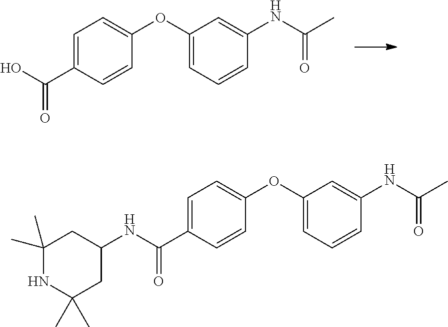

4-(3-Acetamidophenoxy)benzoic acid (71 mg, 0.26 mmol) was dissolved in dry DMF (1 mL) and the solution was cooled to 0° C. 2,2,6,6-Tetramethylpiperidin-4-amine (50 μL, 0.29 mmol), HATU (109 mg, 0.286 mmol), and N-ethyl-N-isopropylpropan-2-amine (91 μL, 0.52 mmol) were added and the reaction mixture was stirred at r.t. until complete disappearance of the starting material. The mixture was filtered and purified by preparative HPLC to afford 4-(3-acetamidophenoxy)-N-(2,2,6,6-tetramethylpiperidin-4-yl)benzamide 2,2,2-trifluoroacetate (35 mg, 26%) as a white solid. [M+H]⁺=410; ¹H NMR (DMSO-d₆) δ: 10.04 (s, 1H), 8.68 (d, J=11.7 Hz, 1H), 8.39 (d, J=7.3 Hz, 1H), 7.78-7.89 (m, 3H), 7.40-7.45 (m, 1H), 7.23-7.34 (m, 2H), 7.03 (d, J=8.7 Hz, 2H), 6.71 (dt, J=7.7, 1.7 Hz, 1H), 4.24-4.38 (m, 1H), 1.99 (s, 3H), 1.94 (dd, J=13.3, 2.7 Hz, 2H), 1.54 (t, J=12.9 Hz, 2H), 1.42 (s, 6H), 1.36 (s, 6H).

The following compounds were prepared in a similar manner as Example 10, above:

| Compound | Structure | Data |
|---|---|---|
| I-29 | 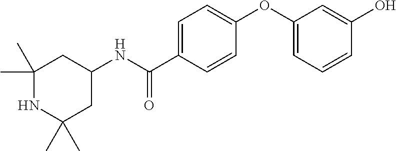 [SMILES: CC1(C)CC(NC(=O)c2ccc(Oc3cccc(O)c3)cc2)CC(C)(C)N1] | [M + H]⁺ = 369; ¹H NMR (DMSO-d₆) δ: 9.60-9.81 (m, 1H), 8.67 (d, J = 12.8 Hz, 1H), 8.38 (d, J = 7.3 Hz, 1H), 7.78-7.89 (m, 3H), 7.17 (t, J = 8.1 Hz, 1H), 6.99-7.05 (m, 2H), 6.57 (ddd, J = 8.1, 2.3, 0.8 Hz, 1H), 6.44 (ddd, J = 8.1, 2.3, 0.8 Hz, 1H), 6.37-6.40 (m, 1H), 4.25-4.37 (m, 1H), 1.94 (dd, J = 13.5, 3.2 Hz, 2H), 1.54 (t, J = 12.9 Hz, 2H), 1.42 (s, 6H), 1.36 (s, 6H) |
| I-71 | 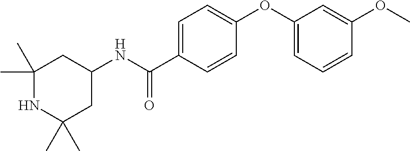 | [M + H]⁺ = 383; ¹H NMR (400 MHz DMSO-d₆) δ = 8.74 (d, J = 11.4 Hz, 1 H), 8.40 (d, J = 7.3 Hz, 1 H), 7.97- 7.78 (m, 3 H), 7.30 (t, J = 8.1 Hz, 1 H), 7.09- 6.95 (m, 2 H), 6.76 (dd, J = 2.4, 8.4 Hz, 1 H), 6.67-6.51 (m, 2 H), 4.42-4.22 (m, 1 H), 3.75- 3.68 (m, 3 H), 1.93 (dd, J = 2.6, 13.4 Hz, 2 H), 1.55 (t, J = 12.8 Hz, 2 H), 1.42 (s, 6 H), 1.36 (s, 6 H) |

### Example 11

### Methyl 3-((4-(2,2,6,6-tetramethyl-4-ylcarbamoyl)phenoxy)methyl)benzoate (I-53)

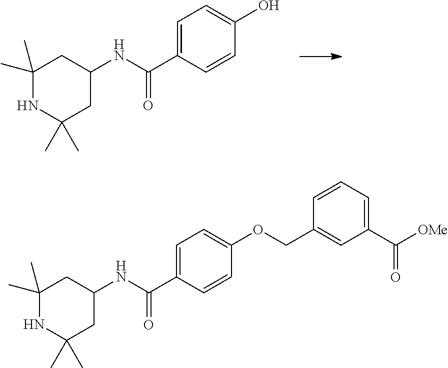 [SMILES: CC1(C)CC(NC(=O)c2ccc(O)cc2)CC(C)(C)N1 ; COC(=O)c1cccc(COc2ccc(C(=O)NC3CC(C)(C)NC(C)(C)C3)cc2)c1]

4-Hydroxy-N-(2,2,6,6-tetramethylpiperidin-4-yl)benzamide (100 mg, 0.362 mmol) and methyl 3-(bromomethyl)benzoate (83 mg, 0.36 mmol) were dissolved in acetone (5 mL). Potassium carbonate (65 mg, 0.47 mmol) was added and the reaction mixture was stirred at 50° C. o.n. The mixture was cooled to r.t., diluted with acetone, filtered, and concentrated under reduced pressure. The residue was dissolved in acetonitrile and purified by preparative HPLC to afford methyl 3-((4-(2,2,6,6-tetramethyl-4-ylcarbamoyl)phenoxy)methyl)benzoate (28 mg, 14%) as a white solid. [M+H]⁺=425; ¹H NMR (400 MHz, DMSO-d₆) δ=8.54 (d, J=12.6 Hz, 1H), 8.27 (d, J=7.3 Hz, 1H), 8.03 (s, 1H), 7.91 (d, J=7.6 Hz, 1H), 7.80 (d, J=8.7 Hz, 2H), 7.76-7.68 (m, 2H), 7.54 (t, J=7.7 Hz, 1H), 7.08 (d, J=8.9 Hz, 2H), 5.24 (s, 2H), 4.36-4.23 (m, 1H), 3.84 (s, 3H), 1.94 (dd, J=3.0, 13.3 Hz, 2H), 1.52 (t, J=12.9 Hz, 2H), 1.42 (s, 6H), 1.35 (s, 6H).

The following compounds were prepared in a similar manner as Example 11.

| Compound | Structure | Data |
|---|---|---|
| I-81 | 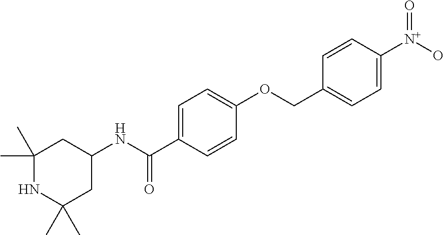 | [M + H]⁺ = 412; ¹H NMR (400 MHz ,DMSO-d₆) δ = 8.61- 8.53 (m, 1 H), 8.31-8.21 (m, 3 H), 7.84- 7.78 (m, 2 H), 7.71 (d, J = 8.9 Hz, 3 H), 7.11- 7.06 (m, 2 H), 5.33 (s, 2 H), 4.35-4.24 (m, 1 H), 1.94 (d, J = 13.7 Hz, 2 H), 1.52 (t, J= 12.9 Hz, 2 H), 1.42 (s, 6 H), 1.35 (s, 6 H) |
| I-88 | 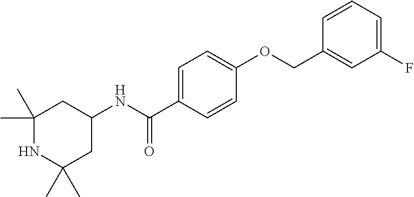 [SMILES: CC1(C)CC(NC(=O)c2ccc(OCc3cccc(F)c3)cc2)CC(C)(C)N1] | [M + H]⁺ = 385; ¹H NMR (400 MHz, DMSO-d₆) δ = 8.54 (d, J = 12.6 Hz, 1 H), 8.27 (d, J = 7.3 Hz, 1 H), 7.80 (d, J = 8.7 Hz, 2 H), 7.73 (d, J = 12.8 Hz, 1 H), 7.42 (dt, J = 6.2, 8.0 Hz, 1 H), 7.30- 7.24 (m, 2 H), 7.18-7.11 (m, 1 H), 7.07 (d, J = 8.9 Hz, 2 H), 5.18 (s, 2 H), 4.35-4.24 (m, 1 H), 1.94 (dd, J = 2.7, 13.5 Hz, 2 H), 1.52 (t, J = 13.0 Hz, 2 H), 1.42 (s, 6 H), 1.35 (s, 6 H) |
| I-90 | 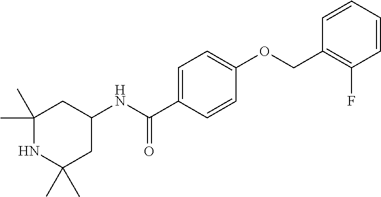 [SMILES: CC1(C)CC(NC(=O)c2ccc(OCc3ccccc3F)cc2)CC(C)(C)N1] | [M + H]⁺ = 385; ¹H NMR (400 MHz, DMSO-d₆) δ = 8.55 (d, J = 11.4 Hz, 1 H), 8.28 (d, J = 7.3 Hz, 1 H), 7.84-7.79 (m, 2 H), 7.73 (d, J = 11.7 Hz, 1 H), 7.55 (dt, J = 1.6, 7.7 Hz, 1 H), 7.45-7.37 (m, 1 H), 7.28-7.18 (m, 2 H), 7.11-7.05 (m, 2 H), 5.18 (s, 2 H), 4.36-4.24 (m, 1 H), 1.94 (dd, J = 3.0, 13.5 Hz, 2 H), 1.52 (t, J = 12.8 Hz, 2 H), 1.42 (s, 6 H), 1.36 (s, 6 H) |
| I-117 | 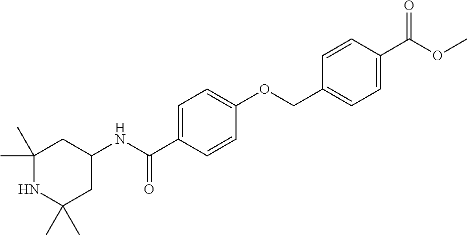 | [M + H]⁺ = 425; ¹H NMR (400 MHz, DMSO-d₆) δ: 8.42-8.60 (m, 1H), 8.26 (d, J = 7.8 Hz, 1H), 7.97 (d, J = 8.5 Hz, 2H), 7.77-7.83 (m, 2H), 7.67-7.75 (m, 1H), 7.58 (d, J = 8.0 Hz, 2H), 7.07 (d, J = 8.9 Hz, 2H), 5.26 (s, 2H), 4.22-4.36 (m, 1H), 3.83 (s, 3H), 1.94 (d, J = 13.3 Hz, 2H), 1.51 (t, J = 12.8 Hz, 2H), 1.42 (s, 6H), 1.35 (s, 6H) |

### Example 12

### 4-(Benzylthio)-N-(2,2,6,6-tetramethylpiperidin-4-yl)benzamide (I-45)

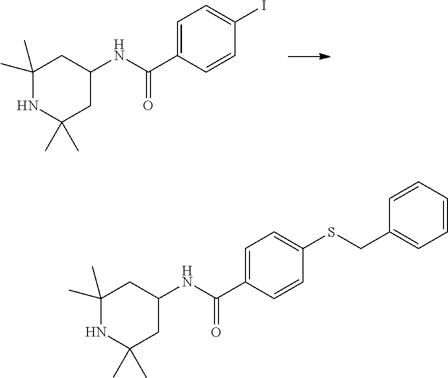 [SMILES: CC1(C)CC(NC(=O)c2ccc(I)cc2)CC(C)(C)N1 ; CC1(C)CC(NC(=O)c2ccc(SCc3ccccc3)cc2)CC(C)(C)N1]

4-Iodo-N-(2,2,6,6-tetramethylpiperidin-4-yl)benzamide (74 mg, 0.19 mmol), potassium carbonate (53 mg, 0.38 mmol), and copper (I) iodide (1.8 mg, 0.009 mmol) were weighed in a test tube equipped with a teflon screw cap. The tube was evacuated with house vacuum and filled with nitrogen. The cycle was repeated twice. Isopropanol (2 μL), phenylmethanethiol (22 mL, 0.19 mmol), and ethylene glycol (21 μL, 0.38 mmol) were added. The reaction mixture was stirred at 80° C. o.n. The resulting mixture was cooled to r.t., diluted with aq. sat. sodium bicarbonate (100 mL), and extracted with ethyl acetate (2×50 mL). The combined organic phases were washed with brine, dried over sodium sulfate, filtered, and concentrated under reduced pressure. The residue was diluted with methanol (1 mL) and water (500 μL), filtered through PTFE and purified by preparative HPLC to afford 4-(benzylthio)-N-(2,2,6,6-tetramethylpiperidin-4-yl)benzamide 2,2,2-trifluoroacetate (34 mg, 0.068 mmol, 36%) as a white solid. [M+H]⁺=383; ¹H NMR (DMSO-d₆) δ 8.68 (d, J=12.4 Hz, 1H), 8.39 (d, J=7.3 Hz, 1H), 7.83 (d, J=12.1 Hz, 1H), 7.74 (d, J=8.2 Hz, 2H), 7.39 (d, J=8.0 Hz, 4H), 7.27-7.33 (m, 2H), 7.20-7.25 (m, 1H), 4.25-4.38 (m, 3H), 1.94 (dd, J=13.5, 2.7 Hz, 2H), 1.54 (t, J=12.9 Hz, 2H), 1.43 (s, 6H), 1.37 (s, 6H).

The following compound was prepared in a manner similar to Example 12.

| Compound | Structure | Data |
|---|---|---|
| I-50 | 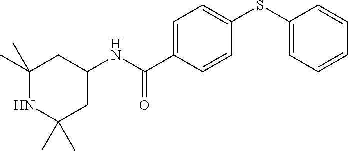 [SMILES: CC1(C)CC(NC(=O)c2ccc(Sc3ccccc3)cc2)CC(C)(C)N1] | [M + H]⁺ = 369; ¹H NMR (DMSO-d₆) δ: 8.78 (d, J = 12.4 Hz, 1H), 8.46 (d, J = 7.3 Hz, 1H), 7.91 (d, J = 11.9 Hz, 1H), 7.79 (d, J = 8.5 Hz, 2H), 7.36-7.47 (m, 5H), 7.29 (d, J = 8.5 Hz, 2H), 4.31 (td, J = 7.9, 4.1 Hz, 1H), 1.94 (dd, J = 13.5, 2.7 Hz, 2H), 1.56 (t, J = 12.8 Hz, 2H), 1.43 (s, 6H), 1.37 (s, 6H) |

### Example 13

### 6-Phenoxy-N-(2,2,6,6-tetramethylpiperidin-4-yl)nicotinamide (I-64)

### Methyl 6-phenoxynicotinamide

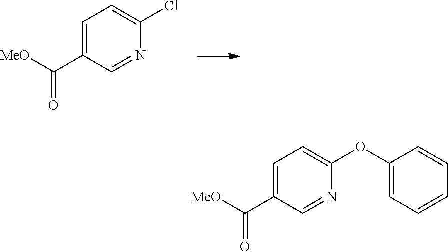 [SMILES: COC(=O)c1ccc(Cl)nc1 ; COC(=O)c1ccc(Oc2ccccc2)nc1]

Methyl 6-chloronicotinamide (500 mg, 2.91 mmol), and phenol (274 mg, 2.91 mmol) were dissolved in dry DMF (10 mL). Potassium carbonate (604 mg, 4.37 mmol) was added and the reaction mixture was stirred at 80° C. o.n. The reaction mixture was cooled to r.t., diluted with water (50 mL) and extracted with ethyl acetate (2×100 mL). The combined organic phases were washed with aq. sat. sodium bicarbonate (100 mL), brine (100 mL) dried over sodium sulfate, filtered and concentrated under reduced pressure. The residue was purified by flash chromatography (1% to 60% EtOAc/Hexanes) to afford methyl 6-phenoxynicotinamide (421 mg, 63%).

### 6-Phenoxynicotinic acid

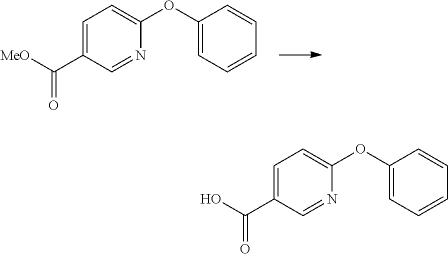 [SMILES: COC(=O)c1ccc(Oc2ccccc2)nc1 ; O=C(O)c1ccc(Oc2ccccc2)nc1]

Methyl 6-phenoxynicotinamide (421 mg, 1.83 mmol) was dissolved in THF/Methanol (3:1, 4 mL) and cooled to 0° C. 1N Aq. lithium hydroxide (1.8 mL, 1.8 mmol) was added dropwise. The reaction mixture was stirred at r.t. until complete disappearance of the starting material, then acidified with aq. 1N HCl, and extracted with DCM (2×100 mL). The combined organic phases were dried over sodium sulfate, filtered and concentrated under reduced pressure to afford 6-phenoxynicotinic acid (395 mg, 100%) as a white solid.

### 6-Phenoxy-N-(2,2,6,6-tetramethylpiperidin-4-yl)nicotinamide (I-64)

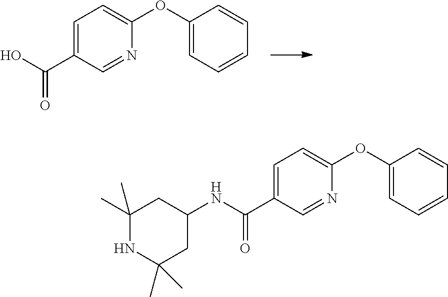 [SMILES: O=C(O)c1ccc(Oc2ccccc2)nc1 ; CC1(C)CC(NC(=O)c2ccc(Oc3ccccc3)nc2)CC(C)(C)N1]

6-Phenoxynicotinic acid (67 mg, 0.31 mmol) was dissolved in dry DMF (1 mL) and the solution was cooled to 0° C. 2,2,6,6-Tetramethylpiperidin-4-amine (60 μL, 0.342 mmol), HATU (109 mg, 0.374 mmol), and N-ethyl-N-isopropylpropan-2-amine (136 μL, 0.778 mmol) were added and the reaction mixture was stirred at r.t. until complete disappearance of the starting material. The mixture was filtered and purified by preparative HPLC to afford 6-phenoxy-N-(2,2,6,6-tetramethylpiperidin-4-yl)nicotinamide 2,2,2-trifluoroacetate (71 mg, 49%) as a white solid. [M+H]⁺=351; ¹H NMR (400 MHz, (DMSO-d₆) δ=8.71 (d, J=11.9 Hz, 1H), 8.56 (d, J=2.1 Hz, 1H), 8.52 (d, J=7.3 Hz, 1H), 8.22 (dd, J=2.5, 8.7 Hz, 1H), 7.86 (d, J=11.9 Hz, 1H), 7.45-7.39 (m, 2H), 7.25-7.20 (m, 1H), 7.16-7.12 (m, 2H), 7.08 (d, J=8.5 Hz, 1H), 4.36-4.25 (m, 1H), 1.95 (dd, J=2.9, 13.4 Hz, 2H), 1.53 (t, J=12.8 Hz, 2H), 1.42 (s, 6H), 1.36 (s, 6H).

### Example 14

### 4-(5-Iodopyridin-2-yloxy)-N-(2,2,6,6-tetramethylpiperidin-4-yl)benzamide (I-68)

### Methyl 4-(5-iodopyridin-2-yloxy)benzoate

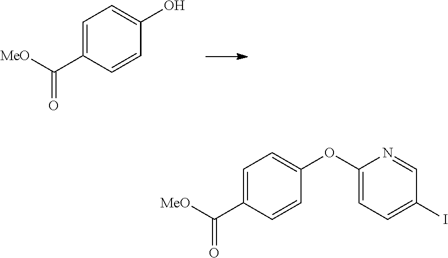 [SMILES: COC(=O)c1ccc(O)cc1 ; COC(=O)c1ccc(Oc2ccc(I)cn2)cc1]

Methyl 4-hydroxybenzoate (500 mg, 3.29 mmol) was dissolved in dry DMF (10 mL). 2-Chloro-4-iodopyridine (866 mg, 3.61 mmol) and potassium carbonate (545 mg, 3.94 mmol) were added. The reaction mixture was stirred at 120° C. until complete disappearance of the starting material. The mixture was cooled to r.t., diluted with aq. sat. sodium bicarbonate (200 mL), and extracted with ethyl acetate (2×100 mL). The combined organic phases were dried over sodium sulfate, filtered and concentrated under reduced pressure to afford methyl 4-(5-iodopyridin-2-yloxy)benzoate (292 mg, 25%) as a white solid.

### 4-(5-Iodopyridin-2-yloxy)benzoic acid

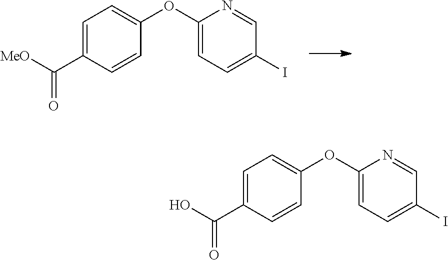 [SMILES: COC(=O)c1ccc(Oc2ccc(I)cn2)cc1 ; O=C(O)c1ccc(Oc2ccc(I)cn2)cc1]

Methyl 4-(5-iodopyridin-2-yloxy)benzoate (292 mg, 0.822 mmol) was dissolved in THF (5 mL) and cooled to 0° C. 1N Aq. sodium hydroxide (1.2 mL, 1.2 mmol) was added dropwise and the reaction mixture was stirred at r.t. until complete disappearance of the starting material. The mixture was acidified with 1N aq. HCl and extracted with DCM (2×100 mL). The combined organic phases were dried over sodium sulfate, filtered and concentrated under reduced pressure to afford 4-(5-iodopyridin-2-yloxy)benzoic acid (223 mg, 80%) as a white solid.

### 4-(5-Iodopyridin-2-yloxy)-N-(2,2,6,6-tetramethylpiperidin-4-yl)benzamide (I-68)

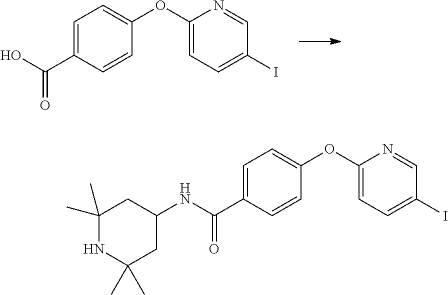 [SMILES: O=C(O)c1ccc(Oc2ccc(I)cn2)cc1 ; CC1(C)CC(NC(=O)c2ccc(Oc3ccc(I)cn3)cc2)CC(C)(C)N1]

4-(5-Iodopyridin-2-yloxy)benzoic acid (89 mg, 0.26 mmol) was dissolved in dry DMF (1 mL) and the solution was cooled to 0° C. 2,2,6,6-Tetramethylpiperidin-4-amine (50 μL, 0.286 mmol), HATU (109 mg, 0.286 mmol), and N-ethyl-N-isopropylpropan-2-amine (91 μL, 0.52 mmol) were added and the reaction mixture was stirred at r.t. until complete disappearance of the starting material. The mixture was filtered and purified by preparative HPLC to afford 4-(5-iodopyridin-2-yloxy)-N-(2,2,6,6-tetramethylpiperidin-4-yl)benzamide 2,2,2-trifluoroacetate (32 mg, 21%) as a white solid. [M+H]⁺=480; ¹H NMR (400 MHz, (DMSO-d₆) δ=8.57 (d, J=12.4 Hz, 1H), 8.42 (d, J=7.3 Hz, 1H), 7.90-7.83 (m, 2H), 7.75 (d, J=12.4 Hz, 1H), 7.59-7.52 (m, 2H), 7.24-7.17 (m, 2H), 4.39-4.25 (m, 1H), 1.96 (dd, J=3.1, 13.6 Hz, 2H), 1.53 (t, J=12.8 Hz, 2H), 1.43 (s, 6H), 1.36 (s, 6H).

The following compounds were prepared in a similar manner as Example 14.

| Compound | Structure | Data |
|---|---|---|
| I-78 | 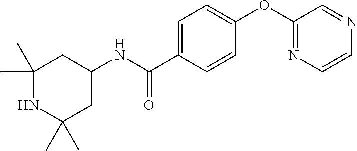 [SMILES: CC1(C)CC(NC(=O)c2ccc(Oc3cnccn3)cc2)CC(C)(C)N1] | [M + H]⁺ = 355; ¹H NMR (DMSO-d₆) δ: 8.60 (br. s., 1H), 8.57 (s, 1H), 8.45 (d, J = 7.3 Hz, 1H), 8.40 (dd, J = 2.7, 0.5 Hz, 1H), 8.20 (dt, J = 2.6, 1.2 Hz, 1H), 7.89 (d, J = 8.0 Hz, 2H), 7.77 (d, J = 11.7 Hz, 1H), 7.28 (d, J = 8.0 Hz, 2H), 4.27-4.39 (m, 1H), 1.92-2.01 (m, 2H), 1.54 (t, J = 12.9 Hz, 2H), 1.43 (s, 6H), 1.36 (s, 6H) |
| I-79 | 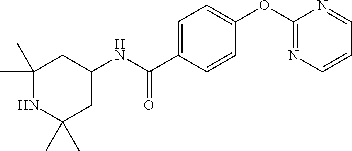 [SMILES: CC1(C)CC(NC(=O)c2ccc(Oc3ncccn3)cc2)CC(C)(C)N1] | [M + H]⁺ = 355; ¹H NMR (DMSO-d₆) δ: 8.64 (d, J = 4.8 Hz, 2H), 8.54 (d, J = 12.1 Hz, 1H), 8.44 (d, J = 7.3 Hz, 1H), 7.84-7.92 (m, 2H), 7.73 (d, J = 12.4 Hz, 1H), 7.24-7.32 (m, 3H), 4.26-4.40 (m, 1H), 1.97 (dd, J = 13.4, 3.1 Hz, 2H), 1.54 (t, J = 12.8 Hz, 2H), 1.43 (s, 6H), 1.36 (s, 6H) |

### Example 15

### 2-(4-(2,2,6,6-Tetramethylpiperidin-4-ylcarbamoyl)phenoxy)nicotinamide (I-103)

### Methyl 4-(3-carbamoylpyridin-2-yloxy)benzoate

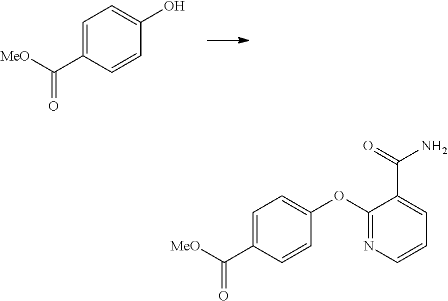 [SMILES: COC(=O)c1ccc(O)cc1 ; COC(=O)c1ccc(Oc2ncccc2C(N)=O)cc1]

2-Chloronicotinamide (1.13 g, 7.23 mmol) and methyl 4-hydroxybenzoate (1.00 g, 6.57 mmol) were dissolved in dry DMF (20 mL). The reaction mixture was cooled to 0° C. and sodium hydride (315 mg, 7.89 mmol) was added. The suspension was warmed to r.t. then stirred at 120° C. o.n. The reaction mixture was cooled to r.t., diluted with aq. sat. sodium bicarbonate (100 mL) and extract with ethyl acetate (2×100 mL). The combined organic phases were dried over sodium sulfate, filtered and concentrated under reduced pressure to afford methyl 4-(3-carbamoylpyridin-2-yloxy)benzoate (110 mg, 6%).

### 4-(3-Carbamoylpyridin-2-yloxy)benzoic acid

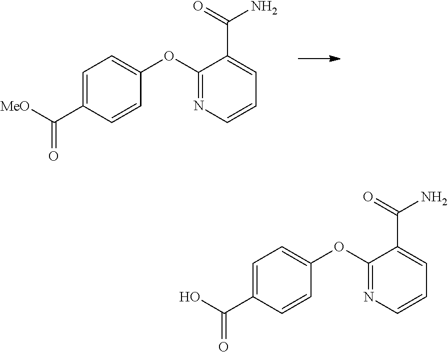 [SMILES: COC(=O)c1ccc(Oc2ncccc2C(N)=O)cc1 ; NC(=O)c1cccnc1Oc1ccc(C(=O)O)cc1]

Methyl 4-(3-carbamoylpyridin-2-yloxy)benzoate (110 mg, 0.404 mmol) was dissolved in THF/MeOH (3:1, 4 mL) and cooled to 0° C. 1N Aq. lithium hydroxide (0.404 mL, 0.404 mmol) was added dropwise and the reaction mixture was stirred at r.t. until complete disappearance of the starting material. The mixture was acidified with 1N aq. HCl and extracted with DCM (2×100 mL). The combined organic phases were dried over sodium sulfate, filtered and concentrated under reduced pressure to afford 4-(3-carbamoylpyridin-2-yloxy)benzoic acid (73 mg, 70%) as a white solid.

### 2-(4-(2,2,6,6-Tetramethylpiperidin-4-ylcarbamoyl)phenoxy)nicotinamide

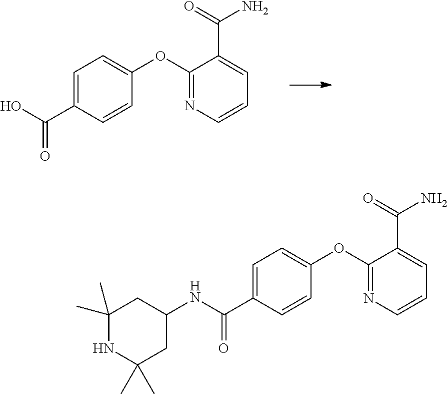 [SMILES: NC(=O)c1cccnc1Oc1ccc(C(=O)O)cc1 ; CC1(C)CC(NC(=O)c2ccc(Oc3ncccc3C(N)=O)cc2)CC(C)(C)N1]

4-(3-Carbamoylpyridin-2-yloxy)benzoic acid (73 mg, 0.28 mmol) was dissolved in dry DMF (1 mL) and the solution was cooled to 0° C. 2,2,6,6-Tetramethylpiperidin-4-amine (54 μL, 0.31 mmol), HATU (129 mg, 0.339 mmol), and N-ethyl-N-isopropylpropan-2-amine (123 μL, 0.707 mmol) were added and the reaction mixture was stirred at r.t. until complete disappearance of the starting material. The mixture was filtered and purified by preparative HPLC to 2-(4-(2,2,6,6-tetramethylpiperidin-4-ylcarbamoyl)phenoxy)nicotinamide (80 mg, 55%) as a white solid. [M+H]⁺=397; ¹H NMR (400 MHz DMSO-d₆) δ=8.61-8.50 (m, 2H), 8.46-8.40 (m, 1H), 8.20-8.11 (m, 2H), 7.87 (d, J=8.7 Hz, 2H), 7.84-7.71 (m, 2H), 7.28-7.19 (m, 3H), 4.32 (dd, J=3.4, 7.8 Hz, 1H), 1.97 (d, J=13.5 Hz, 2H), 1.54 (t, J=12.9 Hz, 2H), 1.43 (s, 6H), 1.36 (s, 6H).

The following compounds were prepared in a manner similar to Example 15.

| Compound | Structure | Data |
|---|---|---|
| I-109 | 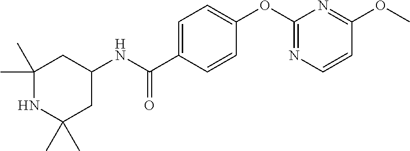 | [M + H]⁺ = 385; ¹H NMR (400 MHz DMSO-d₆) δ = 8.66 (d, J = 12.4 Hz, 1 H), 8.45 (d, J = 7.6 Hz, 1 H), 8.27 (d, J = 5.5 Hz, 1 H), 7.86-7.91 (m, 2 H), 7.82 (d, J = 11.4 Hz, 1 H), 7.26-7.32 (m, 2 H), 6.69 (d, J = 5.7 Hz, 1 H), 4.26-4.39 (m, 1 H), 3.84 (s, 3 H), 1.96 (dd, J = 13.4, 3.1 Hz, 2 H), 1.55 (t, J = 12.8 Hz, 2 H), 1.43 (s, 6 H), 1.37 ppm (s, 6 H) |
| I-111 | 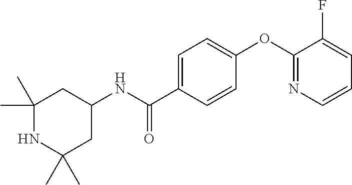 [SMILES: CC1(C)CC(NC(=O)c2ccc(Oc3ncccc3F)cc2)CC(C)(C)N1] | [M + H]⁺ = 372; ¹H NMR (400 MHz DMSO-d₆) δ = 8.68 (d, J = 12.1 Hz, 1 H), 8.45 (d, J = 7.1 Hz, 1 H), 7.95 (dd, J = 1.4, 4.8 Hz, 1 H), 7.91-7.80 (m, 4 H), 7.26-7.21 (m, 3 H), 4.33 (tdd, J = 4.0, 8.2, 15.7 Hz, 1 H), 1.96 (dd, J = 3.0, 13.3 Hz, 2 H), 1.55 (t, J = 12.8 Hz, 2 H), 1.43 (s, 6 H), 1.37 (s, 6 H) |

### Example 16

### 6-(Phenylamino)-N-(2,2,6,6-tetramethylpiperidin-4-yl)nicotinamide (I-104)

### Methyl 6-(phenylamino)nicotinate

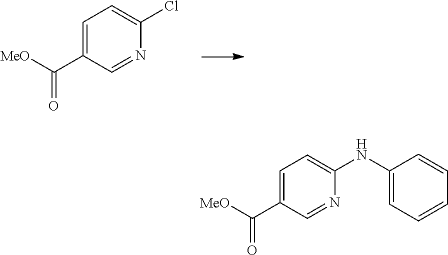 [SMILES: COC(=O)c1ccc(Cl)nc1 ; COC(=O)c1ccc(Nc2ccccc2)nc1]

Methyl 6-chloronicotinate (1.0 g, 5.8 mmol) was weighed in a 50 mL flask. Aniline (2.0 mL, 22 mmol) was added and the mixture was stirred neat at 120° C. until complete disappearance of the starting material. The mixture was diluted with DMF (10 mL), water (20 mL) and sonicated for 10 minutes. The white precipitated was filtered to afford crude methyl 6-(phenylamino)nicotinate (1.3 g, 100%).

### 6-(Phenylamino)nicotinic acid

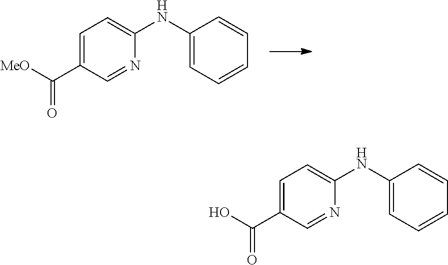 [SMILES: COC(=O)c1ccc(Nc2ccccc2)nc1 ; O=C(O)c1ccc(Nc2ccccc2)nc1]

Methyl 6-(phenylamino)nicotinate (1.3 g, 5.7 mmol) was dissolved in THF/MeOH (3:1, 4 mL) and cooled to 0° C. 1N Aq. lithium hydroxide (5.7 mL, 5.7 mmol) was added dropwise. The reaction mixture was stirred at r.t. overnight, acidified to pH ˜5 and the precipitate was filtered to afford 6-(phenylamino)nicotinic acid as a white solid (800 mg, 65%).

### 6-(Phenylamino)-N-(2,2,6,6-tetramethylpiperidin-4-yl)nicotinamide (I-104)

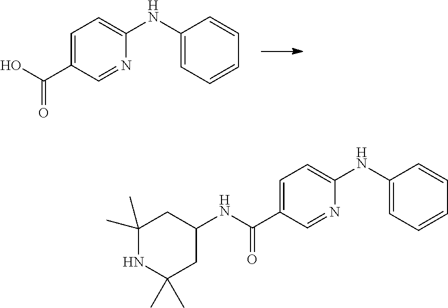 [SMILES: O=C(O)c1ccc(Nc2ccccc2)nc1 ; CC1(C)CC(NC(=O)c2ccc(Nc3ccccc3)nc2)CC(C)(C)N1]

6-(Phenylamino)nicotinic acid (60 mg, 0.28 mmol) was dissolved in dry DMF (1 mL) and the solution was cooled to 0° C. 2,2,6,6-Tetramethylpiperidin-4-amine (54 μL, 0.31 mmol), HATU (129 mg, 0.339 mmol), and N-ethyl-N-isopropylpropan-2-amine (123 μL, 0.707 mmol) were added and the reaction mixture was stirred at r.t. until complete disappearance of the starting material. The mixture was filtered and purified by preparative HPLC to 6-(phenylamino)-N-(2,2,6,6-tetramethylpiperidin-4-yl)nicotinamide (65 mg, 50%) as a white solid. [M+H]⁺=353; ¹H NMR (400 MHz DMSO-d₆) δ=9.78 (br. s., 1H), 8.56 (d, J=2.3 Hz, 2H), 8.36 (d, J=7.1 Hz, 1H), 8.04 (dd, J=2.4, 8.8 Hz, 1H), 7.74 (d, J=12.4 Hz, 1H), 7.60 (d, J=7.6 Hz, 2H), 7.33 (t, J=7.9 Hz, 2H), 7.04 (t, J=7.4 Hz, 1H), 6.91 (d, J=8.9 Hz, 1H), 1.96 (dd, J=2.7, 13.5 Hz, 2H), 1.51 (t, J=12.8 Hz, 2H), 1.42 (s, 6H), 1.36 (s, 6H).

### Example 17

### 4-((Phenylamino)methyl)-N-(2,2,6,6-tetramethylpiperidin-4-yl)benzamide (I-106)

### Methyl 4-((phenylamino)methyl)benzoate

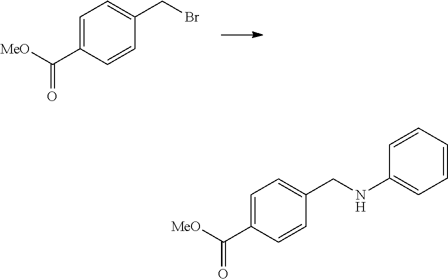 [SMILES: COC(=O)c1ccc(CBr)cc1 ; COC(=O)c1ccc(CNc2ccccc2)cc1]

Methyl 4-(bromomethyl)benzoate (1.0 g, 4.4 mmol) was dissolved in dry DMF (20 mL). Aniline (478 μL, 5.24 mmol) and potassium carbonate (905 mg, 6.55 mmol) were added and the mixture was stirred at 55° C. for 5 hours. The reaction mixture was cooled to r.t., diluted with water (100 mL) and extracted with ethyl acetate (3×100 mL). The combined organic phases were washed with water (2×50 mL), brine (2×50 mL), dried over sodium sulfate, filtered, and concentrated under reduced pressure. The residue was purified by flash chromatography (5% to 80% EtOAc/Hexanes) to afford methyl 4-((phenylamino)methyl)benzoate (891 mg, 85%).

### 4-((Phenylamino)methyl)benzoic acid

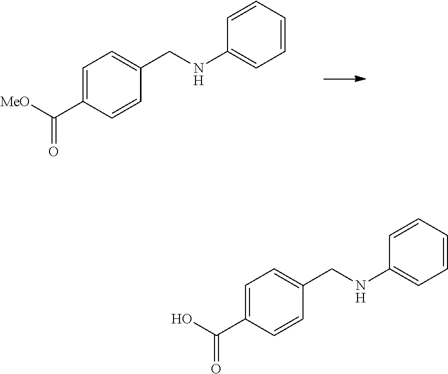 [SMILES: COC(=O)c1ccc(CNc2ccccc2)cc1 ; O=C(O)c1ccc(CNc2ccccc2)cc1]

Methyl 4-((phenylamino)methyl)benzoate (824 mg, 3.42 mmol) was dissolved in THF/MeOH (3:1, 8 mL) and cooled to 0° C. 1N Aq. lithium hydroxide (4.1 mL, 4.1 mmol) was added dropwise. The reaction mixture was stirred at r.t. overnight, acidified to pH˜5 and extracted with DCM (5×50 mL). The combined organic phases were dried over sodium sulfate, filtered, concentrated under pressure to afford crude 4-((phenylamino)methyl)benzoic acid (486 mg, 63%).

### 4-((Phenylamino)methyl)-N-(2,2,6,6-tetramethylpiperidin-4-yl)benzamide (I-106)

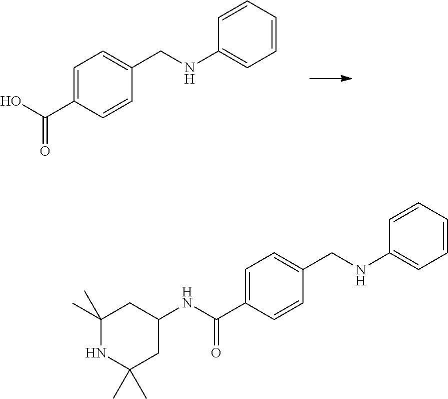 [SMILES: O=C(O)c1ccc(CNc2ccccc2)cc1 ; CC1(C)CC(NC(=O)c2ccc(CNc3ccccc3)cc2)CC(C)(C)N1]

4-((Phenylamino)methyl)benzoic acid (36 mg, 0.16 mmol) was dissolved in dry DMF (1 mL) and the solution was cooled to 0° C. 2,2,6,6-Tetramethylpiperidin-4-amine (30 μL, 0.17 mmol), HATU (71.3 mg, 0.187 mmol), and N-ethyl-N-isopropylpropan-2-amine (68 μL, 0.39 mmol) were added and the reaction mixture was stirred at r.t. until complete disappearance of the starting material. The mixture was filtered and purified by preparative HPLC to 4-((phenylamino)methyl)-N-(2,2,6,6-tetramethylpiperidin-4-yl)benzamide (43 mg, 57%) as a white solid. [M+H]⁺=366; ¹H NMR (400 MHz DMSO-d₆) δ=8.64 (d, J=12.1 Hz, 1H), 8.36 (d, J=7.3 Hz, 1H), 7.80 (d, J=11.7 Hz, 1H), 7.76-7.72 (m, 2H), 7.41 (d, J=8.5 Hz, 2H), 7.03-6.97 (m, 2H), 6.56-6.46 (m, 3H), 4.37-4.23 (m, 3H), 1.93 (dd, J=3.2, 13.5 Hz, 2H), 1.52 (t, J=12.8 Hz, 2H), 1.41 (s, 6H), 1.35 (s, 6H).

### Example 18

### 4-(Phenyl)-N-(2,2,6,6-tetramethylpiperidin-4-yl)benzamide

### Methyl 4-(phenylamino)benzoate (I-107)

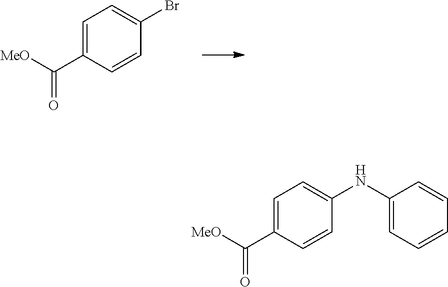 [SMILES: COC(=O)c1ccc(Br)cc1 ; COC(=O)c1ccc(Nc2ccccc2)cc1]

In a sealed tube were weighed methyl 4-bromobenzoate (1.0 g, 4.7 mmol), BretPhos (65 mg, 0.85 mmol), cesium carbonate (2.1 g, 6.3 mmol), and aniline (386 μL, 4.20 mmol). The tube was evacuated with house vacuum and filled back with nitrogen. The cycle was repeated twice and dioxane (5 mL) was added. The reaction mixture was stirred at 110° C. 2 hours, cooled to r.t., diluted with aq. sat. sodium bicarbonate (200 mL) and extracted with ethyl acetate (2×100 mL). The combined organic phases were washed with aq. sat. sodium bicarbonate (100 mL), brine (100 mL) dried over sodium sulfate, filtered and concentrated under reduced pressure. The residue was purified by flash chromatography (1% to 100% EtOAc/Hexanes) to afford methyl 4-(phenylamino)benzoate (961 mg, 100%).

### 4-(Phenylamino)benzoic acid

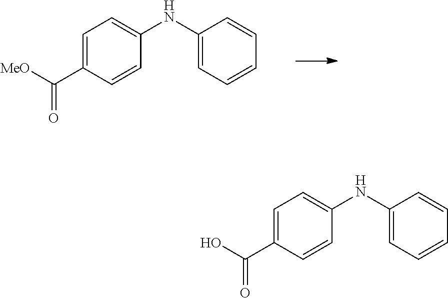 [SMILES: COC(=O)c1ccc(Nc2ccccc2)cc1 ; O=C(O)c1ccc(Nc2ccccc2)cc1]

Methyl 4-(phenylamino)benzoate (711 mg, 3.13 mmol) was dissolved in THF/MeOH (3:1, 4 mL) and cooled to 0° C. 1N Aq. lithium hydroxide (3.7 mL, 3.7 mmol) was added dropwise. The reaction mixture was stirred at r.t. overnight, acidified to pH -5 and extracted with DCM (5×50 mL). The combined organic phases were dried over sodium sulfate, filtered, concentrated under pressure to afford crude 4-(phenylamino)benzoic acid (600 mg, 90%).

### 4-(Phenyl)-N-(2,2,6,6-tetramethylpiperidin-4-yl)benzamide (I-107)

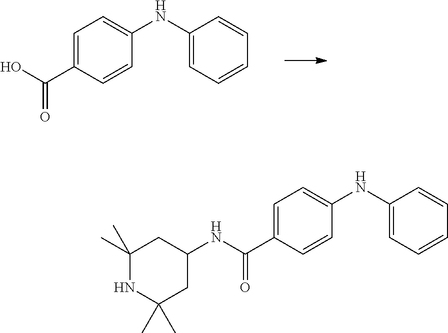 [SMILES: O=C(O)c1ccc(Nc2ccccc2)cc1 ; CC1(C)CC(NC(=O)c2ccc(Nc3ccccc3)cc2)CC(C)(C)N1]

4-(Phenylamino)benzoic acid (33 mg, 0.156 mmol) was dissolved in dry DMF (1 mL) and the solution was cooled to 0° C. 2,2,6,6-Tetramethylpiperidin-4-amine (30 μL, 0.17 mmol), HATU (71.3 mg, 0.187 mmol), and N-ethyl-N-isopropylpropan-2-amine (68 μL, 0.39 mmol) were added and the reaction mixture was stirred at r.t. until complete disappearance of the starting material. The mixture was filtered and purified by preparative HPLC to 4-(phenyl)-N-(2,2,6,6-tetramethylpiperidin-4-yl)benzamide (16 mg, 22%) as a white solid. [M+H]⁺=352; ¹H NMR (400 MHz DMSO-d₆) δ=8.61-8.50 (m, 2H), 8.16-8.10 (m, 1H), 7.73-7.68 (m, 2H), 7.30-7.23 (m, 2H), 7.15-7.09 (m, 2H), 7.06-7.00 (m, 2H), 6.94-6.87 (m, 1H), 4.35-4.24 (m, 1H), 1.97-1.89 (m, 2H), 1.57-1.47 (m, 2H), 1.42 (s, 6H), 1.35 (s, 6H).

### Example 19

### 4-(Methyl(phenyl)amino)-N-(2,2,6,6-tetramethylpiperidin-4-yl)benzamide (I-108)

### Methyl 4-(methyl(phenyl)amino)benzoate

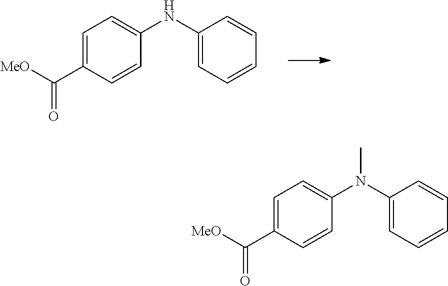 [SMILES: COC(=O)c1ccc(Nc2ccccc2)cc1 ; COC(=O)c1ccc(N(C)c2ccccc2)cc1]

Methyl 4-(methyl(phenyl)amino)benzoate (246 mg, 1.08 mmol) was dissolved in dry DMF (5 mL) and cooled to 0° C. Sodium hydride (60% in mineral oil, 56 mg, 1.4 mmol) was added, followed by iodomethane (74 μL, 1.2 mmol). The mixture was stirred at r.t. o.n., diluted with aq. sat. sodium bicarbonate (100 mL), and extracted with ethyl acetate (2×100 mL). The combined organic phases were washed with aq. sat. sodium bicarbonate (100 mL), brine (100 mL) dried over sodium sulfate, filtered and concentrated under reduced pressure. The residue was purified by flash chromatography (1% to 100% EtOAc/Hexanes) to afford methyl 4-(methyl(phenyl)amino)benzoate (255 mg, 98%).

### 4-(Methyl(phenyl)amino)benzoic acid

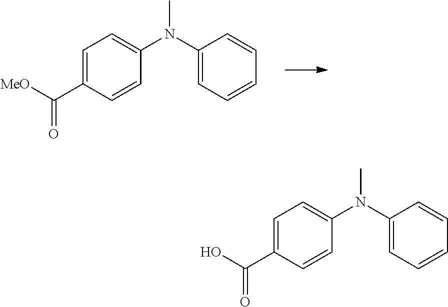 [SMILES: COC(=O)c1ccc(N(C)c2ccccc2)cc1 ; CN(c1ccccc1)c1ccc(C(=O)O)cc1]

Methyl 4-(methyl(phenyl)amino)benzoate (255 mg, 1.06 mmol) was dissolved in THF/MeOH (3:1, 4 mL) and cooled to 0° C. 1N Aq. lithium hydroxide (2.1 mL, 2.1 mmol) was added dropwise. The reaction mixture was stirred at r.t. o.n., acidified to pH -5 and extracted with DCM (5×50 mL). The combined organic phases were dried over sodium sulfate, filtered, concentrated under pressure to afford crude 4-(methyl(phenyl)amino)benzoic acid (190 mg, 79%).

### 4-(Methyl(phenyl)amino)-N-(2,2,6,6-tetramethylpiperidin-4-yl)benzamide (I-108)

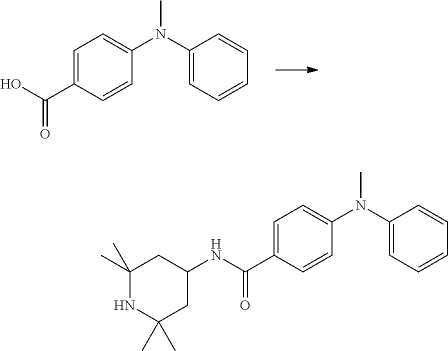 [SMILES: CN(c1ccccc1)c1ccc(C(=O)O)cc1 ; CN(c1ccccc1)c1ccc(C(=O)NC2CC(C)(C)NC(C)(C)C2)cc1]

4-(Methyl(phenyl)amino)benzoic acid (35.5 mg, 0.156 mmol) was dissolved in dry DMF (1 mL) and the solution was cooled to 0° C. 2,2,6,6-Tetramethylpiperidin-4-amine (30 μL, 0.17 mmol), HATU (71.3 mg, 0.187 mmol), and N-ethyl-N-isopropylpropan-2-amine (68 μL, 0.39 mmol) were added and the reaction mixture was stirred at r.t. until complete disappearance of the starting material. The mixture was filtered and purified by preparative HPLC to 4-(methyl(phenyl)amino)-N-(2,2,6,6-tetramethylpiperidin-4-yl)benzamide (27 mg, 36%) as a white solid. [M+H]⁺=366; ¹H NMR (DMSO-d₆) δ: 8.58-8.67 (m, 1H), 8.14 (d, J=7.6 Hz, 1H), 7.75-7.83 (m, 1H), 7.69 (d, J=8.7 Hz, 2H), 7.32-7.42 (m, 2H), 7.08-7.20 (m, 3H), 6.82 (d, J=8.7 Hz, 2H), 4.22-4.36 (m, 1H), 3.28 (s, 3H), 1.92 (dd, J=13.5, 2.7 Hz, 2H), 1.52 (t, J=12.9 Hz, 2H), 1.41 (s, 6H), 1.35 (s, 6H).

### Example 20

### 4-Benzyl-N-(2,2,6,6-tetramethylpiperidin-4-yl)benzamide (I-127)

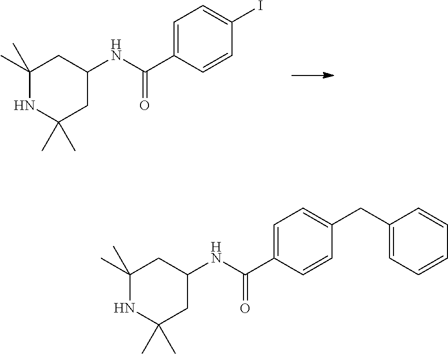 [SMILES: CC1(C)CC(NC(=O)c2ccc(I)cc2)CC(C)(C)N1 ; CC1(C)CC(NC(=O)c2ccc(Cc3ccccc3)cc2)CC(C)(C)N1]

A flame dried flask filled with nitrogen was charged with zinc chloride (3.1 mL, 1.5 mmol, 0.5M in THF). The solution was cooled to 0° C. and benzylmagnesium chloride (777 μL, 1.55 mmol. 2.0M in THF) was slowly added dropwise. The reaction mixture was stirred at r.t. 1 hour and 4-iodo-N-(2,2,6,6-tetramethylpiperidin-4-yl)benzamide (150 mg, 0.388 mmol) was added followed by PEPPSI-iPr (13 mg, 0.019 mmol). The reaction mixture was stirred at r.t. 2 hours, diluted with aq. sat. sodium bicarbonate (100 mL) and extracted with ethyl acetate (2×100 mL). The combined organic phases were washed with aq. sat. sodium bicarbonate (100 mL), brine (100 mL) dried over sodium sulfate, filtered and concentrated under reduced pressure. The residue was purified by preparative HPLC to afford 4-benzyl-N-(2,2,6,6-tetramethylpiperidin-4-yl)benzamide (58 mg, 32%) as a white solid. [M+H]⁺=351; ¹H NMR (DMSO-d₆) δ: 8.85 (d, J=11.9 Hz, 1H), 8.39 (d, J=7.3 Hz, 1H), 7.96 (d, J=12.4 Hz, 1H), 7.76 (d, J=8.2 Hz, 2H), 7.14-7.35 (m, 7H), 4.31 (td, J=7.8, 4.2 Hz, 2H), 3.98 (s, 2H), 1.93 (dd, J=13.4, 2.9 Hz, 2H), 1.57 (t, J=12.9 Hz, 2H), 1.34-1.46 (m, 12H).

### Example 21

### N-Methyl-4-phenoxy-N-(piperidin-4-yl)benzamide 2,2,2-trifluoroacetate (I-125)

### tert-Butyl 4-(N-methyl-4-phenoxybenzamido)piperidine-1-carboxylate

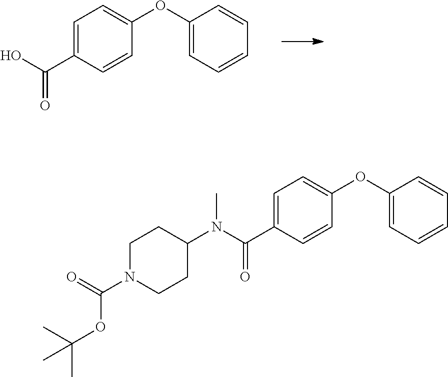 [SMILES: O=C(O)c1ccc(Oc2ccccc2)cc1 ; CN(C(=O)c1ccc(Oc2ccccc2)cc1)C1CCN(C(=O)OC(C)(C)C)CC1]

4-Phenoxybenzoic acid (100 mg, 0.467 mmol) was suspended in DCM (5 mL). The solution was cooled to 0° C. and tert-butyl 4-(methylamino)piperidine-1-carboxylate (100 mg, 0.467 mmol), HOBT (71 mg, 0.47 mmol), and EDC (89 mg, 0.47 mmol) were successively added. The reaction mixture was stirred at r.t. until complete disappearance of the starting material. Silica gel was added to the crude reaction mixture and the volatiles were removed under rotary evaporation. The crude material was purified by column chromatography (50% EtOAc/Hexanes) to provide tert-butyl 4-(N-methyl-4-phenoxybenzamido)piperidine-1-carboxylate (121 mg, 0.295 mmol, 63%) as a colorless oil.

### N-Methyl-4-phenoxy-N-(piperidin-4-yl)benzamide 2,2,2-trifluoroacetate (I-125)

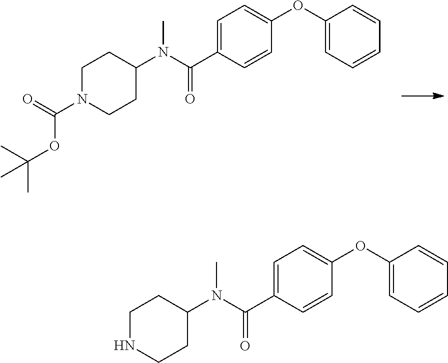 [SMILES: CN(C(=O)c1ccc(Oc2ccccc2)cc1)C1CCN(C(=O)OC(C)(C)C)CC1 ; CN(C(=O)c1ccc(Oc2ccccc2)cc1)C1CCNCC1]

4-(N-Methyl-4-phenoxybenzamido)piperidine-1-carboxylate (60.5 mg, 0.147 mmol) was dissolved in DCM (3 mL). The solution was cooled to 0° C. and trifluoroacetic acid (0.5 mL, 6.5 mmol) was added. Upon consumption of the starting material the volatiles were removed by rotary evaporation, the crude residue was dissolved in MeOH:H₂O (2:1, 3 mL) and purified by preparative HPLC to afford N-methyl-4-phenoxy-N-(piperidin-4-yl)benzamide 2,2,2-trifluoroacetate (59.1 mg, 0.139 mmol, 94%) as a white solid. [M+H]⁺=311; ¹H NMR (400 MHz DMSO-d₆) δ=8.62-8.52 (m, 1H), 8.26-8.15 (m, 1H), 7.47-7.38 (m, 4H), 7.23-7.16 (m, 1H), 7.11-7.05 (m, 2H), 7.03-6.97 (m, 2H), 4.53-4.36 (m, 1H), 3.73 (br. s., 4H), 2.97 (br. s., 1H), 2.80 (s, 3H), 2.02-1.87 (m, 2H), 1.86-1.78 (m, 2H).

The following compound was prepared in a similar manner to Example 21.

| Compound | Structure | Data |
|---|---|---|
| I-126 | 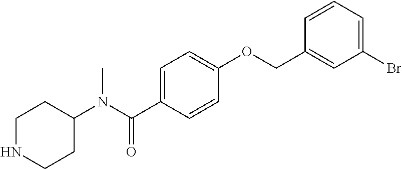 [SMILES: CN(C(=O)c1ccc(OCc2cccc(Br)c2)cc1)C1CCNCC1] | [M + H]⁺ = 403/404; ¹H NMR (400 MHz DMSO-d₆) δ = 8.65 (br. s., 1 H), 8.32 (br. s., 1 H), 7.54-7.47 (m, 2 H), 7.45-7.38 (m, 2 H), 7.29-7.22 (m, 2 H), 7.14 (d, J = 8.7 Hz, 1 H), 7.03 (dd, J = 1.8, 8.2 Hz, 1 H), 5.17 (s, 2 H), 4.54 (br. s., 1 H), 4.19 (br. s., 4 H), 3.05 (br. s., 1 H), 2.78 (br. s., 3 H), 1.96 (d, J = 12.1 Hz, 2H), 1.86-1.78 (m, 2 H) |

### Example 22

### 3-Chloro-4-(2-cyano-3-(pyrimidin-4-yl)phenoxy)-N-(2,2,6,6-tetramethylpiperidin-4-yl)benzamide (I-480)

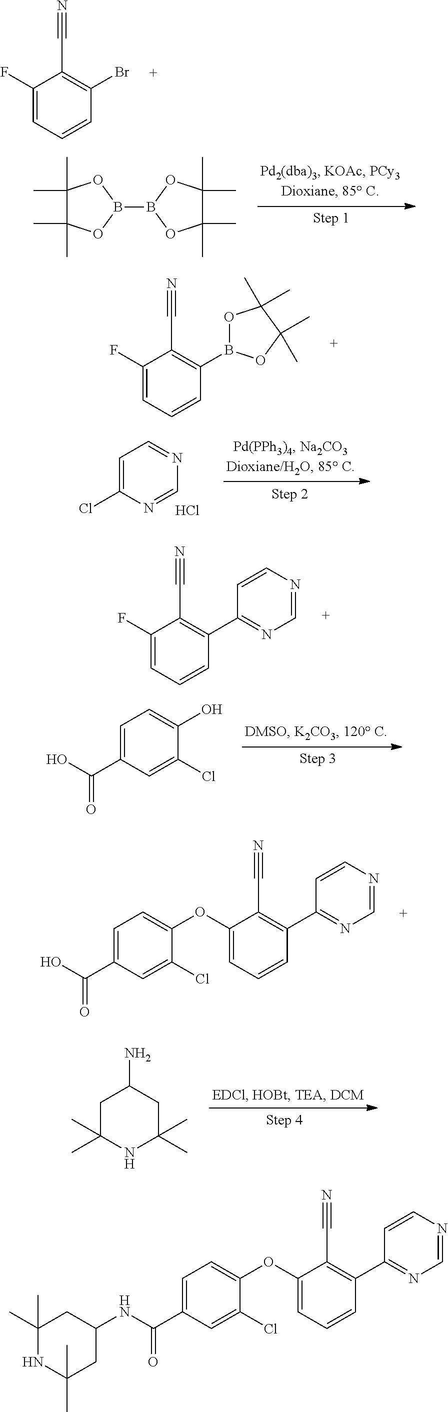

### 2-Fluoro-6-(4,4,5,5-tetramethyl-1,3,2-dioxaborolan-2-yl)benzonitrile

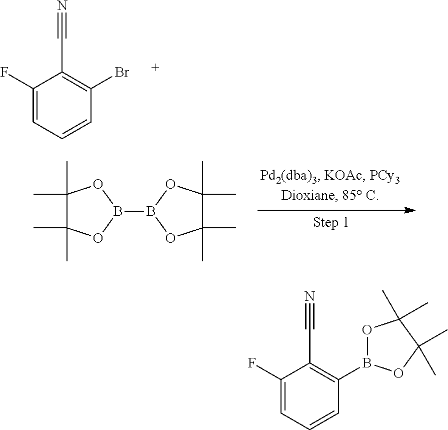

A mixture of 2-bromo-6-fluorobenzonitrile (198 mg, 1 mmol), tris(dibenzylideneacetone) dipalladium(0) (90 mg, 0.1 mmol), 4,4,4′,4′,5,5,5′,5′-octamethyl-2,2′-bi(1,3,2-dioxaborolane) (507 mg, 2 mmol), potassium acetate (0.3 g, 3 mmol) and tricyclohexyl phosphine (28 mg, 0.1 mmol) in dioxane (10 mL) was stirred at 85° C. for 12 hours, then filtered the solid. The filtrate was concentrated in vacuum. To the residue, ethyl acetate (20 mL) was added. The mixture was washed with water (20 mL). The organic phase was concentrated and the residue was purified by column chromatography (ethyl acetate/petroleum ether=1:5) to give 2-Fluoro-6-(4,4,5,5-tetramethyl-1,3,2-dioxaborolan-2-yl)benzonitrile as a yellow solid (98 mg, 39.6%). ¹H NMR (300 MHz, CDCl₃): δ 7.52 (m, 2H), 7.10 (m, 1H), 1.33 (d, J=4.69 Hz, 12H).

### 2-Fluoro-6-(pyrimidin-4-yl)benzonitrile

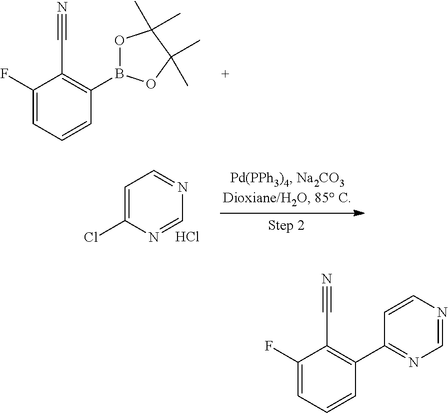 [SMILES: CC1(C)OB(c2cccc(F)c2C#N)OC1(C)C ; Clc1ccncn1 ; Cl ; N#Cc1c(F)cccc1-c1ccncn1]

2-Fluoro-6-(4,4,5,5-tetramethyl-1,3,2-dioxaborolan-2-yl)benzonitrile (247 mg, 1 mmol), 4-chloropyrimidine hydrochloride (180 mg, 1.2 mmol), palladium-tetrakis(triphenylphosphine) (116 mg, 0.1 mmol) and sodium carbonate (212 mg, 2 mmol) was dissolved in the mixture solvent of dioxane/water (20 mL/4 mL). And then the reaction mixture was stirred at 80° C. for 12 hours. The mixture was filtered, the filtrate was concentrated in vacuum, the residue was purified by preparative-HPLC to give 2-Fluoro-6-(pyrimidin-4-yl)benzonitrile as a pale yellow solid (40 mg, 20%). LRMS (M+H⁺) m/z: calcd 200.05. found 200.

### 3-Chloro-4-(2-cyano-3-(pyrimidin-4-yl)phenoxy)benzoic acid

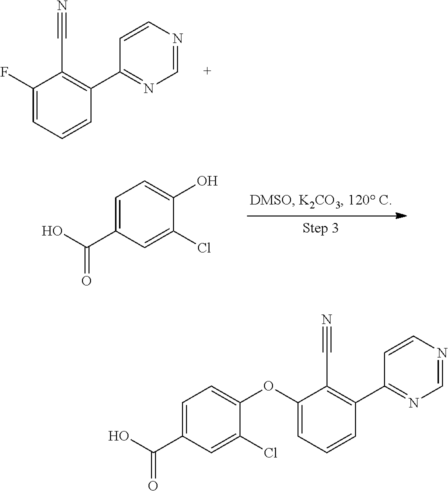 [SMILES: N#Cc1c(F)cccc1-c1ccncn1 ; O=C(O)c1ccc(O)c(Cl)c1 ; N#Cc1c(Oc2ccc(C(=O)O)cc2Cl)cccc1-c1ccncn1]

2-Fluoro-6-(pyrimidin-4-yl)benzonitrile (40 mg, 0.2 mmol), 3-chloro-4-hydroxybenzoic acid (53 mg, 0.3 mmol) and potassium carbonate (138 mg, 1 mmol) was dissolved in dimethyl sulfoxide (20 mL). The mixture was stirred at 120° C. for 12 hours. To the mixture, water (40 mL) was added. The mixture was extracted with ethyl acetate (20 mL×3), the combined organic layer was concentrated in vacuum. The residue was purified by preparative-HPLC to give 3-Chloro-4-(2-cyano-3-(pyrimidin-4-yl)phenoxy)benzoic acid as a white solid (60 mg, 86%). LRMS (M−H) m/z: calcd 351.04. found 351.

### 3-Chloro-4-(2-cyano-3-(pyrimidin-4-yl)phenoxy)-N-(2,2,6,6-tetramethyl piperidin-4-yl)benzamide (I-480)

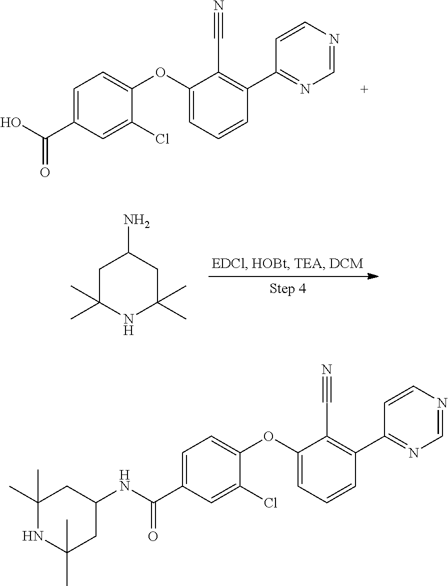

3-Chloro-4-(2-cyano-3-(pyrimidin-4-yl)phenoxy)benzoic acid (45 mg, 0.13 mmol), 1-(3-dimethylaminopropyl)-3-ethylcarbodiimide hydrochloride (48 mg, 0.25 mmol), 1H-benzo[d][1,2,3]triazol-1-ol (34 mg, 0.25 mmol) and triethylamine (50 mg, 0.5 mmol) was dissolved in dichloromethane (10 mL) and then stirred at room temperature for 2 hours. And then 2,2,6,6-tetramethyl-piperidin-4-ylamine(32 mmg, 0.2 mmol) was added, the mixture was stirred at room temperature for 12 hours. To the mixture, water (30 mL) was added. The mixture was extracted with dichloromethane (30 mL×3). The combined organic phase was concentrated in vacuum. The residue was purified by preparative-HPLC to give 3-Chloro-4-(2-cyano-3-(pyrimidin-4-yl)phenoxy)-N-(2,2,6,6-tetramethyl piperidin-4-yl)benzamide as a white solid (8 mg, 14%). ¹H NMR (300 MHz, CD₃OD): δ 9.33 (s, 1H), 8.96 (d, J=5.4 Hz, 1H), 8.53 (s, 1H), 8.08 (d, J=1.8 Hz, 1H), 8.00 (dd, J=1.2 Hz, J=5.1 Hz, 1H), 7.89 (dd, J=1.5 Hz, J=6.3 Hz, 1H), 7.75 (m, 2H), 7.31 (d, J=8.4 Hz, 1H), 7.08 (dd, J=1.5 Hz, J=6.3 Hz, 1H), 4.51 (m, 1H), 2.15 (m, 2H) 1.63 (m, 2H), 1.51 (d, J=30.6 Hz, 12H). LRMS (M+H⁺) m/z: calcd 489.19. found 489.

### Example 23

### 3-chloro-4-(2-cyano-3-(pyridazin-4-yl)phenoxy)-N-(2,2,6,6-tetramethyl piperidin-4-yl)benzamide (I-378)

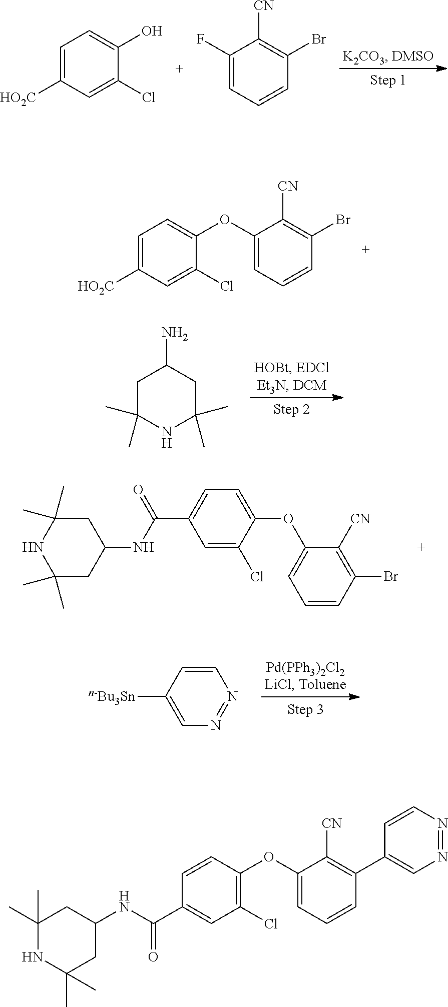

### 4-(3-bromo-2-cyanophenoxy)-3-chlorobenzoic acid

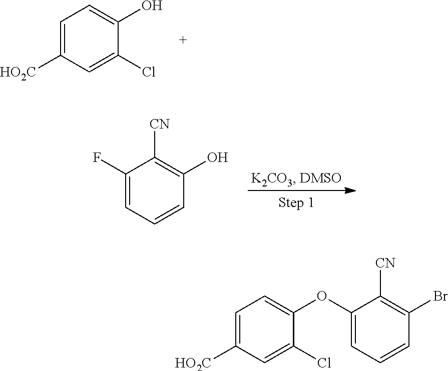 [SMILES: O=C(O)c1ccc(O)c(Cl)c1 ; N#Cc1c(O)cccc1F ; N#Cc1c(Br)cccc1Oc1ccc(C(=O)O)cc1Cl]

To a solution of 3-chloro-4-hydroxybenzoic acid (5.16 g, 30 mmol) and 2-bromo-6-fluorobenzonitrile (7.2 g, 36 mmol) in dimethyl sulfoxide (100 mL) was added potassium carbonate (10.4 g, 75 mmol), and then stirred at 140° C. for 3 hours, cooled to room temperature, water (400 ml) was added and then acidified to pH=2 with concentrated hydrochloric acid, the solid was collected by filtration and washed with water (40 mL), methanol (20 mL) in turns, dried to afford product 4-(3-bromo-2-cyanophenoxy)-3-chlorobenzoic acid 4-(3-bromo-2-cyanophenoxy)-3-chlorobenzoic acid as a white solid (8.5 g, 81%). LRMS (M−H)⁻ m/z: calcd 350.93. found 350. ¹H NMR (300 MHz, d⁶-DMSO): δ 8.11 (d, J=2.1 Hz, 1H), 7.97 (dd, J=8.4 Hz, J=2.1 Hz, 1H), 7.69-7.58 (m, 2H), 7.43 (d, J=8.4 Hz, 2H), 7.01 (dd, J=8.1 Hz, J=1.2 Hz, 1H).

### 4-(3-bromo-2-cyanophenoxy)-3-chloro-N-(2,2,6,6-tetramethylpiperidin-4-yl)benzamide

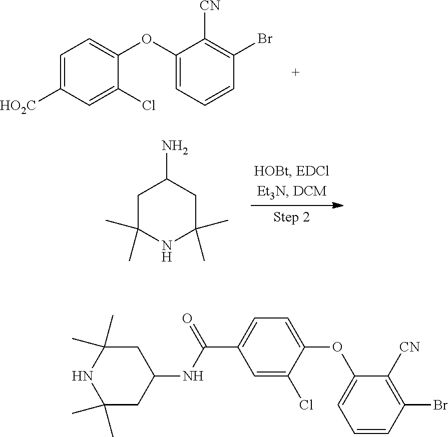

To a solution of 4-(3-bromo-2-cyanophenoxy)-3-chlorobenzoic acid (8.5 g, 24.2 mmol) in anhydrous dichloromethane (200 mL) was added 1H-benzo[d][1,2,3]triazol-1-ol (4.9 g, 36 mmol), 1-ethyl-3-(3-dimethylaminopropyl) carbodiimide hydrochloride (7.1 g, 37 mmol) and triethylamine (8.4 mL, 60 mmol). The mixture was stirred at room temperature for 0.5 hour, 2,2,6,6-tetramethylpiperidin-4-amine (4.7 mg, 30 mmol) was added and stirred at room temperature for 3 hours. To the reaction mixture was added water (200 mL), extracted with dichloromethane (200 mL×2), combined the organic phase and then concentrated, the residue was purified by column chromatography (dichloromethane/methanol=10:1) to afford 4-(3-bromo-2-cyanophenoxy)-3-chloro-N-(2,2,6,6-tetramethyl-piperidin-4-yl)benzamide (3.5 g, 30%). LRMS (M+H⁺) and (M+H⁺+2) m/z: calcd 489.08 and 491.08. found 489 and 491. ¹H NMR (DMSO-d⁶, 300 MHz): δ 8.39 (d, J=7.5 Hz, 1H), 8.16 (s, 1H), 7.93 (d, J=8.4 Hz, 2H), 7.67-7.56 (m, 2H), 7.46 (d, J=8.7 Hz, 1H), 6.92 (d, J=8.1 Hz, 1H), 4.35-4.26 (m, 1H), 1.72 (d, J=12.3 Hz, 2H), 1.20-1.07 (m, 14H).

### 3-chloro-4-(2-cyano-3-(pyridazin-4-yl)phenoxy)-N-(2,2,6,6-tetramethylpiperidin-4-yl)benzamide

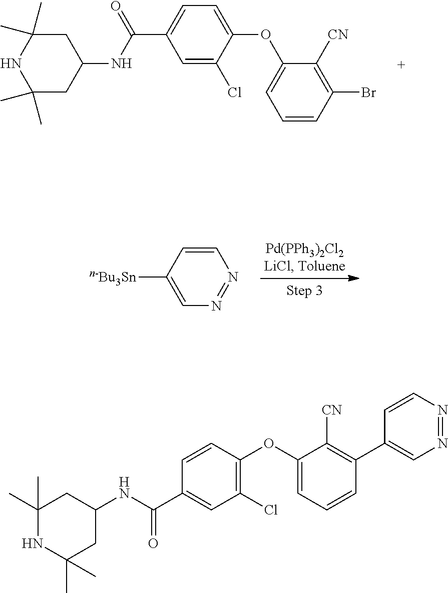

To a solution of 4-(3-bromo-2-cyanophenoxy)-3-chloro-N-(2,2,6,6-tetramethylpiperidin-4-yl)benzamide (0.20 g, 0.40 mmol) and 4-(tributylstannyl)pyridazine (0.30 g, 0.81 mmol) in anhydrous toluene (10 mL) was added bis(triphenylphosphine) palladium(II) dichloride (0.03 g, 0.04 mmol) and lithium chloride (0.06 g, 1.5 mmol). The reaction mixture was stirred for 12 hours at 90° C. After the reaction, the mixture was filtered and the filtrate was concentrated. The residue was purified by column chromatography (dichloromethane/methol=8:1) to give the pure product 3-chloro-4-(2-cyano-3-(pyridazin-4-yl)phenoxy)-N-(2,2,6,6-tetramethyl piperidin-4-yl)benzamide as a white solid (0.12 g, 60%). ¹H NMR (300 MHz, CD₃OD) δ: 9.508 (q, J=1.2 Hz, 1H), 9.377 (dd, J=1.2 Hz, J=5.4 Hz, 1H), 8.105 (d, J=2.1 Hz, 1H), 8.055 (dd, J=2.4 Hz, J=5.4 Hz, 1H), 7.914 (dd, J=1.8 Hz, J=8.7 Hz, 1H), 7.788 (t, J=7.8 Hz, 1H), 7.516 (d, J=7.8 Hz, 1H), 7.357 (d, J=8.7 Hz, 1H) 7.046 (d, J=8.4 Hz, 1H), 4.487-4.568 (m, 1H), 2.155 (dd, J=2.7 Hz, J=13.8 Hz, 2H), 1.670˜1.762 (m, 2H), 1.592 (s, 6H), 1.510 (s, 6H). LRMS (M+H⁺) m/z: calcd for 490. found 490.

### Example 24

### 3-chloro-4-(2-cyano-3-(pyridazin-4-yl)phenoxy)-N-(2,2,6,6-tetramethyltetrahydro-2H-pyran-4-yl)benzamide (I-473)

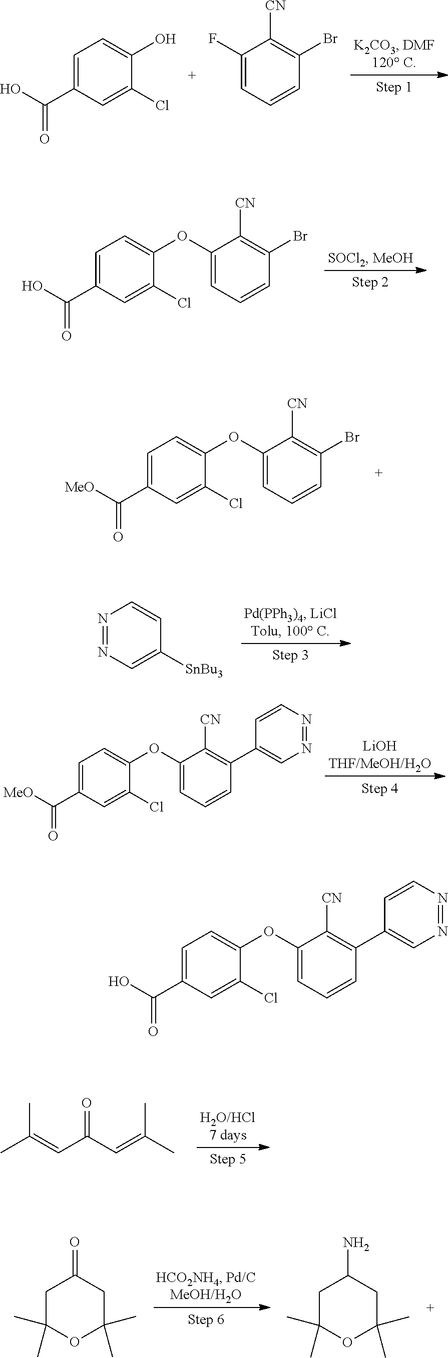

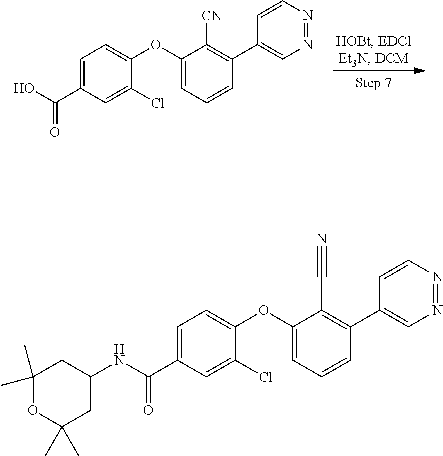 [SMILES: N#Cc1c(Oc2ccc(C(=O)O)cc2Cl)cccc1-c1ccnnc1 ; CC1(C)CC(NC(=O)c2ccc(Oc3cccc(-c4ccnnc4)c3C#N)c(Cl)c2)CC(C)(C)O1]

### 4-(3-bromo-2-cyanophenoxy)-3-chlorobenzoic acid

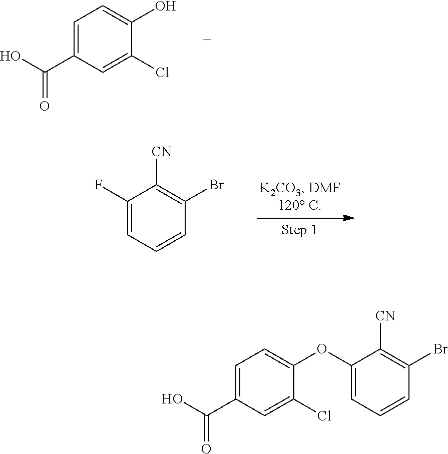 [SMILES: O=C(O)c1ccc(O)c(Cl)c1 ; N#Cc1c(F)cccc1Br ; N#Cc1c(Br)cccc1Oc1ccc(C(=O)O)cc1Cl]

To a solution of 3-chloro-4-hydroxybenzoic acid (8.6 g, 50 mmol) in N,N-dimethylformamide (120 mL) was added 2-bromo-6-fluorobenzonitrile (11 g, 55 mol) and potassium carbonate (13.8 g, 100 mmol). Then the mixture was stirred at 120° C. for 12 hours. The reaction mixture was poured into water, and then the mixture was extracted with ethyl acetate (20 ml*3). The combined organic phase was dried by anhydrous sodium sulphate. And then the mixture was filtered, the filtrate was evaporated and the residue was purified by column chromatography to give 4-(3-bromo-2-cyanophenoxy)-3-chlorobenzoic acid as yellow solid (15.8 g, 89%), which was used for next step directly. LRMS (M+H⁺) m/z: calcd 352.56. found 352.

### Methyl 4-(3-bromo-2-cyanophenoxy)-3-chlorobenzoate

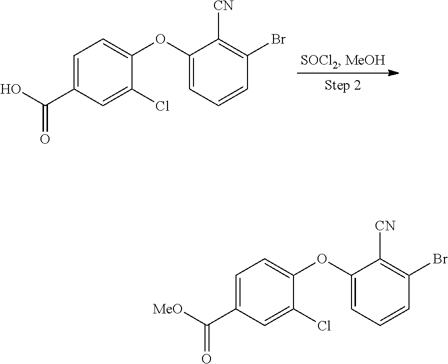 [SMILES: N#Cc1c(Br)cccc1Oc1ccc(C(=O)O)cc1Cl ; COC(=O)c1ccc(Oc2cccc(Br)c2C#N)c(Cl)c1]

To a solution of 4-(3-bromo-2-cyanophenoxy)-3-chlorobenzoic acid (7 g, 20 mmol) in methanol (100 mL) was added thionyl chloride (4.0 g, 22 mmol). The mixture was stirred at 20° C. for 12 hours. The reaction mixture was poured into water, and then the mixture was extracted with ethyl acetate (20 ml*3). The combined organic phase was dried by anhydrous sodium sulphate. And then the mixture was filtered, the filtrate was evaporated and the residue was purified by column chromatography to give methyl 4-(3-bromo-2-cyanophenoxy)-3-chlorobenzoate as white solid (4.4 g, 61%), which was used for next step directly. ¹H NMR (300 MHz, d⁶-DMSO): δ 8.20 (s, 1H), 7.99 (d, J=8.7 Hz, 1H), 7.43 (d, J=7.2 Hz, 1H), 7.37 (t, J=8.4 Hz, 1H), 7.15 (d, J=9.0 Hz, 1H), 6.68 (d, J=8.1 Hz, 1H), 3.95 (s, 3H).

### Methyl 3-chloro-4-(2-cyano-3-(pyridazin-4-yl)phenoxy)benzoate

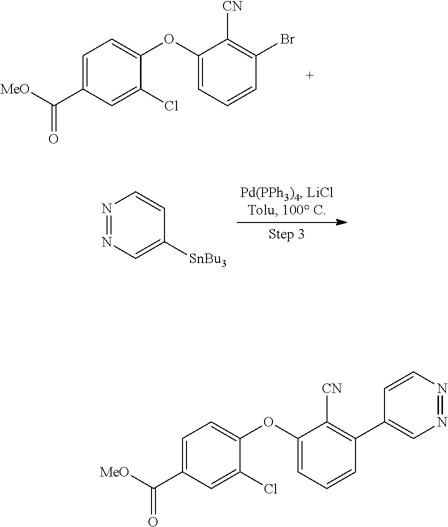

To a solution of methyl 4-(3-bromo-2-cyanophenoxy)-3-chlorobenzoate (4.4 g, 12 mmol) in toluene (100 ml) was added 4-(tributylstannyl)pyridazine (5.5 g, 15 mmol), lithium chloride (1.06 g, 24 mmol) and tetrakis(triphenylphosphine)palladium(0) (1.1 g, 1.0 mmol). The mixture was stirred at 100° C. under nitrogen atmosphere for 12 hours. Then evaporated the solvent and the residue was purified by flash chromatograph give the product methyl 3-chloro-4-(2-cyano-3-(pyridazin-4-yl)phenoxy)benzoate (4.0 g, 91%). ¹H NMR (300 MHz, d⁶-DMSO): δ 9.52 (s, 1H), 9.39 (d, J=5.1 Hz, 1H), 8.23 (s, 1H), 8.08-8.04 (m, 2H), 7.80-7.77 (m, 2H), 7.36 (d, J=7.2 Hz, 1H), 6.68 (d, J=8.1 Hz, 1H), 3.94 (s, 3H).

### 3-chloro-4-(2-cyano-3-(pyridazin-4-yl)phenoxy)benzoic acid

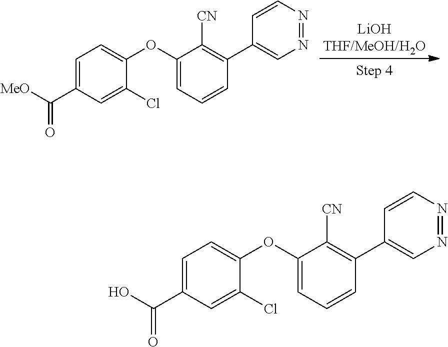 [SMILES: COC(=O)c1ccc(Oc2cccc(-c3ccnnc3)c2C#N)c(Cl)c1 ; N#Cc1c(Oc2ccc(C(=O)O)cc2Cl)cccc1-c1ccnnc1]

To a solution of methyl 3-chloro-4-(2-cyano-3-(pyridazin-4-yl)phenoxy)benzoate (4.0 g, 10.9 mmol) in the mixture solvent of tetrahydrofuran/methanol/water=3:1:1 (120 mL) was added lithium hydroxide (1.15 g, 48 mmol). The mixture was stirred at room temperature for 2 hours. The suspension was concentrated in vacuum and quenched with aqueous 1N hydrochloride acid (50 mL). The mixture was poured into water (50 mL) and then extracted with dichloridemethane (50 mL). The combined organic phase was dried by sodium sulphate. The mixture was filtered, the filtrate was evaporated and the residue was purified by column chromatography to give the product 3-chloro-4-(2-cyano-3-(pyridazin-4-yl)phenoxy)benzoic acid as a white solid (2.2 g, 41%). LRMS (M+H⁺) m/z: calcd 352. found 352.1H NMR (300 MHz, CD₃OD): δ 9.53 (s, 1H), 9.39 (d, J=5.4 Hz, 1H), 8.23 (s, 1H), 8.08-8.05 (m, 2H), 7.08 (t, J=8.7 Hz, 1H), 7.53 (d, J=7.8 Hz, 1H), 7.08 (d, J=8.4 Hz, 1H).

### 2,2,6,6-tetramethyldihydro-2H-pyran-4(3H)-one

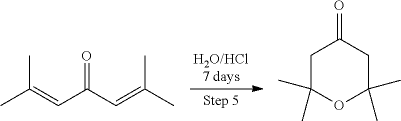

2,6-dimethylhepta-2,5-dien-4-one (25 g, 0.18 mol) was suspended in 1N hydrochloric acid aqueous (250 ml). The mixture was heated at 40° C. for 7 days. And then the mixture was cooled to room temperature. The mixture was extracted with ether (20 ml*3). The organic phase was dried by sodium sulphate. The mixture was filtered, the filtrate was evaporated, the residue was purified by column chromatography to give the product 2,2,6,6-tetramethyldihydro-2H-pyran-4(3H)-one as a yellow oil (5 g, 20%). LRMS (M+H⁺) m/z: calcd 156.12. found 156. ¹H NMR (300 MHz, CD₃OD): δ 2.17 (s, 2H), 1.95 (s, 2H), 1.33 (s, 12H).

### 2,2,6,6-tetramethyltetrahydro-2H-pyran-4-amine

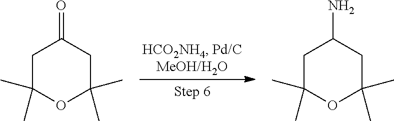

To a solution of 2,2,6,6-tetramethyldihydro-2H-pyran-4(3H)-one (1.5 g, 9.6 mmol) in methanol (20 ml) was added palladium carbon (0.96 mmol) and ammonium anetate (4.4 g, 7 0 mmol) in water (3 ml). The mixture was stirred at room temperature under nitrogen atmosphere, and the reaction was monitored by thin layer chromatography. Then the solvent was evaporated under reduced pressure and the residue was purified by flash column chromatography to give the product 2,2,6,6-tetramethyltetrahydro-2H-pyran-4-amine as an oil (1 g, 67%). LRMS (M+H⁺) m/z: calcd 157.15. found 157. ¹H NMR (300 MHz, d₆-DMSO): δ 2.93-3.03 (m, 1H), 1.65 (dd, J=9 Hz, J=3.6 Hz, 2H), 1.16 (s, 6H), 1.08 (s, 6H), 0.89 (t, J=12 Hz, 2H).

### 3-chloro-4-(2-cyano-3-(pyridazin-4-yl)phenoxy)-N-(2,2,6,6-tetramethyltetrahydro-2H-pyran-4-yl)benzamide (I-473)

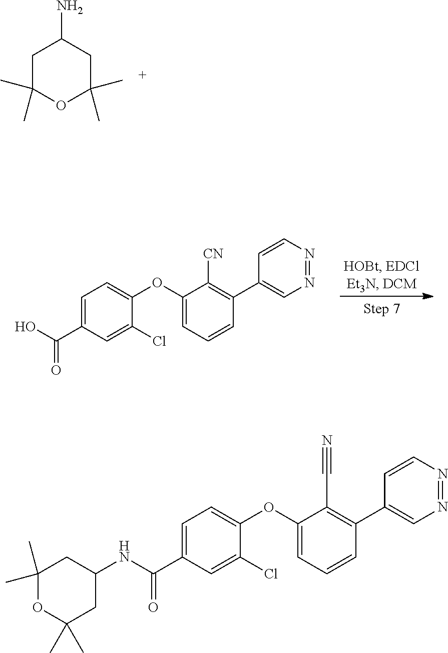

To a solution of 3-chloro-4-(2-cyano-3-(pyridazin-4-yl)phenoxy)benzoic acid (500 mg, 1.42 mmol), 3-(3-dimethylaminopropyl)-(1-ethylcarbodiimide hydrochloride (542 mg, 2.84 mmol) and N-hydroxybenzotrizole (383 mg, 2.84 mmol) in methylene chloride (20 mL) was added triethylamine (0.2 mL). The reaction mixture was stirred at room temperature for 15 minutes, and then 3-c (223 mg, 1.42 mmol) was added. The reaction mixture was stirred at room temperature for 12 hours. After the reaction, the mixture was washed with sodium bicarbonate aqueous, and then filtered, the filtrate was dried by anhydrous sodium sulphate, concentrated the solvent. The residue was purified by preparative-HPLC to give 3-chloro-4-(2-cyano-3-(pyridazin-4-yl)phenoxy)-N-(2,2,6,6-tetramethyltetrahydro-2H-pyran-4-yl)benzamide as a white solid (580 mg, 80%). LRMS (M+H⁺) m/z: calcd 490.18. found 490. ¹H NMR (300 MHz, CD₃OD): δ 9.53 (s, 1H), 9.4 (d, J=5.4 Hz, 1H), 8.06-8.10 (m, 2H), 7.97 (d, J=7.8 Hz, 1H), 7.82 (t, J=7.8 Hz, 1H), 7.53 (d, J=7.8 Hz, 1H), 7.37 (d, J=9 Hz, 1H) 7.04 (d, J=8.7 Hz, 1H), 4.51 (t, J=8.4 Hz, 1H), 2.1 (d, J=10.2 Hz, 2H), 1.6 (t, J=12.6 Hz, 2H), 1.47 (s, 6H), 1.44 (s, J=7.2 Hz, 6H).

### Example 25

### 2-(2-chloro-4-(((2,2,6,6-tetramethylpiperidin-4-yl)oxy)methyl)phenoxy)-6-(pyrimidin-4-yl)benzonitrile (I-591)

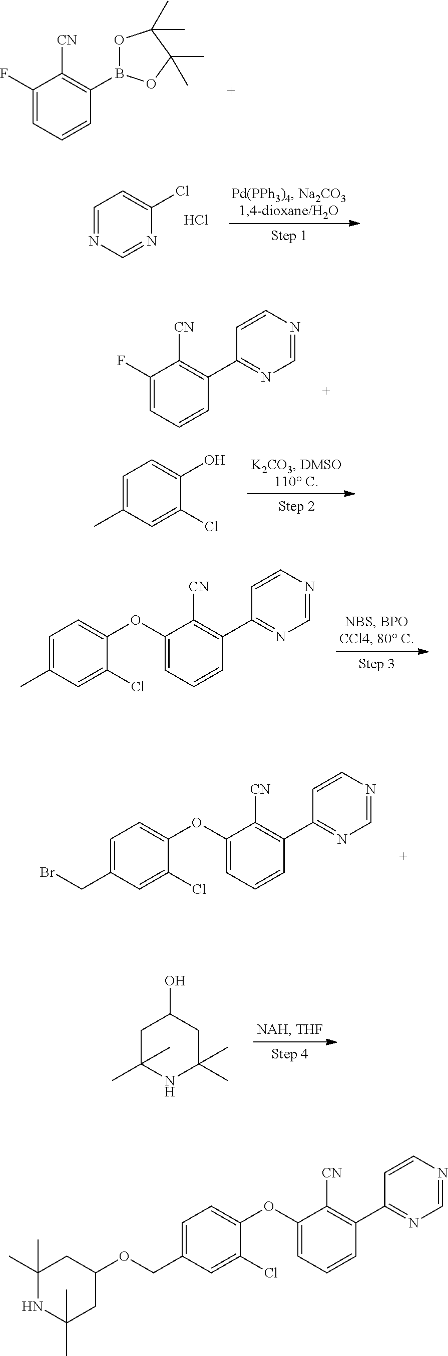

### 2-fluoro-6-(pyrimidin-4-yl)benzonitrile

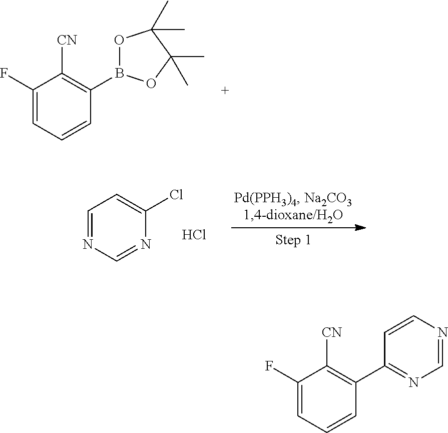 [SMILES: CC1(C)OB(c2cccc(F)c2C#N)OC1(C)C ; Clc1ccncn1 ; Cl ; N#Cc1c(F)cccc1-c1ccncn1]

To a solution of 2-fluoro-6-(4,4,5,5-tetramethyl-1,3,2-dioxaborolan-2-yl)benzonitrile (6.0 g, 24.3 mmol) and 4-chloropyrimidine hydrochloride (4.2 g, 27.8 mmol) in the mixture solvent of 1,4-dioxane (10 mL) and water (1.0 mL) was added tetrakis(triphenylphosphine) palladium(0) (1.0 g, 0.86 mmol) and sodium carbonate (5.3 g, 50 mmol). The mixture was stirred for 12 hours at 90° C. After the reaction, the mixture was poured into water (50 mL), and the mixture was extracted with ethyl acetate (50 m L×3), the combined organic phase was washed with water, dried by anhydrous sodium sulfate. The mixture was filtered and the filtrate was concentrated, the residue was purified by column chromatography (ethyl acetate/petroleum ether=1:2) to give the 2-fluoro-6-(pyrimidin-4-yl)benzonitrile as a yellow solid (0.52 g, 11%). LRMS (M+H⁺): calcd 200. found 200.

### 2-(2-chloro-4-methylphenoxy)-6-(pyrimidin-4-yl)benzonitrile

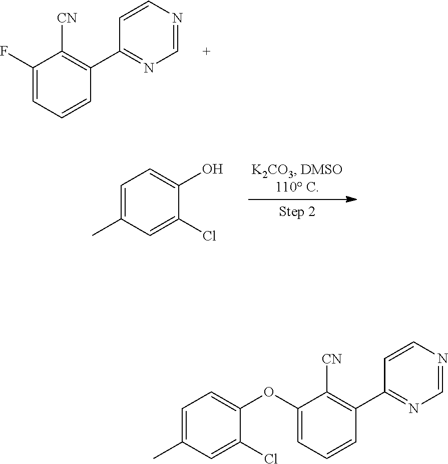

To a solution of (0.15 g, 0.75 mmol) and 2-chloro-4-methylphenol (0.11 g, 0.77 mmol) in N,N-dimethyl formamide (15 mL) was added potassium carbonate (0.2 g, 1.45 mmol). The reaction mixture was stirred for 12 hours at 110° C. After the reaction, the mixture was diluted with water (50 mL) and the mixture was extracted with ethyl acetate (80 mL×3). The combined organic phase was dried by anhydrous sodium sulfate. The mixture was filtered and the filtrate was concentrated and purified by column chromatography (ethyl acetate/petroleum=1:4) to give 2-(2-chloro-4-methylphenoxy)-6-(pyrimidin-4-yl)benzo-nitrile as the yellow solid (0.22 g, 91%). LRMS (M+H⁺): calcd 322. found 322.

### 2-(4-(bromomethyl)-2-chlorophenoxy)-6-(pyrimidin-4-yl)benzonitrile

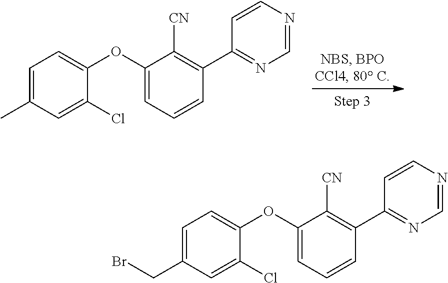 [SMILES: Cc1ccc(Oc2cccc(-c3ccncn3)c2C#N)c(Cl)c1 ; N#Cc1c(Oc2ccc(CBr)cc2Cl)cccc1-c1ccncn1]

To a solution of 2-(2-chloro-4-methylphenoxy)-6-(pyrimidin-4-yl)benzonitrile (0.16 g, 0.5 mmol) and N-bromosuccinimide (0.09 g, 0.5 mmol) in perchloromethane (10 mL) was added benzoic peroxyanhydride (0.03 g, 0.12 mmol). The reaction mixture was stirred at 80° C. for 3 hours. After the reaction, the mixture was quenched by sodium thiosulphate aqueous (30 mL), and then the mixture was extracted with dichloromethane (30 mL×3). The combined organic phase was dried by anhydrous sodium sulfate. The mixture was filtered and the filtrate was concentrated and purified by column chromatography (ethyl acetate/petroleum ether=1:5) to give 2-(4-(bromomethyl)-2-chlorophenoxy)-6-(pyrimidin-4-yl)benzonitrile as a yellow solid (0.12 g, 60%). LRMS (M+H⁺): calcd 402. found 402.

### 2-(2-chloro-4-((2,2,6,6-tetramethylpiperidin-4-yloxy)methyl)phenoxy)-6-(pyrimidin-4-yl)benzonitrile (I-591)

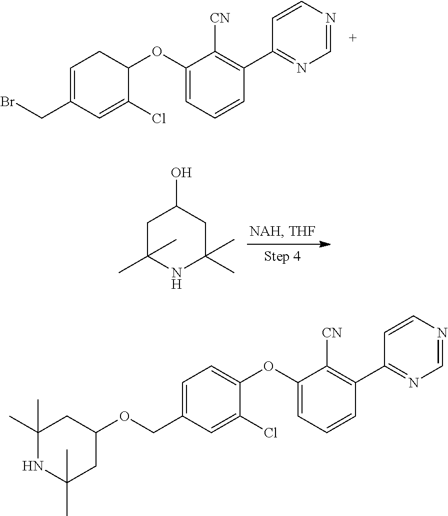

To a solution of 2-(4-(bromomethyl)-2-chlorophenoxy)-6-(pyrimidin-4-yl)-benzonitrile (0.15 g, 1.0 mmol) in tetrahydrofuran (10 mL) was added sodium hydride(60% in oil) (0.04 g, 1.0 mmol). The reaction mixture was stirred for 15 minutes at room temperature, then 2,2,6,6-tetramethylpiperidin-4-ol (0.06 g, 0.15 mmol) was added. The mixture was stirred at room temperature for 12 hours. After the reaction, the reaction was quench by water (20 mL), and the mixture was extracted by dichloromethane (30 mL×3). The organic phase was dried by anhydrous sodium sulfate, and then filtered. The filtrate was concentrated and purified by preparative-HPLC to give 2-(2-chloro-4-(((2,2,6,6-tetramethylpiperidin-4-yl)oxy)methyl)phenoxy)-6-(pyrimidin-4-yl)benzonitrile as a white solid (0.02 g, 27%). ¹H NMR (300 MHz, CD₃OD,): δ 9.372 (d, J=1.5 Hz, 1H), 9.008 (d, J=2.1 Hz, 1H), 8.037 (dd, J=1.5 Hz, J=5.4 Hz, 1H), 7.649-7.789 (m, 3H), 7.473 (dd, J=1.8 Hz, J=8.1 Hz, 1H), 7.320 (d, J=8.1 Hz, 1H), 6.964 (d, J=8.1 Hz, 1H), 4.702 (s, 2H), 4.064-4.131 (m, 1H), 2.247 (dd, J=3.9 Hz, J=14.1 Hz, 2H), 1.687 (dd, J=10.2, J=13.8 Hz, 2H), 1.558 (s, 6H), 1.537 (s, 6H). LR MS (M+H⁺): calcd 477. found 477.

### Example 26

### (E)-2-(2-chloro-4-(2-(2,2,6,6-tetramethylpiperidin-4-yl)vinyl)phenoxy)-6-(pyrimidin-4-yl)benzonitrile (I-592)

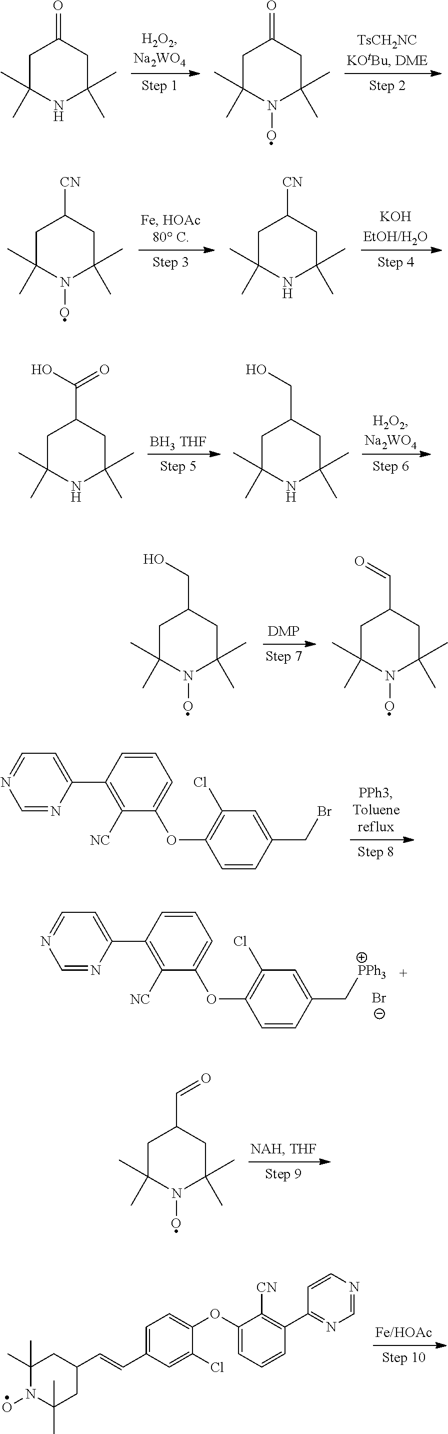

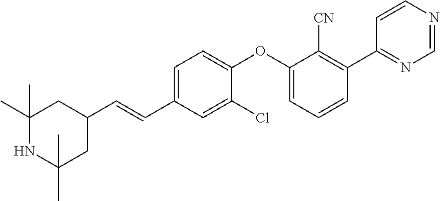

### 1-oxyl-2,2,6,6-tetramethylpiperidin-4-one

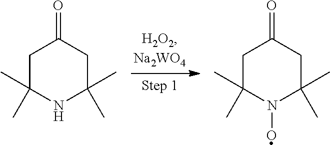

To a solution of 2,2,6,6-tetramethylpiperidin-4-one (11 g, 71 mmol) in hydrogen peroxide (20 mL, 30% in water) was added sodium tungstate dehydrate (1.0 g, 3.0 mmol) under ice bath. The reaction mixture was stirred at 0° C. for 1 hour, then stirred at room temperature for additional 1 hour. After the reaction, the mixture was poured into water (50 mL) and the mixture was extracted with ethyl acetate (100 mL×2). dried by anhydrous sodium sulfate. The mixture was filtered, the filtrate was concentrated and the residue was purified by column chromatography (ethyl acetate/petroleum ether=11:5) to give the pure product 1-oxyl-2,2,6,6-tetramethylpiperidin-4-one as a red solid (9.5 g, 79%).

### 1-oxyl-2,2,6,6-tetramethylpiperidine-4-carbonitrile

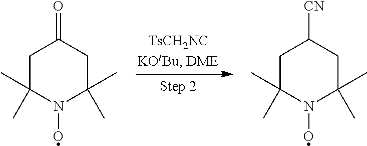

To a solution of 1-oxyl-2,2,6,6-tetramethylpiperidin-4-one (7.5 g, 44 mmol) and 1-(isocyanomethylsulfonyl)-4-methylbenzene (9.4 g, 48 mmol) in 1,2-dimethoxyethane (240 mL) was added the 1,2-dimethoxyethane solution (60 mL) of potassium t-butoxide (10 g, 90 mmol), t-butanol (60 mL) in follow at 0° C. The reaction mixture was stirred at 0° C. for 1 h and stirred at room temperature for additional 2 h. After the reaction, the mixture was poured into water (500 mL) and the mixture was extracted with diethyl ether (300 mL×3), the combined organic phase was dried by anhydrous sodium sulfate. The mixture was filtered, the filtrate was concentrated and the residue was purified by recrystallization with diethyl ether to give the pure product 1-oxyl-2,2,6,6-tetramethylpiperidine-4-carbonitrile as the red solid (5.4 g, 68%).

### 2,2,6,6-tetramethylpiperidine-4-carbonitrile

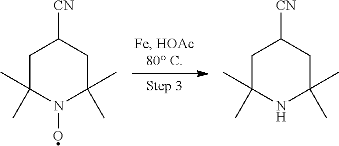

To a solution of 1-oxyl-2,2,6,6-tetramethylpiperidine-4-carbonitrile (5.0 g, 27.5 mmol) acetic acid (20 mL) was added iron powder (8.0 g, 143 mmol). The reaction mixture was stirred at 50° C. for 3 hours. After the reaction, the mixture was filtered and the filtrate was concentrated, then potassium carbonate aqueous was added to make pH=8˜10, and the mixture was extracted with dichloromathane (100 mL×3), washed by water (200 mL). The organic phase was dried by anhydrous sodium sulfate. The solvent was removed in vacuum to give 2,2,6,6-tetramethylpiperidine-4-carbonitrile as the white solid (4.2 g, 91%). LRMS (M+H⁺): calcd 167. found 167.

### 2,2,6,6-tetramethylpiperidine-4-carboxylic acid

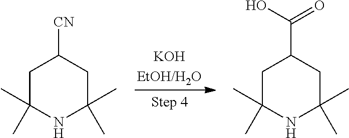

To a solution of 2,2,6,6-tetramethylpiperidine-4-carbonitrile (2.0 g, 12 mmol) in the mixture solvent of water (10.5 mL) and ethanol (12 mL) was added potassium hydroxide (4.5 g, 80 mmol). The reaction mixture was stirred at 100° C. for 12 hours. After the reaction, hydrochloric acid (3 mol/L) was added to make pH=5-6. Then the product was extracted by dichloromethane (100 mL×3). The solvent was removed in vacuo to give the crude product 2,2,6,6-tetramethylpiperidine-4-carboxylic acid as a white solid (1.9 g, 83%). LRMS (M+H⁺): calcd 186. found 186.

### (2,2,6,6-tetramethylpiperidin-4-yl)methanol

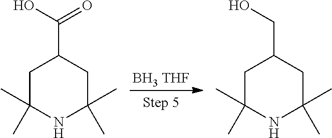

Borane (15 mL, 1M in tetrahydrofuran) was added to a solution of 2,2,6,6-tetramethylpiperidine-4-carboxylic acid (1.8 g, 1.0 mmol) in tetrahydrofuran (20 mL) at 0° C. under nitrogen atmosphere. The reaction mixture was stirred at room temperature for 3 hours, then hydrochloric acid (10 mL, 3 mol/L) was added to quench the reaction. Then potassium carbonate aqueous was added to make pH=8˜10. The mixture was extracted by dichloromethane (100 mL×3). The organic phase was dried by anhydrous sodium sulfate. The mixture was filtered, the filtrate was concentrated and the residue was purified by column chromatography(methanol/dichloromethane=15:1 with 1% NH3 aqueous) to give the pure product (2,2,6,6-tetramethylpiperidin-4-yl)methanol as a white solid (1.2 g, 70%). ¹H NMR (300 MHz, CDCl₃): δ 3.487 (d, J=6.3 Hz, 2H), 2.000˜2.041 (m, 1H), 1.660 (dd, 2H, J=3.0 Hz, J=12.9 Hz, 2H), 1.213 (s, 6H), 1.135 (s, 6H), 0.817 (t, J=11.7 Hz, 2H). LR MS (M+H⁺): calcd 172. found 172.

### (1-oxyl-2,2,6,6-tetramethylpiperidin-4-yl)methanol

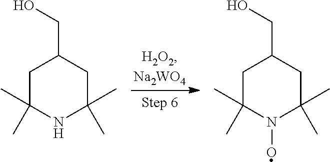

To a solution of (2,2,6,6-tetramethylpiperidin-4-yl)methanol (0.5 g, 2.9 mmol) in hydrogen peroxide (2 mL, aq, 30%) and water (10 mL) was added sodium tungstate dehydrate (0.10 g, 0.3 mmol) under ice bath. The reaction mixture was stirred at 0° C. for 1 hour, then stirred at room temperature for additional 1 hour. After the reaction, the mixture was poured into water (20 mL) and the mixture was extracted with ethyl acetate (50 mL×3). The organic phase was dried by anhydrous sodium sulfate. The mixture was filtered and the filtrate was concentrated, the residue was purified by column chromatography(dichloromethane/methanol=1:50) to give the pure product (1-oxyl-2,2,6,6-tetramethylpiperidin-4-yl)methanol (0.41 g, 76%) as a red solid. LRMS (M(NOH)+H⁺): calcd 188. found 188.

### 1-oxyl-2,2,6,6-tetramethylpiperidine-4-carbaldehyde

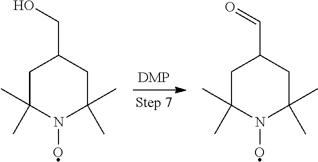

Dess-Martin periodinane (0.70 g, 1.6 5 mmol) was added to a solution of (1-oxyl-2,2,6,6-tetramethylpiperidin-4-yl)methanol (0.26 g, 1.40 mmol) in anhydrous dichloromethane (10 mL). The reaction mixture was stirred at room temperature for 1 hour, After the reaction, water (20 mL) was added, and the mixture was extracted with dichloromethane (50 mL×3). The organic phase was dried by anhydrous sodium sulfate. The mixture was filtered and the filtrate was concentrated. The residue was purified by column chromatography(ethyl acetate/petroleum ether=1:5) to give the pure product 1-oxyl-2,2,6,6-tetramethylpiperidine-4-carbaldehyde as the red solid (0.21 g, 81%). LRMS (M(NOH)+H⁺): calcd 186. found 186.

### (3-chloro-4-(2-cyano-3-(pyrimidin-4-yl)phenoxy)benzyl)triphenyl phosphonium bromide

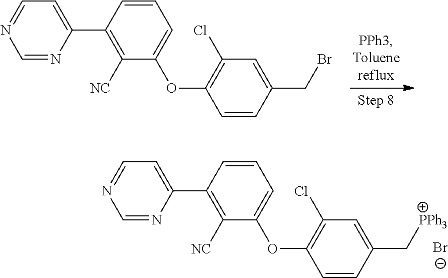

Triphenylphosphine (0.15 g, 0.57 mmol) was added to a solution of 2-(4-(bromomethyl)-2-chlorophenoxy)-6-(pyrimidin-4-yl)benzonitrile (0.15 g, 0.37 mmol) in toluene (10 mL). The reaction mixture was stirred at 100° C. for 20 hours under nitrogen protected. After the reaction, the mixture was filtered, the solid was collected, washed with ether, and dried under vacuum, the crude product (3-chloro-4-(2-cyano-3-(pyrimidin-4-yl)phenoxy)benzyl)triphenyl phosphonium bromide (0.19 g, 76%) was used directly for the next step without further purification. LR MS (M+H): calcd for 582. found 582.

### (E)-2-(2-chloro-4-(2-(1-oxyl-2,2,6,6-tetramethylpiperidin-4-yl)vinyl)-phenoxy)-6-(pyrimidin-4-yl)benzonitrile

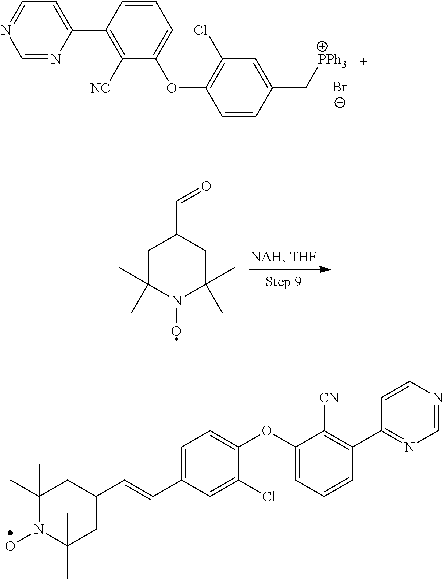

Sodium hydride (0.04 g, 1 mmol, 60% in oil) was added to a solution of (3-chloro-4-(2-cyano-3-(pyrimidin-4-yl)phenoxy)benzyl)triphenyl phosphonium bromide (0.19 g, 0.29 mmol) in tetrahydrofuran (10 mL). The mixture was stirred at room temperature for 1 hour, then 6-h (0.07 g, 0.38 mmol) was added, and the mixture was stirred at room temperature for additional 2 hour. After the reaction, the reaction was quenched by water (20 mL), and the mixture was extracted with dichloromethane (50 mL×3). The organic phase was dried by anhydrous sodium sulfate. The mixture was filtered, the filtrate was concentrated and the residue was purified by column chromatography (dichloromethane/methanol=40:1) to give the pure product (E)-2-(2-chloro-4-(2-(1-oxyl-2,2,6,6-tetramethylpiperidin-4-yl)vinyl)phenoxy)-6-(pyrimidin-4-yl)benzonitrile as a red solid (0.08 g, 55%). LRMS (M+H⁺): calcd for 488. found 488.

### (E)-2-(2-chloro-4-(2-(2,2,6,6-tetramethylpiperidin-4-yl)vinyl)phenoxy)-6-(pyrimidin-4-yl)benzonitrile (I-592)

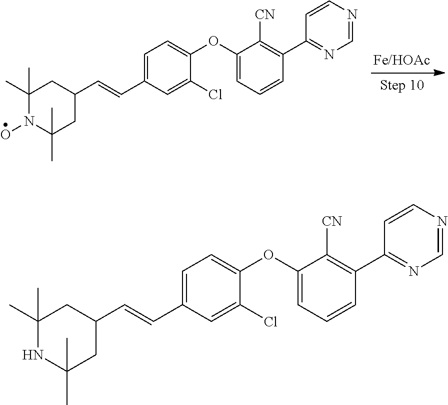

To a solution of (E)-2-(2-chloro-4-(2-(1-oxyl-2,2,6,6-tetramethylpiperidin-4-yl)vinyl)phenoxy)-6-(pyrimidin-4-yl)benzonitrile (0.08 g, 0.16 mmol) in acetate acid (5 mL) was added iron powder (0.10 g, 1.8 mmol). The reaction mixture was stirred for 2 hours at 60° C. After the reaction, the mixture was filtered and the filtrate was concentrated, then potassium carbonate aqueous was added to make pH=8˜10. The mixture was extracted with dichloromethane (50 mL×3), washed by water (50 mL). The organic phase was dried by anhydrous sodium sulfate. Then the mixture was filtered, the filtrate was concentrated and the residue was purified by preparative-HPLC to give pure product 6 as the white solid (0.05 g, 70%). ¹H NMR (300 MHz, CD₃OD): δ 9.373 (d, J=1.2 Hz, 1H), 9.008 (d, J=5.1 Hz, 1H), 8.035 (dd, J=1.5 Hz, J=5.4 Hz, 1H), 7.668-7.785 (m, 3H), 7.507 (dd, J=2.1 Hz, J=8.7 Hz, 1H), 7.281 (d, J=8.4 Hz, 1H), 6.969 (d, J=8.4 Hz, 1H), 6.614 (d, J=15.9 Hz, 1H), 6.317 (dd, J=6.6 Hz, J=15.9 Hz, 1H), 2.002 (dd, J=3.0 Hz, J=14.1 Hz, 2H), 2.920-2.955 (m, 1H), 1.580 (s, 6H), 1.509 (s, 6H), 1.483-1.544 (m, 2H). LRMS (M+H⁺): calcd 473. found 473.

### Example 27

### 3-chloro-4-(2-cyanophenoxy)-N-(1-(3-hydroxypropyl)-2,2,6,6-tetramethylpiperidin-4-yl)benzamide (I-489)

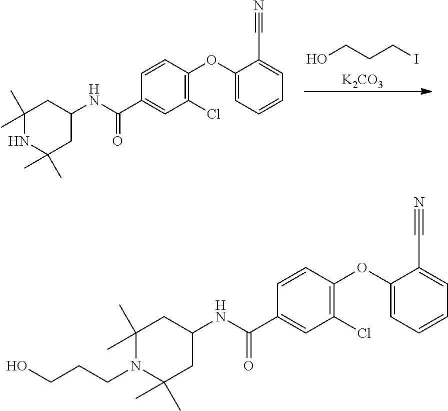

In a 5 mL microwave reaction vial, 3-chloro-4-(2-cyanophenoxy)-N-(2,2,6,6-tetramethylpiperidin-4-yl)benzamide (109 mg, 0.265 mmol) was dissolved in dry acetonitrile (3 mL), followed by the addition of 3-iodopropan-1-ol (738 mg, 3.97 mmol), and potassium carbonate (73.1 mg, 0.529 mmol). The reaction was vac/purged with nitrogen, and heated in the microwave at 150° C. for 30 minutes. Additional 3-iodopropan-1-ol (738 mg, 3.97 mmol) and more potassium carbonate (73.1 mg, 0.529 mmol) were added and the reaction was sealed and heated in the microwave for 1 hour at 150° C. The reaction solution was diluted with sat. aq. sodium bicarbonate solution and extracted with ethyl acetate (3×). The organic layer was washed with brine, dried over sodium sulfate, filtered, concentrated. The crude residue was dissolved in DMF (1 mL), water (1 mL), and MeOH (2 mL), then filtered through PTFE acrodisc, and purified by prepatory HPLC (20%-95% gradient of water-1% TFA:acetonitrile-1% TFA). The product containing fractions were combined, diluted with sat. aq. sodium bicarbonate, extracted with dichloromethane (3×), dried over sodium sulfate, filtered and concentrated. The purified product was lyophilized to provide a white powder (51 mg, 41%). ¹H NMR (300 MHz, CD₃OD): δ 8.34-8.29 (m, 1H), 8.12 (d, J=2.06 Hz, 1H), 7.97-7.92 (m, 1H), 7.91-7.86 (m, 1H), 7.71-7.65 (m, 1H), 7.37-7.31 (m, 2H), 6.95-6.91 (m, 1H), 4.37-4.31 (m, 1H), 4.26-4.14 (m, 1H), 3.41-3.34 (m, 2H), 1.71-1.63 (m, 2H), 1.57-1.47 (m, 2H), 1.43-1.33 (m, 2H), 1.06 (d, J=12.36 Hz, 10H). LRMS (M+H⁺) m/z: calcd 470.21. found 470.3.

### Example 28

### 2,2,6,6-tetramethylpiperidin-4-yl 3-chloro-4-(2-cyano-3-(pyridazin-4-yl)phenoxy)benzoate (I-504)

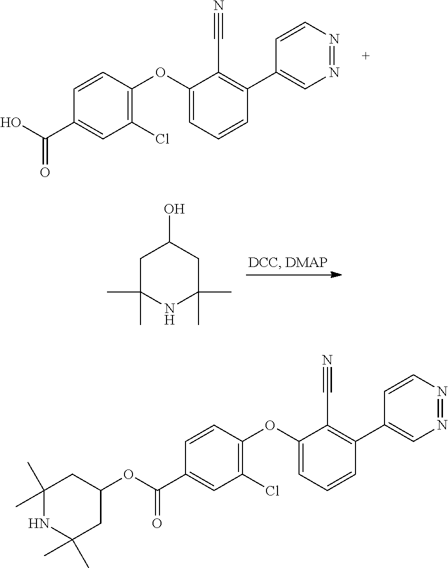

A 25 mL round bottom flask was charged with 3-chloro-4-(2-cyano-3-(pyridazin-4-yl)phenoxy)benzoic acid (110 mg, 0.313 mmol) and dissolved in dichloromethane (10 mL). To this solution was added 2,2,6,6-tetramethylpiperidin-4-ol (49.2 mg, 0.313 mmol), N,N′-methanediylidenedicyclohexanamine (77 mg, 0.375 mmol), and DMAP (45.8 mg, 0.375 mmol). The reaction was allowed to stir overnight at ambient temperature. The reaction was diluted with sat. aq. sodium bicarbonate, extracted with dichloromethane (3×). The combined organic layer was washed sat. aq. sodium bicarbonate, brine (2×), dried over sodium sulfate, filtered, and concentrated. The crude residue was purified on a Biotage system using a gradient of 5% to 80% MeOH in DCM. The purified product fractions were concentrated and the product was lyophilized to provide white solid (30 mg, 20%). ¹H NMR (300 MHz, CD₃OD): δ 9.56 (dd, J=1.26, 2.40 Hz, 1H), 9.46 (dd, J=1.14, 5.49 Hz, 1H), 8.14 (d, J=2.06 Hz, 1H), 8.04 (dd, J=2.40, 5.38 Hz, 1H), 7.98 (dd, J=2.06, 8.47 Hz, 1H), 7.88-7.82 (m, 1H), 7.62 (dd, J=0.69, 7.78 Hz, 1H), 7.43 (d, J=8.70 Hz, 1H), 7.21 (dd, J=0.80, 8.58 Hz, 1H), 5.34 (br. s., 1H), 1.94 (br. s., 2H), 1.43-1.04 (m, 14H). LRMS (M+H⁺) m/z: calcd 491.18. found 491.2.

### Example 29

### IC₅₀ Measurements for Inhibitors Using EZH2

EZH2 Assay:

Assays were carried out by mixing rPRC2 together with biotinylated oligonucleosome substrates in the presence of the radio-labeled enzyme co-factor, S-adenosyl-L-methionine (³H SAM) (Perkin Elmer) and monitoring the enzymatically mediated transfer of tritiated methyl groups from ³H SAM to histone lysine residues. The amount of resulting tritated methylhistone product was measured by first capturing the biotinylated oligonuclesomes in streptavidin (SAV) coated FlashPlates (Perkin Elmer), followed by a wash step to remove un-reacted ³H SAM, and then counting on a TopCount NXT 384 well plate scintillation counter (Perkin Elmer). The final assay conditions for EZH2 were as follows: 50 mM Tris Buffer pH 8.5, 1 mM DTT, 69 uM Brij-35 detergent, 5.0 mM MgCl₂, 0.1 mg/mL BSA, 0.2 uM ³H SAM, 0.2 uM biotinylated oligonucleosomes, 3.6 uM H3K27me3 peptide and 2 nM EZH2.

Compound IC₅₀ measurements were obtained as follows: Compounds were first dissolved in 100% DMSO as 10 mM stock solutions. Ten point dose response curves were generated by dispensing varying amounts of the 10 mM compound solution in 10 wells of the 384 well plate (Echo; Labcyte), pure DMSO was then used to backfill the wells to insure all wells have the same amount of DMSO. A 12.5 uL volume of the HMT enzyme, H3K27me3 peptide and oligonucleosome substrate in assay buffer was added to each well of the assay plate using a Multidrop Combi (ThermoFisher). Compounds were pre-incubated with the enzyme for 20 min, followed by initiation of the methyltransferase reaction by addition of 12.5 uL of 3H SAM in assay buffer (final volume=25 uL). The final concentrations of compounds ranged from a top default concentration of 80 uM down to 0.16 uM in ten 2-fold dilution steps. Reactions were carried out for 60 minutes and quenched with 20 uL per well of 1.96 mM SAH, 50 mM Tris PH 8.5, 200 mM EDTA. Stopped reactions were transferred to SAV coated Flashplates (Perkin Elmer), incubated for 120 min, washed with a plate washer, and then read on the TopCount NXT (1.0 min/well) to measure the amount of methylhistone product formed during the reaction. The amount of methylhistone product was compared with the amount of product formed in the 0% and 100% inhibition control wells allowing the calculation of % Inhibition in the presence of the individual compounds at various concentrations. IC₅₀'s were computed using a 4 parameter fit non-linear curve fitting software package (XLFIT, part of the database package, ActivityBase (IDBS)) where the four parameters were IC₅₀, Hill slope, pre-transitional baseline (0% INH), and post-transitional baseline (100% INH); with the latter two parameters being fixed to zero and 100%, respectively, by default.

Assay for Y641N EZH2 was performed as above using reconstituted H3K27Me2 oligonucleosomes as substrate.

Table 2 shows the activity of selected compounds of this invention in the EZH2 and Y641N EZH2 inhibition assay. The compound numbers correspond to the compound numbers in Table 1. Compounds having an activity designated as “A” provided an IC₅₀≦5 μM; compounds having an activity designated as “B” provided an IC₅₀ of 5-20 μM; compounds having an activity designated as “C” provided an IC₅₀ of 20-80 μM; and compounds having an activity designated as “D” provided an IC₅₀≧80 μM. “NA” stands for “not assayed.”

TABLE 2
EZH2 and Y641N EZH2 Activity Inhibition Data

| Compound | | Y641N EZH2 |
|---|---|---|
| No. | EZH2 IC₅₀ | IC₅₀ |
| I-1 | A | NA |
| I-2 | A | NA |
| I-3 | A | NA |
| I-4 | A | NA |
| I-5 | A | NA |
| I-6 | A | NA |
| I-7 | B | NA |
| I-8 | B | NA |
| I-9 | B | NA |
| I-10 | B | NA |
| I-11 | B | NA |
| I-12 | B | NA |
| I-13 | B | NA |
| I-14 | B | NA |
| I-15 | B | NA |
| I-16 | B | NA |
| I-17 | B | NA |
| I-18 | B | NA |
| I-19 | B | NA |
| I-20 | B | NA |
| I-21 | C | NA |
| I-22 | C | NA |
| I-23 | C | NA |
| I-24 | C | NA |
| I-25 | C | NA |
| I-26 | C | NA |
| I-27 | C | NA |
| I-28 | C | NA |
| I-29 | C | NA |
| I-30 | C | NA |
| I-31 | C | NA |
| I-32 | C | NA |
| I-33 | C | NA |
| I-34 | C | NA |
| I-35 | C | NA |
| I-36 | C | NA |
| I-37 | C | NA |
| I-38 | C | NA |
| I-39 | C | NA |
| I-40 | C | NA |
| I-41 | C | NA |
| I-42 | C | NA |
| I-43 | C | NA |
| I-44 | C | NA |
| I-45 | C | NA |
| I-46 | C | NA |
| I-47 | C | NA |
| I-48 | C | NA |
| I-49 | C | NA |
| I-50 | C | NA |
| I-51 | C | NA |
| I-52 | C | NA |
| I-53 | C | NA |
| I-54 | C | NA |
| I-55 | C | NA |
| I-56 | C | NA |
| I-57 | C | NA |
| I-58 | C | NA |
| I-59 | C | NA |
| I-60 | C | NA |
| I-61 | C | NA |
| I-62 | C | NA |
| I-63 | C | NA |
| I-64 | C | NA |
| I-65 | C | NA |
| I-66 | C | NA |
| I-67 | C | NA |
| I-68 | D | NA |
| I-69 | D | NA |
| I-70 | D | NA |
| I-71 | D | NA |
| I-72 | D | NA |
| I-73 | D | NA |
| I-74 | D | NA |
| I-75 | D | NA |
| I-76 | D | NA |
| I-77 | D | NA |
| I-78 | D | NA |
| I-79 | D | NA |
| I-80 | D | NA |
| I-81 | D | NA |
| I-82 | D | NA |
| I-83 | D | NA |
| I-84 | D | NA |
| I-85 | D | NA |
| I-86 | D | NA |
| I-87 | D | NA |
| I-88 | D | NA |
| I-89 | D | NA |
| I-90 | D | NA |
| I-91 | D | NA |
| I-92 | D | NA |
| I-93 | D | NA |
| I-94 | D | NA |
| I-95 | D | NA |
| I-96 | D | NA |
| I-97 | D | NA |
| I-98 | D | NA |
| I-99 | D | NA |
| I-100 | D | NA |
| I-101 | D | NA |
| I-102 | D | NA |
| I-103 | D | NA |
| I-104 | D | NA |
| I-105 | D | NA |
| I-106 | D | NA |
| I-107 | D | NA |
| I-108 | D | NA |
| I-109 | D | NA |
| I-110 | D | NA |
| I-111 | D | NA |
| I-112 | D | NA |
| I-113 | D | NA |
| I-114 | D | NA |
| I-115 | D | NA |
| I-116 | D | NA |
| I-117 | D | NA |
| I-118 | D | NA |
| I-119 | D | NA |
| I-120 | D | NA |
| I-121 | D | NA |
| I-122 | D | NA |
| I-123 | D | NA |
| I-124 | D | NA |
| I-125 | D | NA |
| I-126 | D | NA |
| I-127 | D | NA |
| I-128 | D | NA |
| I-129 | D | NA |
| I-130 | D | NA |
| I-131 | D | NA |
| I-132 | D | NA |
| I-133 | D | NA |
| I-134 | D | NA |
| I-135 | D | NA |
| I-136 | D | NA |
| I-137 | D | NA |
| I-138 | D | NA |
| I-139 | D | NA |
| I-140 | D | NA |
| I-141 | D | NA |
| I-143 | D | NA |
| I-144 | B | NA |
| I-145 | C | NA |
| I-147 | C | NA |
| I-148 | B | NA |
| I-149 | B | NA |
| I-150 | B | NA |
| I-151 | B | NA |
| I-152 | A | NA |
| I-153 | C | NA |
| I-154 | C | C |
| I-155 | C | NA |
| I-156 | D | NA |
| I-157 | C | NA |
| I-158 | C | NA |
| I-159 | C | NA |
| I-160 | D | NA |
| I-161 | D | NA |
| I-162 | C | NA |
| I-163 | C | D |
| I-164 | D | NA |
| I-165 | D | NA |
| I-166 | D | NA |
| I-167 | C | C |
| I-168 | C | NA |
| I-169 | B | NA |
| I-170 | B | NA |
| I-171 | A | C |
| I-172 | D | NA |
| I-173 | B | B |
| I-174 | C | NA |
| I-175 | D | NA |
| I-176 | C | NA |
| I-177 | D | NA |
| I-178 | D | NA |
| I-179 | C | NA |
| I-180 | C | NA |
| I-181 | B | NA |
| I-182 | B | NA |
| I-183 | C | C |
| I-184 | A | NA |
| I-185 | C | D |
| I-186 | A | B |
| I-187 | C | NA |
| I-188 | A | NA |
| I-189 | C | NA |
| I-190 | B | NA |
| I-191 | D | NA |
| I-192 | D | NA |
| I-193 | D | NA |
| I-194 | A | NA |
| I-195 | B | NA |
| I-196 | A | NA |
| I-197 | A | NA |
| I-198 | A | NA |
| I-199 | A | B |
| I-200 | C | NA |
| I-201 | A | NA |
| I-202 | B | NA |
| I-203 | D | NA |
| I-204 | D | NA |
| I-205 | B | NA |
| I-206 | A | NA |
| I-207 | A | C |
| I-208 | B | NA |
| I-209 | B | NA |
| I-210 | C | NA |
| I-211 | A | NA |
| I-212 | A | NA |
| I-213 | A | NA |
| I-214 | C | NA |
| I-215 | C | NA |
| I-216 | B | C |
| I-217 | A | C |
| I-218 | A | NA |
| I-219 | A | NA |
| I-220 | A | NA |
| I-221 | A | NA |
| I-222 | D | NA |
| I-223 | A | B |
| I-224 | B | NA |
| I-225 | B | NA |
| I-226 | A | NA |
| I-227 | A | NA |
| I-228 | B | D |
| I-229 | B | NA |
| I-230 | B | NA |
| I-231 | B | NA |
| I-232 | C | NA |
| I-233 | D | NA |
| I-234 | D | NA |
| I-235 | B | NA |
| I-236 | B | NA |
| I-237 | A | NA |
| I-238 | B | NA |
| I-239 | A | NA |
| I-240 | D | NA |
| I-241 | D | NA |
| I-242 | C | NA |
| I-243 | C | NA |
| I-244 | C | NA |
| I-245 | B | NA |
| I-246 | C | NA |
| I-247 | C | NA |
| I-248 | C | NA |
| I-249 | B | NA |
| I-250 | C | NA |
| I-251 | C | NA |
| I-252 | A | NA |
| I-253 | A | NA |
| I-254 | A | A |
| I-255 | A | NA |
| I-256 | A | A |
| I-257 | A | A |
| I-258 | A | A |
| I-259 | A | A |
| I-260 | A | C |
| I-261 | A | NA |
| I-262 | A | A |
| I-263 | A | NA |
| I-264 | A | NA |
| I-265 | A | NA |
| I-266 | A | A |
| I-267 | A | NA |
| I-268 | C | NA |
| I-269 | C | NA |
| I-270 | A | NA |
| I-271 | B | NA |
| I-272 | C | NA |
| I-273 | C | NA |
| I-274 | D | NA |
| I-275 | D | NA |
| I-276 | B | NA |
| I-277 | A | NA |
| I-278 | A | NA |
| I-279 | A | NA |
| I-280 | D | NA |
| I-281 | D | NA |
| I-282 | D | NA |
| I-283 | B | NA |
| I-284 | B | NA |
| I-285 | C | NA |
| I-286 | B | NA |
| I-287 | C | NA |
| I-288 | A | NA |
| I-289 | A | NA |
| I-290 | D | NA |
| I-291 | A | NA |
| I-292 | D | NA |
| I-293 | B | NA |
| I-294 | A | NA |
| I-295 | A | NA |
| I-296 | A | NA |
| I-297 | B | NA |
| I-298 | D | NA |
| I-299 | D | NA |
| I-300 | C | NA |
| I-302 | C | NA |
| I-303 | D | NA |
| I-304 | B | D |
| I-305 | C | NA |
| I-306 | D | NA |
| I-307 | A | A |
| I-308 | A | A |
| I-309 | A | NA |
| I-310 | A | NA |
| I-311 | A | NA |
| I-312 | A | A |
| I-313 | D | NA |
| I-314 | B | NA |
| I-315 | A | NA |
| I-316 | C | NA |
| I-317 | D | NA |
| I-318 | C | NA |
| I-319 | D | NA |
| I-320 | A | B |
| I-321 | D | NA |
| I-322 | A | NA |
| I-323 | A | NA |
| I-324 | A | B |
| I-325 | B | NA |
| I-326 | B | D |
| I-327 | A | B |
| I-328 | C | NA |
| I-329 | D | NA |
| I-330 | C | D |
| I-331 | C | NA |
| I-332 | B | NA |
| I-333 | A | B |
| I-334 | A | B |
| I-335 | C | NA |
| I-336 | A | NA |
| I-337 | A | NA |
| I-338 | A | NA |
| I-339 | A | A |
| I-340 | A | B |
| I-341 | D | NA |
| I-342 | C | D |
| I-343 | C | C |
| I-344 | B | D |
| I-345 | A | NA |
| I-346 | B | NA |
| I-347 | A | A |
| I-348 | A | NA |
| I-349 | B | NA |
| I-350 | B | NA |
| I-351 | A | NA |
| I-352 | A | NA |
| I-353 | D | NA |
| I-354 | D | NA |
| I-355 | D | NA |
| I-356 | D | D |
| I-357 | D | NA |
| I-359 | B | NA |
| I-360 | A | NA |
| I-361 | B | NA |
| I-362 | A | B |
| I-363 | A | NA |
| I-364 | A | A |
| I-365 | A | NA |
| I-366 | A | NA |
| I-367 | A | A |
| I-368 | D | NA |
| I-369 | A | A |
| I-370 | C | NA |
| I-371 | A | NA |
| I-372 | B | NA |
| I-373 | D | NA |
| I-374 | A | NA |
| I-375 | A | A |
| I-376 | C | NA |
| I-377 | B | NA |
| I-378 | A | A |
| I-379 | C | C |
| I-380 | C | NA |
| I-381 | A | C |
| I-382 | D | NA |
| I-383 | C | NA |
| I-384 | C | NA |
| I-385 | B | D |
| I-386 | A | NA |
| I-387 | A | NA |
| I-388 | B | NA |
| I-389 | D | NA |
| I-390 | D | NA |
| I-391 | A | NA |
| I-392 | D | NA |
| I-393 | D | NA |
| I-394 | B | NA |
| I-395 | A | B |
| I-396 | D | NA |
| I-397 | B | D |
| I-398 | A | B |
| I-399 | A | NA |
| I-400 | B | NA |
| I-401 | A | A |
| I-402 | D | NA |
| I-403 | D | NA |
| I-404 | D | C |
| I-405 | C | D |
| I-406 | D | NA |
| I-407 | D | NA |
| I-408 | D | NA |
| I-409 | C | NA |
| I-410 | D | NA |
| I-411 | D | NA |
| I-412 | D | NA |
| I-413 | C | D |
| I-415 | C | D |
| I-416 | D | NA |
| I-417 | C | D |
| I-418 | C | NA |
| I-419 | D | NA |
| I-420 | A | NA |
| I-421 | D | NA |
| I-422 | C | NA |
| I-423 | A | C |
| I-424 | D | NA |
| I-425 | D | NA |
| I-426 | D | NA |
| I-427 | D | D |
| I-428 | A | B |
| I-429 | C | D |
| I-430 | A | NA |
| I-431 | C | D |
| I-432 | D | NA |
| I-433 | D | NA |
| I-434 | C | NA |
| I-435 | A | NA |
| I-436 | D | NA |
| I-437 | D | NA |
| I-438 | D | NA |
| I-439 | A | NA |
| I-440 | D | NA |
| I-442 | D | NA |
| I-443 | B | NA |
| I-444 | A | NA |
| I-445 | A | NA |
| I-446 | B | NA |
| I-447 | B | NA |
| I-448 | D | NA |
| I-449 | B | NA |
| I-450 | D | NA |
| I-451 | A | NA |
| I-452 | D | NA |
| I-453 | A | NA |
| I-454 | D | NA |
| I-455 | B | NA |
| I-456 | A | NA |
| I-457 | D | NA |
| I-458 | A | NA |
| I-459 | A | NA |
| I-460 | D | NA |
| I-461 | B | NA |
| I-462 | C | NA |
| I-463 | A | NA |
| I-464 | D | NA |
| I-465 | D | NA |
| I-466 | D | NA |
| I-467 | C | NA |
| I-469 | A | NA |
| I-470 | D | NA |
| I-471 | D | NA |
| I-472 | A | NA |
| I-474 | C | NA |
| I-475 | D | NA |
| I-476 | A | NA |
| I-477 | A | NA |
| I-478 | A | NA |
| I-479 | C | NA |
| I-480 | A | A |
| I-482 | C | NA |
| I-483 | D | NA |
| I-484 | D | NA |
| I-485 | C | NA |
| I-486 | B | NA |
| I-487 | A | NA |
| I-488 | D | NA |
| I-489 | A | NA |
| I-490 | B | NA |
| I-491 | D | NA |
| I-492 | A | NA |
| I-493 | C | D |
| I-494 | C | NA |
| I-496 | D | NA |
| I-498 | B | NA |
| I-499 | C | D |
| I-500 | A | NA |
| I-501 | A | NA |
| I-502 | A | NA |
| I-503 | B | NA |
| I-504 | A | A |
| I-505 | A | NA |
| I-506 | A | NA |
| I-507 | C | NA |
| I-508 | A | NA |
| I-517 | B | NA |
| I-518 | C | NA |
| I-519 | C | NA |
| I-520 | A | NA |
| I-522 | C | NA |
| I-525 | B | NA |
| I-526 | A | NA |
| I-527 | A | NA |
| I-528 | A | NA |
| I-529 | A | NA |
| I-530 | A | NA |
| I-531 | A | NA |
| I-533 | A | D |
| I-534 | A | NA |
| I-535 | A | NA |
| I-536 | A | C |
| I-537 | A | NA |
| I-538 | A | NA |
| I-548 | A | C |
| I-549 | A | NA |
| I-550 | A | NA |
| I-551 | A | NA |
| I-552 | A | NA |
| I-553 | A | NA |
| I-554 | A | NA |
| I-555 | A | NA |
| I-556 | A | D |
| I-557 | A | NA |
| I-558 | A | A |
| I-559 | A | NA |
| I-560 | A | D |
| I-561 | A | A |
| I-562 | A | NA |
| I-564 | A | D |
| I-570 | D | NA |
| I-571 | A | A |
| I-572 | A | A |
| I-573 | A | B |
| I-583 | A | A |
| I-584 | A | A |
| I-585 | A | B |
| I-586 | A | A |
| I-590 | C | D |
| I-591 | A | C |
| I-592 | A | B |
| I-593 | A | NA |

1. A method of inhibiting EZH2 activity comprising administering a compound of formula I: 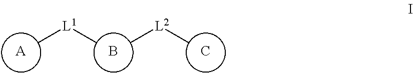
or a pharmaceutically acceptable salt thereof, wherein:
Ring A is an optionally substituted group selected from a 5-6 membered monocyclic heteroaryl ring having 1-3 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, an 8-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
Ring B is an optionally substituted bivalent ring selected from phenylene, a 4-7 membered saturated or partially unsaturated heterocyclic ring having 1-2 heteroatoms independently selected from nitrogen, oxygen, or sulfur, 5-6 membered monocyclic heteroarylene ring having 1-3 heteroatoms independently selected from nitrogen, oxygen, or sulfur; an 8-10 membered bicyclic aryl carbocyclic ring, an 8-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
Ring C is an optionally substituted group selected from phenyl, a 3-7 membered saturated or partially unsaturated carbocyclic ring, a 4-7 membered saturated or partially unsaturated heterocyclic ring having 1-2 heteroatoms independently selected from nitrogen, oxygen, or sulfur, an 8-10 membered bicyclic aryl carbocyclic ring, a 5-6 membered monocyclic heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
each of L¹ and L² is independently a covalent bond or an optionally substituted bivalent C₁₋₆ hydrocarbon chain, wherein one or more methylene units of L¹ or L² are optionally and independently replaced by -Cy-, —O—, —S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —OC(O)—, or —C(O)O;
each R′ is independently —R, —C(O)R, —CO₂R, or —SO₂R, or:
two R′ on the same nitrogen are taken together with their intervening atoms to form a 4-7 membered heterocyclic ring having 1-2 heteroatoms independently selected from nitrogen, oxygen, and sulfur;
each R is hydrogen, or an optionally substituted group selected from C₁₋₆ aliphatic, phenyl, a 3-7 membered saturated or partially unsaturated carbocyclic ring, an 8-10 membered bicyclic saturated, partially unsaturated or aryl carbocyclic ring, a 5-6 membered monocyclic heteroaryl ring having 1-3 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, an 8-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur; and
-Cy- is an optionally substituted bivalent ring selected from phenylene, a 3-7 membered saturated or partially unsaturated carbocyclylene, a 4-7 membered saturated or partially unsaturated heterocyclylene having 1-2 heteroatoms independently selected from nitrogen, oxygen, or sulfur, or a 5-6 membered heteroarylene having 1-3 heteroatoms independently selected from nitrogen, oxygen, or sulfur.

2-24. (canceled)

25. A compound of formula II: 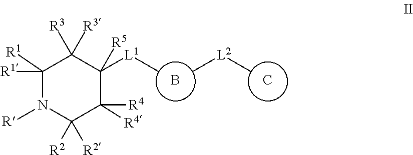
or a pharmaceutically acceptable salt thereof, wherein:
Ring B is an optionally substituted bivalent ring selected from phenylene, a 4-7 membered saturated or partially unsaturated heterocyclic ring having 1-2 heteroatoms independently selected from nitrogen, oxygen, or sulfur, 5-6 membered monocyclic heteroarylene ring having 1-3 heteroatoms independently selected from nitrogen, oxygen, or sulfur; an 8-10 membered bicyclic aryl carbocyclic ring, an 8-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
Ring C is an optionally substituted group selected from phenyl, a 3-7 membered saturated or partially unsaturated carbocyclic ring, a 4-7 membered saturated or partially unsaturated heterocyclic ring having 1-2 heteroatoms independently selected from nitrogen, oxygen, or sulfur, an 8-10 membered bicyclic aryl carbocyclic ring, a 5-6 membered monocyclic heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
each of L¹ and L² is independently a covalent bond or an optionally substituted bivalent C₁₋₆ hydrocarbon chain, wherein one or more methylene units of L¹ or L² are optionally and independently replaced by -Cy-, —O—, —S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —OC(O)—, or —C(O)O—;
each R′ is independently —R, —C(O)R, —CO₂R, or —SO₂R, or:
two R′ on the same nitrogen are taken together with their intervening atoms to form a 4-7 membered heterocyclic ring having 1-2 heteroatoms independently selected from nitrogen, oxygen, and sulfur;
each R is hydrogen, or an optionally substituted group selected from C₁₋₆ aliphatic, phenyl, a 3-7 membered saturated or partially unsaturated carbocyclic ring, an 8-10 membered bicyclic saturated, partially unsaturated or aryl carbocyclic ring, a 5-6 membered monocyclic heteroaryl ring having 1-3 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, an 8-10 membered bicyclic saturated or partially unsaturated heterocyclic ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
-Cy- is an optionally substituted bivalent ring selected from phenylene, a 3-7 membered saturated or partially unsaturated carbocyclylene, a 4-7 membered saturated or partially unsaturated heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
each R¹, R^{1′}, R², R^{2′}, R³, R^{3′}, R⁴, R^{4′} and R⁵ is independently —R′, halogen, —CN, —NO₂, —OR, —N(R′), —SR; or
each of R¹ and R^{1′}, R² and R^{2′}, R³ and R^{3′}, or R⁴ and R^{4′} is optionally and independently taken together to form ═X, wherein X is ═O, ═S, ═NR′, ═N—N—OR or ═N—NR′; or
each of R¹ or R^{1′} and R² or R^{2′}, R³ or R^{3′} and R⁴ or R^{4′}, R¹ or R^{1′} and R³ or R^{3′}, R² or R^{2′} and R⁴ or R^{4′}, R² or R^{2′} and R³ or R^{3′}, R¹ or R^{1′} and R⁴ or R^{4′}, R¹ or R^{1′} and R′, R² or R^{2′} and R′, and R′ and R⁵ is optionally and independently taken together with their intervening atoms to form a 3-7 membered saturated or partially unsaturated carbocyclic ring, or a 4-7 membered saturated or partially unsaturated heterocyclic ring having 1-2 heteroatoms independently selected from nitrogen, oxygen, and sulfur.

26. The compound of claim 25, wherein R¹, R^{1′}, R² and R^{2′} are methyl.

27. The compound of claim 26, wherein R³, R^{3′}, R⁴, R^{4′}, R⁵ and R′ are hydrogen.

28. The compound of claim 26, wherein L¹ is selected from —OCH₂—, —CH₂O—, —OC(O)—, —N(R′)C(O)—, —C(O)N(R′)—, and optionally substituted ethenylene.

29.-30. (canceled)

31. The compound of claim 28, wherein L¹ is —NH—C(O)—, —OCH₂—, —CH₂O—, —OC(O)—, and —CH═CH—.

32. The compound of claim 25, wherein Ring B is optionally substituted phenyl or optionally substituted pyridinyl.

33.-36. (canceled)

37. The compound of claim 32, wherein L² is selected from —CH₂O—, —O—, and —CH(CH₃)O.

38. The compound of claim 25, wherein Ring C is optionally substituted phenyl.

39.-40. (canceled)

41. The compound of claim 25, wherein Ring C is optionally substituted pyridinyl, pyrimidinyl or pyrazinyl.

42. The compound of claim 41, wherein Ring C is selected from

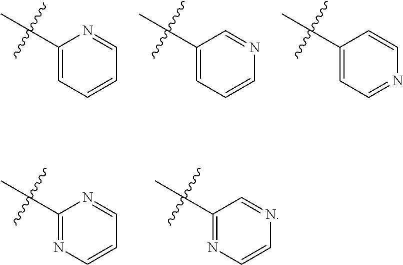 [SMILES: *c1ccccn1 ; *c1cccnc1 ; *c1ccncc1 ; *c1ncccn1 ; *c1cnccn1]

43. (canceled)

44. The compound of claim 25, wherein Ring C is selected from the group consisting of cyclopropyl, cyclobutyl, cyclopentyl, cyclohexyl and cycloheptyl.

45. (canceled)

46. The compound of claim 25, wherein Ring C is selected from pyrrolidinyl, furanyl, pyrazolidinyl, imidazolidinyl, thiazolidinyl, piperidinyl, piperazinyl and morpholinyl.

47. (canceled)

48. The compound of claim 25, wherein Ring C is optionally substituted indolyl, quinolinyl, isoquinolinyl or naphthyl.

49. (canceled)

50. A pharmaceutical composition comprising a compound of claim 25 and a pharmaceutically acceptable excipient.

51-56. (canceled)

57. The compound of claim 25, selected from any one of the compounds set forth below: 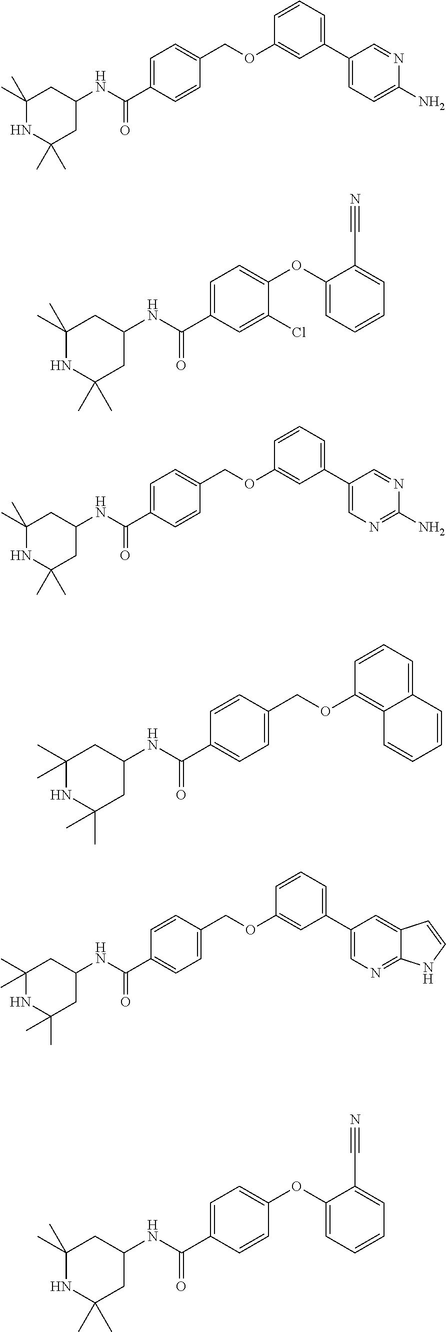 [SMILES: CC1(C)CC(NC(=O)c2ccc(COc3cccc(-c4ccc(N)nc4)c3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(COc3cccc(-c4cnc(N)nc4)c3)cc2)CC(C)(C)N1 ; CC1(C)CC(NC(=O)c2ccc(COc3cccc4ccccc34)cc2)CC(C)(C)N1 ; CC1(C)CC(NC(=O)c2ccc(COc3cccc(-c4cnc5[nH]ccc5c4)c3)cc2)CC(C)(C)N1 ; CC1(C)CC(NC(=O)c2ccc(Oc3ccccc3C#N)cc2)CC(C)(C)N1] 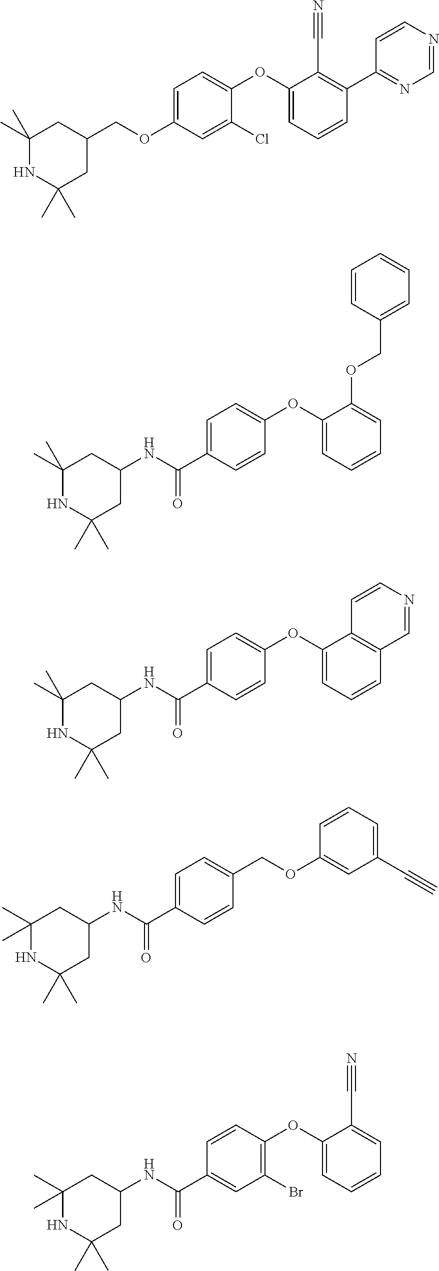 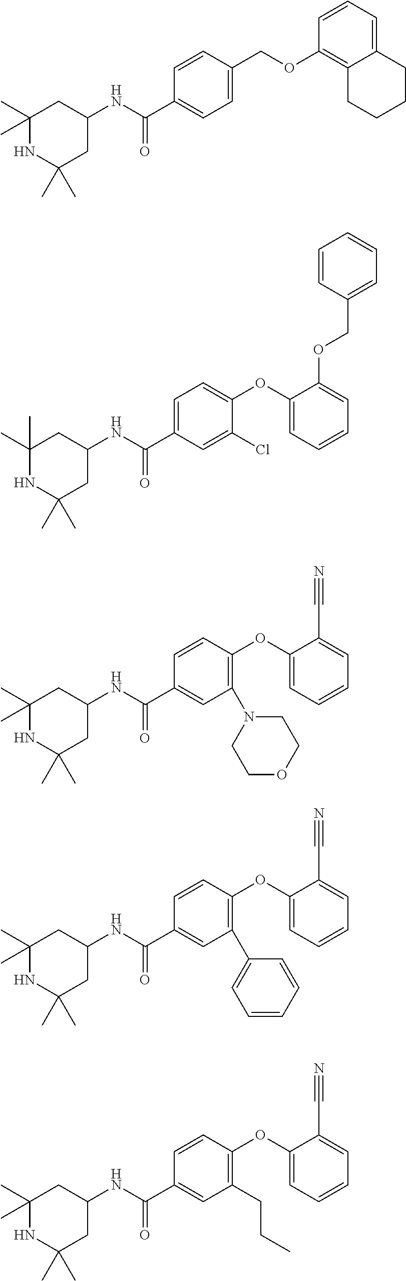 [SMILES: CC1(C)CC(NC(=O)c2ccc(COc3cccc4c3CCCC4)cc2)CC(C)(C)N1 ; CC1(C)CC(NC(=O)c2ccc(Oc3ccccc3OCc3ccccc3)c(Cl)c2)CC(C)(C)N1 ; CC1(C)CC(NC(=O)c2ccc(Oc3ccccc3C#N)c(N3CCOCC3)c2)CC(C)(C)N1 ; CC1(C)CC(NC(=O)c2ccc(Oc3ccccc3C#N)c(-c3ccccc3)c2)CC(C)(C)N1 ; CCCc1cc(C(=O)NC2CC(C)(C)NC(C)(C)C2)ccc1Oc1ccccc1C#N] 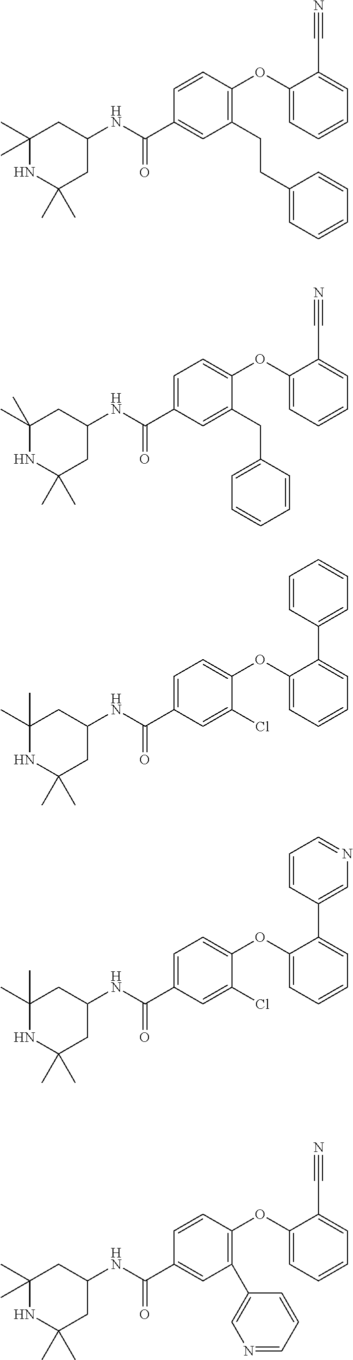 [SMILES: CC1(C)CC(NC(=O)c2ccc(Oc3ccccc3C#N)c(CCc3ccccc3)c2)CC(C)(C)N1 ; CC1(C)CC(NC(=O)c2ccc(Oc3ccccc3C#N)c(Cc3ccccc3)c2)CC(C)(C)N1 ; CC1(C)CC(NC(=O)c2ccc(Oc3ccccc3-c3ccccc3)c(Cl)c2)CC(C)(C)N1 ; CC1(C)CC(NC(=O)c2ccc(Oc3ccccc3-c3cccnc3)c(Cl)c2)CC(C)(C)N1 ; CC1(C)CC(NC(=O)c2ccc(Oc3ccccc3C#N)c(-c3cccnc3)c2)CC(C)(C)N1] 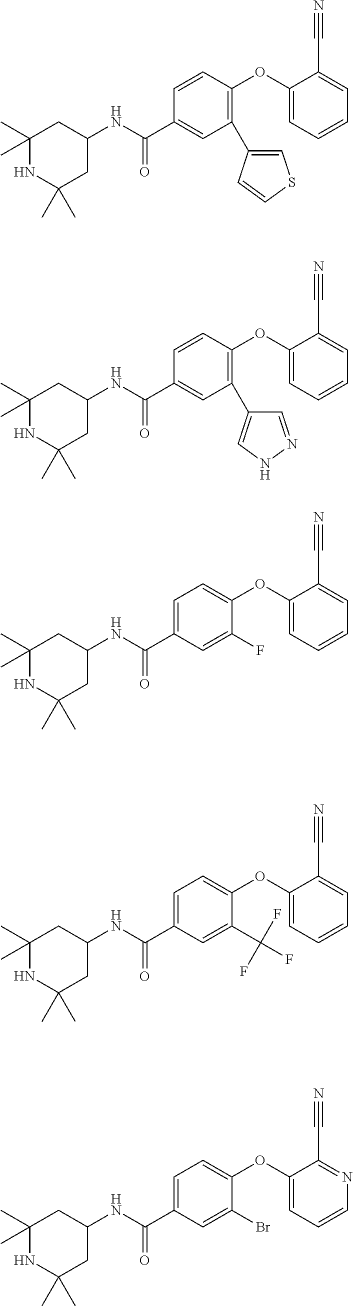 [SMILES: CC1(C)CC(NC(=O)c2ccc(Oc3ccccc3C#N)c(-c3ccsc3)c2)CC(C)(C)N1 ; CC1(C)CC(NC(=O)c2ccc(Oc3ccccc3C#N)c(-c3cn[nH]c3)c2)CC(C)(C)N1 ; CC1(C)CC(NC(=O)c2ccc(Oc3ccccc3C#N)c(F)c2)CC(C)(C)N1 ; CC1(C)CC(NC(=O)c2ccc(Oc3ccccc3C#N)c(C(F)(F)F)c2)CC(C)(C)N1 ; CC1(C)CC(NC(=O)c2ccc(Oc3cccnc3C#N)c(Br)c2)CC(C)(C)N1] 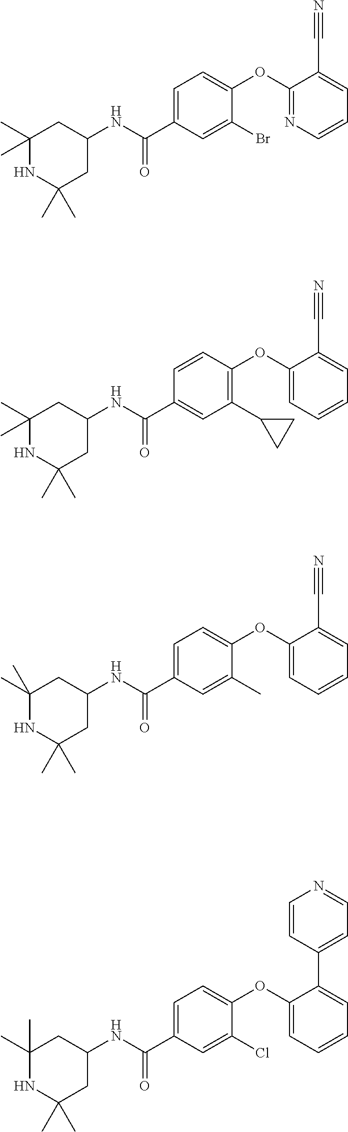 [SMILES: CC1(C)CC(NC(=O)c2ccc(Oc3ncccc3C#N)c(Br)c2)CC(C)(C)N1 ; CC1(C)CC(NC(=O)c2ccc(Oc3ccccc3C#N)c(C3CC3)c2)CC(C)(C)N1 ; Cc1cc(C(=O)NC2CC(C)(C)NC(C)(C)C2)ccc1Oc1ccccc1C#N ; CC1(C)CC(NC(=O)c2ccc(Oc3ccccc3-c3ccncc3)c(Cl)c2)CC(C)(C)N1] 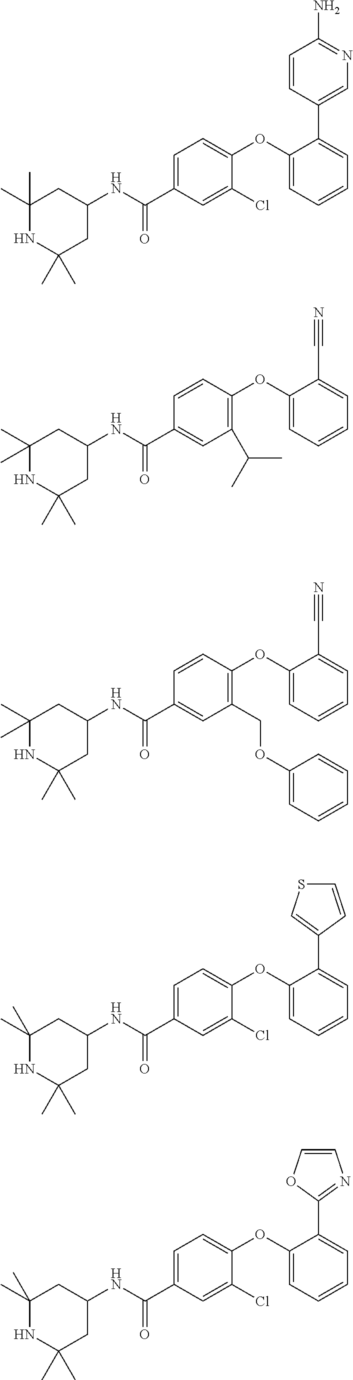 [SMILES: CC1(C)CC(NC(=O)c2ccc(Oc3ccccc3-c3ccc(N)nc3)c(Cl)c2)CC(C)(C)N1 ; CC(C)c1cc(C(=O)NC2CC(C)(C)NC(C)(C)C2)ccc1Oc1ccccc1C#N ; CC1(C)CC(NC(=O)c2ccc(Oc3ccccc3C#N)c(COc3ccccc3)c2)CC(C)(C)N1 ; CC1(C)CC(NC(=O)c2ccc(Oc3ccccc3-c3ccsc3)c(Cl)c2)CC(C)(C)N1 ; CC1(C)CC(NC(=O)c2ccc(Oc3ccccc3-c3ncco3)c(Cl)c2)CC(C)(C)N1] 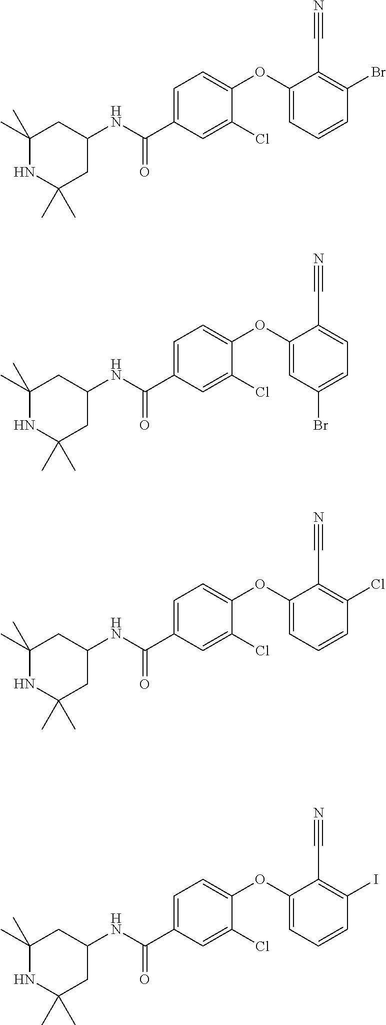 [SMILES: CC1(C)CC(NC(=O)c2ccc(Oc3cccc(Br)c3C#N)c(Cl)c2)CC(C)(C)N1 ; CC1(C)CC(NC(=O)c2ccc(Oc3cc(Br)ccc3C#N)c(Cl)c2)CC(C)(C)N1 ; CC1(C)CC(NC(=O)c2ccc(Oc3cccc(Cl)c3C#N)c(Cl)c2)CC(C)(C)N1 ; CC1(C)CC(NC(=O)c2ccc(Oc3cccc(I)c3C#N)c(Cl)c2)CC(C)(C)N1] 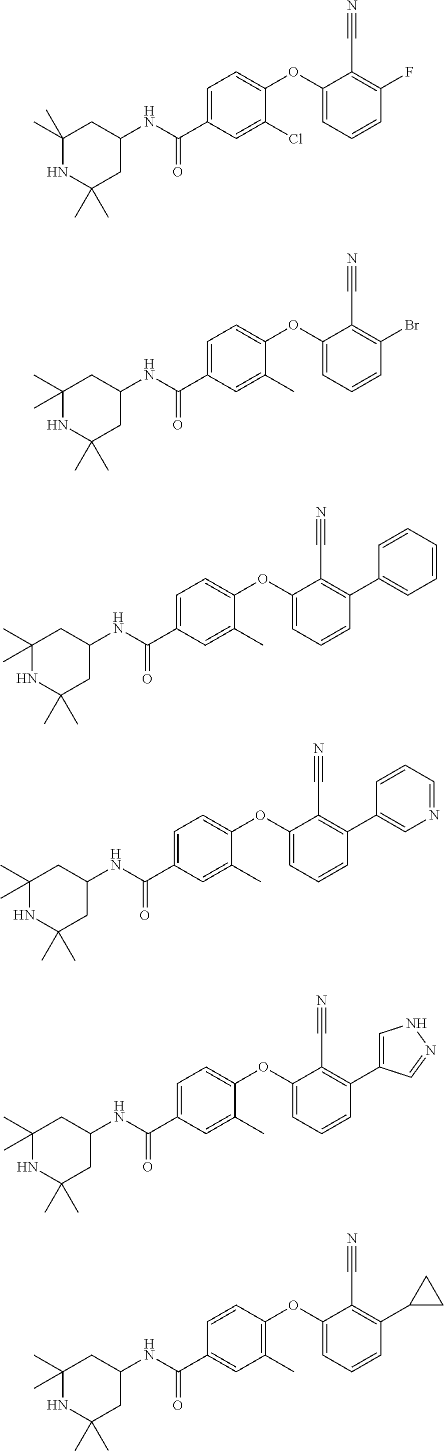 [SMILES: CC1(C)CC(NC(=O)c2ccc(Oc3cccc(F)c3C#N)c(Cl)c2)CC(C)(C)N1 ; Cc1cc(C(=O)NC2CC(C)(C)NC(C)(C)C2)ccc1Oc1cccc(Br)c1C#N ; Cc1cc(C(=O)NC2CC(C)(C)NC(C)(C)C2)ccc1Oc1cccc(-c2ccccc2)c1C#N ; Cc1cc(C(=O)NC2CC(C)(C)NC(C)(C)C2)ccc1Oc1cccc(-c2cccnc2)c1C#N ; Cc1cc(C(=O)NC2CC(C)(C)NC(C)(C)C2)ccc1Oc1cccc(-c2cn[nH]c2)c1C#N ; Cc1cc(C(=O)NC2CC(C)(C)NC(C)(C)C2)ccc1Oc1cccc(C2CC2)c1C#N] 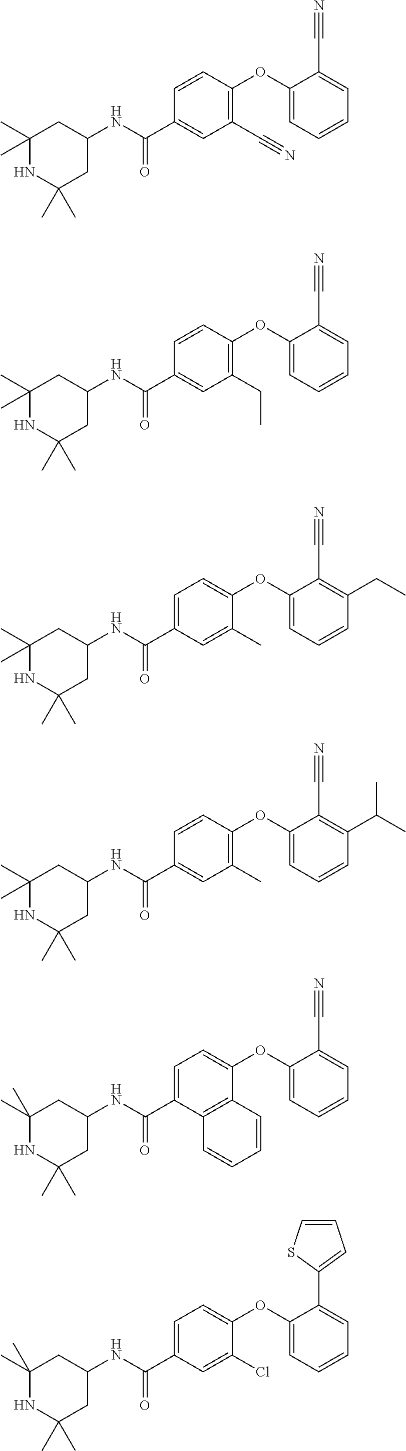 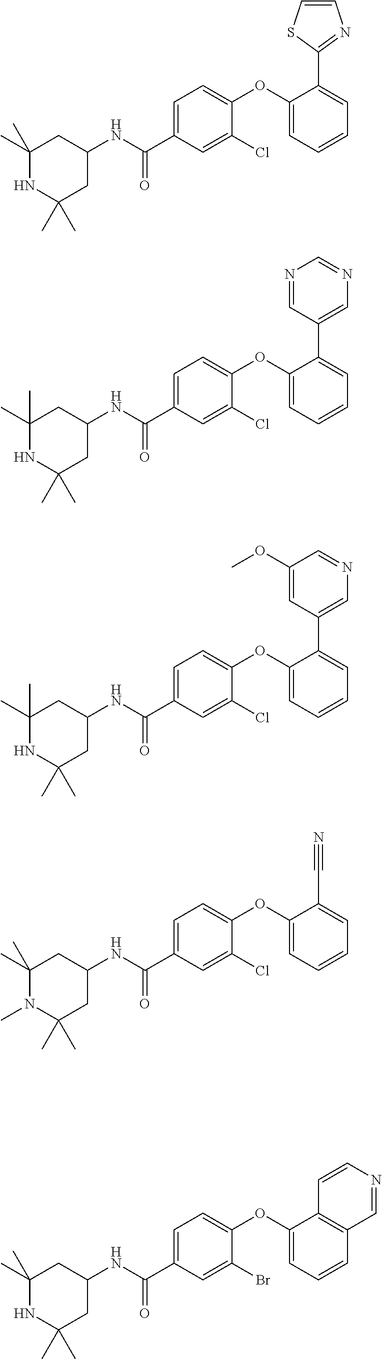 [SMILES: CC1(C)CC(NC(=O)c2ccc(Oc3ccccc3-c3nccs3)c(Cl)c2)CC(C)(C)N1 ; CC1(C)CC(NC(=O)c2ccc(Oc3ccccc3-c3cncnc3)c(Cl)c2)CC(C)(C)N1 ; COc1cncc(-c2ccccc2Oc2ccc(C(=O)NC3CC(C)(C)NC(C)(C)C3)cc2Cl)c1 ; CN1C(C)(C)CC(NC(=O)c2ccc(Oc3ccccc3C#N)c(Cl)c2)CC1(C)C ; CC1(C)CC(NC(=O)c2ccc(Oc3cccc4cnccc34)c(Br)c2)CC(C)(C)N1] 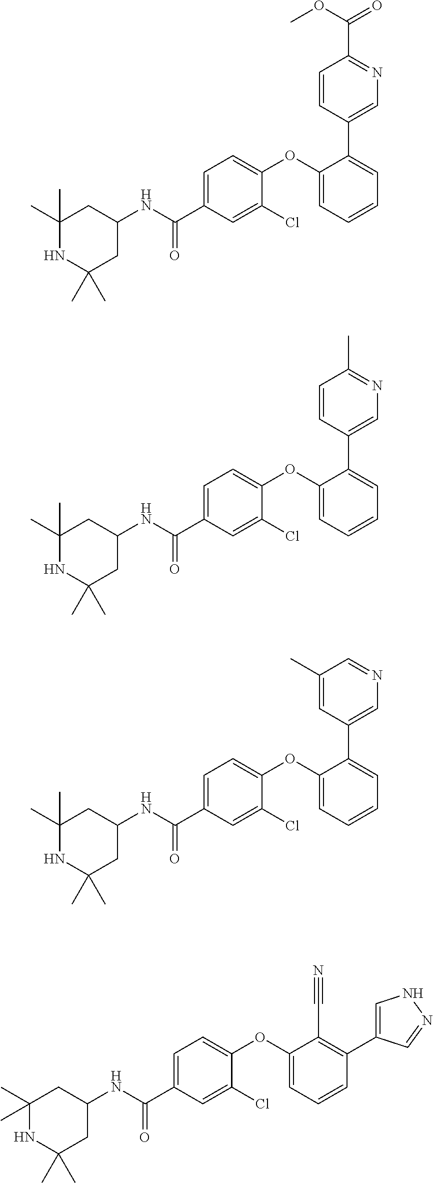 [SMILES: COC(=O)c1ccc(-c2ccccc2Oc2ccc(C(=O)NC3CC(C)(C)NC(C)(C)C3)cc2Cl)cn1 ; Cc1ccc(-c2ccccc2Oc2ccc(C(=O)NC3CC(C)(C)NC(C)(C)C3)cc2Cl)cn1 ; Cc1cncc(-c2ccccc2Oc2ccc(C(=O)NC3CC(C)(C)NC(C)(C)C3)cc2Cl)c1 ; CC1(C)CC(NC(=O)c2ccc(Oc3cccc(-c4cn[nH]c4)c3C#N)c(Cl)c2)CC(C)(C)N1] 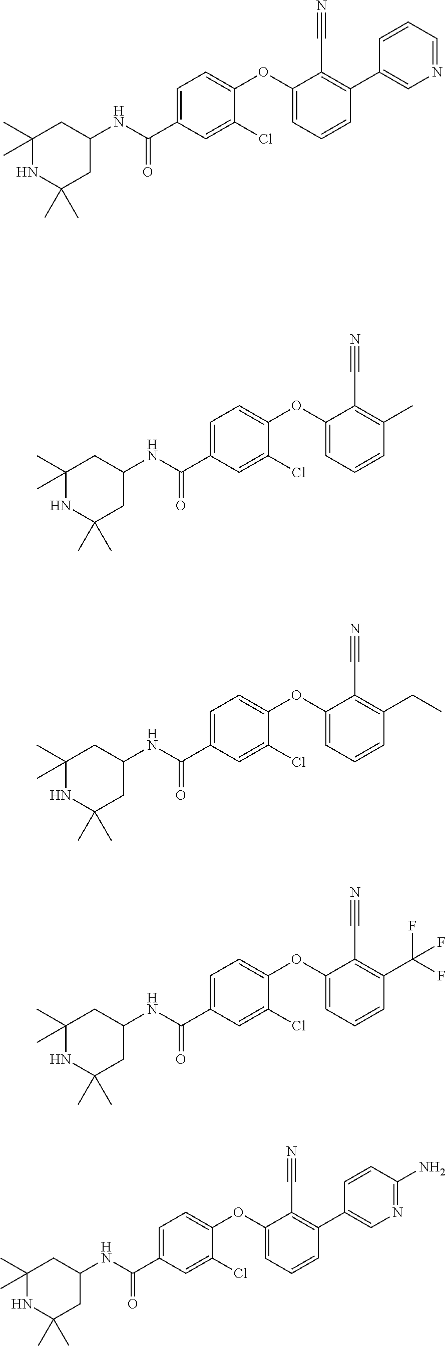 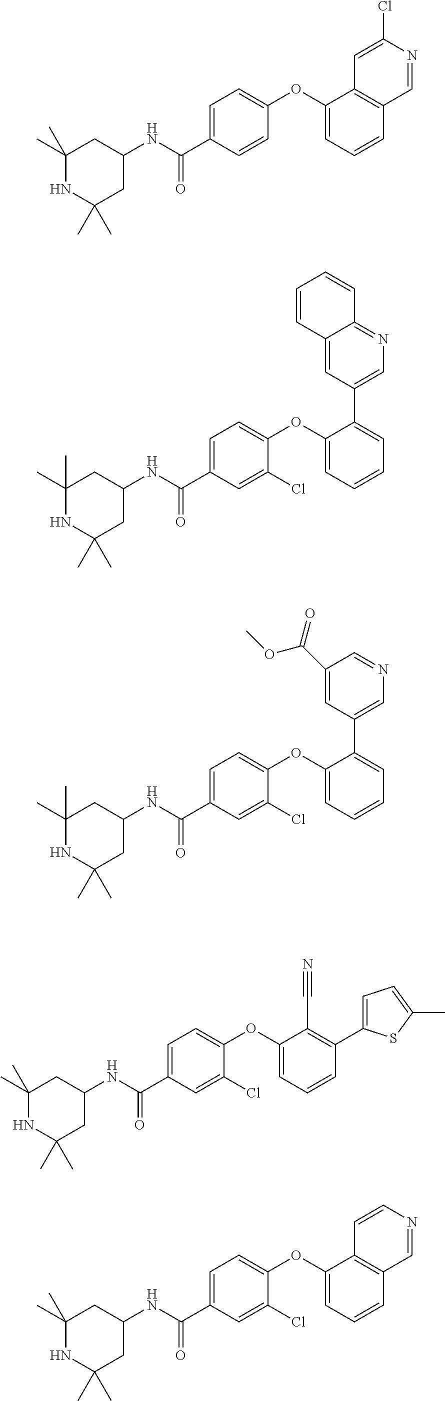 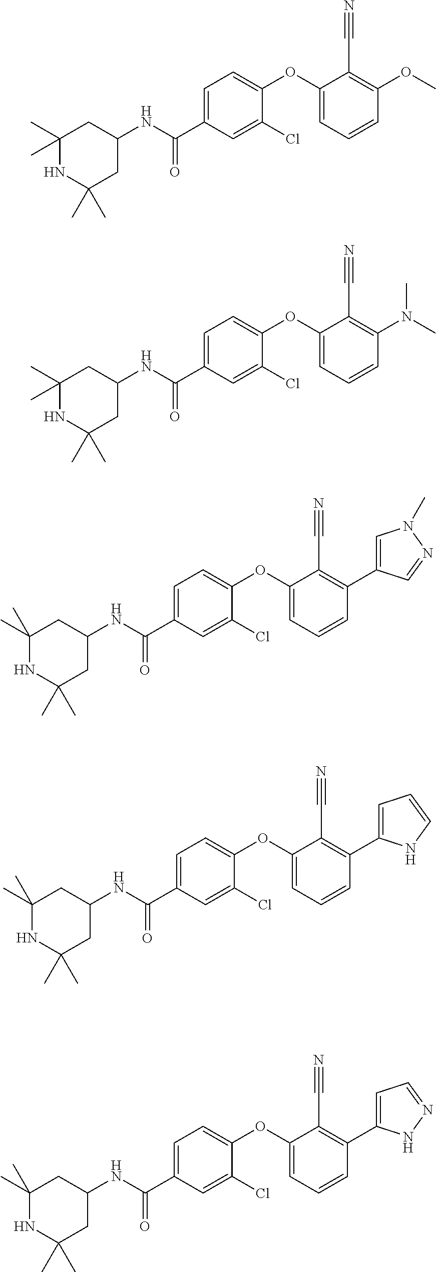 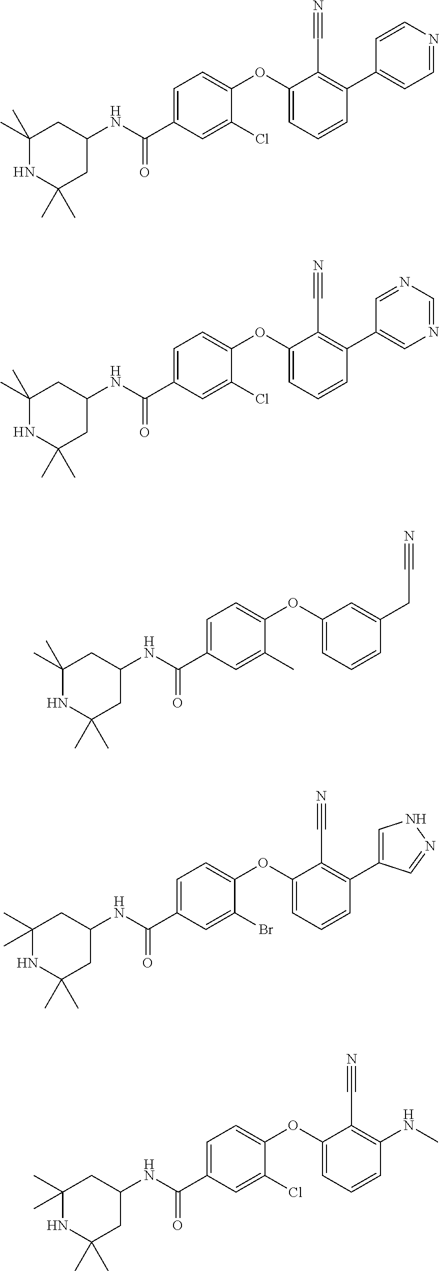 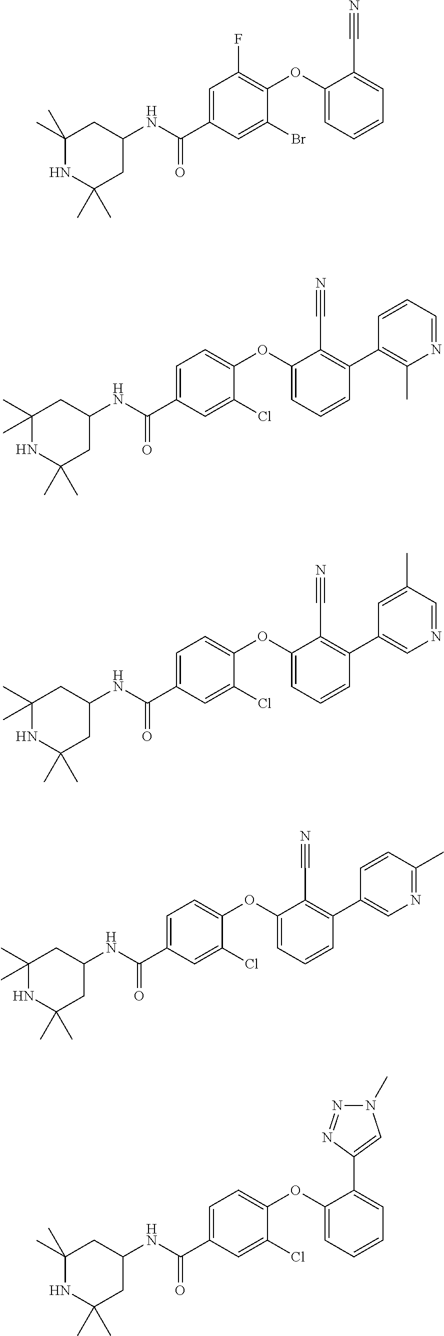 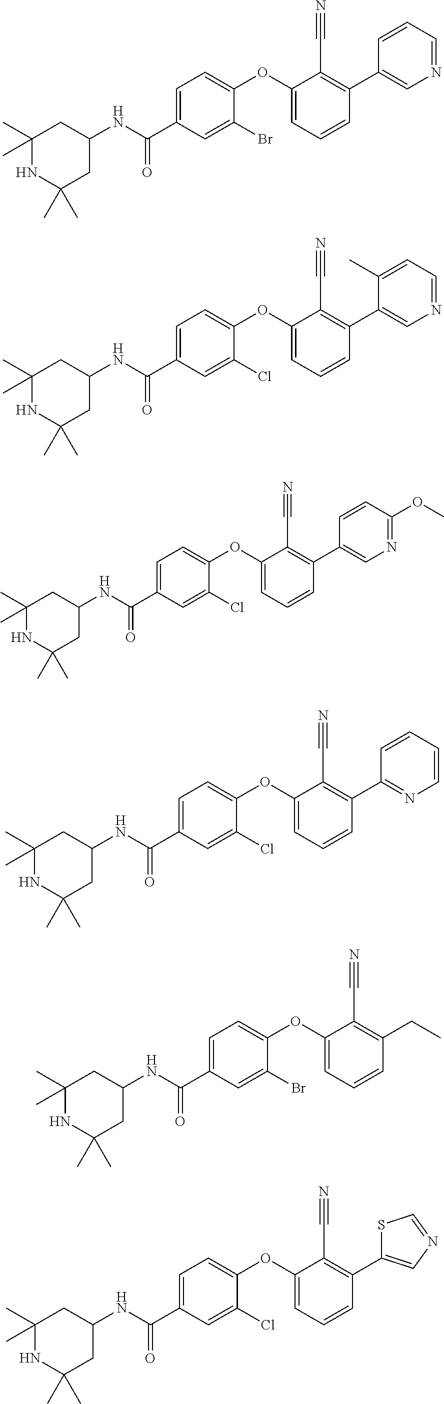 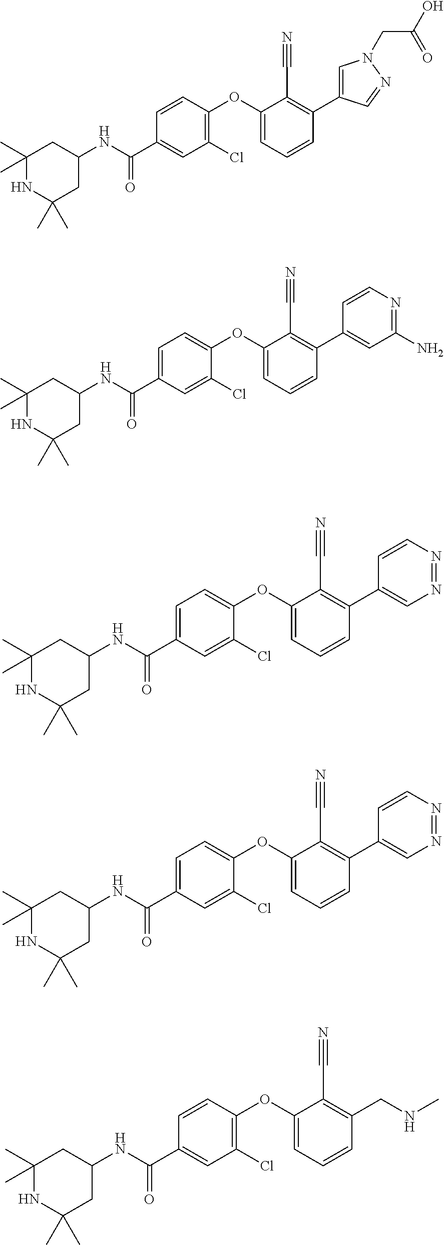 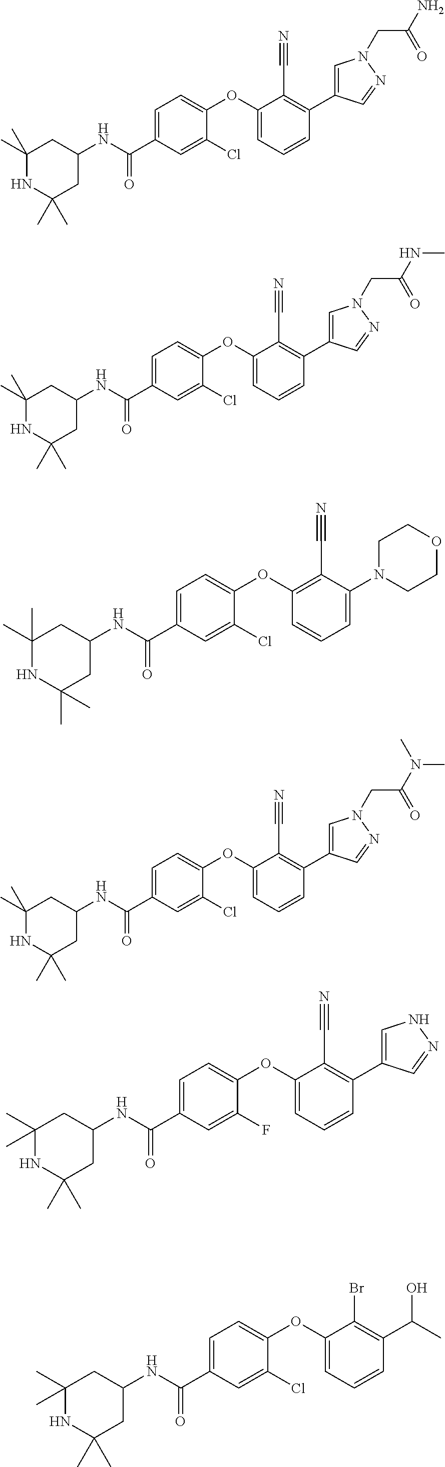 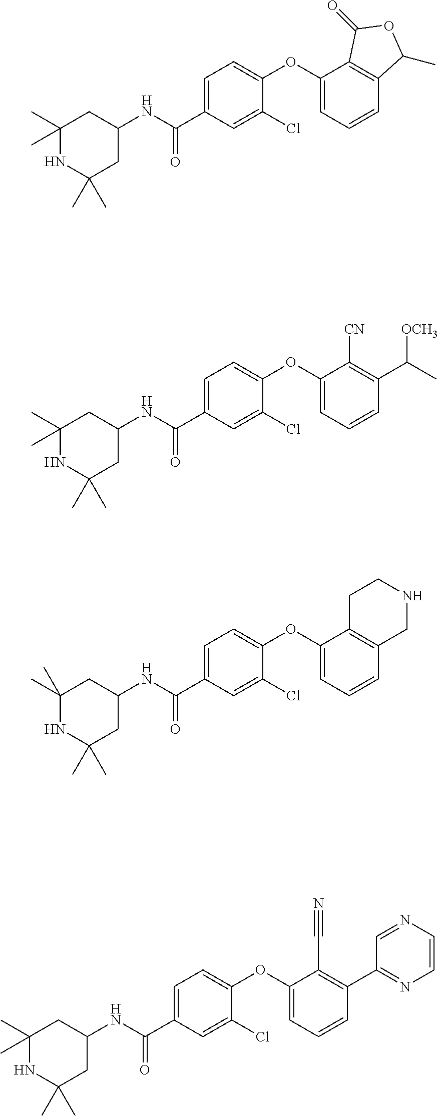 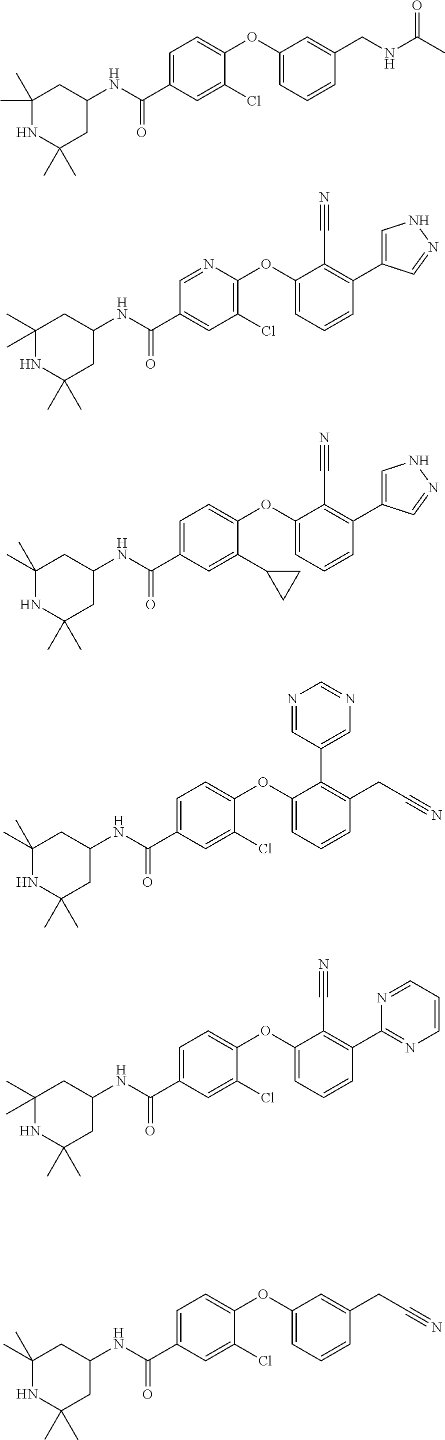 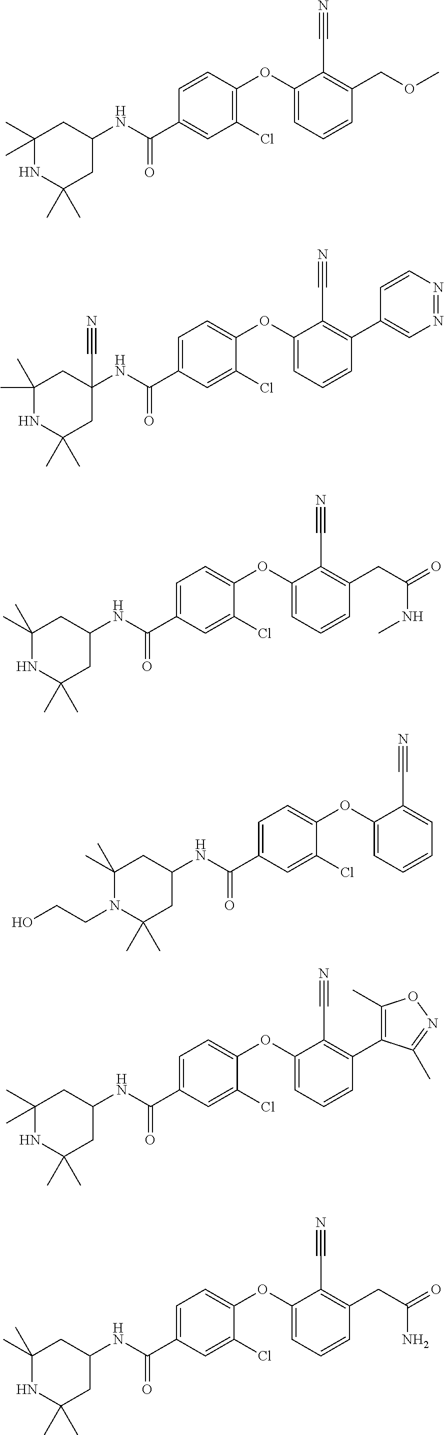 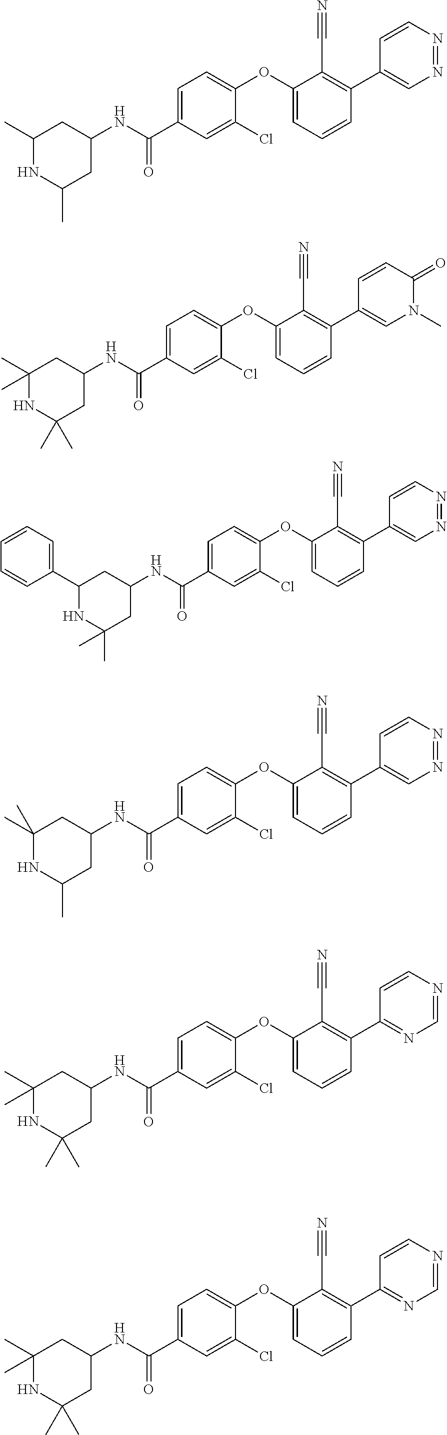 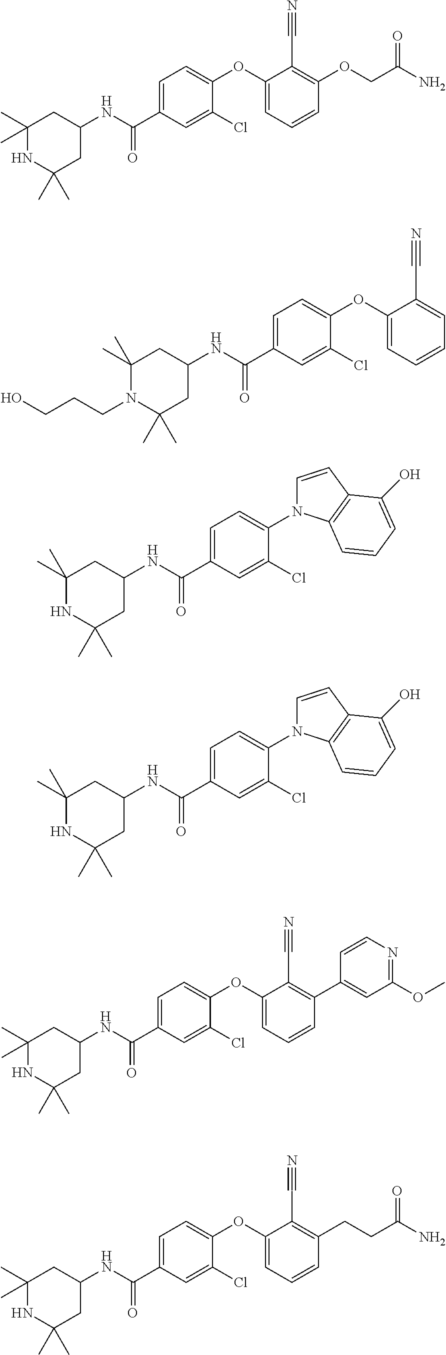 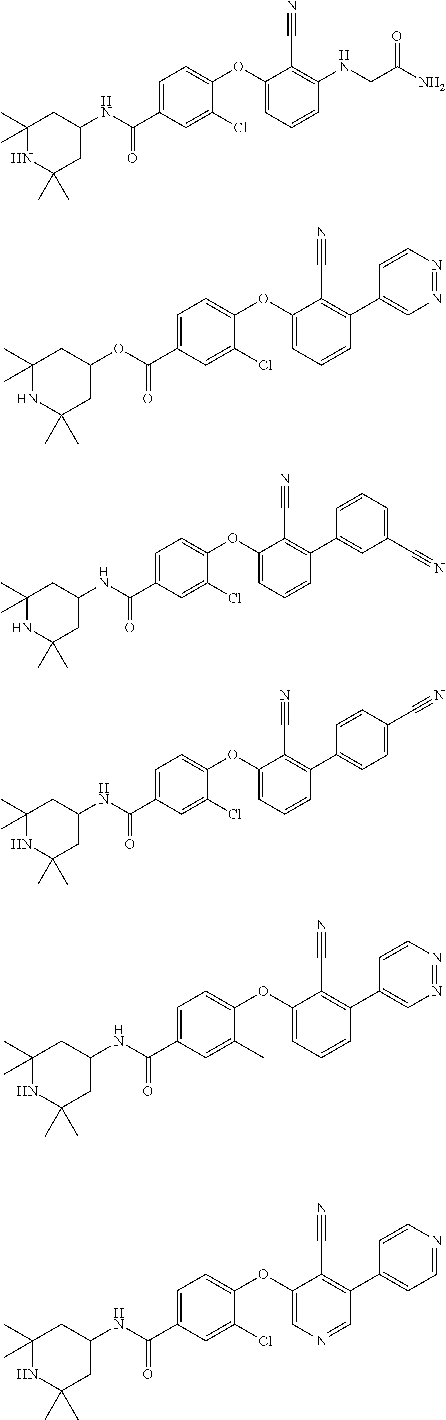 [SMILES: CC1(C)CC(NC(=O)c2ccc(Oc3cccc(NCC(N)=O)c3C#N)c(Cl)c2)CC(C)(C)N1 ; CC1(C)CC(OC(=O)c2ccc(Oc3cccc(-c4ccnnc4)c3C#N)c(Cl)c2)CC(C)(C)N1 ; CC1(C)CC(NC(=O)c2ccc(Oc3cccc(-c4cccc(C#N)c4)c3C#N)c(Cl)c2)CC(C)(C)N1 ; CC1(C)CC(NC(=O)c2ccc(Oc3cccc(-c4ccc(C#N)cc4)c3C#N)c(Cl)c2)CC(C)(C)N1 ; Cc1cc(C(=O)NC2CC(C)(C)NC(C)(C)C2)ccc1Oc1cccc(-c2ccnnc2)c1C#N ; CC1(C)CC(NC(=O)c2ccc(Oc3cncc(-c4ccncc4)c3C#N)c(Cl)c2)CC(C)(C)N1] 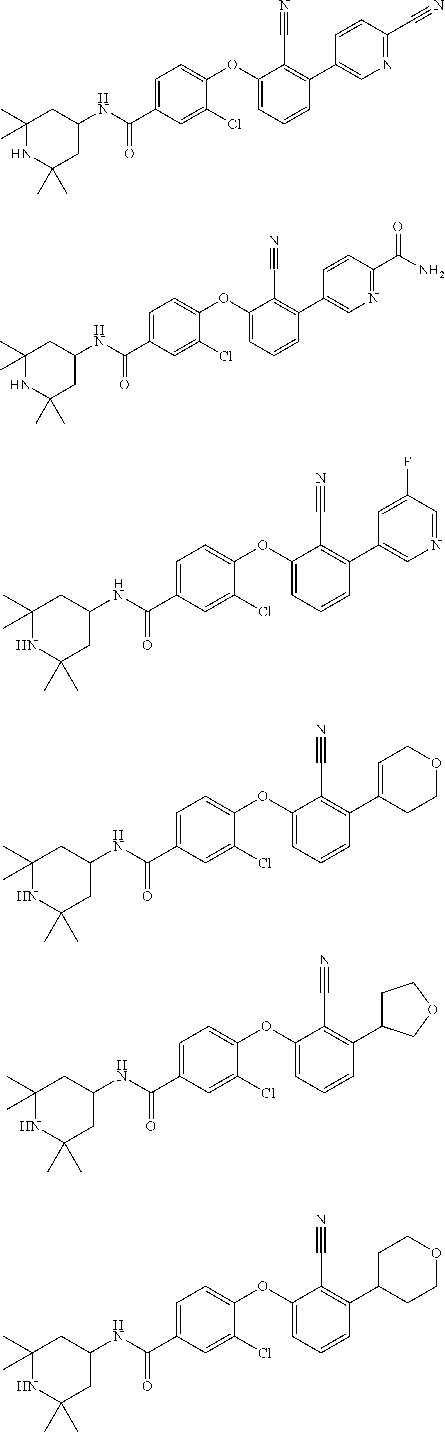 [SMILES: CC1(C)CC(NC(=O)c2ccc(Oc3cccc(-c4ccc(C#N)nc4)c3C#N)c(Cl)c2)CC(C)(C)N1 ; CC1(C)CC(NC(=O)c2ccc(Oc3cccc(-c4ccc(C(N)=O)nc4)c3C#N)c(Cl)c2)CC(C)(C)N1 ; CC1(C)CC(NC(=O)c2ccc(Oc3cccc(-c4cncc(F)c4)c3C#N)c(Cl)c2)CC(C)(C)N1 ; CC1(C)CC(NC(=O)c2ccc(Oc3cccc(C4=CCOCC4)c3C#N)c(Cl)c2)CC(C)(C)N1 ; CC1(C)CC(NC(=O)c2ccc(Oc3cccc(C4CCOC4)c3C#N)c(Cl)c2)CC(C)(C)N1 ; CC1(C)CC(NC(=O)c2ccc(Oc3cccc(C4CCOCC4)c3C#N)c(Cl)c2)CC(C)(C)N1] 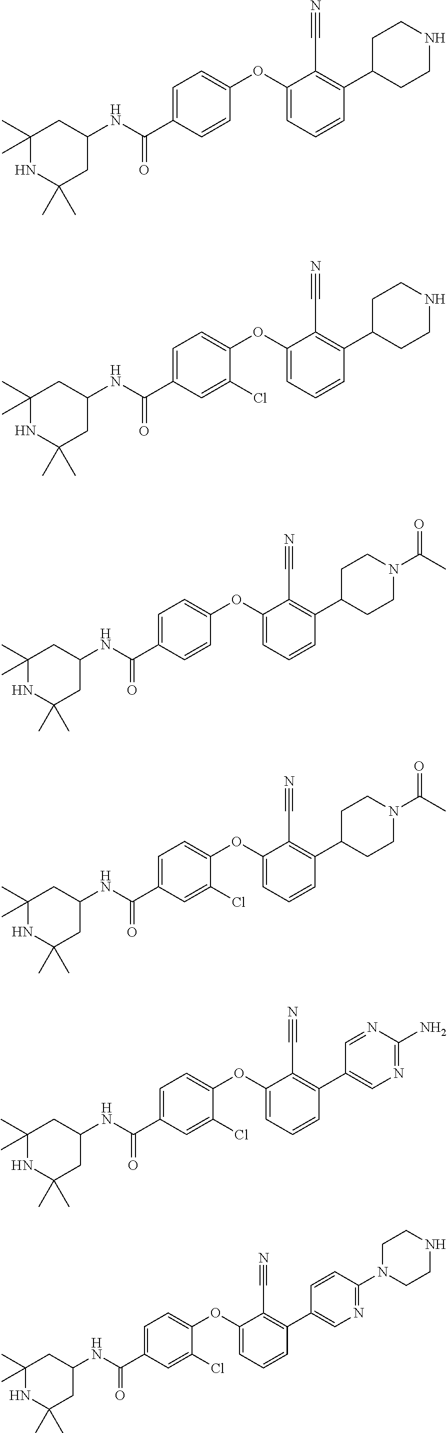 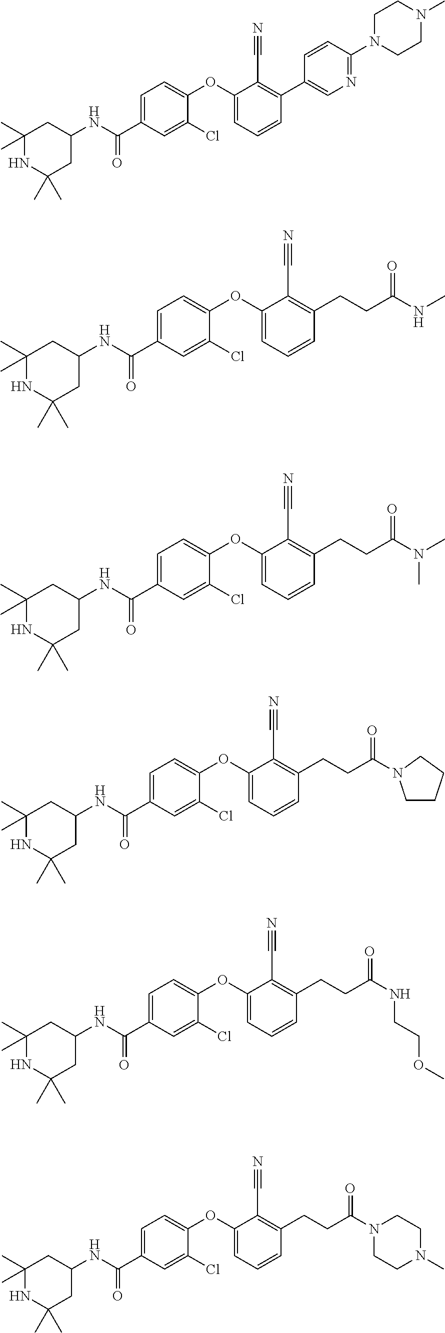 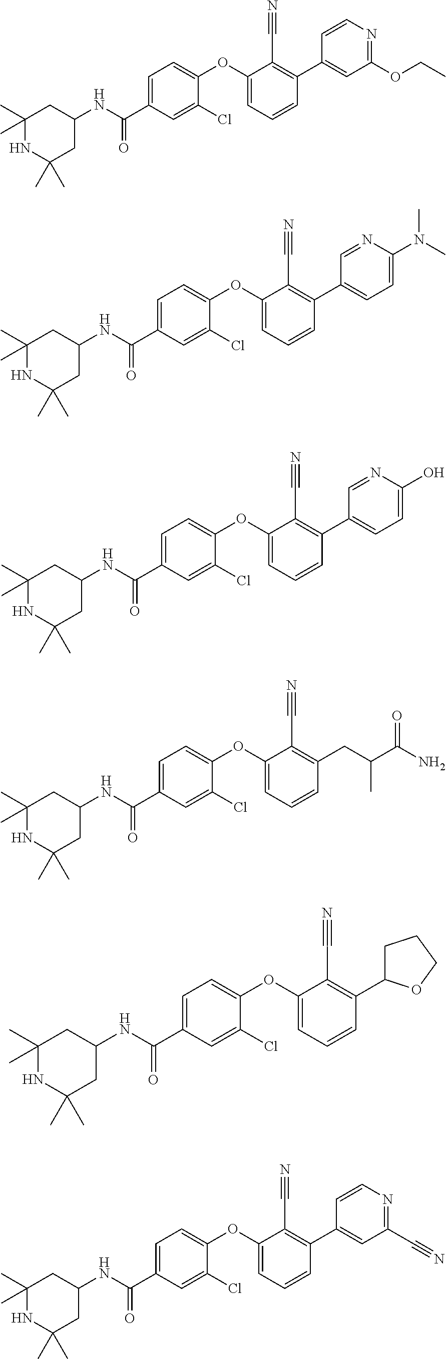 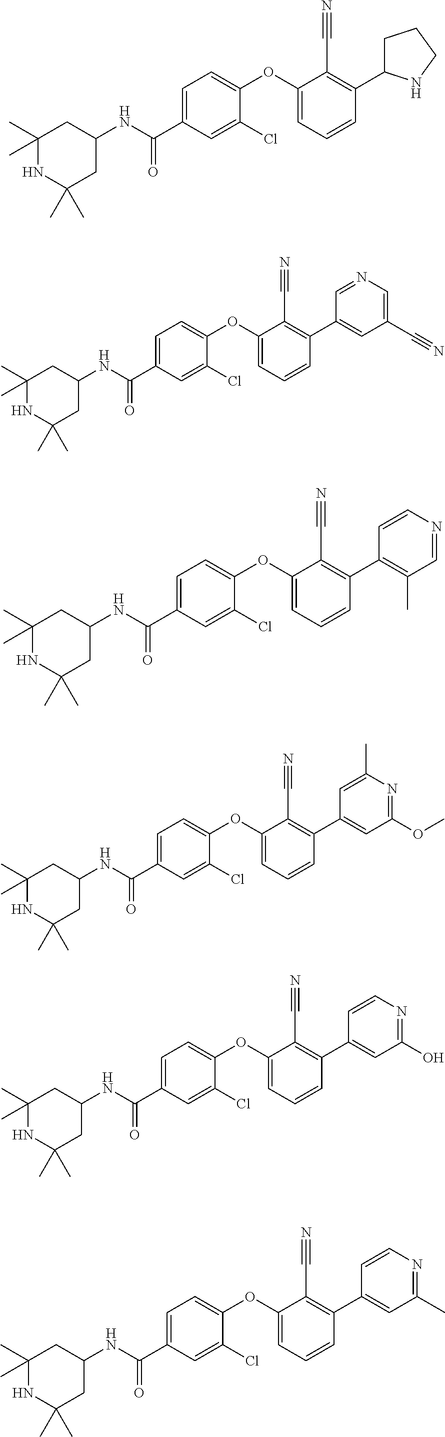 [chemical structure]
